(19) United States
(12) Patent Application Publication
(10) Pub. No.: US 2026/0242394 A1
(43) Pub. Date: Aug. 20, 2026

(54) USP2 INHIBITORS AND METHODS OF USING THE SAME FOR THE TREATMENT OF DISEASES

(71) Applicants: ICAHN SCHOOL OF MEDICINE AT MOUNT SINAI, New York, NY (US); BETH ISRAEL DEACONESS MEDICAL CENTER, INC., Boston, MA (US); THE TRUSTEES OF COLUMBIA UNIVERSITY IN THE CITY OF NEW YORK, New York, NY (US)

(72) Inventors: Jian Jin, New York, NY (US); Wenyi Wei, Boston, MA (US); Wei Gu, New York, NY (US); Yan Xiong, New York, NY (US); Xiaoping Hu, New York, NY (US); Fabin Dang, Boston, MA (US); Jingjie Yi, New York, NY (US)

(21) Appl. No.: 19/472,280

(22) PCT Filed: Apr. 5, 2024
(86) PCT No.: PCT/US2024/023375

§ 371 Date: Oct. 3, 2025

Related U.S. Application Data
(60) Provisional application No. 63/494,654, filed on Apr. 6, 2023.

(51) Int. Cl. C07D 495/04; A61K 31/18; A61K 31/381; A61K 31/4168; A61K 31/426; A61K 31/427; A61K 31/428; A61K 31/4439; A61K 31/517; A61K 31/519; A61K 31/5375; A61K 45/06; A61P 31/14; C07C 311/21; C07D 233/88; C07D 277/46; C07D 277/82; C07D 295/13; C07D 333/36; C07D 417/04; C07D 417/12; C07D 491/048

(52) U.S. Cl. CPC C07D 495/04; A61K 31/18; A61K 31/381; A61K 31/4168; A61K 31/426; A61K 31/427; A61K 31/428; A61K 31/4439; A61K 31/517; A61K 31/519; A61K 31/5375; A61K 45/06; A61P 31/14; C07C 311/21; C07D 233/88; C07D 277/46; C07D 277/82; C07D 295/13; C07D 333/36; C07D 417/04; C07D 417/12; C07D 491/048

(57) ABSTRACT

Disclosed are small molecule ubiquitin carboxyl-terminal hydrolase 2 (USP2) inhibitors which display great potency in inhibiting cancer cell proliferation by elevating wild type P53 levels, reducing cyclin D levels or blocking ACE2 dependent virus from entering host cells by reducing ACE2 levels. The results indicate that these USP2 inhibitors have potential to be used in treating various diseases, including cancer and virus infection depending on ACE2.

### STATEMENT REGARDING GOVERNMENT FUNDING

This invention was made with government support under R01CA258390 awarded by the National Institutes of Health. The government has certain rights in the invention.

### TECHNICAL FIELD

The present disclosure is directed to small molecule inhibitors of ubiquitin carboxyl-terminal hydrolase 2 (also known as USP2). The compounds disclosed are useful in the treatment of various diseases including cancer and virus infection dependent on ACE2.

### BACKGROUND

Ubiquitylation and deubiquitylation are reversible protein post-translational modification (PTM) processes. They play critical roles in the regulation of protein degradation under physiological conditions. Deubiquitylation is catalyzed by a series of ubiquitin-specific proteases (DUBs), which regulate gene expression, apoptosis, cell cycle, DNA repair, and cytokines (Frappier and Verrijzer, 2011). Recently, more and more evidence indicates DUBs are associated with a variety of diseases, including cancer, diabetes, neurodegenerative diseases, and infectious diseases (Komander et al., 2009).

Ubiquitin carboxyl-terminal hydrolase 2 (also known as USP2), a DUBs family member, is able to remove ubiquitin from a range of protein substrates such as fatty acid synthase, MDM2, cyclin D1 and Aurora-A (Chuang et al., 2018).

Compared to the great progress in the development of small molecules targeting other DUBs, such as USP7, only a few USP2 inhibitors have been reported so far. These USP2 inhibitors include Q29 (Ohayon et al., 2015), ML364 (Davis et al., 2016), LCAHA (Magiera et al., 2017), STD1T (Tomala et al., 2018), 6TG (Chuang et al., 2018), NSC632839 (Nicholson et al., 2008), PR-619 (Altun et al., 2011) and compound 14 (Vamisetti et al., 2019). However, these compounds showed either low potency or poor selectivity. Therefore, potent and selective USP2 inhibitors to effectively treat USP2-mediated diseases are needed.

### SUMMARY

Described herein are novel small molecules that inhibit UPS2. These novel inhibitors show potent activity in cells and have significant potential for the treatment of patients with USP2 related diseases, such as cancers and virus infections dependent on ACE2.

The severe acute respiratory syndrome coronavirus 2 (SARS-CoV-2) is a positive-sense single-stranded RNA virus, causing the ongoing global coronavirus disease 2019 (COVID-19) pandemic. Studies of the molecular mechanisms by which SARS-CoV-2 enter into human cells revealed that the virus predominantly relies on the interaction of its surface spike (S) protein with the host cell receptor angiotensin-converting enzyme 2 (ACE2). Based on these findings, multiple approaches had been developed to block SARS-CoV-2 infection by preventing S protein from binding to ACE2. Recently, we found USP2 serves as a physiological deubiquitinase of endogenous ACE2 protein, governing its deubiquitination and stabilization. Therefore, developing potent USP2 inhibitor can provide a novel therapeutic method for treating coronavirus infections depending on ACE2.

In addition, cancer is the second leading cause of death in the world. The tumor suppression activity of the p53 pathway is impaired in many human tumors. Thus, restoration of p53 function remains an important objective for treating human cancers. USP2 can affect the functions of p53 through regulating Mdm2 and VPRBP (also known as DCAF1) protein polyubiquitination status. Furthermore, the well-known oncoprotein, cyclin D is the also the substrate of USP2. Therefore, inhibition of USP2 could lead to upregulation of P53 and downregulation of cyclin D. In many cancers, including prostate cancer and ovarian carcinoma, USP2 is overexpressed. Furthermore, because USP2 is a non-essential gene, pharmacological inhibition of USP2 is not expected to result in on-target toxicity. Accordingly, USP2 is a promising target for p53 or cyclin D related cancer treatment.

As discussed in the following examples, this disclosure provides specific examples of novel USP2 inhibitors and examines the effect of exemplary inhibitors in inhibiting the activity of USP2. Through USP2 inhibition, these exemplary inhibitors display great potency in inhibiting cancer cell proliferation by elevating wild type P53 levels, reducing cyclin D levels or blocking ACE2 dependent virus from entering host cells by reducing ACE2 levels. The results indicate that these novel small molecules have potential to be used in treating various diseases, including cancer and virus infection depending on ACE2.

In one aspect, the present disclosure features small molecule USP2 inhibitors of Formula (I) below:

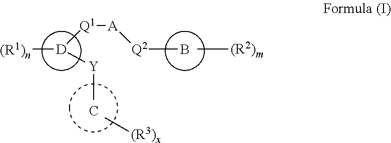

or a pharmaceutically acceptable salt of the compound or the tautomer therefor, wherein
A is C═O, CH₂ or absent;
Y is a bond, or a bivalent moiety selected from —N(R⁴)—, —N(R⁴)—C₁-C₃ alkylene-, —N(R⁴)—S(O)₂—, —N(R⁴)—C(O)—, —S(O)₂— N(R⁴)—, or —C(O)—N(R⁴)—;
Ring B is a ring structure selected from C₃-C₁₂ cycloalkyl, 3-12 membered heterocyclic, C₆-C₁₀ aryl, or 5-10 membered heteroaryl;
Ring C is absent, or a ring structure selected from C₆-C₁₀ aryl, or 5-10 membered heteroaryl;
Ring D is a ring structure selected from C₅-C₁₀ aryl, 5-10 membered heteroaryl, 3-12 membered heterocyclic;
Q¹ and Q² are independently selected from absent, O, NR⁵, C₁-C₃ alkylene, C₂-C₆ alkenylene, or C₂-C₆ alkynylene, where each said C₁-C₃ alkylene, C₂-C₆ alkenylene, or C₂-C₆ alkynylene is optionally substituted with one or more of halogen, cyano, hydroxyl, oxo, C₁-C₆ alkyl, C₃-C₆ cycloalkyl, C₁-C₆ alkoxyl, or C₃-C₆ cycloalkoxy,
each R¹, each R², and each R³ are independently selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, two adjacent R², or two adjacent R³, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
each R⁴, each R⁵, each R⁶ and each R⁷ are independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
each R⁸ and each R⁹ are independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-C₁₀ aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form 4-8 membered heterocyclic;
n is 0, 1, 2, 3, or 4;
m is 0, 1, 2, 3, 4, 5, 6, 7, 8, or 9; and
x is 0, 1, 2, 3, 4, 5, 6, 7, 8, or 9;
   In Some Embodiments, Ring C in Formula (I) is Absent. The Formula (I) is Formula (I-A):

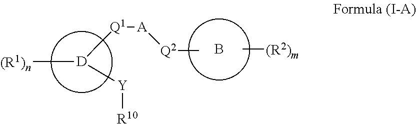

wherein
R¹⁰ is selected from H, OR⁶, NR⁶R⁷, C₁-C₆ alkyl, C₁-C₆ heteroalkyl, C₂-C₆ alkenyl, C₂-C₆ alkynyl, C₃-C₁₂ cycloalkyl, or 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, or 3-12 membered heterocyclic is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; and
A, Y, Ring B, D, Q¹, Q², R¹, R², R⁶, R⁷, R⁸, R⁹, n and m are defined in Formula (I).

In some embodiments, D is C₅-C₆ aryl, or 5-6 membered heteroaryl; In some embodiments, D is phenyl; In some embodiments, D is 5-membered heteroaryl; In some embodiments, D is 6-membered heteroaryl. In some embodiments, D is furan, pyrrole, thiophene, pyrazole, thiazole, or imidazole. In some embodiments, D is pyridine or pyrimidine. In some embodiments, D is pyridine.

In some embodiments, R⁴ is H. In some embodiments, R⁴ is C₁-C₆ alkyl.

In some embodiments, R⁵ is H. In some embodiments, R⁵ is C₁-C₆ alkyl.

In some embodiments, Y is —N(R⁴)—C₁-C₃ alkylene-, —N(R⁴)—S(O)₂—, or —N(R⁴)—C(O)—. In some embodiments, Y is —N(R⁴)—C₁-C₃ alkylene-. In some embodiments, Y is —N(R⁴)—CH₂—. In some embodiments, Y is —N(H)—CH₂—. In some embodiments, Y is —N(R⁴)—S(O)₂—. In some embodiments, Y is —N(H)—S(O)₂—. In some embodiments, Y is —N(R⁴)—C(O)—. In some embodiments, Y is —N(H)—C(O)—.

In some embodiments, Q¹ is absent or NR⁵. In some embodiments, Q¹ is absent. In some embodiments, Q¹ is NR⁵. In some embodiments, Q¹ is NH or NCH₃. In some embodiments, Q¹ is NH. In some embodiments, Q² is absent or NR⁵. In some embodiments, Q² is absent. In some embodiments, Q² is NR⁵. In some embodiments, Q² is NH or NCH₃. In some embodiments, Q² is NH. In some embodiments, Q¹ is absent and Q² is NR⁵. In some embodiments, Q¹ is absent and Q² is NH. In some embodiments, Q¹ is NR⁵ and Q² is absent. In some embodiments, Q¹ is NH and Q² is absent. In some embodiments, A is C═O. In some embodiments, A is CH₂. In some embodiments, Q¹AQ² is Q¹A. In some embodiments, Q¹AQ² is Q¹C(═O). In some embodiments, Q¹AQ² is NR⁵C(═O). In some embodiments, Q¹AQ² is NHC(═O). In some embodiments, Q¹AQ² is Q¹CH₂. In some embodiments, Q¹AQ² is NR⁵CH₂. In some embodiments, Q¹AQ² is NHCH₂. In some embodiments, Q¹AQ² is AQ². In some embodiments, Q¹AQ² is C(═O)Q². In some embodiments, Q¹AQ² is C(═O)NR⁵. In some embodiments, Q¹AQ² is C(═O)NH. In some embodiments, Q¹AQ² is CH₂Q2. In some embodiments, Q¹AQ² is CH₂NR⁵. In some embodiments, Q¹AQ² is CH₂NH.

### In Some Embodiments, Small Molecule USP2 Inhibitors Comprises Formula (I-A-1):

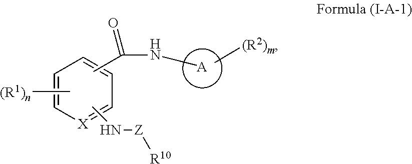

Wherein
X is CH or N;
Z is C═O or SO₂;
X, Ring B, R¹, R², n and m are defined in Formula (I); and R¹⁰ is defined in Formula (I-A).

In some embodiments, Z is C═O. In some embodiments, Z is SO₂.

In some embodiments, Ring B is selected from C₆-C₁₀ aryl or 5-10 membered heteroaryl.

In some embodiments, small molecule USP2 inhibitors comprises Formula (I-A-2):

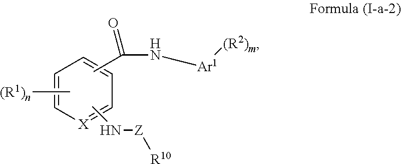

wherein
Ar¹ is selected from C₆-C₁₀ aryl or 5-10 membered heteroaryl; R¹, R², n and m are defined in Formula (I); and R¹⁰ is defined in Formula (I-A); X and Z are defined in Formula (I-A-1).

In some embodiments, Ar¹ is 5-10 membered heteroaryl. In some embodiments, Ar¹ is 5-6 membered monocyclic heteroaryl. In some embodiments, Ar¹ is selected from

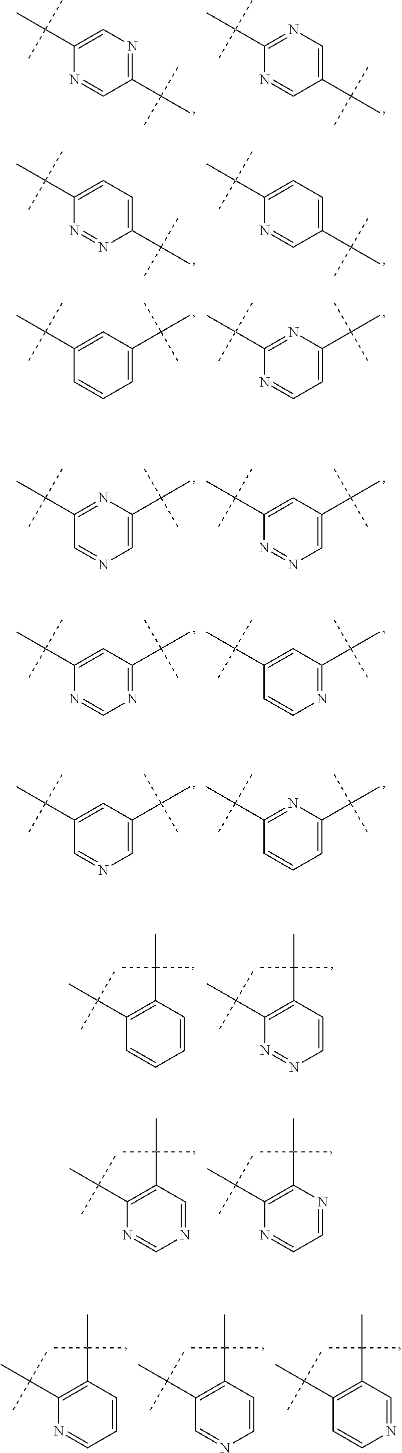

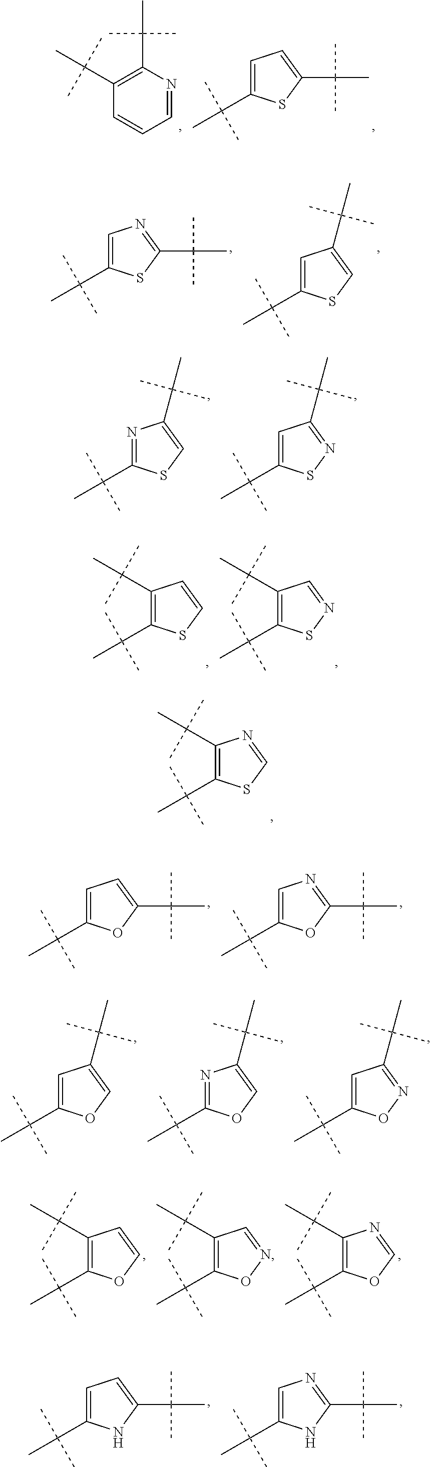

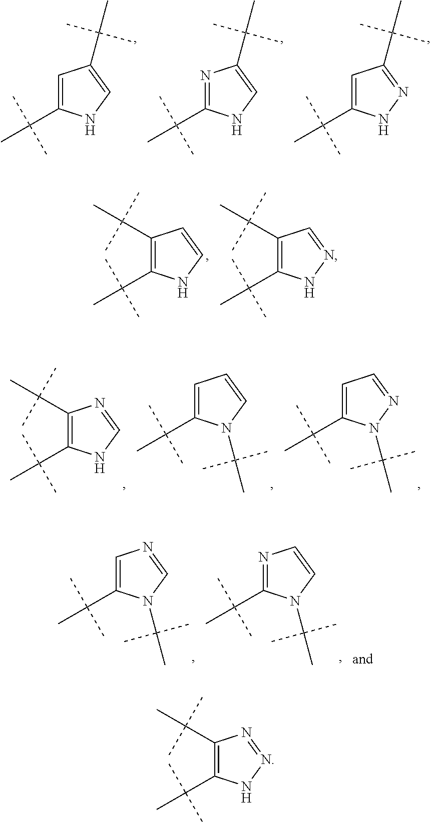

In some embodiments, Ar¹ is selected from

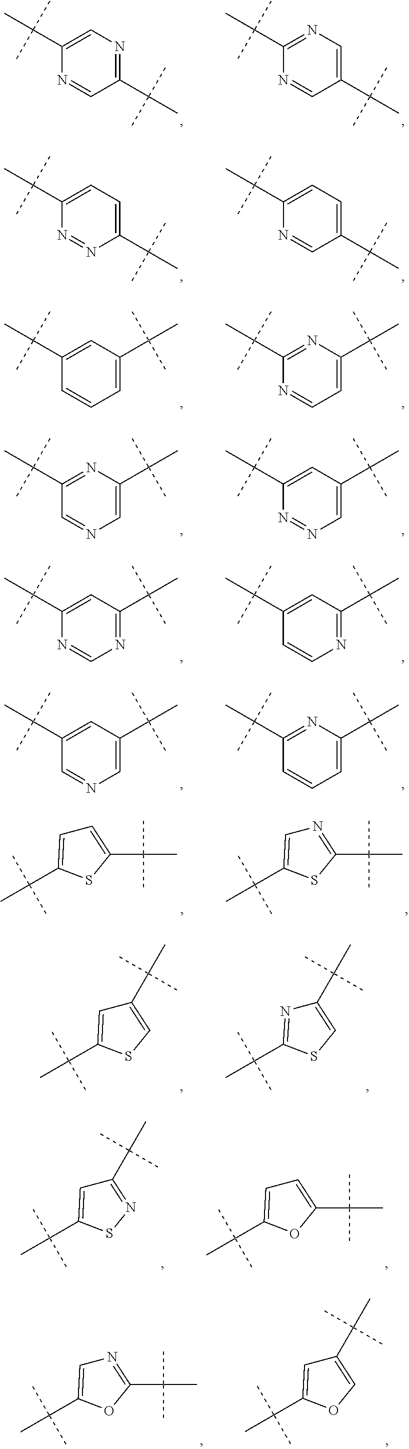

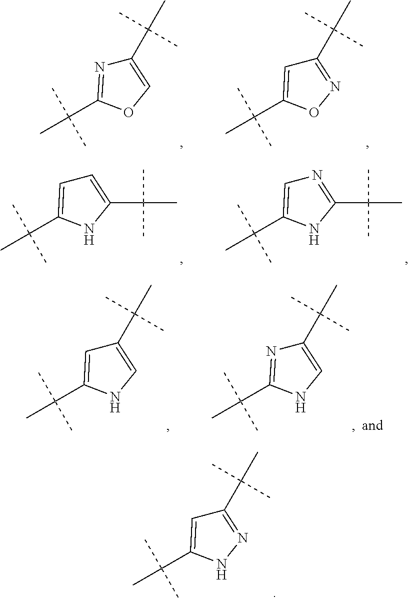

In some embodiments, Ar¹ is thiazole. In some embodiments, Ar¹ is

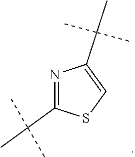

In some embodiments, Ar¹ is 7-10 membered bicyclic heteroarylene. In some embodiments, Ar¹ is 8 membered bicyclic heteroarylene comprising two fused 5 membered rings. In some embodiments, Ar¹ is 9 membered bicyclic heteroarylene comprising fused 5 and 6 membered rings. In some embodiments, Ar¹ is 10 membered bicyclic heteroarylene comprising two fused six membered rings. In some embodiments, Ar¹ is a thiazole fused with a 5 membered ring. In some embodiments, Ar¹ is a thiazole fused with a 6 membered ring. In some embodiments, Ar¹ is a benzothiazole. In some embodiments, Ar¹ is

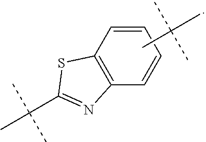

In some embodiments, each R² is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₃-C₆ cycloalkyl, 3-6 membered heterocyclic, C₆-C₁₀ aryl, or 5-10 membered heteroaryl, where each said C₁-C₆ alkyl, C₁-C₆ heteroalkyl, C₃-C₆ cycloalkyl, 3-6 membered heterocyclic, C₆-C₁₀ aryl, or 5-10 membered heteroaryl is optionally substituted with halogen, cyano, C₁-C₆ alkyl, C₁-C₆ heteroalkyl, C₃-C₆ cycloalkyl, 3-6 membered heterocyclic. In some embodiments, each R² is independently selected from H, halogen, cyano, hydroxy, C₁-C₆ alkyl, C₃-C₆ cycloalkyl, and 3-6 membered heterocyclic, where each said C₁-C₆ alkyl, C₃-C₆ cycloalkyl, and 3-6 membered heterocyclic, is optionally substituted with halo or C₁-C₆ alkyl. In some embodiments, each R² is independently selected from H, CN, OH, F, Cl, Br, CH₃, and CF₃. In some embodiments, each R² is independently selected from C₆-C₁₀ aryl, or 5-10 membered heteroaryl, where each said C₆-C₁₀ aryl, or 5-10 membered heteroaryl is optionally substituted with halogen, cyano, OR⁸, NR⁸R⁹, C₁-C₆ alkyl, C₁-C₆ heteroalkyl, C₃-C₆ cycloalkyl, or 3-6 membered heterocyclic. In some embodiments, each R² is independently selected from C₆-C₁₀ aryl, which is optionally substituted with halogen, cyano, OR⁸, NR⁸R⁹, C₁-C₆ alkyl, C₁-C₆ heteroalkyl, C₃-C₆ cycloalkyl, or 3-6 membered heterocyclic. In some embodiments, each R² is independently selected from phenyl, which is optionally substituted with F, Cl, Br, CN, OH, CH₃, or CF₃. In some embodiments, each R² is independently selected from

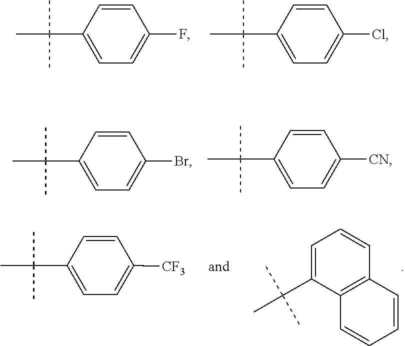

In some embodiments, small molecule USP2 inhibitors comprises Formulae (I-A-3), (I-A-4) and (I-A-5):

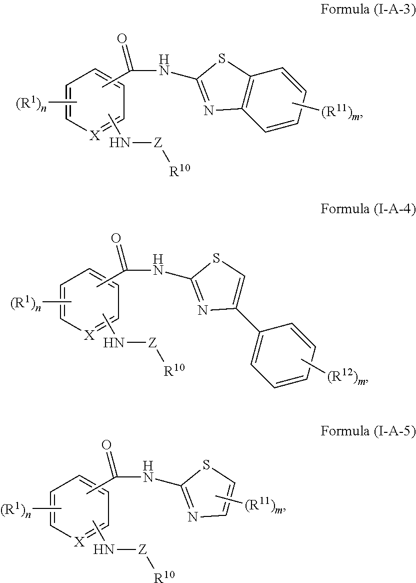

wherein
each R¹¹ is independently selected from H, halogen, cyano, OR⁶, NR⁶R⁷, C(O)OR⁸, C(O)NR⁸R⁹, S(O)₂NR⁸R⁹, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁a aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
each R¹² is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹², together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R¹, R⁶, R⁷, R⁸, R⁹, n and m are defined in Formula (I); X and Z are defined in Formula (I-A-1);
and R¹⁰ is defined in Formula (I-A);

In some embodiments, X is CH. In some embodiments, X is N.

In some embodiments, small molecule USP2 inhibitors comprises Formulae (I-A-6), (I-A-7) and (I-A-8):

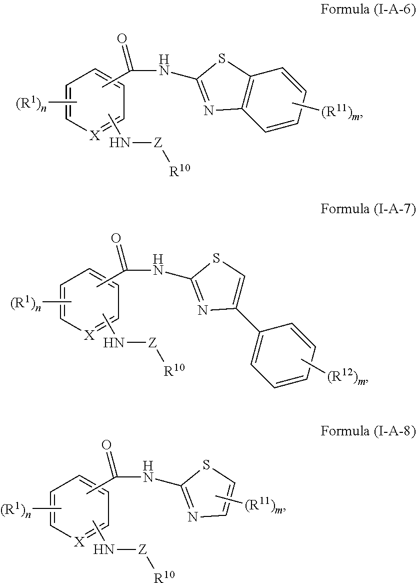

wherein
R¹, n and m are defined in Formula (I); R¹⁰ is defined in Formula (I-A); Z is defined in Formula (I-A-1); and R¹¹ and R¹² are defined in Formulae (I-A-3), (I-A-4) and (I-A-5).

In some embodiments, small molecule USP2 inhibitors comprises Formulae (I-A-9), (I-A-10) and (I-A-11):

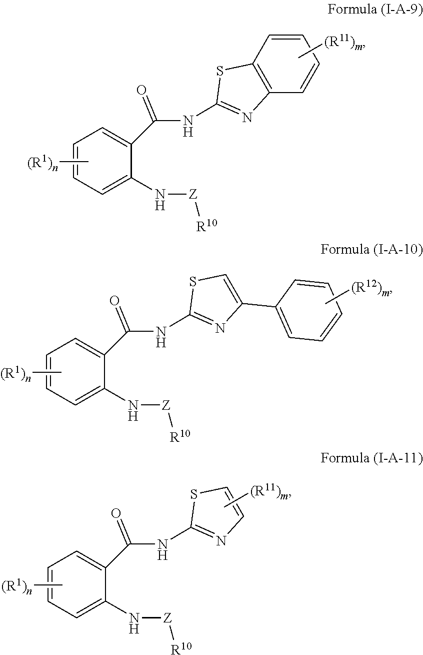

wherein
R¹, n, and m are defined in Formula (I); R¹⁰ is defined in Formula (I-A); Z is defined in Formula (I-A-1); and R¹¹ and R¹² are defined in Formulae (I-A-3), (I-A-4) and (I-A-5).

In some embodiments, small molecule USP2 inhibitors comprises Formulae (I-A-12), (I-A-13), (I-A-14), (I-A-15), (I-A-16), and (I-A-17):

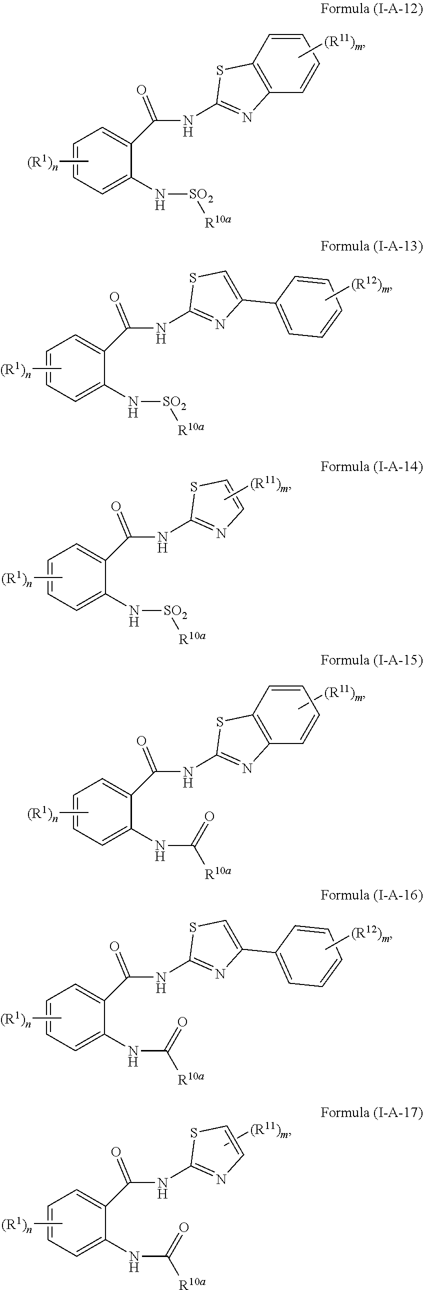

wherein
R¹, n, and m are defined in Formula (I); R^{10a} is selected from H, C₁-C₆ alkyl, C₂-C₆ alkenyl, C₃-C₁₂ cycloalkyl, or 3-12 membered heterocyclic, where each said C₁-C₆ alkyl, C₂-C₆ alkenyl, C₃-C₁₂ cycloalkyl, or 3-12 membered heterocyclic is optionally substituted with halogen, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl.
and R⁸ and R⁹ are defined in Formulae (I),
R¹¹ and R¹² are defined in Formulae (I-A-3), (I-A-4) and (I-A-5).

In some embodiments, each R¹ is independently selected from H, halogen, cyano, ORS, NR⁸R⁹, C(O)OR⁸, C(O)NR⁸R⁹, C₁-C₆ alkyl, C₁-C₆ heteroalkyl, C₃-C₆ cycloalkyl, and 3-6 membered heterocyclic, where each said C₁-C₆ alkyl, C₁-C₆ heteroalkyl, C₃-C₆ cycloalkyl, or 3-6 membered heterocyclic is optionally substituted with halogen, C(O)NR⁸R⁹, C₁-C₆ alkyl, or C₁-C₆ heteroalkyl. In some embodiments, each R¹ is independently selected from H, halogen, cyano, C₁-C₆ alkyl, C₃-C₆ cycloalkyl, and C₁-C₆ alkoxy, where each said C₁-C₆ alkyl, C₃-C₆ cycloalkyl or C₁-C₆ alkoxy is optionally substituted with halo or C₁-C₆ alkyl. In some embodiments, each R¹ is independently selected from H, halogen, C₁-C₆ alkyl, and C₁-C₆ alkoxy, where each said C₁-C₆ alkyl or C₁-C₆ alkoxy is optionally substituted with halo. In some embodiments, each R¹ is independently selected from H, F, Cl, CH₃, CF₃, OCH₃.

In some embodiments, R^{10a} is selected from H, C₁-C₆ alkyl, C₂-C₆ alkenyl, C₃-C₁₂ cycloalkyl, or 3-12 membered heterocyclic, where each said C₁-C₆ alkyl, C₂-C₆ alkenyl, C₃-C₁₂ cycloalkyl, or 3-12 membered heterocyclic is optionally substituted with halogen, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl. In some embodiments, R^{10a} is selected from CH₃, CH═CH, propyl, isopropyl, cyclopropyl, cyclohexyl and trifluoromethyl.

In some embodiments, each R¹¹ is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₃-C₆ cycloalkyl, 3-6 membered heterocyclic, C₆-C₁₀ aryl, or 5-10 membered heteroaryl, where each said C₁-C₆ alkyl, C₁-C₆ heteroalkyl, C₃-C₆ cycloalkyl, 3-6 membered heterocyclic, C₆-C₁₀ aryl, or 5-10 membered heteroaryl is optionally substituted with halogen, cyano, C₁-C₆ alkyl, C₁-C₆ heteroalkyl, C₃-C₆ cycloalkyl, 3-6 membered heterocyclic. In some embodiments, each R¹¹ is independently selected from H, halogen, cyano, hydroxy, C₁-C₆ alkyl, C₃-C₆ cycloalkyl, and 3-6 membered heterocyclic, where each said C₁-C₆ alkyl, C₃-C₆ cycloalkyl, and 3-6 membered heterocyclic, is optionally substituted with halo or C₁-C₆ alkyl. In some embodiments, each R¹¹ is independently selected from H, CN, OH, F, Cl, Br, CH₃, and CF₃. In some embodiments, each R¹¹ is independently selected from C₆-C₁₀ aryl, or 5-10 membered heteroaryl, where each said C₆-C₁₀ aryl, or 5-10 membered heteroaryl is optionally substituted with halogen, cyano, OR⁸, NR⁸R⁹, C₁-C₆ alkyl, C₁-C₆ heteroalkyl, C₃-C₆ cycloalkyl, or 3-6 membered heterocyclic. In some embodiments, each R¹¹ is C₆-C₁₀ aryl, which is optionally substituted with halogen, cyano, OR⁸, NR⁸R⁹, C₁-C₆ alkyl, C₁-C₆ heteroalkyl, C₃-C₆ cycloalkyl, or 3-6 membered heterocyclic. In some embodiments, each R¹¹ is 5-10 membered heteroaryl, which is optionally substituted with halogen, cyano, OR⁸, NR⁸R⁹, C₁-C₆ alkyl, C₁-C₆ heteroalkyl, C₃-C₆ cycloalkyl, or 3-6 membered heterocyclic.

In some embodiments, each R¹² is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₃-C₆ cycloalkyl, or 3-6 membered heterocyclic, where each said C₁-C₆ alkyl, C₁-C₆ heteroalkyl, C₃-C₆ cycloalkyl or 3-6 membered heterocyclic, is optionally substituted with halogen, cyano, C₁-C₆ alkyl, C₁-C₆ heteroalkyl, C₃-C₆ cycloalkyl, 3-6 membered heterocyclic. In some embodiments, each R¹² is independently selected from H, halogen, cyano, hydroxy, C₁-C₆ alkyl, C₃-C₆ cycloalkyl, and 3-6 membered heterocyclic, where each said C₁-C₆ alkyl, C₃-C₆ cycloalkyl, and 3-6 membered heterocyclic, is optionally substituted with halo or C₁-C₆ alkyl. In some embodiments, each R¹¹ is independently selected from H, CN, OH, F, Cl, Br, CH₃, and CF₃.

In some embodiments, each R⁶ and each R⁷, are independently selected from H and C₁-C₆ alkyl, or R⁶ and R⁷, together with the atoms to which they are attached, optionally form 4-6 membered heterocyclic.

In some embodiments, each R⁸ and each R⁹ are independently selected from H and C₁-C₆ alkyl, or R⁸ and R⁹, together with the atoms to which they are attached, optionally form 4-6 membered heterocyclic.

In some embodiments, n is 0, 1, 2. In some embodiments, n is 0. In some embodiments, n is 1. In some embodiments, m is 0, 1, 2. In some embodiments, m is 0. In some embodiments, m is 1. In some embodiments, x is 0, 1, 2. In some embodiments, x is 0. In some embodiments, x is 1. In some embodiments, x is 2. In some embodiments, Ring C in Formula (I) is selected from C₆-C₁₀ aryl, or 5-10 membered heteroaryl; and Ring B is selected from C₆-C₁₀ aryl, or 5-10 membered heteroaryl. In some embodiments, Ring C in Formula (I) is selected from C₆-C₁₀ aryl, or 5-10 membered heteroaryl; and Ring B is selected from 5-10 membered heteroaryl. In some embodiments, Ring C in Formula (I) is selected from C₆-C₁₀ aryl, or 5-10 membered heteroaryl; and Ring B is selected from 5-6 membered monocyclic heteroaryl. In some embodiments, Ring C in Formula (I) is selected from C₆-C₁₀ aryl, or 5-10 membered heteroaryl; and Ring B is selected from 7-10 membered bicyclic heteroaryl.

In some embodiments, the small molecule USP2 inhibitors comprises Formulae (I-B1), (I-B2) and (I-B3):

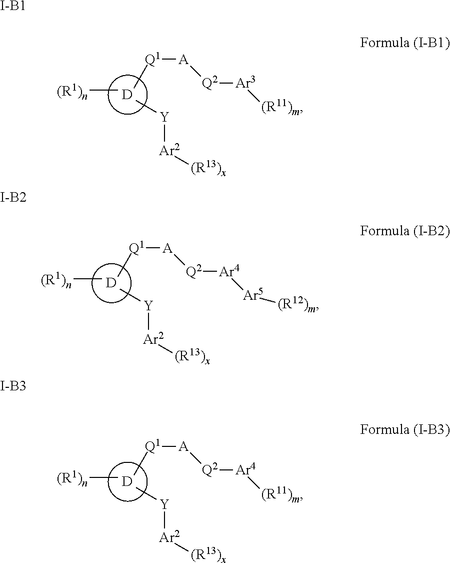

wherein
Ar² is selected from C₆-C₁₀ aryl, or 5-10 membered heteroaryl;
Ar³ is selected from 7-10 membered bicyclic heteroaryl;
Ar⁴ is selected from 5-6 membered monocyclic heteroaryl;
Ar⁵ is selected from C₆-C₁₀ aryl, or 5-10 membered heteroaryl;
each R¹³ is independently selected from H, halogen, cyano, OR⁶, N⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹³, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
A, D, Y, Q¹, Q², R¹, R⁶, R⁷, R⁸, R⁹, n, m, and x are defined in Formula (I); and R¹¹ and R¹² are defined in Formulae (I-A-3), (I-A-4) and (I-A-5).

In some embodiments, the small molecule USP2 inhibitors comprises Formulae (I-B1-1), (I-B2-1) and (I-B3-1):

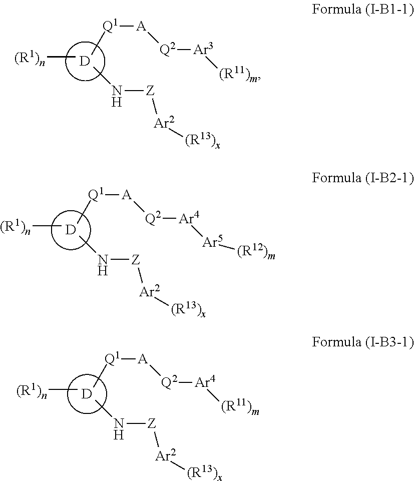

wherein
Ar², Ar³, Ar⁴ and Ar⁵ are defined in Formulae (I-B1), (I-B2) and (I-B3); A, D, Q¹, Q², R¹, n, m, and x are defined in Formula (I); Z are defined in Formula (I-A-1); R¹¹ and R¹² are defined in Formulae (I-A-3), (I-A-4) and (I-A-5); and R¹³ is defined in Formulae (I-B1), (I-B2) and (I-B3).

In some embodiments, the small molecule USP2 inhibitors comprise Formulae (I-B1-2), (I-B2-2) and (I-B2-3):

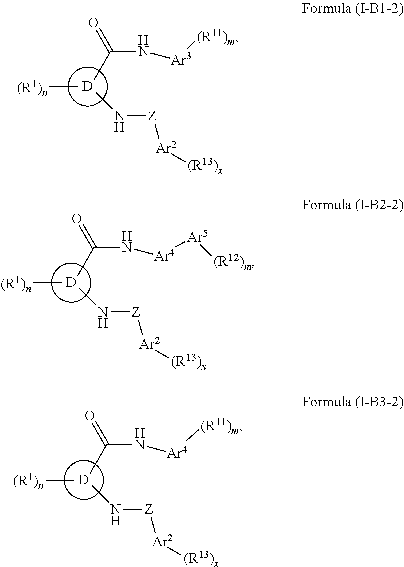

wherein
Ar², Ar³, Ar⁴ and Ar⁵ are defined in Formulae (I-B1), (I-B2) and (I-B3); D, R¹, n, m, and x are defined in Formula (I); Z are defined in Formula (I-A-1); R¹¹ and R¹² are defined in Formulae (I-A-3), (I-A-4) and (I-A-5), and R¹³ is defined in Formulae (I-B1), (I-B2) and (I-B3).

In some embodiments, Ar³ is 8 membered bicyclic heteroaryl comprising two fused 5 membered rings. In some embodiments, Ar³ is 9 membered bicyclic heteroaryl comprising fused 5 and 6 membered rings. In some embodiments, Ar³ is 10 membered bicyclic heteroaryl comprising two fused six membered rings. In some embodiments, Ar³ is a thiazole fused with a 5 membered ring. In some embodiments, Ar³ is a thiazole fused with a 6 membered ring. In some embodiments, Ar³ is a benzothiazole. In some embodiments, Ar³ is

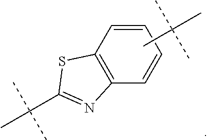

In some embodiments, Ar⁴ is optionally substituted 5-6 membered monocyclic heteroaryl. In some embodiments, Ar⁴ is selected from optionally substituted

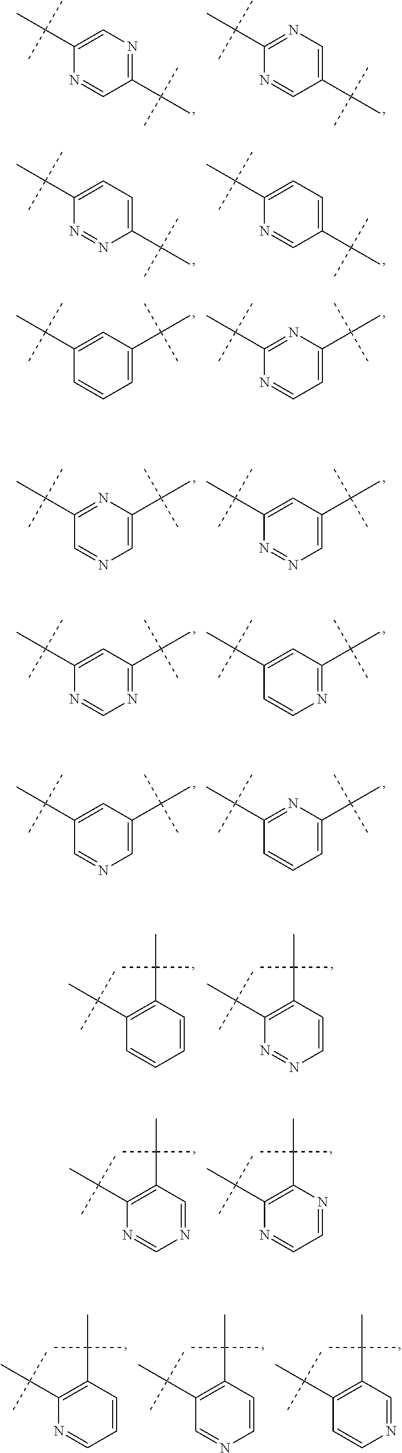

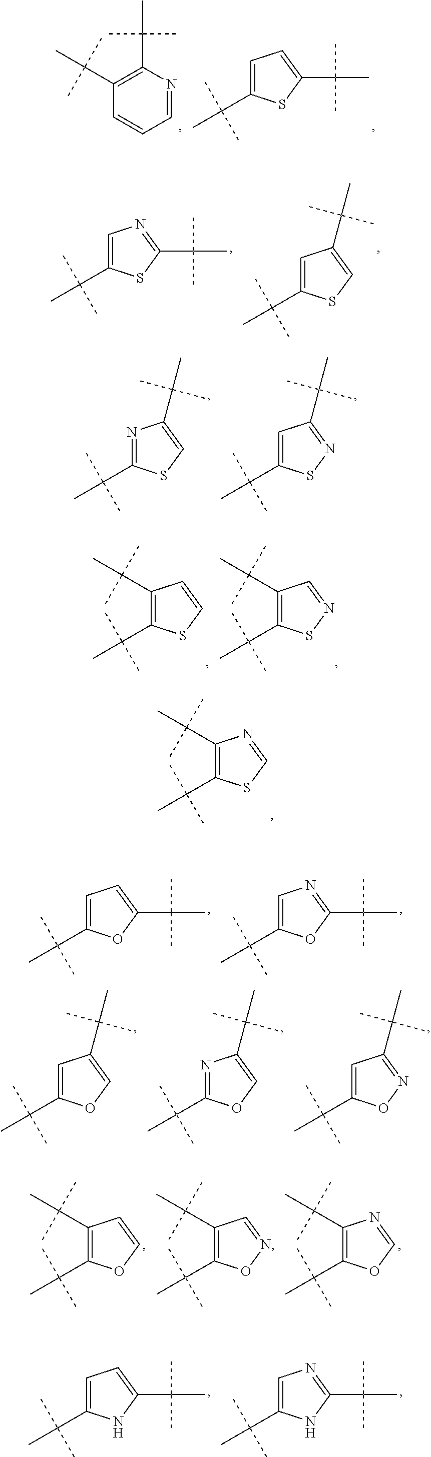

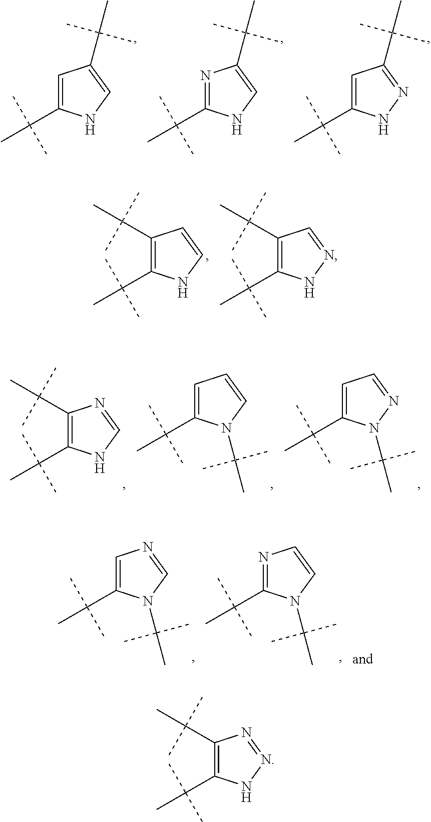

In some embodiments, Ar⁴ selected from optionally substituted

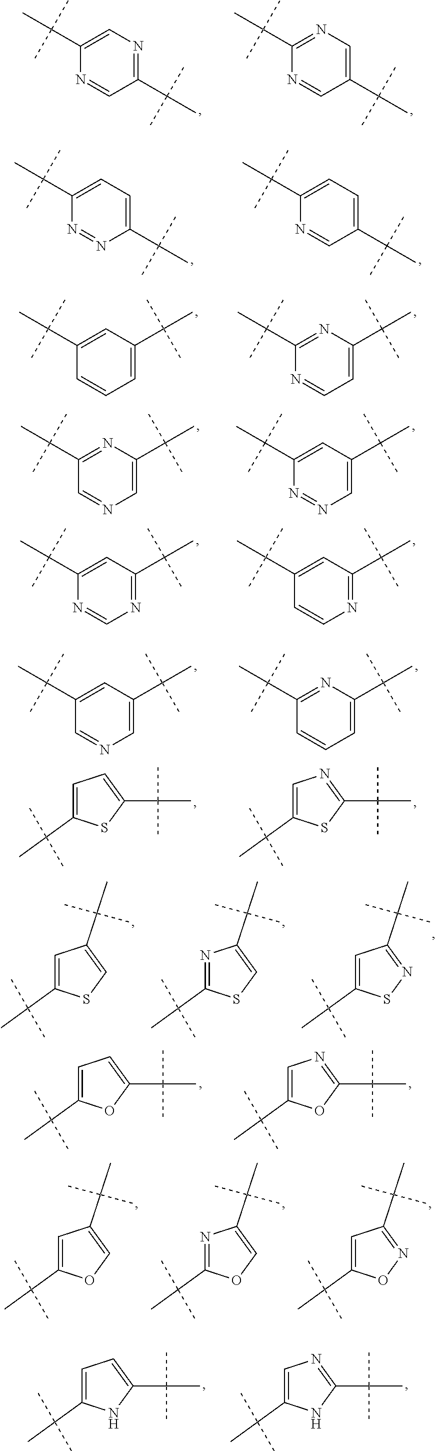

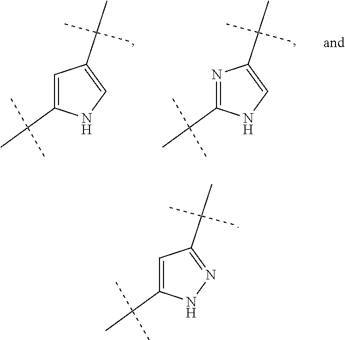

In some embodiments, Ar⁴ is optionally substituted thiazole. In some embodiments, Ar⁴ is

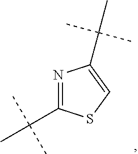

In some embodiments, Ar⁵ is phenylene, or 5-6 membered monocyclic heteroaryl. In some embodiments, Ar⁵ is phenylene. In some embodiments, Ar⁵ is selected from

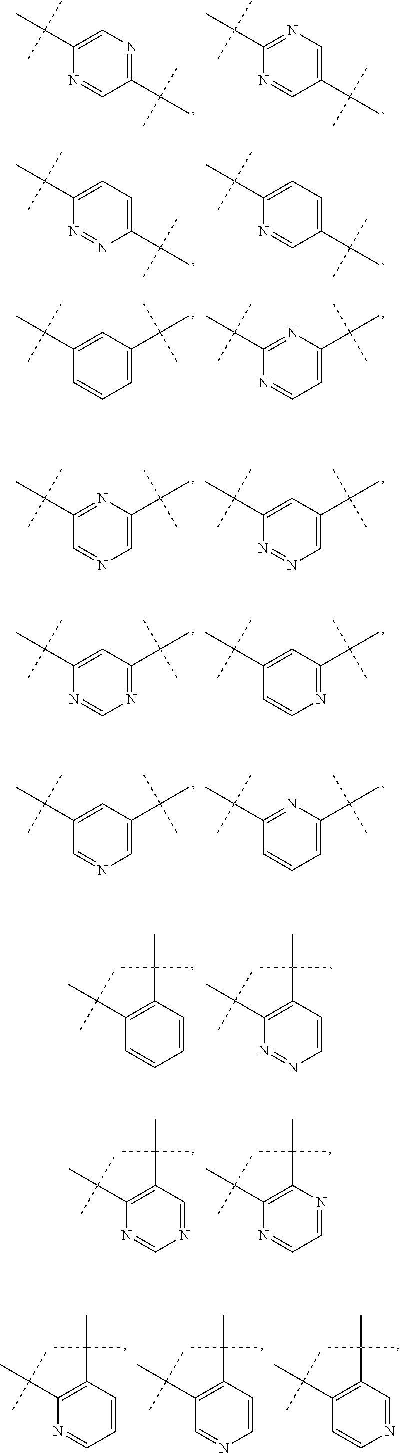

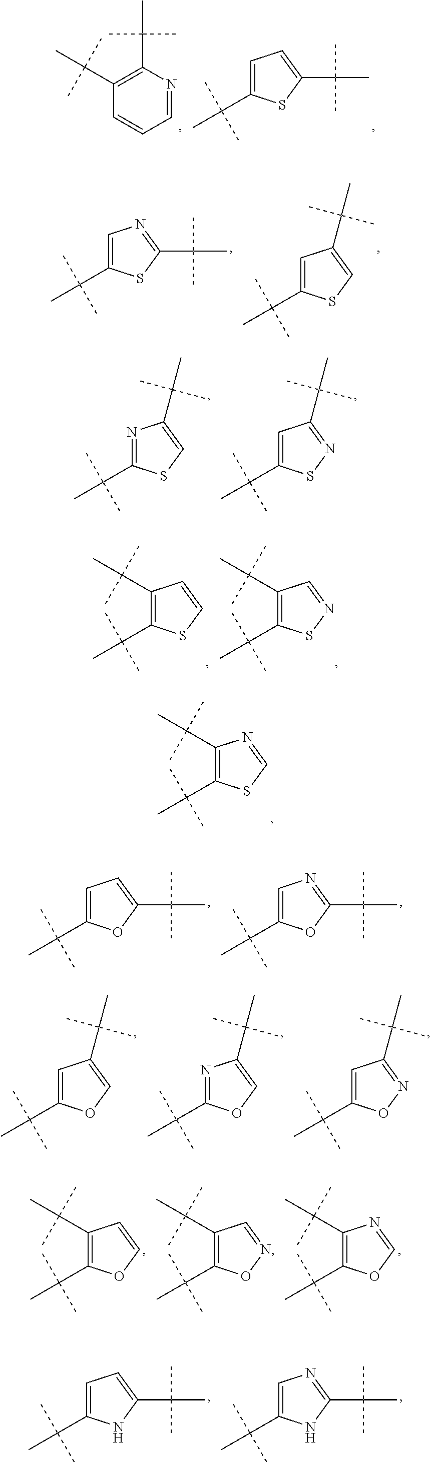

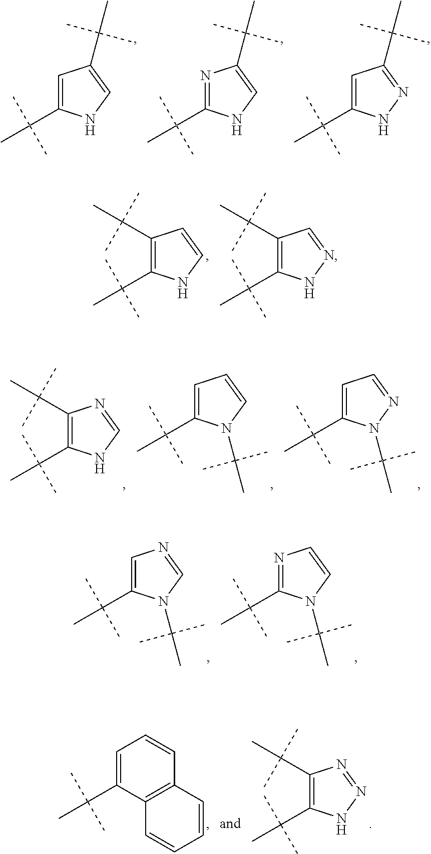

In some embodiments, the small molecule USP2 inhibitors comprises Formulae (I-B1-3), (I-B2-3) and (I-B3-3):

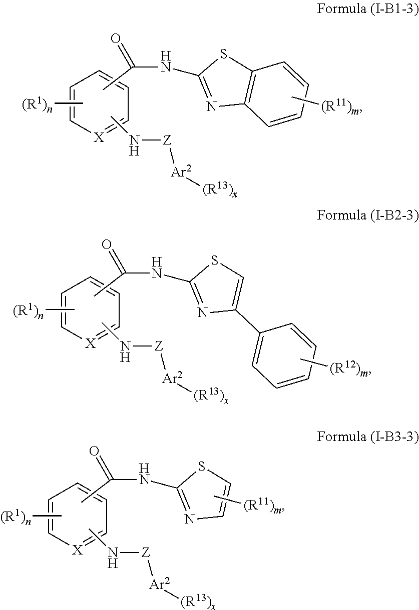

wherein
Ar² is defined in Formulae (I-B1), (I-B2) and (I-B3); R¹, n, m, and x are defined in Formula (I); X, Z is defined in Formula (I-A-1); R¹¹ and R¹² are defined in Formulae (I-A-3), (I-A-4) and (I-A-5); and R¹³ is defined in Formulae (I-B1), (I-B2) and (I-B3).

In some embodiments, the small molecule USP2 inhibitors comprises Formulae (I-B1-4), (I-B2-4) and (I-B2-4):

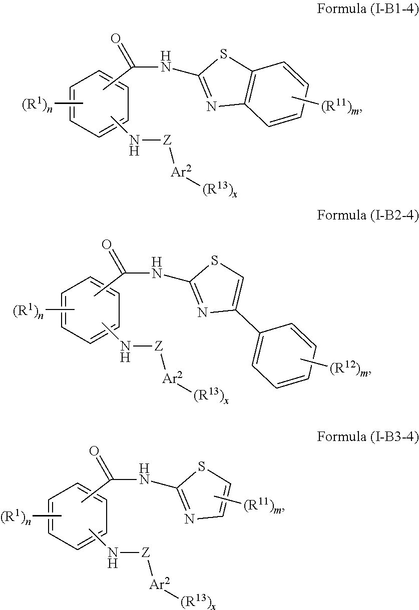

wherein
Ar² is defined in Formulae (I-B1), (I-B2) and (I-B3); R¹, n, m, and x are defined in Formula (I); Z is defined in Formula (I-A-1); R¹¹ and R¹² are defined in Formulae (I-A-3), (I-A-4) and (I-A-5); and R¹¹ is defined in Formulae (I-B1), (I-B2) and (I-B3).

In some embodiments, the small molecule USP2 inhibitors comprises Formulae (I-B1-5), (I-B2-5) and (I-B2-5):

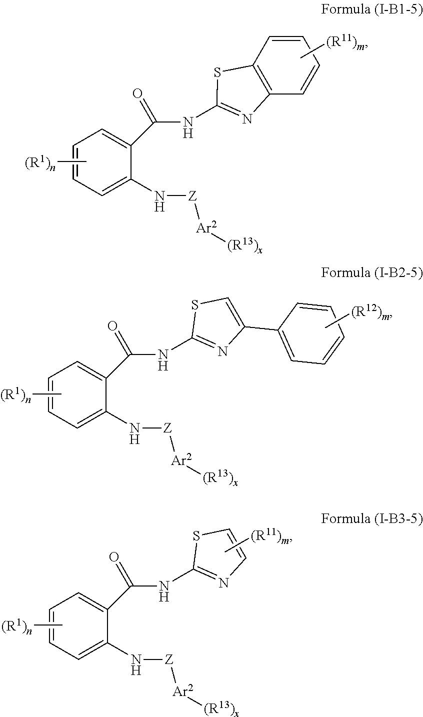

wherein
Ar² is defined in Formulae (I-B1), (I-B2) and (I-B3); R¹, n, m, and x are defined in Formula (I); Z is defined in Formula (I-A-1); R¹¹ and R¹² are defined in Formulae (I-A-3), (I-A-4) and (I-A-5); and R¹³ is defined in Formulae (I-B1), (I-B2) and (I-B3).

In some embodiments, the small molecule USP2 inhibitors comprises Formula (I-B2-6) and (I-B2-7):

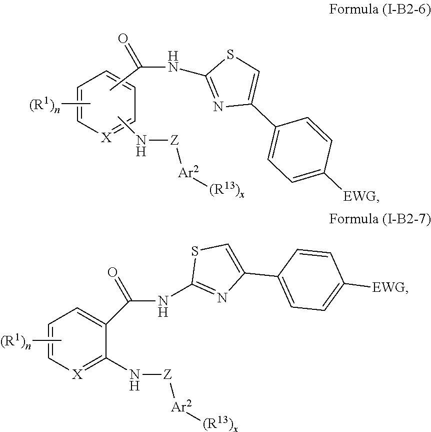

wherein
EWG is an electron withdrawing group; Z is defined in Formula (I-A-1); Ar² is defined in Formulae (I-B1), (I-B2) and (I-B3); X, R¹, n, m, and x are defined in Formula (I); and R¹³ is defined in Formulae (I-B1), (I-B2) and (I-B3).

In some embodiments, EWG is selected from halogen, cyano, C(O)OR⁶, C(O)NR⁶R⁷, S(O)₂NR⁶R⁷, and C₁-C₆ haloalkyl. In some embodiments, EWG is selected from halogen, cyano, and C₁-C₆ haloalkyl. In some embodiments, EWG is selected from F, Cl, Br, CN, CF₃.

In some embodiments, the small molecule USP2 inhibitors comprises Formulae (I-B2-8), (I-B2-9), (I-B2-10), (I-B2-11), (I-B2-12), (I-B2-13), (I-B2-14), (I-B2-15), (I-B2-16); and (I-B2-17):

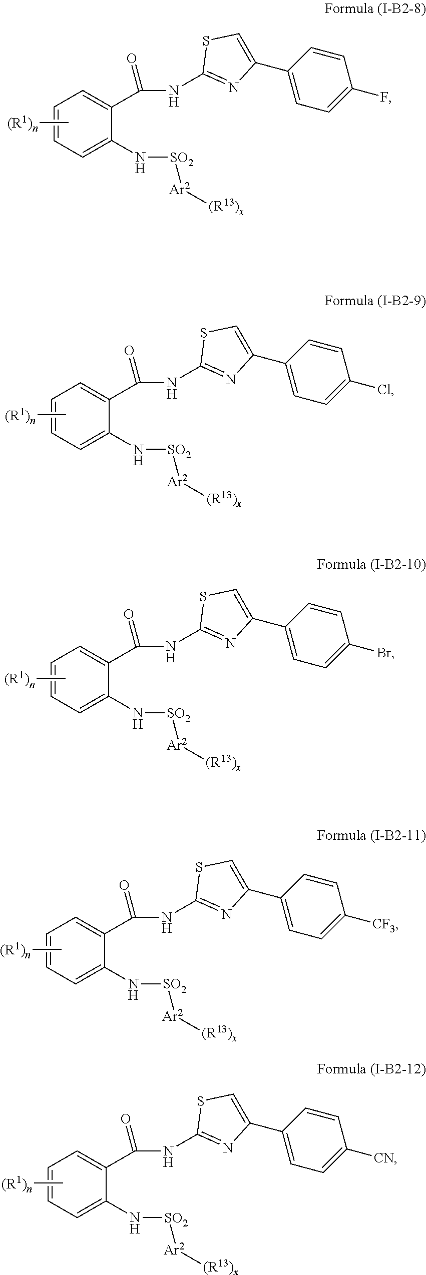

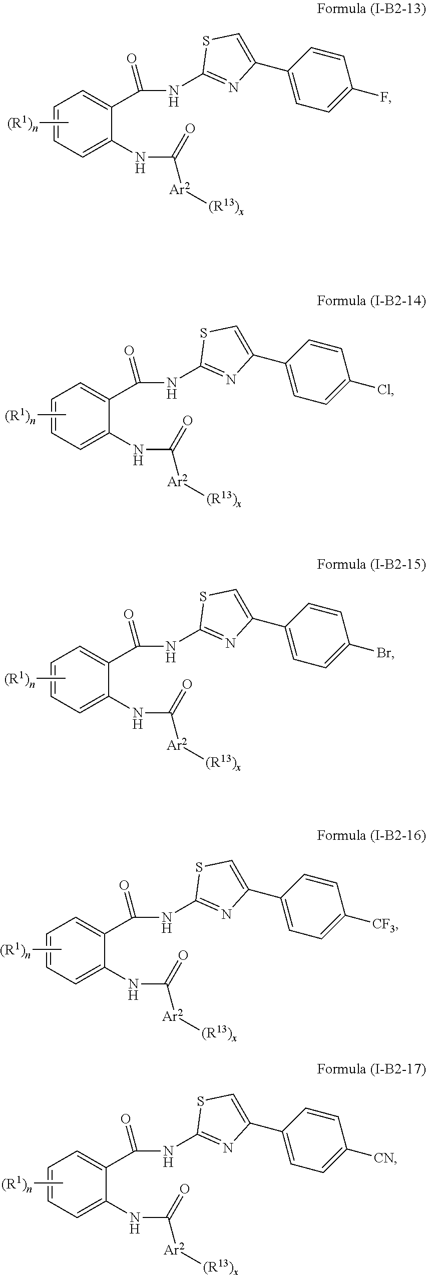

wherein
Ar² is defined in Formulae (I-B1), (I-B2) and (I-B3); R¹, n, and x are defined in Formula (I); and R¹³ is defined in Formulae (I-B1), (I-B2) and (I-B3).

In some embodiments, each R¹ is independently selected from H, halogen, cyano, ORS, NR⁸R⁹, C(O)OR⁸, C(O)NR⁸R⁹, C₁-C₆ alkyl, C₁-C₆ heteroalkyl, C₃-C₆ cycloalkyl, and 3-6 membered heterocyclic, where each said C₁-C₆ alkyl, C₁-C₆ heteroalkyl, C₃-C₆ cycloalkyl, or 3-6 membered heterocyclic is optionally substituted with halogen, C(O)NR⁸R⁹, C₁-C₆ alkyl, or C₁-C₆ heteroalkyl. In some embodiments, each R¹ is independently selected from H, halogen, cyano, C₁-C₆ alkyl, C₃-C₆ cycloalkyl, and C₁-C₆ alkoxy, where each said C₁-C₆ alkyl, C₃-C₆ cycloalkyl or C₁-C₆ alkoxy is optionally substituted with halo or C₁-C₆ alkyl. In some embodiments, each R¹ is independently selected from H, halogen, C₁-C₆ alkyl, and C₁-C₆ alkoxy, where each said C₁-C₆ alkyl or C₁-C₆ alkoxy is optionally substituted with halo. In some embodiments, each R¹ is independently selected from H, F, Cl, CH₃, CF₃, OCH₃.

In some embodiments, Ar² is selected from optionally substituted phenylene, or 5-6 membered heteroaryl. In some embodiments, Ar² is selected from optionally substituted phenylene. In some embodiments, Ar² is selected from 5-6 membered heteroaryl. In some embodiments, is selected from optionally substituted

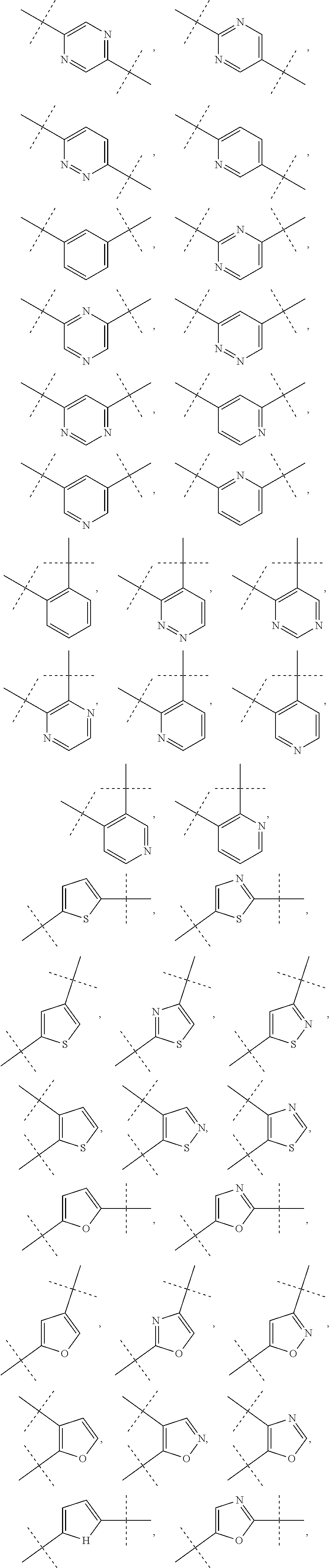

In some embodiments, each R¹³ is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₃-C₆ cycloalkyl, or 3-6 membered heterocyclic, where each said C₁-C₆ alkyl, C₁-C₆ heteroalkyl, C₃-C₆ cycloalkyl or 3-6 membered heterocyclic, is optionally substituted with halogen, cyano, C₁-C₆ alkyl, C₁-C₆ heteroalkyl, C₃-C₆ cycloalkyl, 3-6 membered heterocyclic. In some embodiments, each R¹³ is independently selected from H, halogen, cyano, hydroxy, C₁-C₆ alkyl, C₃-C₆ cycloalkyl, and 3-6 membered heterocyclic, where each said C₁-C₆ alkyl, C₃-C₆ cycloalkyl, and 3-6 membered heterocyclic, is optionally substituted with halo or C₁-C₆ alkyl. In some embodiments, each R¹³ is independently selected from H, CN, NO₂, OH, F, Cl, Br, CH₃, CF₃ and OCH₃. In some embodiments, each R¹³ is independently selected from CN, Br, CH₃, and CF₃.

In some embodiments, the small molecule USP2 inhibitors comprises Formulae (I-C1), (I-C2), (I-C3):

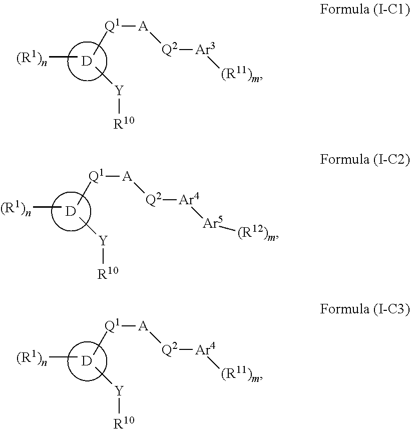

Wherein,
A, D, Y, Q¹, Q², R¹, n, and m are defined in Formula (I); R¹⁰ is defined in Formula (I-A); R¹¹ is defined in Formula (I-A-3); Ar³, Ar⁴, and Ar⁵ are defined in Formulae (I-B1), (I-B2), and (I-B3).

In some embodiments, D is 9-10 membered bicycle heteroaryl; In some embodiments, D is 9 membered bicycle heteroaryl; In some embodiments, D is 10 membered bicycle heteroaryl; In some embodiments, D is selected from

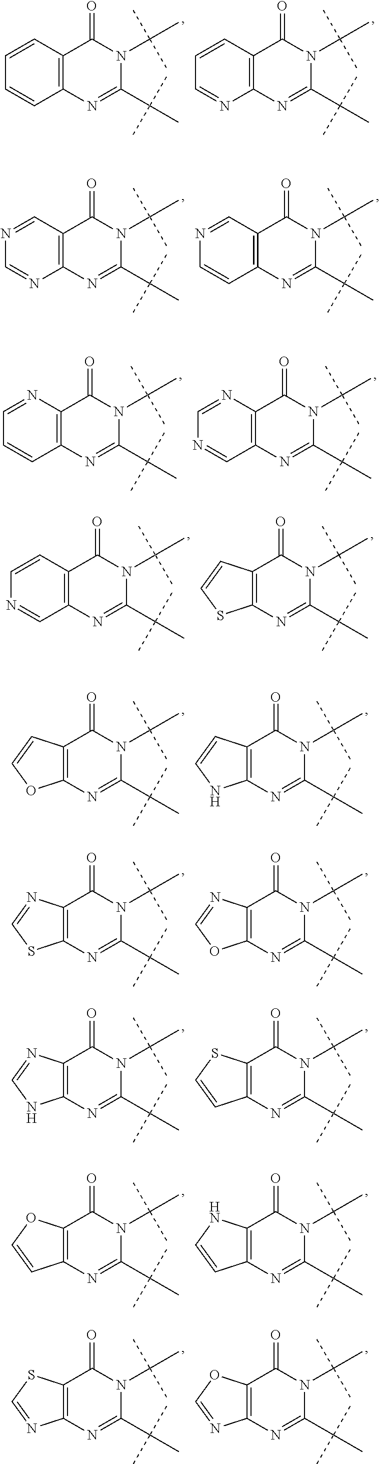

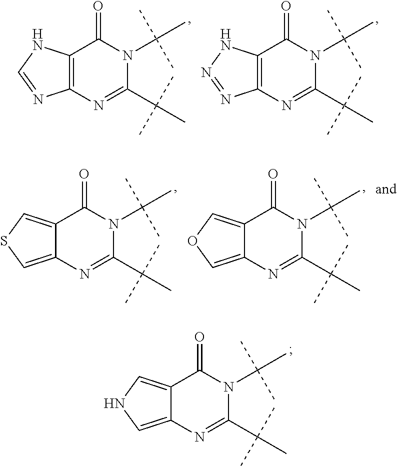

In some embodiments, D is

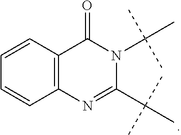

In some embodiments, the small molecule USP2 inhibitors comprises Formulae (I-C1-1), (I-C2-1), (I-C3-1), (I-C1-2), (I-C2-2), and (I-C3-2):

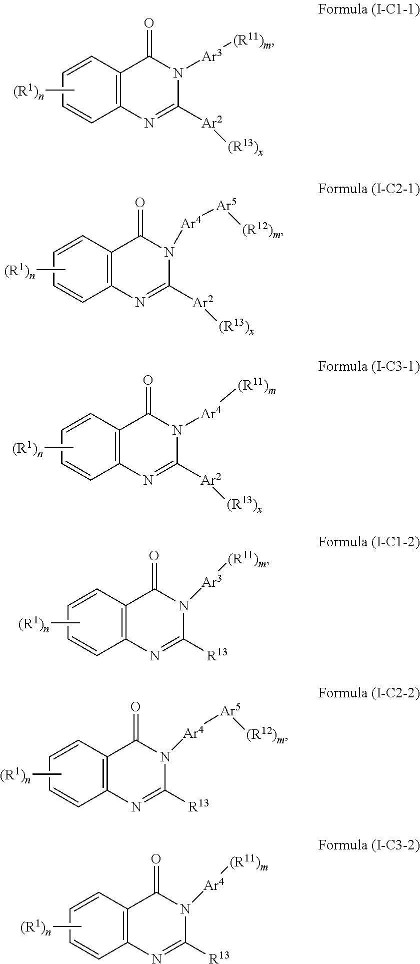

Wherein,
R¹, n, m, x are defined in Formula (I); R¹¹ and R¹² are defined in Formulae (I-A-3), (I-A-4) and (I-A-5); Ar², Ar³, Ar⁴, Ar⁵ and R¹¹ are defined in Formulae (I-B1), (I-B2), and (I-B3).

In some embodiments, Ar³ is 8 membered bicyclic heteroaryl comprising two fused 5 membered rings. In some embodiments, Ar³ is 9 membered bicyclic heteroaryl comprising fused 5 and 6 membered rings. In some embodiments, Ar³ is 10 membered bicyclic heteroaryl comprising two fused six membered rings. In some embodiments, Ar³ is a thiazole fused with a 5 membered ring. In some embodiments, Ar³ is a thiazole fused with a 6 membered ring. In some embodiments, Ar³ is a benzothiazole. In some embodiments, Ar³ is

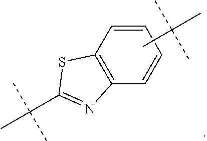

In some embodiments, Ar⁴ is optionally substituted 5-10 membered heteroaryl. In some embodiments, Ar⁴ is optionally substituted 5-6 membered monocyclic heteroaryl. In some embodiments, Ar⁴ is selected from optionally substituted

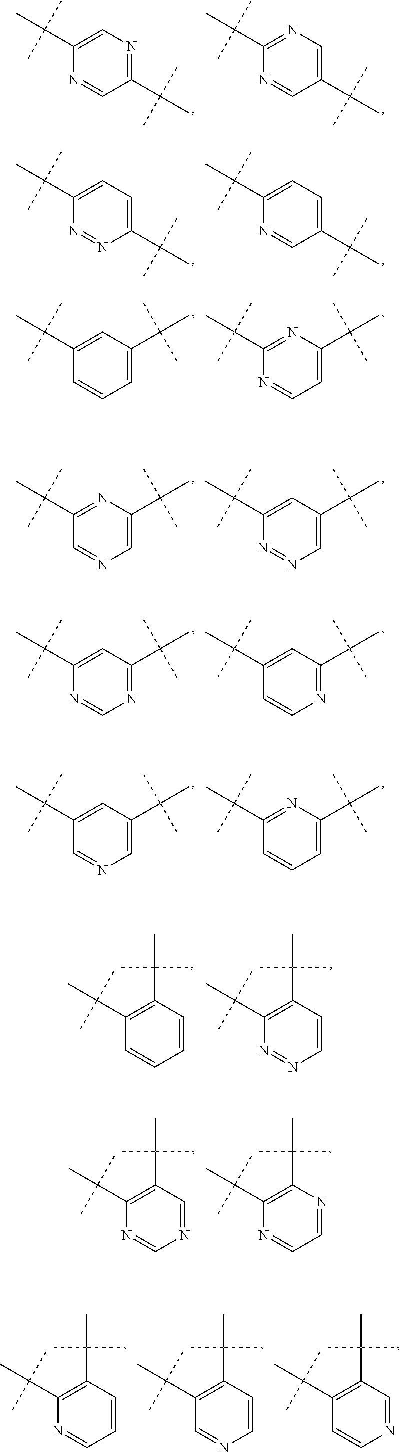

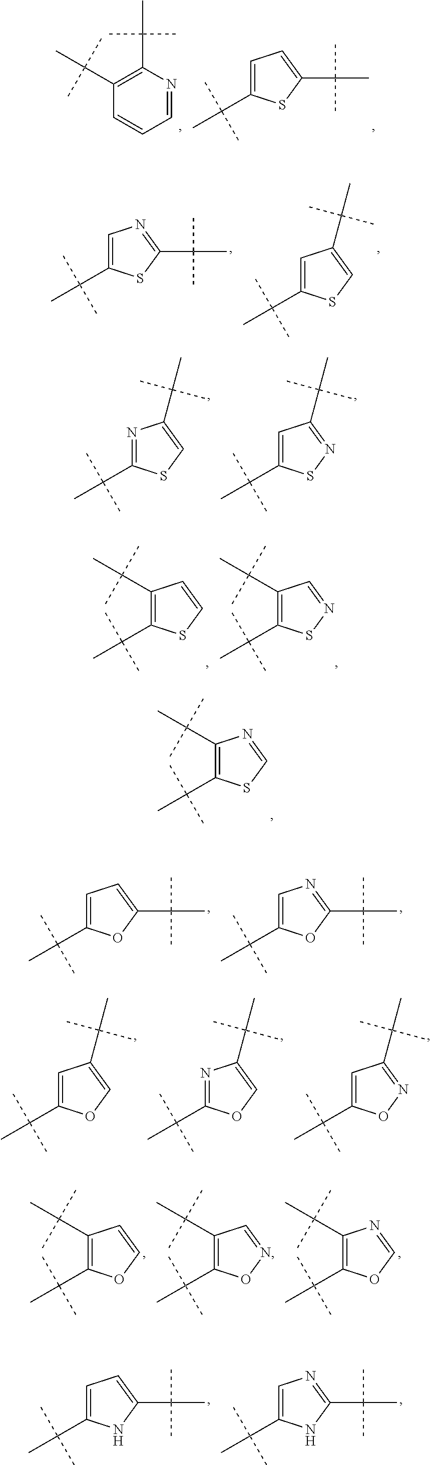

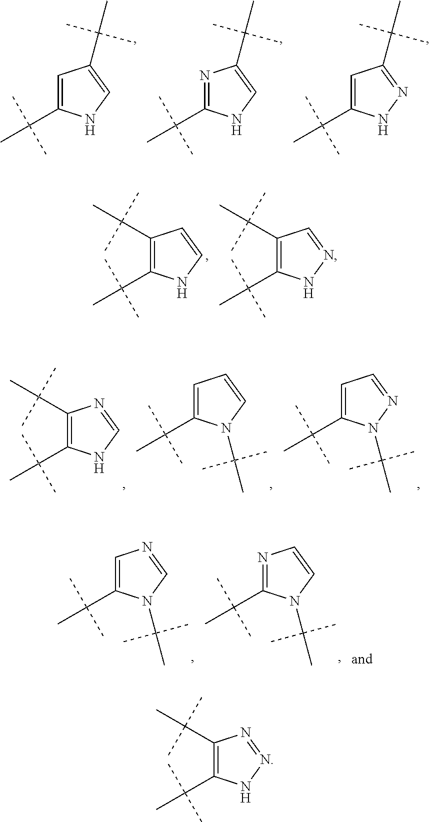

In some embodiments, Ar⁴ is selected from optionally substituted

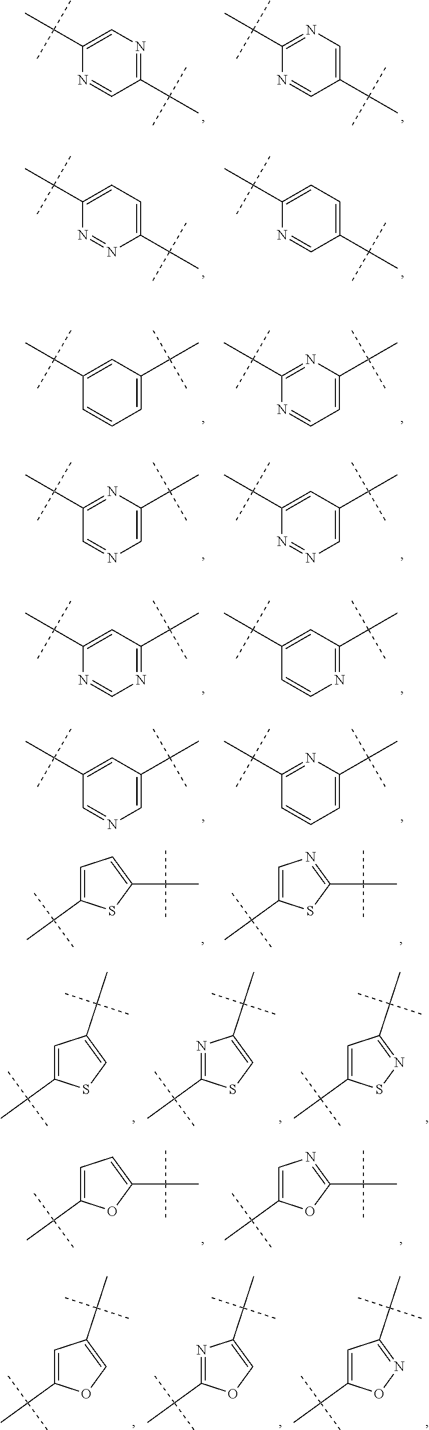

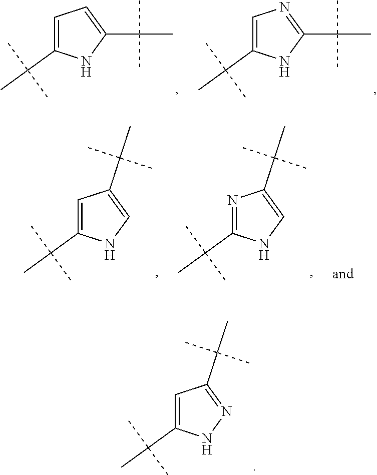

In some embodiments, Ar⁴ is optionally substituted thiazole. In some embodiments, Ar⁴ is

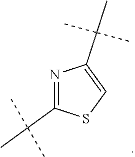

In some embodiments, Ar⁵ is phenylene, or 5-6 membered monocyclic heteroaryl. In some embodiments, Ar⁵ is phenyl. In some embodiments, Ar⁵ is selected from

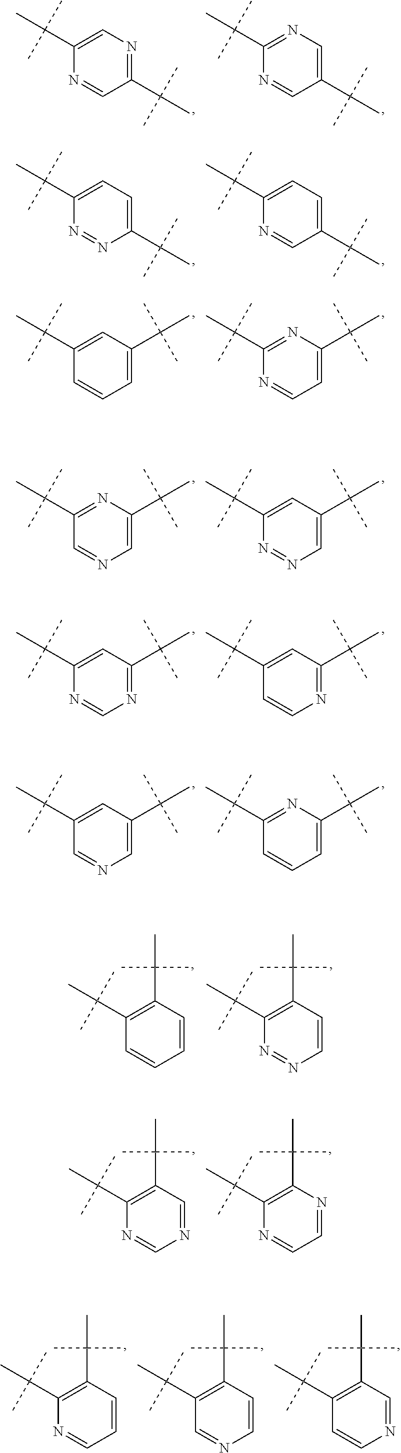

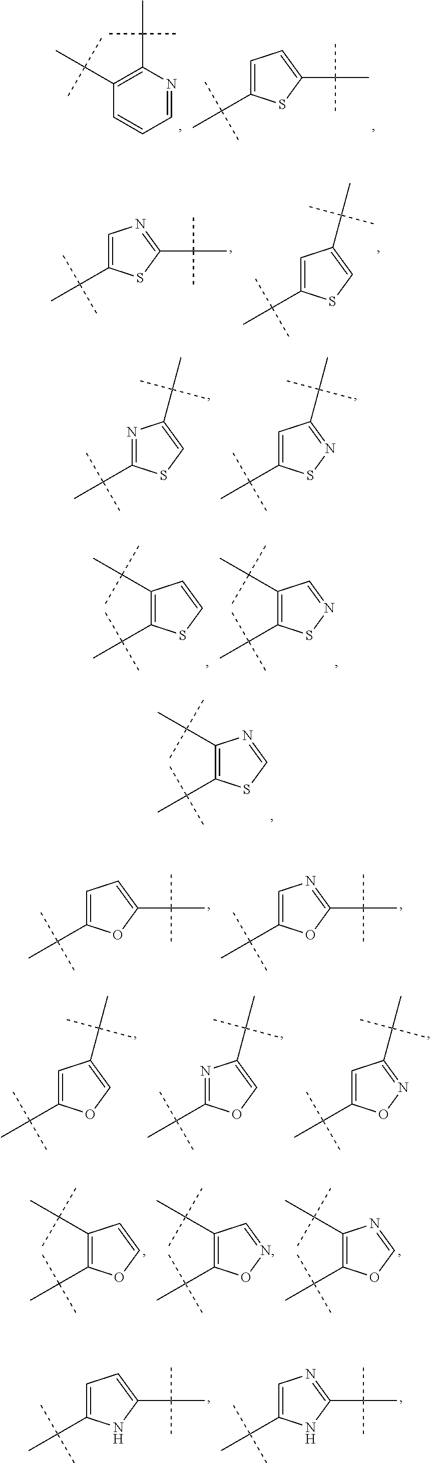

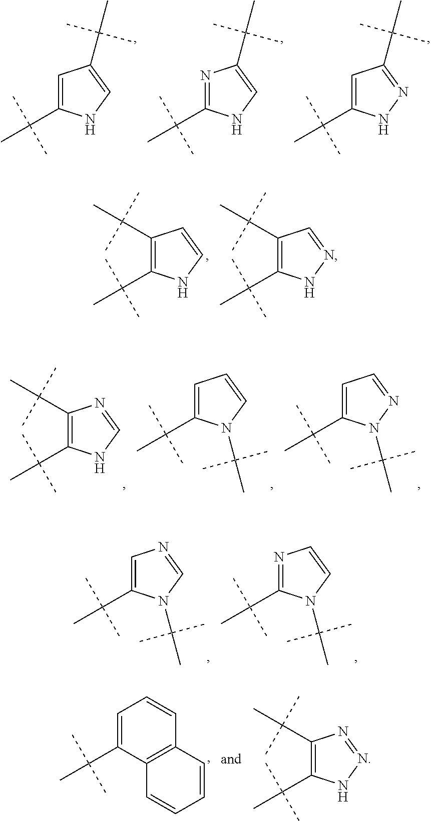

In some embodiments, the small molecule USP2 inhibitors comprises Formulae (I-C2-3), and (I-C2-4).

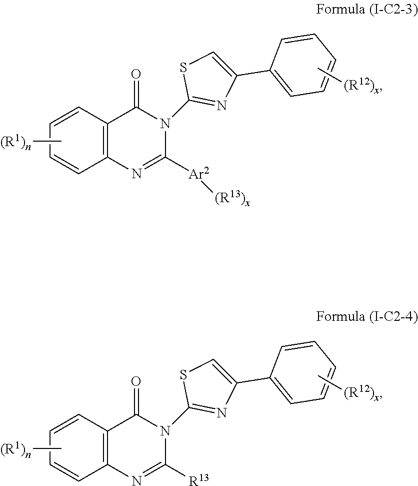

Wherein,
R¹, n, and x are defined in Formula (I); R¹² is defined in Formulae (I-A-3), (I-A-4) and (I-A-5); Ar², and R¹³ are defined in Formulae (I-B1), (I-B2) and (I-B3).

In some embodiments, Ar² is selected from optionally substituted phenylene, or 5-6 membered heteroaryl. In some embodiments, Ar² is selected from optionally substituted phenylene. In some embodiments, Ar² is selected from 5-6 membered heteroaryl. In some embodiments, Ar² is selected from optionally substituted

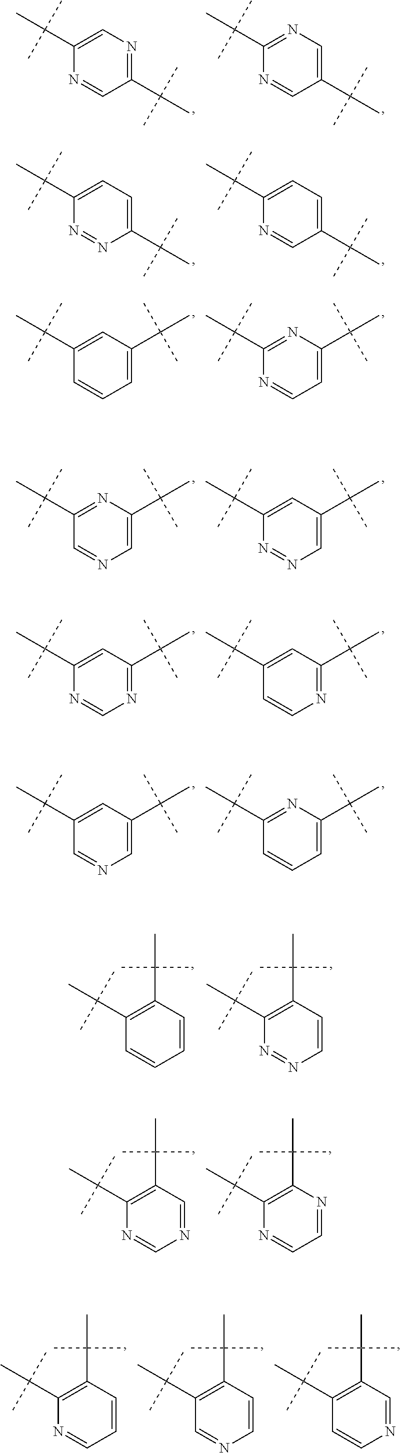

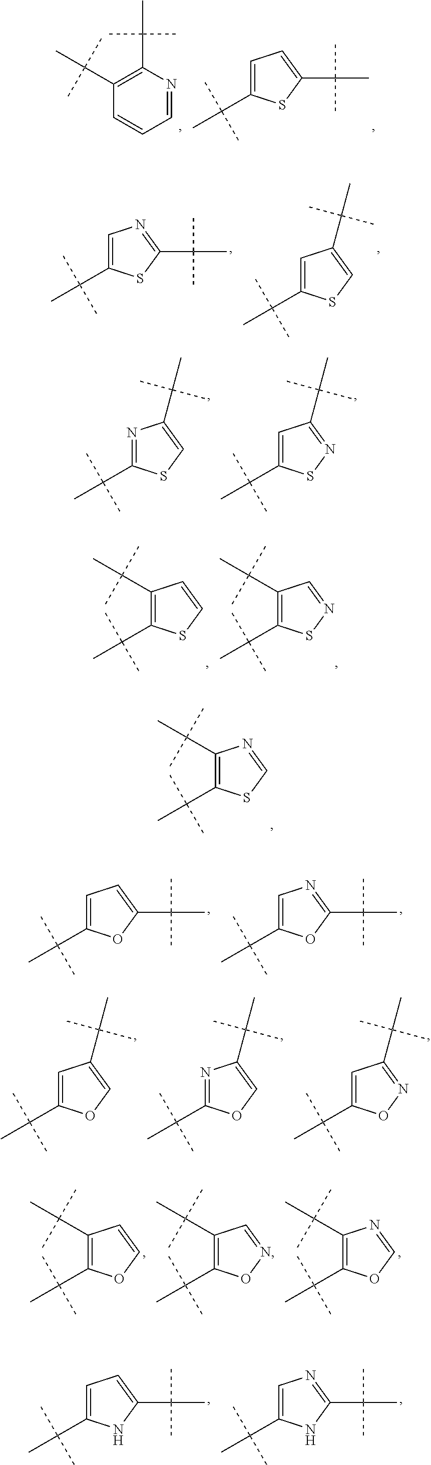

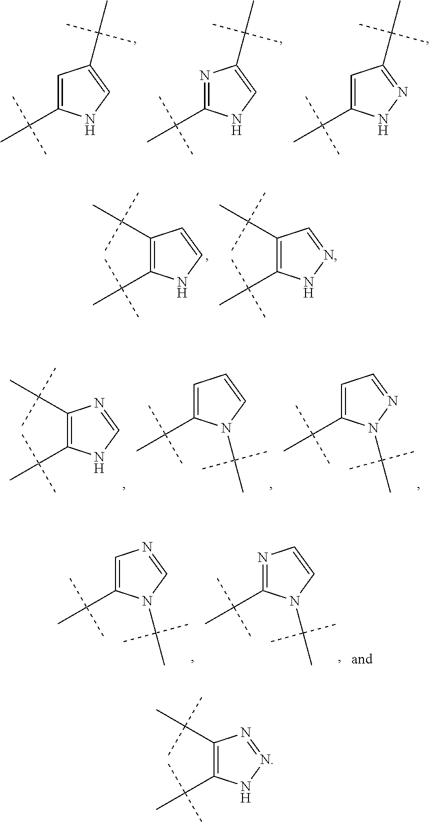

In some embodiments, each R¹ is independently selected from H, halogen, cyano, OR⁸, NR⁸R⁹, C(O)ORs, C(O)NR⁸R⁹, C₁-C₆ alkyl, C₁-C₆ heteroalkyl, C₃-C₆ cycloalkyl, and 3-6 membered heterocyclic, where each said C₁-C₆ alkyl, C₁-C₆ heteroalkyl, C₃-C₆ cycloalkyl, or 3-6 membered heterocyclic is optionally substituted with halogen, C(O)NR⁸R⁹, C₁-C₆ alkyl, or C₁-C₆ heteroalkyl. In some embodiments, each R¹ is independently selected from H, halogen, cyano, C₁-C₆ alkyl, C₃-C₆ cycloalkyl, and C₁-C₆ alkoxy, where each said C₁-C₆ alkyl, C₃-C₆ cycloalkyl or C₁-C₆ alkoxy is optionally substituted with halo or C₁-C₆ alkyl. In some embodiments, each R¹ is independently selected from H, halogen, C₁-C₆ alkyl, and C₁-C₆ alkoxy, where each said C₁-C₆ alkyl or C₁-C₆ alkoxy is optionally substituted with halo. In some embodiments, each R¹ is independently selected from H, F, Cl, CH₃, CF₃, OCH₃.

In some embodiments, each R¹² is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₃-C₆ cycloalkyl, or 3-6 membered heterocyclic, where each said C₁-C₆ alkyl, C₁-C₆ heteroalkyl, C₃-C₆ cycloalkyl or 3-6 membered heterocyclic, is optionally substituted with halogen, cyano, C₁-C₆ alkyl, C₁-C₆ heteroalkyl, C₃-C₆ cycloalkyl, 3-6 membered heterocyclic. In some embodiments, each R¹² is independently selected from H, halogen, cyano, hydroxy, C₁-C₆ alkyl, C₃-C₆ cycloalkyl, and 3-6 membered heterocyclic, where each said C₁-C₆ alkyl, C₃-C₆ cycloalkyl, and 3-6 membered heterocyclic, is optionally substituted with halo or C₁-C₆ alkyl. In some embodiments, each R¹¹ is independently selected from H, CN, OH, F, Cl, Br, CH₃, and CF₃.

In some embodiments, each R¹³ is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₃-C₆ cycloalkyl, or 3-6 membered heterocyclic, where each said C₁-C₆ alkyl, C₁-C₆ heteroalkyl, C₃-C₆ cycloalkyl or 3-6 membered heterocyclic, is optionally substituted with halogen, cyano, C₁-C₆ alkyl, C₁-C₆ heteroalkyl, C₃-C₆ cycloalkyl, 3-6 membered heterocyclic. In some embodiments, each R¹³ is independently selected from H, halogen, cyano, hydroxy, C₁-C₆ alkyl, C₃-C₆ cycloalkyl, and 3-6 membered heterocyclic, where each said C₁-C₆ alkyl, C₃-C₆ cycloalkyl, and 3-6 membered heterocyclic, is optionally substituted with halo or C₁-C₆ alkyl. In some embodiments, each R¹³ is independently selected from H, CN, NO₂, OH, F, Cl, Br, CH₃, CF₃ and OCH₃. In some embodiments, each R¹³ is independently selected from CN, Br, CH₃, and CF₃.

In some embodiments, the small molecule USP2 inhibitors comprises Formulae (I-C2-5), and (I-C2-6).

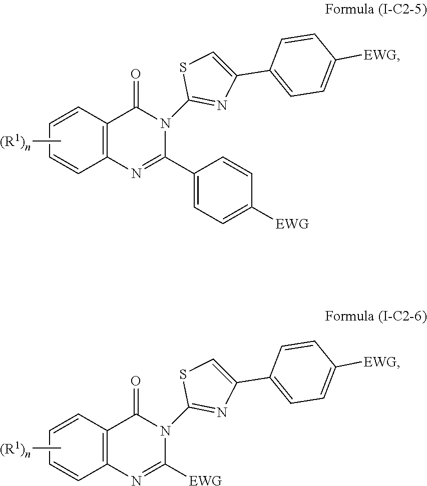

Wherein
R¹ is defined in Formula (I); EWR is defined in Formulae (I-B2-6), and (I-B2-7).

In some embodiments, each EWG is selected from halogen, cyano, C(O)OR⁶, C(O)NR⁶R⁷, S(O)₂NR⁶R⁷, and C₁-C₆ haloalkyl. In some embodiments, each EWG is selected from halogen, cyano, and C₁-C₆ haloalkyl. In some embodiments, each EWG is selected from F, Cl, Br, CN, CF₃.

In some embodiments, the USP2 inhibitor is a compound selected from XH161-102, XH153-7, XH153-10, XH153-74, XH146-120, XH146-123, XH153-144, XH146-180, XH153-6, XH 153-168, XH 153-157, XH 153-148, XH 153-147, XH 153-167, XH 153-145, XH153-182, X1153-155, XH153-150, XH161-26, XH161-2, X1161-4, XH161-5, XH161-6, X1161-3, XH161-19, XH161-37, XH161-32, XH161-33, XH161-25, XH161-31, XH161-36, XH161-94, XH161-98, XH161-95, XH161-35, XH161-96, XH161-134, XH161-135, XH161-132, XH161-133, XH161-101, XH161-136, XH161-137, XH161-169, XH161-170, XH161-153, XH161-154, XH168-77, XH181-20, XH181-38, XH181-39, XH161-177, XH161-176, XH161-172, XH161-173, XH168-72-1, XH168-72-2, XH168-72-3, XH168-79, XH168-63, XH168-76, XH161-180, XH153-79-D, XH153-83, XH153-84-pl, XH153-84-p2, XH153-20, XH181-103, XH181-104, XH188-105, XH188-111, XH188-113, XH188-117, XH188-119, XH188-122, XH188-123, XH188-124, XH188-125, XH188-137, XH188-146, XH188-149, XH188-150, XH188-151, XH188-170, XH188-171, XH188-172, XH188-173, XH188-174, XH188-175, XH188-176, XH188-183, XH188-184, XH188-185, XH188-186, XH198-1, XH198-2, XH198-3, XH198-4, XH198-62, XH193-63, XH198-65, XH198-66, XH198-67, XH198-70, XH198-71, XH188-135p1, XH188-135p2, XH198-8, XH198-9, XH198-38P1, XH198-38P2, XH198-43P1, XH198-43P2, XH198-44, XH198-45, XH198-46, XH198-48, XH198-68P1, XH198-68P2, XH198-69P1, XH198-69P2, XH208-34P1, XH208-34P2, XH208-39P1, XH208-39P2, XH208-40P1, XH208-40P2, XH208-41P1, XH208-41P2, XH208-77P1, XH208-77P2, XH208-79P1, XH208-79P2, XI208-80P1, XH208-80P2, XH208-81P1, XH208-81P2, XH208-85P1, XH208-85P2, XH208-86P1, XH208-86P2, XH208-87P1, XH208-87P2, XH208-92P1, XH208-92P2, XH208-93P1, XH208-93P2, XH208-94P1, XH208-94P2, XH198-92P1, XH208-167p1, XH208-167p2, XH208-168p1, XH208-168p2, XH216-30p1, XH216-30p2, XH216-31p1, XH216-31p2, XH1216-33p1, XH216-33p2, XH216-57p1, XH216-57p2, XH216-58p1, XH216-58p2, XH216-59p1, XH216-59p2, XH216-60p1, XH216-60p2, XH216-76p1, XH216-76p2, XH216-77p1, XH216-77p2, XH216-100P1, XH216-100P2, XH216-118p2, XH216-119p2, XH216-125, XH216-126, XH216-127, XH216-128, XH216-129, or a compound of Examples 181-208 and analogs thereof.

In some aspects, this disclosure provides a method of treating a USP2-mediated diseases. The method includes administering to a subject in need thereof with a USP2-mediated diseases one or more compounds inhibiting USP2. The USP2-mediated disease may be a cancer or other diseases, such as virus infection, depending on the host cell receptor angiotensin-converting enzyme 2 (ACE2). The USP2-mediated disease overexpresses USP2 relative to a wild-type tissue of the same species and tissue type. The USP2-mediated diseases can have elevated USP2 enzymatic activity relative to a wildtype tissue of the same species and tissue type. Non-limiting examples of USP2-mediated cancer include mesothelioma, hepatocellular cancer, central nervous system neoplasm, lung cancer, bone cancer, pancreatic cancer, skin cancer, head and neck cancer, melanoma, ovarian cancer, colon cancer, rectal cancer, anal cancer, stomach cancer, gastrointestinal cancer, breast cancer (e.g., estrogen receptor positive (ER+) breast cancer), uterine cancer, fallopian tube cancer, endometrial cancer, cervical cancer, vaginal cancer, vulvar cancer, esophageal cancer, gastrointestinal cancer, endocrine cancer, thyroid cancer, parathyroid cancer, adrenal cancer, soft tissue sarcoma, urethral cancer, penile cancer, prostate cancer, testicular cancer, leukemia, lymphoma, bladder cancer, renal cell cancer, brain stem glioma, pituitary cancer, adrenocortical cancer, gallbladder cancer, multiple myeloma, cholangiocarcinoma, fibrosarcoma, neuroblastoma, and/or retinoblastoma. The USP2-mediated cancer can be a relapsed cancer. The USP2-mediated cancer can have been refractory to one or more previous treatments. The USP2-mediated virus infection includes, but is not limited to hCoV-NL63, SARS-CoV-1 and SARS-CoV-2.

In any of the above described methods, the inhibitor can be XH161-102, XH153-7, XH153-10, XH153-74, XH146-120, XH146-123, XH153-144, XH146-180, XH153-6, XH 153-168, XH 153-157, XH 153-148, XH 153-147, XH 153-167, XH 153-145, XH153-182, XH153-155, XH153-150, XH161-26, XH161-2, XH161-4, XH161-5, XH161-6, XH161-3, XH161-19, XH161-37, X1161-32, XH161-33, X1161-25, XH161-31, XH161-36, XH161-94, XH161-98, XH161-95, XH161-35, XH161-96, XH161-134, XH161-135, XH161-132, XH161-133, XH161-101, XH161-136, XH161-137, XH161-169, XH161-170, XH161-153, XH161-154, XH168-77, XH181-20, XH181-38, XH181-39, XH161-177, XH161-176, XH161-172, XH161-173, XH168-72-1, XH168-72-2, XH168-72-3, XH168-79, XH168-63, XH168-76, XH161-180, XH153-79-D, XH153-83, XH153-84-pl, XH153-84-p2, XH153-20, XH181-103, XH181-104, XH188-105, XH188-111, XH188-113, XH188-117, XH188-119, XH188-122, XH188-123, XH188-124, XH188-125, XH188-137, XH188-146, XH188-149, XH188-150, XH188-151, XH188-170, XH188-171, XH188-172, XH188-173, XH188-174, XH188-175, XH188-176, XH188-183, XH188-184, XH188-185, XH188-186, XH198-1, XH198-2, XH198-3, XH198-4, XH198-62, XH193-63, XH198-65, XH198-66, XH198-67, XH198-70, XH198-71, XH188-135p1, XH188-135p2, XH1198-8, XH198-9, XH198-38P1, XH198-38P2, XH198-43P1, XH198-43P2, XH198-44, XH198-45, XH198-46, XH198-48, XH198-68P1, XH198-68P2, XH198-69P1, XH198-69P2, XH208-34P1, XH208-34P2, XH208-39P1, XH208-39P2, XH208-40P1, XH208-40P2, XH208-41P1, XH208-41P2, XH208-77P1, XH208-77P2, XH208-79P1, XH208-79P2, XH208-80P1, XH208-80P2, XH208-81P1, XH208-81P2, XH208-85P1, XH208-85P2, XH208-86P1, XH208-86P2, XH208-87P1, XH208-87P2, XH208-92P1, XH208-92P2, XH208-93P1, XH208-93P2, XH208-94P1, XH208-94P2, XH198-92P1, XH208-167p1, XH208-167p2, XH208-168p1, XH208-168p2, XH216-30p1, XH216-30p2, XH216-31p1, XH216-31p2, XH216-33p1, XH216-33p2, XH216-57p1, XH216-57p2, XH216-58p1, XH216-58p2, XH216-59p1, XH216-59p2, XH216-60p1, XH216-60p2, XH216-76p1, XH216-76p2, XH216-77p1, XH216-77p2, XH216-100P1, XH216-100P2, XH216-118p2, XH216-119p2, XH216-125, XH216-126, XH216-127, X1216-128, XH216-129, or a compound of Examples 181-208 and analogs thereof.

In some embodiments of the disclosed methods, the compounds can be administered, e.g., orally, parenterally, intradermally, subcutaneously, topically, and/or rectally.

Any of the above-described methods can further include treating the subject with one or more additional therapeutic regimens for treating cancer. The one or more additional therapeutic regimens for treating cancer can be, e.g., one or more of surgery, chemotherapy, radiation therapy, hormone therapy, or immunotherapy.

As used herein, the terms “about” and “approximately” are defined as being within plus or minus 10% of a given value or state, preferably within plus or minus 5% of said value or state.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. Other features and advantages of the invention will be apparent from the following detailed description and figures, and from the claims.

### BRIEF DESCRIPTION OF THE FIGURES

FIG. 1. A schematic model showing the regulation of ACE2 expression on transcriptional and post-translational levels, respectively, as well as strategies targeting ACE2 expression modulation against viral infection.

FIG. 2a-2h. Identification of USP2 as a physiological deubiquitinase of ACE2.

FIG. 3a-3c. Screening of USP2 inhibitors by USP2 activity assays.

FIG. 4a-4c. IC₅₀ determination of selected USP2 inhibitors by USP2 activity assays.

FIG. 5a-5j. Screening of selected USP2 inhibitors in HepG2, Vero, Calu-3 and Sum159 cells by immunoblotting experiments.

FIG. 6. Screening of selected USP2 inhibitors by multiple doses in HepG2 cell line by immunoblotting experiments.

FIG. 7a-7c. MS102 showing improved efficacy in antivirus infection with less toxicity.

FIG. 8a-8l. Identification of VPRBP as a bonafide substrate of USP2.

FIG. 9. Screening of selected USP2 inhibitors by cell viability assays in H460 cell line.

FIG. 10. Screening of selected USP2 inhibitors by cell viability assays in A375 cell line.

FIG. 11. GI₅₀ determination of indicated USP2 inhibitors by cell viability assay in A375 cells.

FIG. 12. Screening of selected USP2 inhibitors in H460 cell line by immunoblotting experiments.

FIG. 13. Screening of selected USP2 inhibitors in A375 cell line by immunoblotting experiments.

FIG. 14a-14e. PK results of selected compounds.

### DETAILED DESCRIPTION

### Synthesis and Testing of Compounds

The activity of novel synthesized compounds can be assessed using standard biochemical assays (based on DUB Activity Assay Kit (701490, Cayman)). Cellular assays can then be used to assess the compound's ability to reduce ACE2 and cyclin D1 level, block virus infection, increase p53 and p21 level, and inhibit cancer cell proliferation. Assays suitable for use in any or all of these steps are known in the art, and include, e.g., Western blotting, quantitative mass spectrometry (MS) analysis, flow cytometry, enzymatic inhibition, cell growth inhibition and xenograft and PDX models. Suitable cell lines for use in any or all of these steps are known in the art and include, H460, HepG2, A375, SUM159, A549, H2228, H1975, H1299, H157, H1650, CAL-120, SUM149, MDA-MB-157, HCC38, HCC1143, HCC1806, MDA-MB-468, BT-549, AU565, BT-474, HCC1569, ZR-75-1, CAMA-1, HCC1428, 786-0, DLD-1, RCC4 LIM2405, RKO, DiFi, SW480, Lim1215, LoVo, LS411N, SW1463, SW48, SNU-C2B, HCT-8, SW837, Calu-1, Calu-3, Vero, 16HBE14o- and Beas-2B. Suitable mouse models for use in any or all of these steps are known in the art and include patient-derived xenograft models of triple negative breast cancer.

By way of non-limiting example, detailed synthesis protocols are described in the Examples for specific exemplary USP2 inhibitors.

Pharmaceutically acceptable isotopic variations of the compounds disclosed herein are contemplated and can be synthesized using conventional methods known in the art or methods corresponding to those described in the Examples (substituting appropriate reagents with appropriate isotopic variations of those reagents). Specifically, an isotopic variation is a compound in which at least one atom is replaced by an atom having the same atomic number, but an atomic mass different from the atomic mass usually found in nature. Useful isotopes are known in the art and include, for example, isotopes of hydrogen, carbon, nitrogen, oxygen, phosphorus, sulfur, fluorine, and chlorine. Exemplary isotopes thus include, e.g., ²H, ³H, ¹³C, ¹⁴C, ¹N, ¹⁷O, ¹⁸O, ³²P, ³⁵S, ¹⁸F, and ³⁶Cl.

Isotopic variations (e.g., isotopic variations containing ²H) can provide therapeutic advantages resulting from greater metabolic stability, e.g., increased in vivo half-life or reduced dosage requirements. In addition, certain isotopic variations (particularly those containing a radioactive isotope) can be used in drug or substrate tissue distribution studies. The radioactive isotopes tritium (³H) and carbon-14 (¹⁴C) are particularly useful for this purpose in view of their ease of incorporation and ready means of detection.

Pharmaceutically acceptable solvates of the compounds disclosed herein are contemplated. A solvate can be generated, e.g., by substituting a solvent used to crystallize a compound disclosed herein with an isotopic variation (e.g., D₂O in place of H₂O, d₆-acetone in place of acetone, or d₆-DMSO in place of DMSO).

Pharmaceutically acceptable fluorinated variations of the compounds disclosed herein are contemplated and can be synthesized using conventional methods known in the art or methods corresponding to those described in the Examples (substituting appropriate reagents with appropriate fluorinated variations of those reagents). Specifically, a fluorinated variation is a compound in which at least one hydrogen atom is replaced by a fluoro atom. Fluorinated variations can provide therapeutic advantages resulting from greater metabolic stability, e.g., increased in vivo half-life or reduced dosage requirements.

Pharmaceutically acceptable prodrugs of the compounds disclosed herein are contemplated and can be synthesized using conventional methods known in the art or methods corresponding to those described in the Examples (e.g., converting hydroxyl groups or carboxylic acid groups to ester groups). As used herein, a “prodrug” refers to a compound that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e., an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

### Characterization of Exemplary USP2 Inhibitors

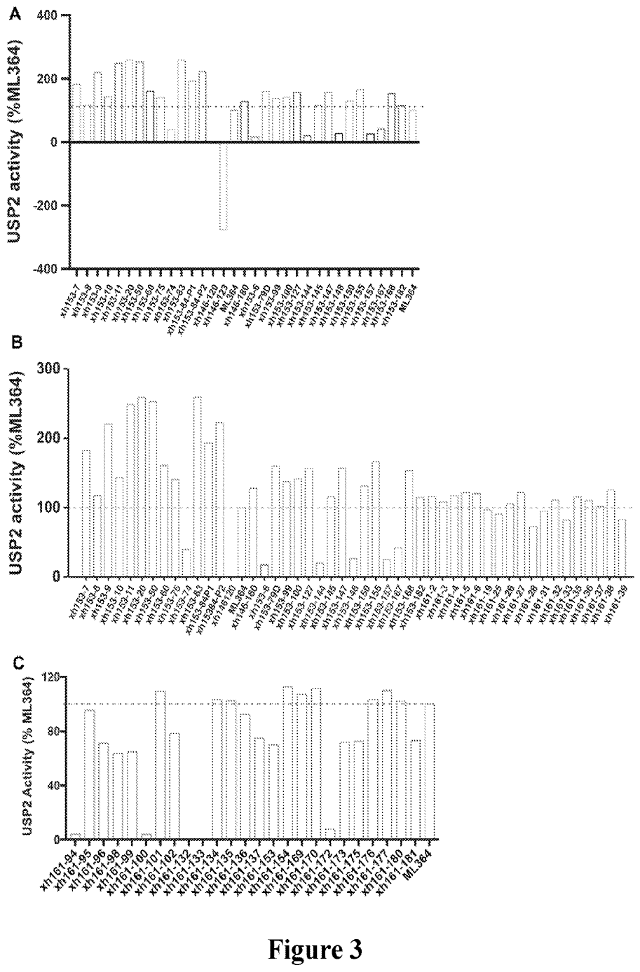

Specific exemplary USP2 inhibitors were characterized in biochemical assays, WB assays, cellular virus infection assays, cell viability assays and pharmacokinetic (PK) studies (Examples 211-215 and 217-222, FIGS. 3-7 and 9-14).

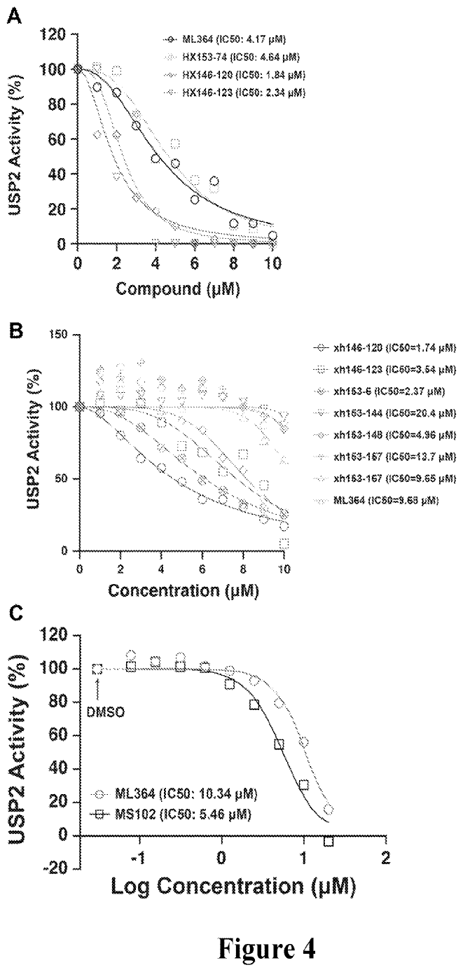

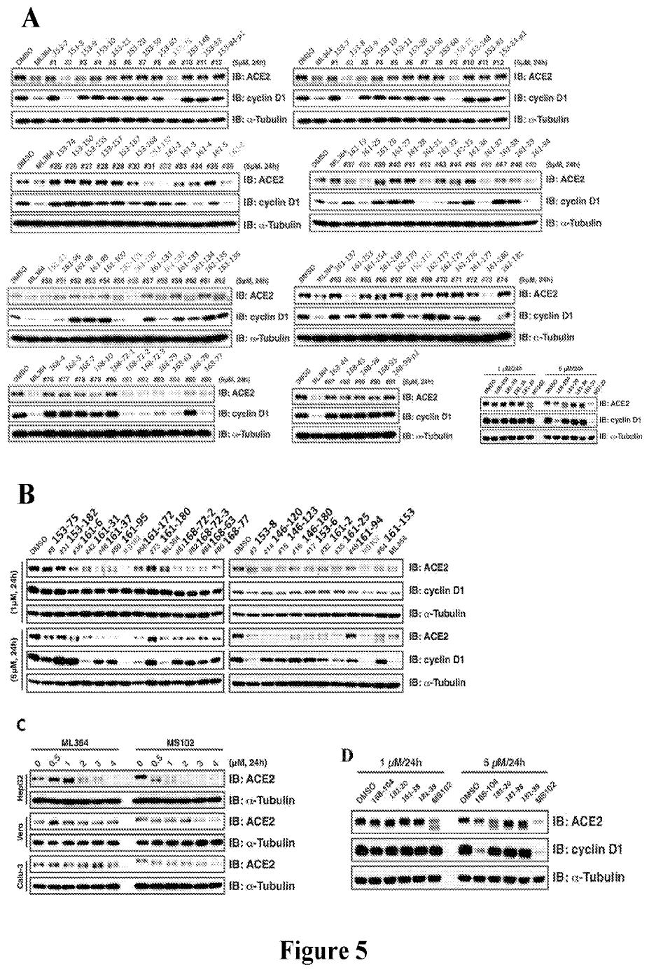

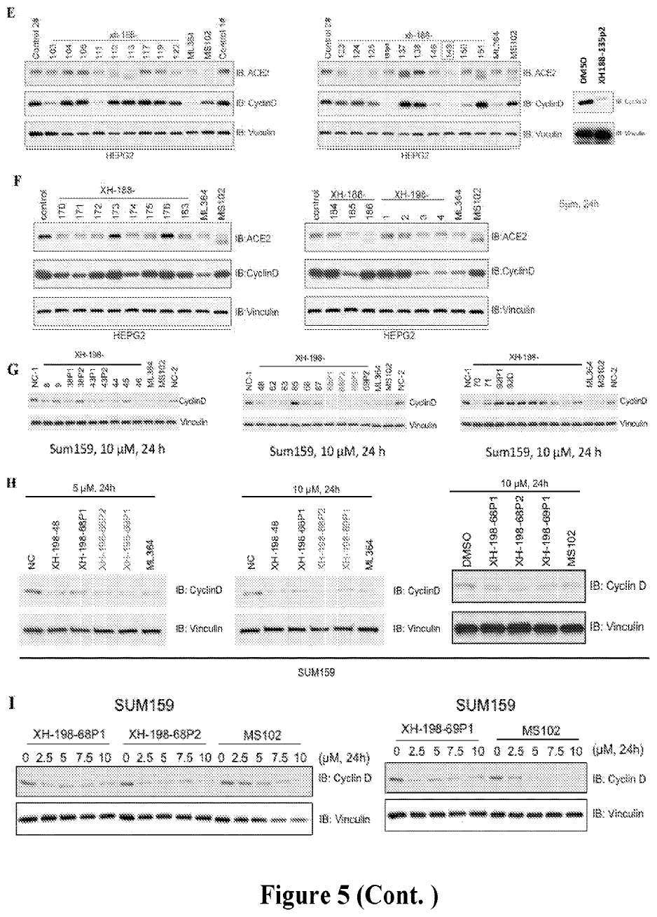

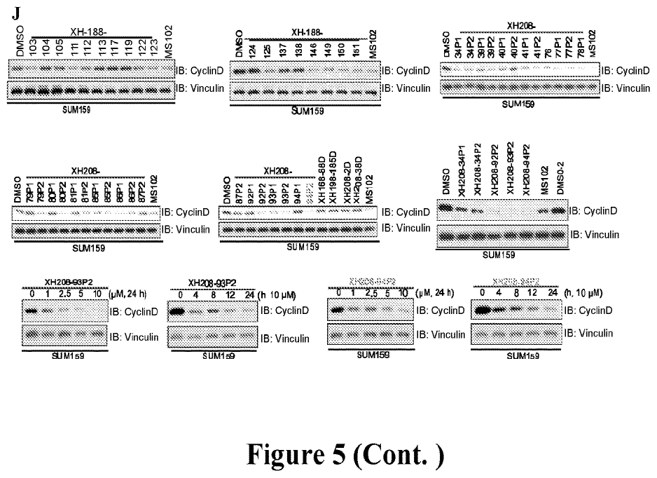

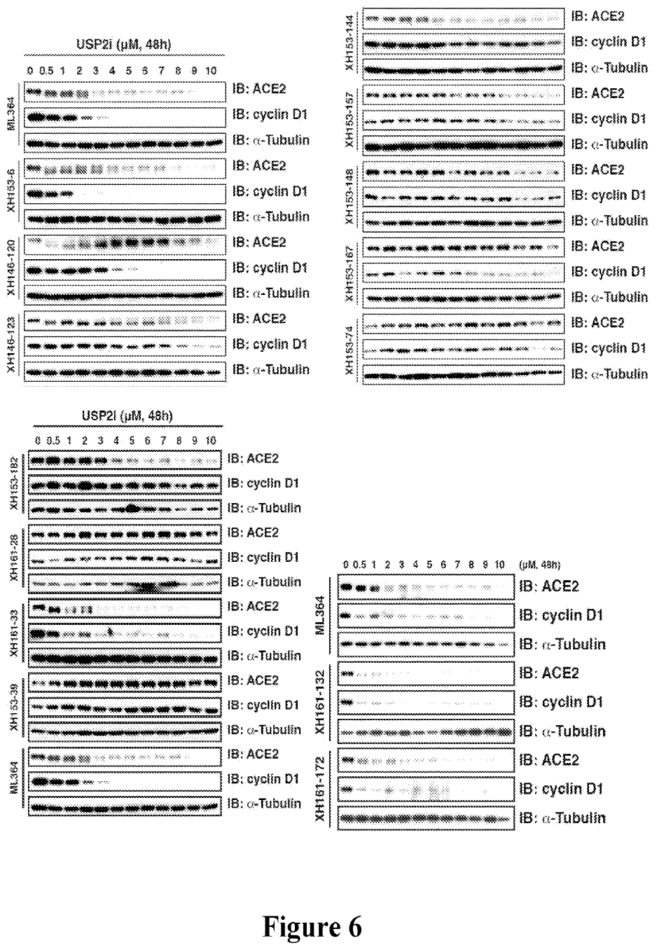

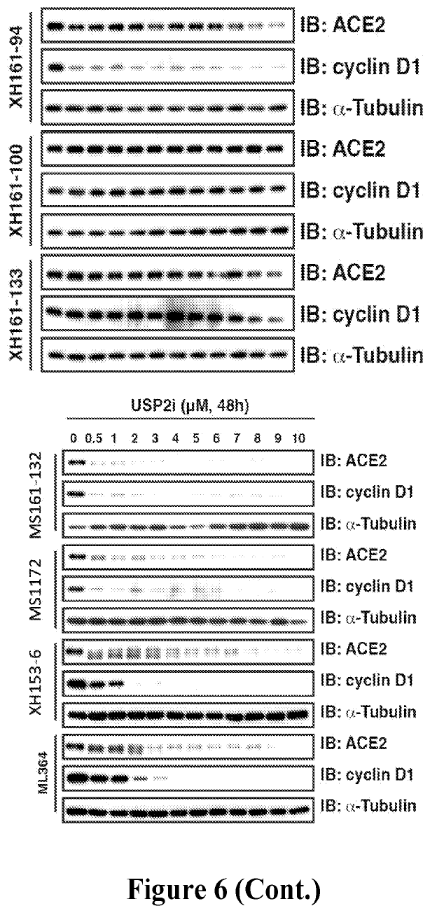

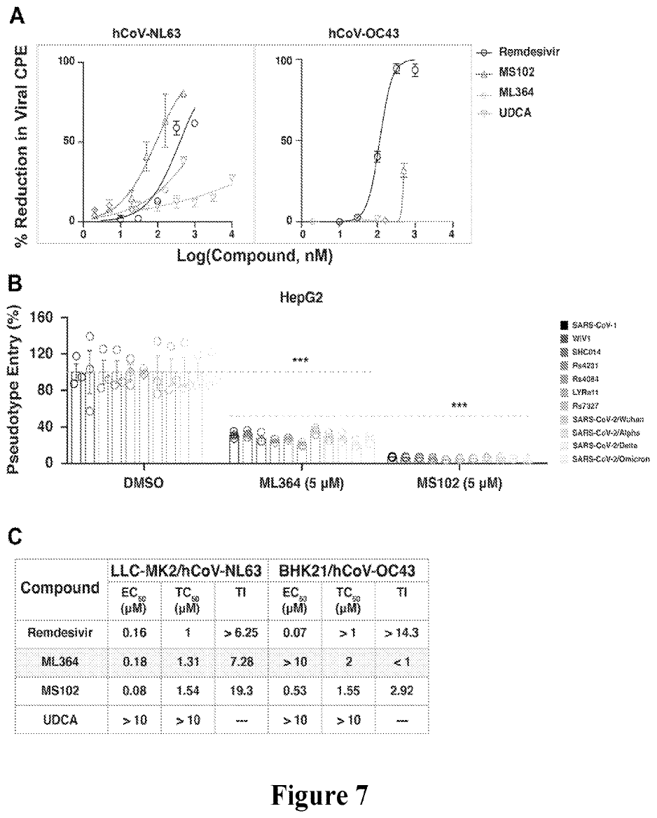

180 novel compounds (examples 1-180) were screened using USP2 enzymatic assays (FIG. 3, example 211) at 5 M concentration, and examined cyclin D1 and ACE2 protein abundance in HepG2, or SUM159 cells using immunoblotting experiments (FIG. 5-6, example 213-214). XH161-102 (MS102) (example 1) was identified as the leading compound, which can potent inhibit the USP enzymatic activity with IC50 of 5 μM (FIG. 4, example 212) and dose-dependent reduce ACE2 protein level (FIG. 5, example 213). In the cellular virus infection assays, XH161-102 (MS102) exhibited enhanced activity in preventing cell entry of ACE2-dependent pseudotypes tested than the reported USP2 inhibitor ML364 (FIG. 7, example 215).

In the cytopathic effect assay (CPE) using authentic hCoV-NL63, an ACE2-dependent coronavirus, and hCoV-OC43, an ACE2-independent viral strain, MS102 was confirmed active at inhibiting hCoV-NL63 and not active against hCoV-OC43 at the concentration evaluated (FIG. 7, example 215).

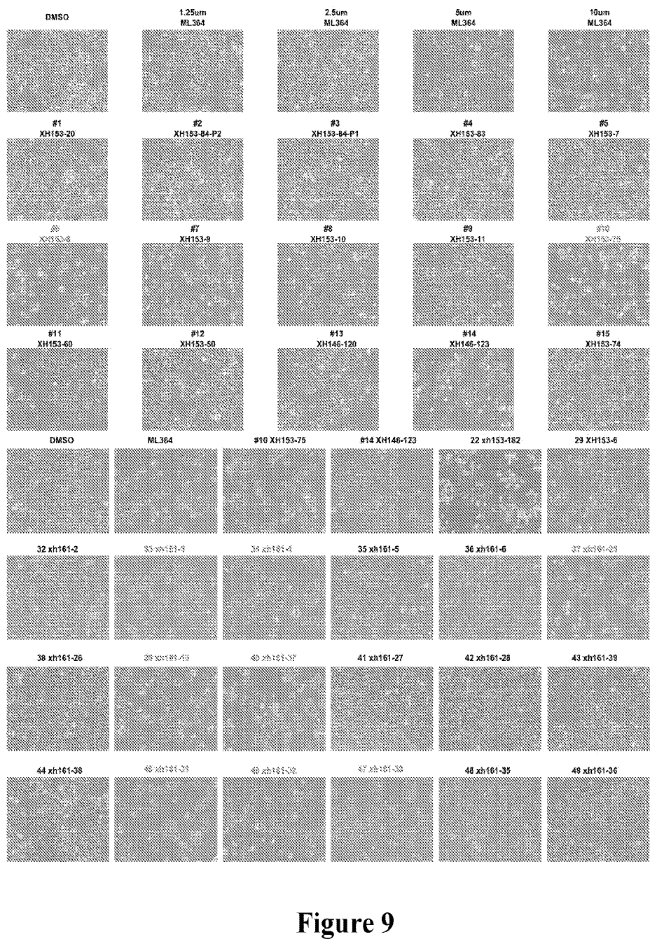

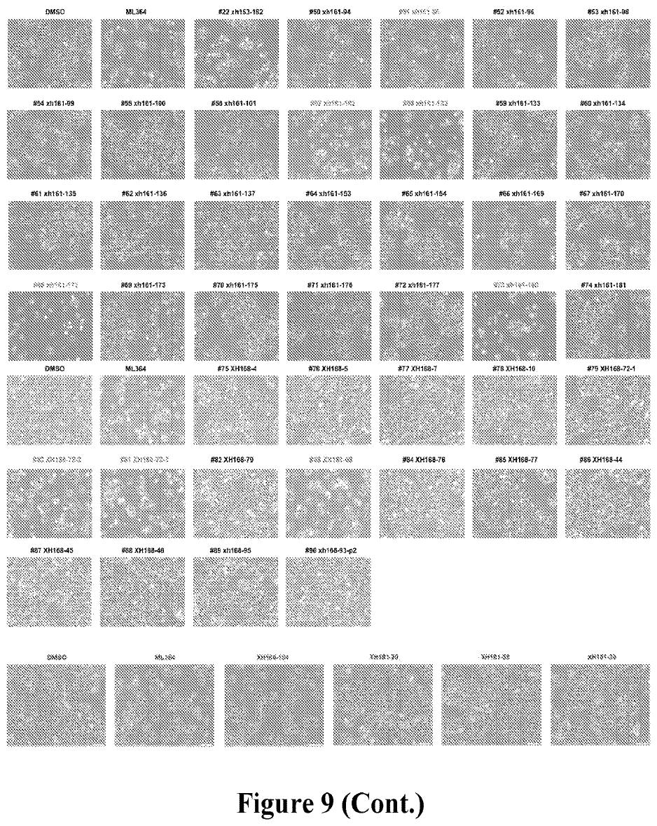

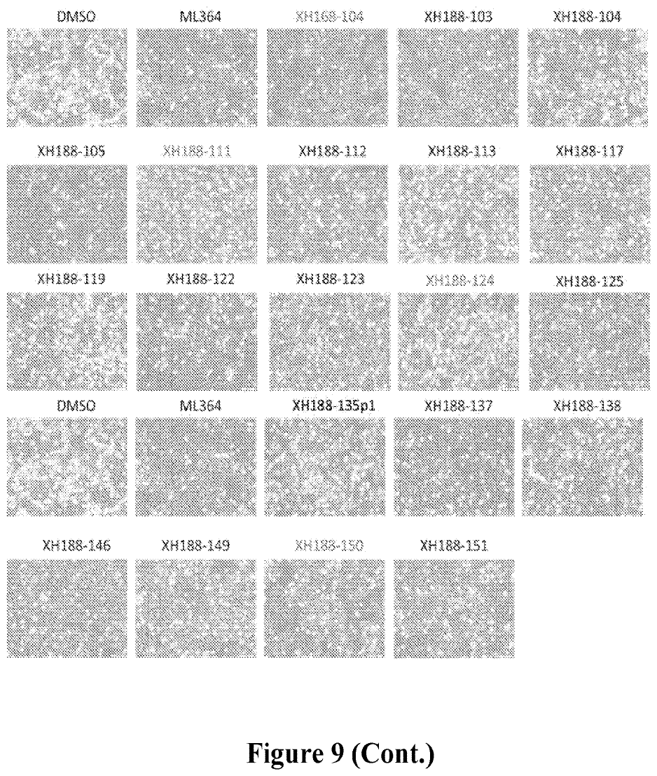

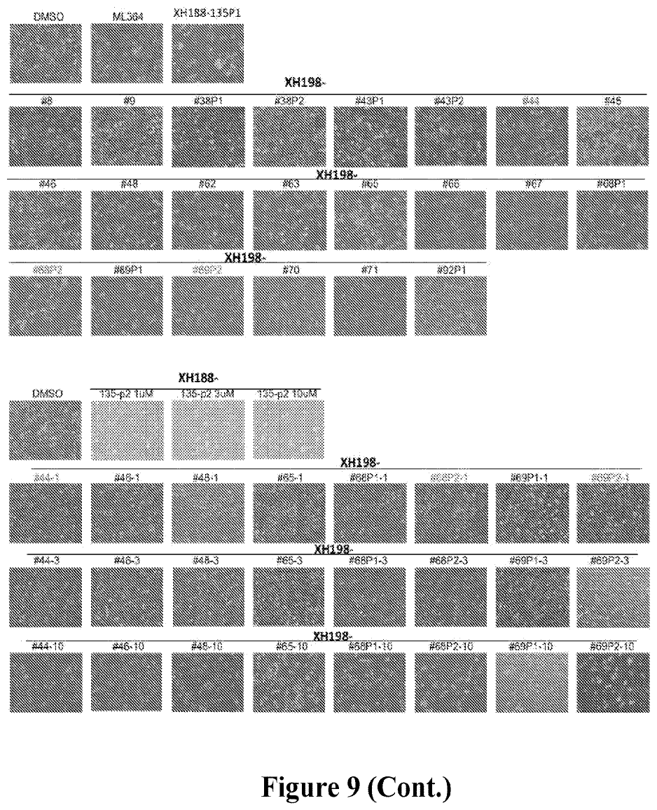

The same batch of compounds were also screened in cell viability assays and immunoblotting assay. MS102, XH153-182, MS1172, MS1180, XH188-135p1, XH188-135p2, XH198-68p2, and XH208-93p2 were identified as the leading compounds by antiproliferation effect and elevating tumor suppressors P53 and P21 protein levels (FIGS. 9-13, examples 217-221).

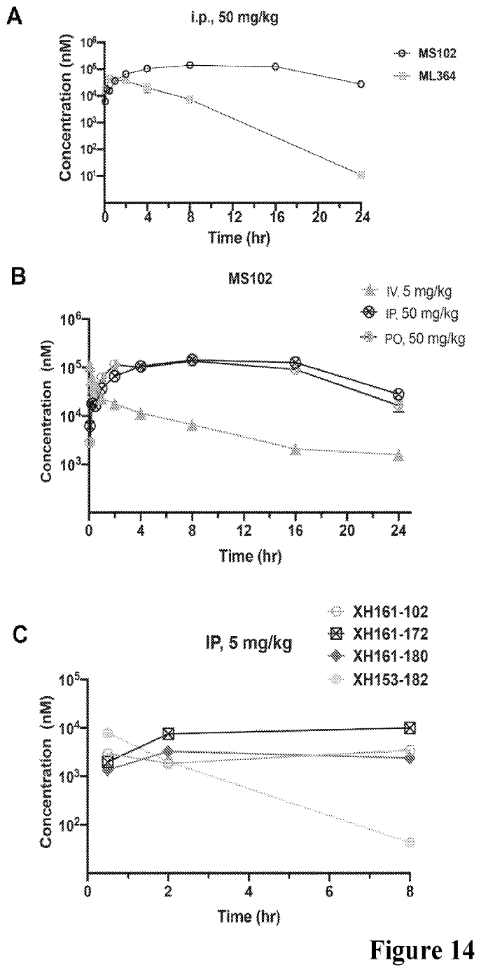

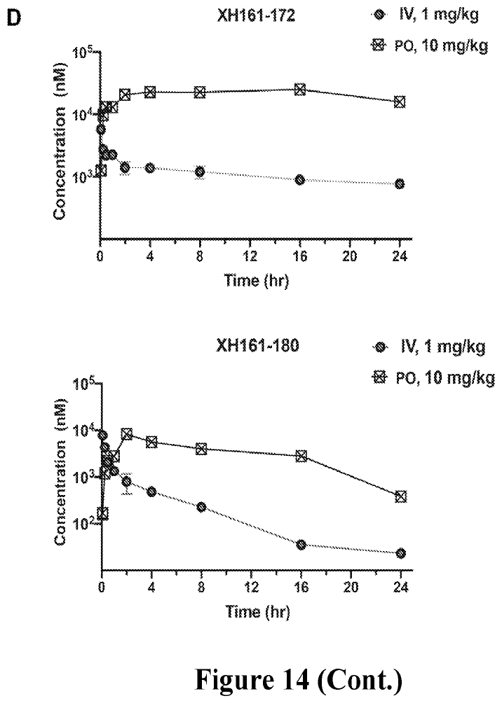

In in vivo mouse PK studies, MS102 displayed much higher peak plasma concentration (approximately 3 times over ML364) and enhanced exposure (approximately 2,500 times over ML364 at 24 hours post-administration). MS1172 and MS1180 also showed similar plasma exposure level as MS102 at the same dosage under i.p. injection condition. All three compounds are orally bioavailable in mouse PK studies, with 100% oral bioavailability for MS102 and MS1172, and 95% oral bioavailability for MS1180 (FIG. 14, example 222).

### Definition of Terms

“Alkyl” refers to a straight or branched hydrocarbon chain radical consisting solely of carbon and hydrogen atoms, containing no unsaturation. An alkyl may comprise one, two, three, four, five, six, seven, eight, nine, ten, eleven, twelve, thirteen, fourteen, fifteen, or sixteen carbon atoms. In certain embodiments, an alkyl comprises one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), pentyl, 3-methylhexyl, 2-methylhexyl, and the like.

“Alkylene” refers to a bivalent saturated aliphatic radical (such as ethylene) regarded as derived from an alkene by opening of the double bond or from an alkane by removal of two hydrogen atoms from different carbon atoms.

“Alkenyl” refers to a straight or branched hydrocarbon chain radical group consisting solely of carbon and hydrogen atoms, containing at least one double bond. An alkenyl may comprise two, three, four, five, six, seven, eight, nine, ten, eleven, twelve, thirteen, fourteen, fifteen, or sixteen carbon atoms. In certain embodiments, an alkenyl comprises two to twelve carbon atoms (e.g., C₂-C₁₂ alkenyl). In certain embodiments, an alkenyl comprises two to eight carbon atoms (e.g., C₂-C₈ alkenyl). In certain embodiments, an alkenyl comprises two to six carbon atoms (e.g., C₂-C₆ alkenyl). In other embodiments, an alkenyl comprises two to four carbon atoms (e.g., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

The term “alkenylene” refers to linear or branched-chain divalent hydrocarbon radical of two to eight carbon atoms (C₂-C₈) with at least one site of unsaturation, i.e., a carbon-carbon, SP2 double bond, wherein the alkenylene radical may be optionally substituted independently with one or more substituents described herein, and includes radicals having “cis” and “trans” orientations, or alternatively, “E” and “Z” orientations. Examples include, but are not limited to, ethynylene or vinylene (—CH═CH—), allyl (—CH₂CH═CH—), and the like.

The term “allyl,” as used herein, means a —CH₂CH═CH₂ group.

As used herein, the term “alkynyl” refers to a straight or branched hydrocarbon chain radical group consisting solely of carbon and hydrogen atoms, containing at least one triple bond. An alkynyl may comprise two, three, four, five, six, seven, eight, nine, ten, eleven, twelve, thirteen, fourteen, fifteen, or sixteen carbon atoms. In certain embodiments, an alkynyl comprises two to twelve carbon atoms (e.g., C₂-C₁₂ alkynyl). In certain embodiments, an alkynyl comprises two to eight carbon atoms (e.g., C₂-C₈ alkynyl). In other embodiments, an alkynyl has two to six carbon atoms (e.g., C₂-C₆ alkynyl). In other embodiments, an alkynyl has two to four carbon atoms (e.g., C₂-C₄ alkynyl). The alkynyl is attached to the rest of the molecule by a single bond. Examples of such groups include, but are not limited to, ethynyl, propynyl, 1-butynyl, 2-butynyl, 1-pentynyl, 2-pentynyl, 1-hexynyl, 2-hexynyl, 3-hexynyl, and the like.

The term “alkynylene” refers to a linear or branched divalent hydrocarbon radical of two to eight carbon atoms (C₂-C₈) with at least one site of unsaturation, i.e., a carbon-carbon, sp triple bond, wherein the alkynylene radical may be optionally substituted independently with one or more substituents described herein. Examples include, but are not limited to, ethynylene (—C≡C—), propynylene (propargylene, —CH₂C≡C—), and the like.

The term “alkoxy”, as used herein, means an alkyl group as defined herein which is attached to the rest of the molecule via an oxygen atom. Examples of such groups include, but are not limited to, methoxy, ethoxy, n-propyloxy, iso-propyloxy, n-butoxy, iso-butoxy, tert-butoxy, pentyloxy, hexyloxy, and the like.

The term “heteroalkyl”, as used herein, means a saturated or unsaturated chain of carbon atoms and at least one heteroatom, wherein no two heteroatoms are adjacent.

The term “aryl”, as used herein, “refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon atoms. An aryl may comprise from six to eighteen carbon atoms, where at least one of the rings in the ring system is fully unsaturated, i.e., it contains a cyclic, delocalized (4n+2) π-electron system in accordance with the Hickel theory. In certain embodiments, an aryl comprises six to fourteen carbon atoms (C₆-C₁₄ aryl). In certain embodiments, an aryl comprises six to ten carbon atoms (C₆-C₁₀ aryl). Examples of such groups include, but are not limited to, phenyl, fluorenyl and naphthyl. The terms “Ph” and “phenyl,” as used herein, mean a —C₆H₅ group.

The term “arylene” means a divalent aromatic hydrocarbon radical of 6-20 carbon atoms (C₆-C₂₀) derived by the removal of two hydrogen atom from two carbon atoms of a parent aromatic ring system. Some arylene groups are represented in the exemplary structures as “Ar”. Arylene includes bicyclic radicals comprising an aromatic ring fused to a saturated, partially unsaturated ring, or aromatic carbocyclic ring. Typical arylene groups include, but not limited to, radicals derived from benzene (phenylene), substituted benzenes, naphthalene, anthracene, biphenylene indenylene, indenylene, 1,2-dihydronaphthalene, 1,2,3,4,-tetrahydronaphthyl, and the like. Arylene groups are optionally substituted with one or more substituents described herein.

The term “phenylene” means any of three bivalent radicals C₆H₄ derived from benzene by removal of two hydrogen atoms from the ortho, meta, or para positions.

The term “heteroaryl”, refers to a radical derived from a 3- to 18-membered aromatic ring radical that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Hickel theory. Heteroaryl includes fused or bridged ring systems. In certain embodiments, a heteroaryl refers to a radical derived from a 3- to 10-membered aromatic ring radical (3-10 membered heteroaryl). In certain embodiments, a heteroaryl refers to a radical derived from 5- to 7-membered aromatic ring (5-7 membered heteroaryl). Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of such groups include, but not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. In certain embodiments, an heteroaryl is attached to the rest of the molecule via a ring carbon atom. In certain embodiments, an heteroaryl is attached to the rest of the molecule via a nitrogen atom (N-attached) or a carbon atom (C-attached). For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

The term “heterocyclic”, as used herein, means a non-aromatic, monocyclic, bicyclic, tricyclic, or tetracyclic radical having a total of from 4, 5, 6, 7, 8, 9, 10, 11, 12, or 13 atoms in its ring system, and containing from 3 to 12 carbon atoms and from 1 to 4 heteroatoms each independently selected from O, S and N, and with the proviso that the ring of said group does not contain two adjacent O atoms or two adjacent S atoms. A heterocyclic group may include fused, bridged or spirocyclic ring systems. In certain embodiments, a heterocyclic group comprises 3 to 10 ring atoms (3-10 membered heterocyclic). In certain embodiments, a heterocyclic group comprises 3 to 8 ring atoms (3-8 membered heterocyclic). In certain embodiments, a heterocyclic group comprises 4 to 8 ring atoms (4-8 membered heterocyclic). In certain embodiments, a heterocyclic group comprises 3 to 6 ring atoms (3-6 membered heterocyclic). A heterocyclic group may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a heterocyclic group contains a sulfur atom, said sulfur atom may be oxidized with one or two oxygen atoms to afford either a sulfoxide or sulfone. An example of a 4 membered heterocyclic group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such heterocyclic groups include, but are not limited to, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, 3-oxopiperazinyl, 4-methylpiperazinyl, 4-ethylpiperazinyl, and 1-oxo-2,8,diazaspiro[4.5]dec-8-yl. A heteroaryl group may be attached to the rest of molecular via a carbon atom (C-attached) or a nitrogen atom (N-attached). For instance, a group derived from piperazine may be piperazin-1-yl (N-attached) or piperazin-2-yl (C-attached).

The term “cycloalkyl” means a saturated, monocyclic, bicyclic, tricyclic, or tetracyclic radical having a total of from 4, 5, 6, 7, 8, 9, 10, 11, 12, or 13 carbon atoms in its ring system. A cycloalkyl may be fused, bridged or spirocyclic. In certain embodiments, a cycloalkyl comprises 3 to 8 carbon ring atoms (C₃-C₈ cycloalkyl). In certain embodiments, a cycloalkyl comprises 3 to 6 carbon ring atoms (C₃-C₆ cycloalkyl). Examples of such groups include, but are not limited to, cyclopropyl, cyclobutyl, cyclopentyl, cyclopentenyl, cyclohexyl, cycloheptyl, adamantyl, and the like.

The term “cycloalkylene” is a bidentate radical obtained by removing a hydrogen atom from a cycloalkyl ring as defined above. Examples of such groups include, but are not limited to, cyclopropylene, cyclobutylene, cyclopentylene, cyclopentenylene, cyclohexylene, cycloheptylene, and the like.

The term “spirocyclic” as used herein has its conventional meaning, that is, any ring system containing two or more rings wherein two of the rings have one ring carbon in common. Each ring of the spirocyclic ring system, as herein defined, independently comprises 3 to 20 ring atoms. Preferably, they have 3 to 10 ring atoms. Non-limiting examples of a spirocyclic system include spiro[3.3]heptane, spiro[3.4]octane, and spiro[4.5]decane.

The term cyano” refers to a —C≡N group.

An “aldehyde” group refers to a —C(O)H group.

An “alkoxy” group refers to both an —O-alkyl, as defined herein.

An “alkoxycarbonyl” refers to a —C(O)-alkoxy, as defined herein.

An “alkylaminoalkyl” group refers to an -alkyl-NR-alkyl group, as defined herein.

An “alkylsulfonyl” group refer to a —SO₂ alkyl, as defined herein.

An “amino” group refers to an optionally substituted —NH₂.

An “aminoalkyl” group refers to an -alky-amino group, as defined herein.

An “aminocarbonyl” refers to a —C(O)-amino, as defined herein.

An “arylalkyl” group refers to -alkylaryl, where alkyl and aryl are defined herein.

An “aryloxy” group refers to both an —O-aryl and an —O-heteroaryl group, as defined herein.

An “aryloxycarbonyl” refers to —C(O)-aryloxy, as defined herein.

An “arylsulfonyl” group refers to a —SO₂ aryl, as defined herein.

A “carbonyl” group refers to a —C(O)— group, as defined herein.

A “carboxylic acid” group refers to a —C(O)OH group.

A “cycloalkoxy” refers to a —O-cycloalkyl group, as defined herein.

A “halo” or “halogen” group refers to fluorine, chlorine, bromine or iodine.

A “haloalkyl” group refers to an alkyl group substituted with one or more halogen atoms.

A “hydroxy” group refers to an —OH group.

A “nitro” group refers to a —NO₂ group.

An “oxo” group refers to the ═O substituent.

A “trihalomethyl” group refers to a methyl substituted with three halogen atoms.

A “electron withdrawing” group refers to an atom or group that draws electron density from neighboring atoms towards itself.

The term “substituted,” means that the specified group or moiety bears one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “null” means the absence of an atom or moiety, and there is a bond between adjacent atoms in the structure.

The term “optionally substituted” means that the specified group may be either unsubstituted or substituted by one or more substituents as defined herein. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one at which the remainder of the compound of the present invention is attached to and an additional substituent, remaining 4 positions open). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies.

As used herein, when m (or n or x) is defined by a range, for example, “m is 0 to 15” or “m=0-3” mean that m is an integer from 0 to 15 (i.e. m is 0, 1, 2, 3, 4, 5, 6, 7, 8, 9, 10, 11, 12, 13, 14, or 15) or m is an integer from 0 to 3 (i.e. m is 0, 1,2, or 3) or is any integer in the defined range.

“Pharmaceutically acceptable salt” includes both acid and base addition salts. A pharmaceutically acceptable salt of any one of the bivalent compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, the same symbol used in different formulae has a definition linked to that formula. For example, the definition of R¹ in Formula 1 is as defined with respect to Formula 1 and the definition of R¹ in Formula 6 is as defined with respect to Formula 6.

### Pharmaceutical Compositions

In some aspects, the compositions and methods described herein include the manufacture and use of pharmaceutical compositions and medicaments that include one or more bivalent compounds as disclosed herein. Also included are the pharmaceutical compositions themselves.

In some aspects, the compositions disclosed herein can include other compounds, drugs, or agents used for the treatment of cancer. For example, in some instances, pharmaceutical compositions disclosed herein can be combined with one or more (e.g., one, two, three, four, five, or less than ten) compounds. Such additional compounds can include, e.g., conventional chemotherapeutic agents known in the art. When co-administered, USP2 inhibitors disclosed herein can operate in conjunction with conventional chemotherapeutic agents to produce mechanistically additive or synergistic therapeutic effects.

In some aspects, the pH of the compositions disclosed herein can be adjusted with pharmaceutically acceptable acids, bases, or buffers to enhance the stability of the USP2 inhibitors or its delivery form.

Pharmaceutical compositions typically include a pharmaceutically acceptable carrier, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable carrier, adjuvant, or vehicle is a composition that can be administered to a patient, together with a compound of the invention, and which does not destroy the pharmacological activity thereof and is nontoxic when administered in doses sufficient to deliver a therapeutic amount of the compound. Exemplary conventional nontoxic pharmaceutically acceptable carriers, adjuvants, and vehicles include saline, solvents, dispersion media, coatings, antibacterial and antifungal agents, isotonic and absorption delaying agents, and the like, compatible with pharmaceutical administration.

In particular, pharmaceutically acceptable carriers, adjuvants, and vehicles that can be used in the pharmaceutical compositions of this invention include, but are not limited to, ion exchangers, alumina, aluminum stearate, lecithin, self-emulsifying drug delivery systems (SEDDS) such as d-a-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

As used herein, the USP2 inhibitors disclosed herein are defined to include pharmaceutically acceptable derivatives or prodrugs thereof. A “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein. Such derivatives are recognizable to those skilled in the art without undue experimentation. Nevertheless, reference is made to the teaching of Burger's Medicinal Chemistry and Drug Discovery, 5^{th} Edition, Vol. 1: Principles and Practice, which is incorporated herein by reference to the extent of teaching such derivatives.

The USP2 inhibitors disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivatives thereof. USP2 inhibitors disclosed herein include, e.g., those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate, trifluoromethylsulfonate, and undecanoate. Salts derived from appropriate bases include, e.g., alkali metal (e.g., sodium), alkaline earth metal (e.g., magnesium), ammonium and N-(alkyl)4+ salts. The invention also envisions the quaternization of any basic nitrogen-containing groups of the USP2 inhibitors disclosed herein. Water or oil-soluble or dispersible products can be obtained by such quaternization.

In some aspects, the pharmaceutical compositions disclosed herein can include an effective amount of one or more USP2 inhibitors. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment or prevention of cell growth, cell proliferation, or cancer). In some aspects, pharmaceutical compositions can further include one or more additional compounds, drugs, or agents used for the treatment of cancer (e.g., conventional chemotherapeutic agents) in amounts effective for causing an intended effect or physiological outcome (e.g., treatment or prevention of cell growth, cell proliferation, or cancer).

In some aspects, the pharmaceutical compositions disclosed herein can be formulated for sale in the United States, import into the United States, or export from the United States.

### Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM) (available at http://www.fda.gov/Drugs/DevelopmentApprovalProcess/

FormsSubmissionRequirements/ElectronicSubmissions/DataStandardsManualmonograph s). In particular, the pharmaceutical compositions can be formulated for and administered via oral, parenteral, or transdermal delivery. The term “parenteral” as used herein includes subcutaneous, intracutaneous, intravenous, intramuscular, intraperitoneal, intra-articular, intra-arterial, intrasynovial, intrasternal, intrathecal, intralesional, and intracranial injection or infusion techniques.

For example, the pharmaceutical compositions disclosed herein can be administered, e.g., topically, rectally, nasally (e.g., by inhalation spray or nebulizer), buccally, vaginally, subdermally (e.g., by injection or via an implanted reservoir), or ophthalmically.

For example, pharmaceutical compositions of this invention can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening, flavoring, or coloring agents can be added.

For example, the pharmaceutical compositions of this invention can be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

For example, the pharmaceutical compositions of this invention can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, or other solubilizing or dispersing agents known in the art.

For example, the pharmaceutical compositions of this invention can be administered by injection (e.g., as a solution or powder). Such compositions can be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil can be employed, including synthetic mono- or diglycerides. Fatty acids, such as oleic acid and its glyceride derivatives are useful in the preparation of injectables, as are natural pharmaceutically-acceptable oils, e.g., olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, or other similar emulsifying agents or bioavailability enhancers which are commonly used in the manufacture of pharmaceutically acceptable solid, liquid, or other dosage forms can also be used for the purposes of formulation.

In some aspects, an effective dose of a pharmaceutical composition of this invention can include, but is not limited to, e.g., about 0.00001, 0.0001, 0.001, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, 3, 4, 5, 6, 7, 8, 9, 10, 15, 20, 30, 40, 50, 60, 70, 80, 90, 100, 200, 300, 400, 500, 600, 700, 800, 900, 1000, 2500, 5000, or 10000 mg/kg/day, or according to the requirements of the particular pharmaceutical composition.

When the pharmaceutical compositions disclosed herein include a combination of a compound of the formulae described herein (e.g., an USP2 inhibitors) and one or more additional compounds (e.g., one or more additional compounds, drugs, or agents used for the treatment of cancer or any other condition or disease, including conditions or diseases known to be associated with or caused by cancer), both the compound and the additional compound should be present at dosage levels of between about 1 to 100%, and more preferably between about 5 to 95% of the dosage normally administered in a monotherapy regimen. The additional agents can be administered separately, as part of a multiple dose regimen, from the compounds of this invention. Alternatively, those agents can be part of a single dosage form, mixed together with the compounds of this invention in a single composition.

In some aspects, the pharmaceutical compositions disclosed herein can be included in a container, pack, or dispenser together with instructions for administration.

### Methods of Treatment

The methods disclosed herein contemplate administration of an effective amount of a compound or composition to achieve the desired or stated effect. Typically, the compounds or compositions of the invention will be administered from about 1 to about 6 times per day or, alternately or in addition, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations can contain from about 20% to about 80% active compound.

In some aspects, the present disclosure provides methods for using a composition comprising a USP2 inhibitor, including pharmaceutical compositions (indicated below as ‘X’) disclosed herein in the following methods:

Substance X for use as a medicament in the treatment of one or more diseases or conditions disclosed herein (e.g., cancer, referred to in the following examples as ‘Y’). Use of substance X for the manufacture of a medicament for the treatment of Y; and substance X for use in the treatment of Y.

In some aspects, the methods disclosed include the administration of a therapeutically effective amount of one or more of the compounds or compositions described herein to a subject (e.g., a mammalian subject, e.g., a human subject) who is in need of, or who has been determined to be in need of, such treatment. In some aspects, the methods disclosed include selecting a subject and administering to the subject an effective amount of one or more of the compounds or compositions described herein, and optionally repeating administration as required for the prevention or treatment of cancer.

In some aspects, subject selection can include obtaining a sample from a subject (e.g., a candidate subject) and testing the sample for an indication that the subject is suitable for selection. In some aspects, the subject can be confirmed or identified, e.g., by a health care professional, as having had or having a condition or disease. In some aspects, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some aspects, exhibition of a positive immune response towards a condition or disease can be made from patient records, family history, or detecting an indication of a positive immune response. In some aspects, multiple parties can be included in subject selection. For example, a first party can obtain a sample from a candidate subject and a second party can test the sample. In some aspects, subjects can be selected or referred by a medical practitioner (e.g., a general practitioner). In some aspects, subject selection can include obtaining a sample from a selected subject and storing the sample or using the in the methods disclosed herein. Samples can include, e.g., cells or populations of cells.

In some aspects, methods of treatment can include a single administration, multiple administrations, and repeating administration of one or more compounds disclosed herein as required for the prevention or treatment of the disease or condition from which the subject is suffering (e.g., an USP2-mediated disease). In some aspects, methods of treatment can include assessing a level of disease in the subject prior to treatment, during treatment, or after treatment. In some aspects, treatment can continue until a decrease in the level of disease in the subject is detected.

The term “subject,” as used herein, refers to any animal. In some instances, the subject is a mammal. In some instances, the term “subject,” as used herein, refers to a human (e.g., a man, a woman, or a child).

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any manner in which one or more of the symptoms of a disease or disorder (e.g., cancer) are ameliorated or otherwise beneficially altered. As used herein, amelioration of the symptoms of a particular disorder (e.g., cancer) refers to any lessening, whether permanent or temporary, lasting or transient that can be attributed to or associated with treatment by the compositions and methods of the present invention. In some embodiments, treatment can promote or result in, for example, a decrease in the number of tumor cells (e.g., in a subject) relative to the number of tumor cells prior to treatment; a decrease in the viability (e.g., the average/mean viability) of tumor cells (e.g., in a subject) relative to the viability of tumor cells prior to treatment; a decrease in the rate of growth of tumor cells; a decrease in the rate of local or distant tumor metastasis; or reductions in one or more symptoms associated with one or more tumors in a subject relative to the subject's symptoms prior to treatment.

As used herein, the term “treating cancer” means causing a partial or complete decrease in the rate of growth of a tumor, and/or in the size of the tumor and/or in the rate of local or distant tumor metastasis, and/or the overall tumor burden in a subject, and/or any decrease in tumor survival, in the presence of an inhibitor (e.g., an USP2 inhibitor) described herein.

As used herein, the term “virus infection” means when an organism's body is invaded by pathogenic viruses and infectious virus particle attach to and enter susceptible cells.

The terms “prevent,” “preventing,” “prevention,” “block”, and “blocking” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present invention. Exemplary USP2-mediated diseases that can be treated with USP2 inhibitors include, for example, virus infection, breast cancer, ovarian cancer, prostate cancer, colon cancer, pancreatic cancer, bladder cancer, liver cancer and cervical cancer.

As used herein, the term “preventing a disease” (e.g., preventing cancer) in a subject means for example, to stop the development of one or more symptoms of a disease in a subject before they occur or are detectable, e.g., by the patient or the patient's doctor. Preferably, the disease (e.g., cancer) does not develop at all, i.e., no symptoms of the disease are detectable. However, it can also result in delaying or slowing of the development of one or more symptoms of the disease. Alternatively, or in addition, it can result in the decreasing of the severity of one or more subsequently developed symptoms.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

The present disclosure is also described and demonstrated by way of the following examples. However, the use of these and other examples anywhere in the specification is illustrative only and in no way limits the scope and meaning of the invention or of any exemplified term. Likewise, the invention is not limited to any particular preferred embodiment or aspect described herein. Indeed, many modifications and variations may be apparent to those skilled in the art upon reading this specification, and such variations can be made without departing from the invention in spirit or in scope. The invention is therefore to be limited only by the terms of the appended claims along with the full scope of equivalents to which those claims are entitled.

Example compounds are set forth in Table 1 below.

TABLE 1

| Ex- | | | |
|---|---|---|---|
| ample | | | |
| No. | Cpd ID | Structure | name |
| 1 | XH161-102 (MS102) | 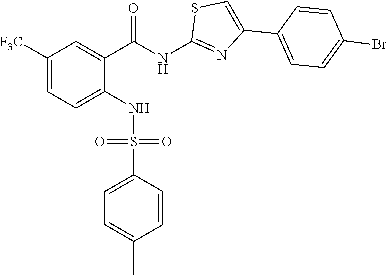 [SMILES: Cc1ccc(S(=O)(=O)Nc2ccc(C(F)(F)F)cc2C(=O)Nc2nc(-c3ccc(Br)cc3)cs2)cc1] | N-(4-(4- bromophenyl)thiazol- 2-yl)-2-((4- methylphenyl) sulfonamido)-5- (trifluoromethyl) benzamide |
| 2 | XH153-7 | 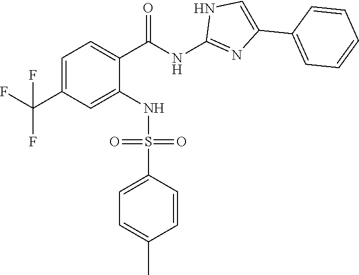 [SMILES: Cc1ccc(S(=O)(=O)Nc2cc(C(F)(F)F)ccc2C(=O)Nc2nc(-c3ccccc3)c[nH]2)cc1] | 2-((4-methylphenyl) sulfonamido)- N-(4-phenyl-1H- imidazol-2-yl)-4- (trifluoromethyl) benzamide |
| 3 | XH153-10 | 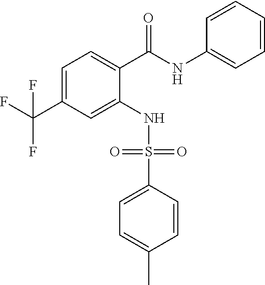 [SMILES: Cc1ccc(S(=O)(=O)Nc2cc(C(F)(F)F)ccc2C(=O)Nc2ccccc2)cc1] | 2-((4-methylphenyl) sulfonamido)- N-phenyl-4- (trifluoromethyl) benzamide |
| 4 | XH153-74 | 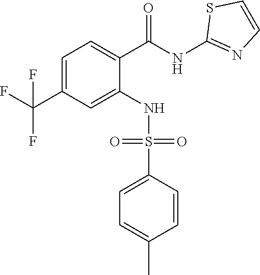 [SMILES: Cc1ccc(S(=O)(=O)Nc2cc(C(F)(F)F)ccc2C(=O)Nc2nccs2)cc1] | 2-((4-methylphenyl) sulfonamido)- N-(thiazol-2-yl)- 4- (trifluoromethyl) benzamide |
| 5 | Xh146-120 | 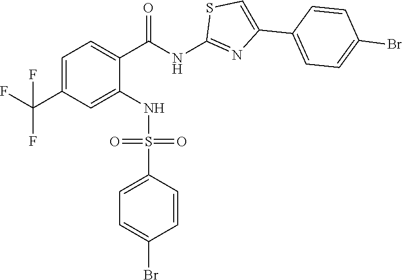 [SMILES: O=C(Nc1nc(-c2ccc(Br)cc2)cs1)c1ccc(C(F)(F)F)cc1NS(=O)(=O)c1ccc(Br)cc1] | 2-((4-bromophenyl) sulfonamido)- N-(4-(4- bromophenyl)thiazol- 2-yl)-4- (trifluoromethyl) benzamide |
| 6 | XH146-123 | 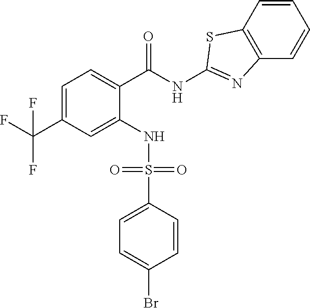 [SMILES: O=C(Nc1nc2ccccc2s1)c1ccc(C(F)(F)F)cc1NS(=O)(=O)c1ccc(Br)cc1] | N-(benzo[d]thiazol-2- yl)-2-((4- methylphenyl) sulfonamido)-4- (trifluoromethyl) benzamide |
| 7 | XH153-144 | 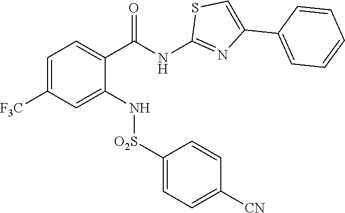 [SMILES: N#Cc1ccc(S(=O)(=O)Nc2cc(C(F)(F)F)ccc2C(=O)Nc2nc(-c3ccccc3)cs2)cc1] | 2-((4- cyanophenyl) sulfonamido)-N-(4- phenylthiazol-2-yl)-4- (trifluoromethyl) benzamide |
| 8 | XH146-180 | 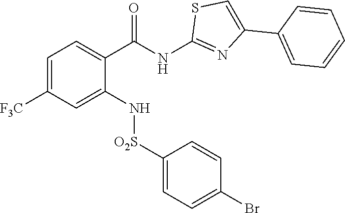 [SMILES: O=C(Nc1nc(-c2ccccc2)cs1)c1ccc(C(F)(F)F)cc1NS(=O)(=O)c1ccc(Br)cc1] | 2-((4- bromophenyl) sulfonamido)-N-(4- phenylthiazol-2-yl)-4- (trifluoromethyl) benzamide |
| 9 | XH153-6 | 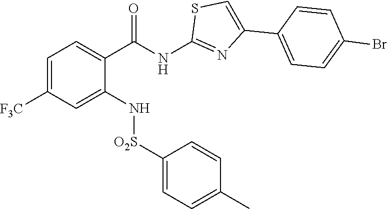 [SMILES: Cc1ccc(S(=O)(=O)Nc2cc(C(F)(F)F)ccc2C(=O)Nc2nc(-c3ccc(Br)cc3)cs2)cc1] | N-(4-(4- bromophenyl)thiazol- 2-yl)-2-((4- methylphenyl) sulfonamido)-4- (trifluoromethyl) benzamide |
| 10 | XH153-168 | 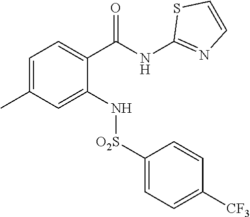 [SMILES: Cc1ccc(C(=O)Nc2nccs2)c(NS(=O)(=O)c2ccc(C(F)(F)F)cc2)c1] | 4-Methyl-N-(thiazol-2- yl)-2-((4- (trifluoromethyl) phenyl)sulfonamido) benzamide |
| 11 | XH153-157 | 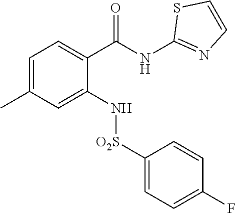 [SMILES: Cc1ccc(C(=O)Nc2nccs2)c(NS(=O)(=O)c2ccc(F)cc2)c1] | 2-((4- Fluorophenyl) sulfonamido)-4- methyl-N-(thiazol-2- yl)benzamide |
| 12 | XH153-148 | 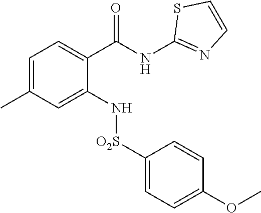 | 2-((4- methoxyphenyl) sulfonamido)-4- methyl-N-(thiazol-2- yl)benzamide |
| 13 | XH153-147 | 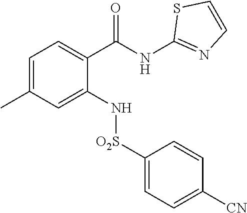 [SMILES: Cc1ccc(C(=O)Nc2nccs2)c(NS(=O)(=O)c2ccc(C#N)cc2)c1] | 2-((4-cyanophenyl) sulfonamido)- 4-methyl-N- (thiazol-2- yl)benzamide |
| 14 | XH153-167 | 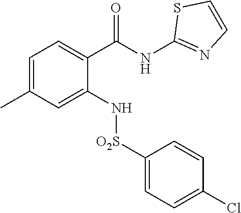 [SMILES: Cc1ccc(C(=O)Nc2nccs2)c(NS(=O)(=O)c2ccc(Cl)cc2)c1] | 2-((4-chlorophenyl) sulfonamido)- 4-methyl-N- (thiazol-2- yl)benzamide |
| 15 | XH153-145 | 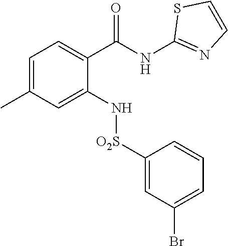 [SMILES: Cc1ccc(C(=O)Nc2nccs2)c(NS(=O)(=O)c2cccc(Br)c2)c1] | 2-((3- bromophenyl) sulfonamido)-4- methyl-N-(thiazol-2- yl)benzamide |
| 16 | XH153-182 | 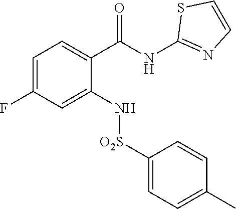 [SMILES: Cc1ccc(S(=O)(=O)Nc2cc(F)ccc2C(=O)Nc2nccs2)cc1] | 4-fluoro-2-((4- methylphenyl) sulfonamido)-N- (thiazol-2- yl)benzamide |
| 17 | XH153-155 | 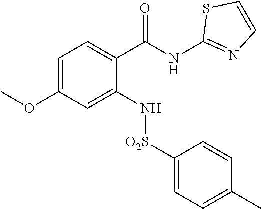 | 4-methoxy-2-((4- methylphenyl) sulfonamido)-N- (thiazol-2- yl)benzamide |
| 18 | XH153-150 | 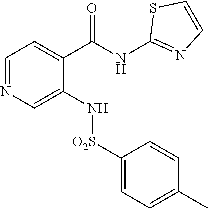 [SMILES: Cc1ccc(S(=O)(=O)Nc2cnccc2C(=O)Nc2nccs2)cc1] | 3-((4- methylphenyl) sulfonamido)-N- (thiazol-2- yl)isonicotinamide |
| 19 | XH161-26 | 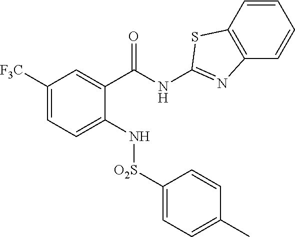 [SMILES: Cc1ccc(S(=O)(=O)Nc2ccc(C(F)(F)F)cc2C(=O)Nc2nc3ccccc3s2)cc1] | N-(benzo[d]thiazol-2- yl)-2-((4- methylphenyl) sulfonamido)-5- (trifluoromethyl) benzamide |
| 20 | XH161-2 | 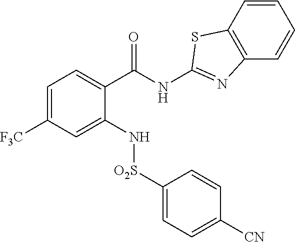 [SMILES: N#Cc1ccc(S(=O)(=O)Nc2cc(C(F)(F)F)ccc2C(=O)Nc2nc3ccccc3s2)cc1] | N-(benzo[d]thiazol-2- yl)-2-((4- cyanophenyl) sulfonamido)-4- (trifluoromethyl) benzamide |
| 21 | XH161-4 | 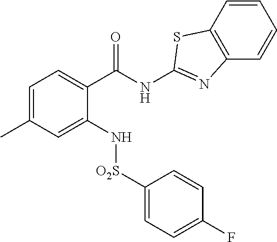 [SMILES: Cc1ccc(C(=O)Nc2nc3ccccc3s2)c(NS(=O)(=O)c2ccc(F)cc2)c1] | N-(benzo[d]thiazol-2- yl)-2-((4- fluorophenyl) sulfonamido)-4- methylbenzamide |
| 22 | XH161-5 | 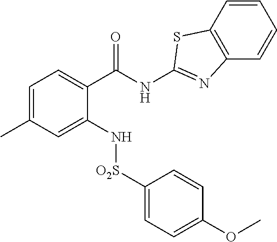 [SMILES: COc1ccc(S(=O)(=O)Nc2cc(C)ccc2C(=O)Nc2nc3ccccc3s2)cc1] | N-(benzo[d]thiazol-2- yl)-2-((4- methoxyphenyl) sulfonamido)-4- methylbenzamide |
| 23 | XH161-6 | 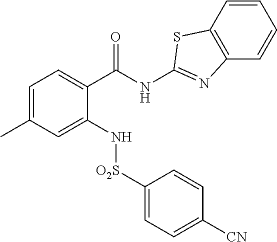 [SMILES: Cc1ccc(C(=O)Nc2nc3ccccc3s2)c(NS(=O)(=O)c2ccc(C#N)cc2)c1] | N-(benzo[d]thiazol-2- yl)-2-((4- cyanophenyl) sulfonamido)-4- methylbenzamide |
| 24 | XH161-3 | 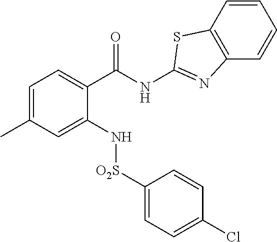 [SMILES: Cc1ccc(C(=O)Nc2nc3ccccc3s2)c(NS(=O)(=O)c2ccc(Cl)cc2)c1] | N-(benzo[d]thiazol-2- yl)-2-((4- chlorophenyl) sulfonamido)-4- methylbenzamide |
| 25 | XH161-19 | 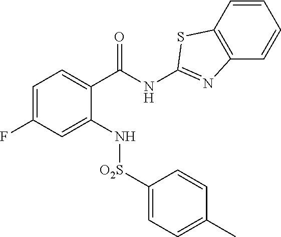 [SMILES: Cc1ccc(S(=O)(=O)Nc2cc(F)ccc2C(=O)Nc2nc3ccccc3s2)cc1] | N-(benzo[d]thiazol-2- yl)-4-fluoro-2-((4- methylphenyl) sulfonamido) benzamide |
| 26 | XH161-37 | 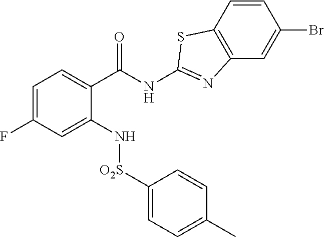 [SMILES: Cc1ccc(S(=O)(=O)Nc2cc(F)ccc2C(=O)Nc2nc3cc(Br)ccc3s2)cc1] | N-(5- bromobenzo[d]thiazol- 2-yl)-4-fluoro-2-((4- methylphenyl) sulfonamido) benzamide |
| 27 | XH161-32 | 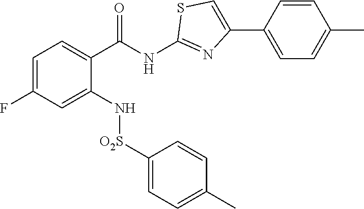 [SMILES: Cc1ccc(-c2csc(NC(=O)c3ccc(F)cc3NS(=O)(=O)c3ccc(C)cc3)n2)cc1] | 4-fluoro-2-((4- methylphenyl) sulfonamido)-N-(4- (p-tolyl)thiazol-2- yl)benzamide |
| 28 | XH161-33 | 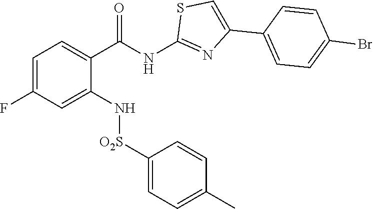 [SMILES: Cc1ccc(S(=O)(=O)Nc2cc(F)ccc2C(=O)Nc2nc(-c3ccc(Br)cc3)cs2)cc1] | N-(4-(4- bromophenyl)thiazol- 2-yl)-4-fluoro-2-((4- methylphenyl) sulfonamido) benzamide |
| 29 | XH161-25 | 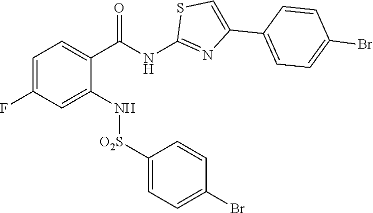 [SMILES: O=C(Nc1nc(-c2ccc(Br)cc2)cs1)c1ccc(F)cc1NS(=O)(=O)c1ccc(Br)cc1] | 2-((4- bromophenyl) sulfonamido)-N-(4-(4- bromophenyl)thiazol- 2-yl)-4- fluorobenzamide |
| 30 | XH161-31 | 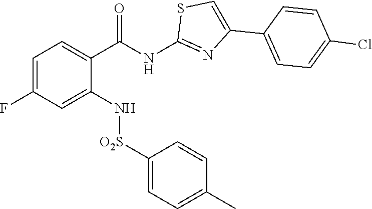 [SMILES: Cc1ccc(S(=O)(=O)Nc2cc(F)ccc2C(=O)Nc2nc(-c3ccc(Cl)cc3)cs2)cc1] | N-(4-(4- chlorophenyl)thiazol- 2-yl)-4-fluoro-2-((4- methylphenyl) sulfonamido)benzamide |
| 31 | XH161-36 | 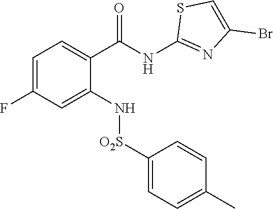 [SMILES: Cc1ccc(S(=O)(=O)Nc2cc(F)ccc2C(=O)Nc2nc(Br)cs2)cc1] | N-(4-bromothiazol-2- yl)-4-fluoro-2-((4- methylphenyl) sulfonamido)benzamide |
| 32 | XH161-94 | 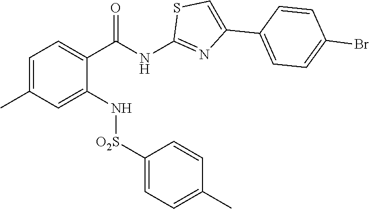 [SMILES: Cc1ccc(S(=O)(=O)Nc2cc(C)ccc2C(=O)Nc2nc(-c3ccc(Br)cc3)cs2)cc1] | N-(4-(4- bromophenyl)thiazol- 2-yl)-4-methyl-2-((4- methylphenyl) sulfonamido)benzamide |
| 33 | XH161-98 | 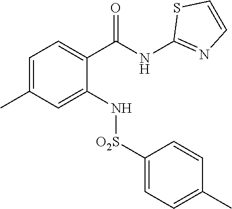 | 4-methyl-2-((4- methylphenyl) sulfonamido)-N- (thiazol-2-yl) benzamide |
| 34 | XH161-95 | 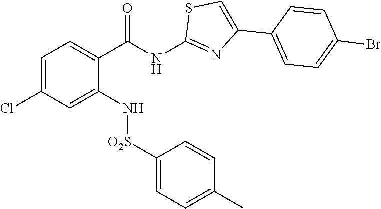 [SMILES: Cc1ccc(S(=O)(=O)Nc2cc(Cl)ccc2C(=O)Nc2nc(-c3ccc(Br)cc3)cs2)cc1] | N-(4-(4- bromophenyl)thiazol- 2-yl)-4-chloro-2-((4- methylphenyl) sulfonamido) benzamide |
| 35 | XH161-35 | 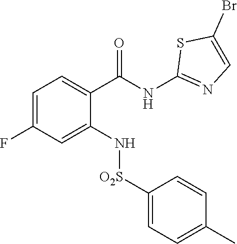 [SMILES: Cc1ccc(S(=O)(=O)Nc2cc(F)ccc2C(=O)Nc2ncc(Br)s2)cc1] | N-(5-bromothiazol-2- yl)-4-fluoro-2-((4- methylphenyl) sulfonamido) benzamide |
| 36 | XH161-96 | 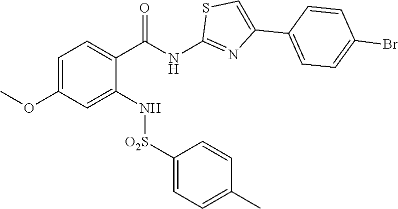 [SMILES: COc1ccc(C(=O)Nc2nc(-c3ccc(Br)cc3)cs2)c(NS(=O)(=O)c2ccc(C)cc2)c1] | N-(4-(4- bromophenyl)thiazol- 2-yl)-4-methoxy-2-((4- methylphenyl) sulfonamido)benzamide |
| 37 | XH161-134 | 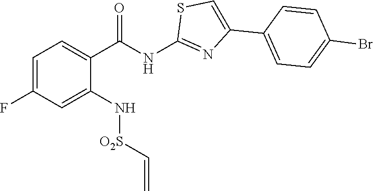 [SMILES: C=CS(=O)(=O)Nc1cc(F)ccc1C(=O)Nc1nc(-c2ccc(Br)cc2)cs1] | N-(4-(4- bromophenyl)thiazol- 2-yl)-4-fluoro-2- (vinylsulfonamido) benzamide |
| 38 | XH161-135 | 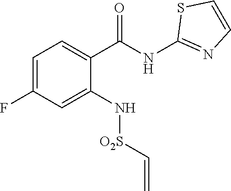 [SMILES: C=CS(=O)(=O)Nc1cc(F)ccc1C(=O)Nc1nccs1] | 4-fluoro-N-(thiazol-2- yl)-2- (vinylsulfonamido) benzamide |
| 39 | XH161-132 | 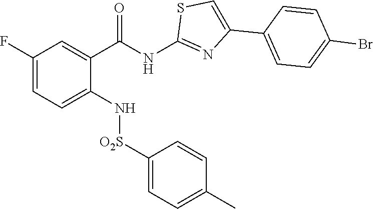 [SMILES: Cc1ccc(S(=O)(=O)Nc2ccc(F)cc2C(=O)Nc2nc(-c3ccc(Br)cc3)cs2)cc1] | N-(4-(4- bromophenyl)thiazol- 2-yl)-5-fluoro-2-((4- methylphenyl) sulfonamido)benzamide |
| 40 | XH161-133 | 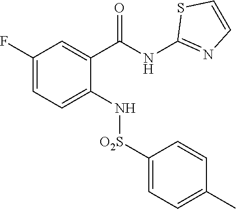 [SMILES: Cc1ccc(S(=O)(=O)Nc2ccc(F)cc2C(=O)Nc2nccs2)cc1] | 5-fluoro-2-((4- methylphenyl) sulfonamido)-N- (thiazol-2- yl)benzamide |
| 41 | XH161-101 | 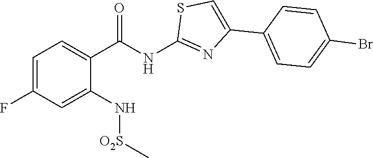 [SMILES: CS(=O)(=O)Nc1cc(F)ccc1C(=O)Nc1nc(-c2ccc(Br)cc2)cs1] | N-(4-(4- bromophenyl)thiazol- 2-yl)-4-fluoro-2- (methylsulfonamido) benzamide |
| 42 | XH161-136 | 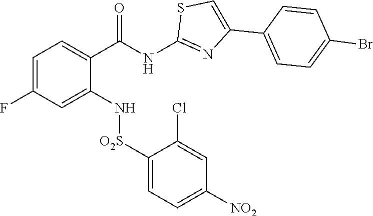 [SMILES: O=C(Nc1nc(-c2ccc(Br)cc2)cs1)c1ccc(F)cc1NS(=O)(=O)c1ccc([N+](=O)[O-])cc1Cl] | N-(4-(4- bromophenyl)thiazol- 2-yl)-2-((2-chloro-4- nitrophenyl)sulfonamido)- 4-fluorobenzamide |
| 43 | XH161-137 | 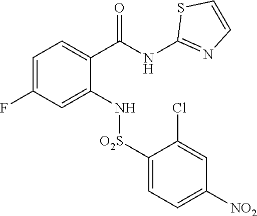 [SMILES: O=C(Nc1nccs1)c1ccc(F)cc1NS(=O)(=O)c1ccc([N+](=O)[O-])cc1Cl] | 2-((2-chloro-4- nitrophenyl)sulfonamido)- 4-fluoro-N- (thiazol-2- yl)benzamide |
| 44 | XH161-169 | 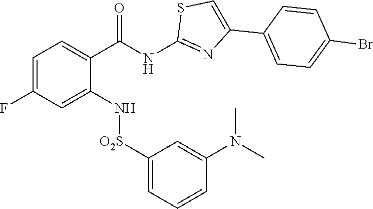 [SMILES: CN(C)c1cccc(S(=O)(=O)Nc2cc(F)ccc2C(=O)Nc2nc(-c3ccc(Br)cc3)cs2)c1] | N-(4-(4- bromophenyl)thiazol- 2-yl)-2-((3- (dimethylamino)phenyl) sulfonamido)-4- fluorobenzamide |
| 45 | XH161-170 | 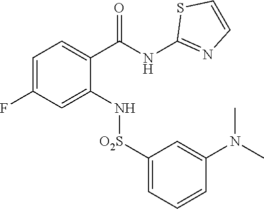 [SMILES: CN(C)c1cccc(S(=O)(=O)Nc2cc(F)ccc2C(=O)Nc2nccs2)c1] | 2-((3- (dimethylamino)phenyl) sulfonamido)-4- fluoro-N-(thiazol-2- yl)benzamide |
| 46 | XH161-153 | 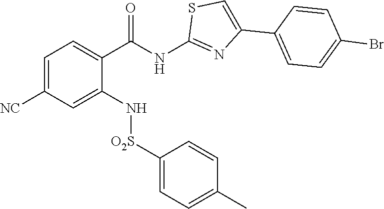 [SMILES: Cc1ccc(S(=O)(=O)Nc2cc(C#N)ccc2C(=O)Nc2nc(-c3ccc(Br)cc3)cs2)cc1] | N-(4-(4- bromophenyl)thiazol- 2-yl)-4-cyano-2-((4- methylphenyl) sulfonamido) benzamide |
| 47 | XH161-154 | 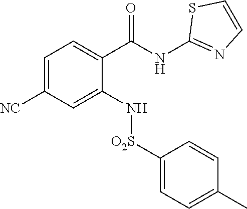 [SMILES: Cc1ccc(S(=O)(=O)Nc2cc(C#N)ccc2C(=O)Nc2nccs2)cc1] | 4-cyano-2-((4- methylphenyl) sulfonamido)- N-(thiazol-2- yl)benzamide |
| 48 | XH168-77 | 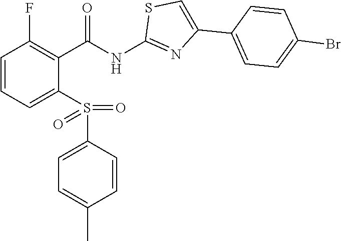 [SMILES: Cc1ccc(S(=O)(=O)c2cccc(F)c2C(=O)Nc2nc(-c3ccc(Br)cc3)cs2)cc1] | N-(4-(4- bromophenyl)thiazol- 2-yl)-2-fluoro-6-((4- methylphenyl) sulfonamido) benzamide |
| 49 | XH181-20 | 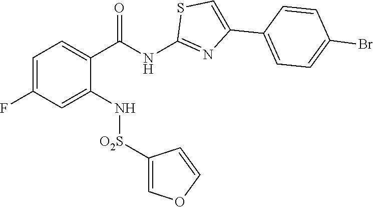 [SMILES: O=C(Nc1nc(-c2ccc(Br)cc2)cs1)c1ccc(F)cc1NS(=O)(=O)c1ccoc1] | N-(4-(4- bromophenyl)thiazol- 2-yl)-4-fluoro-2-(furan- 3- sulfonamido)benzamide |
| 50 | XH181-38 | 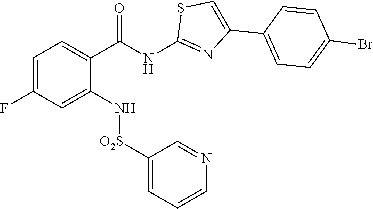 [SMILES: O=C(Nc1nc(-c2ccc(Br)cc2)cs1)c1ccc(F)cc1NS(=O)(=O)c1cccnc1] | N-(4-(4- bromophenyl)thiazol- 2-yl)-4-fluoro-2- (pyridine-3- sulfonamido)benzamide |
| 51 | XH181-39 | 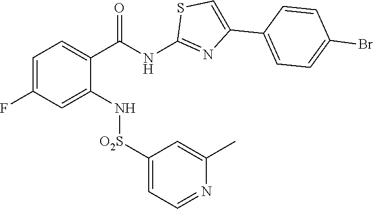 [SMILES: Cc1cc(S(=O)(=O)Nc2cc(F)ccc2C(=O)Nc2nc(-c3ccc(Br)cc3)cs2)ccn1] | N-(4-(4- bromophenyl)thiazol- 2-yl)-4-fluoro-2-((2- methylpyridine)-4- sulfonamido)benzamide |
| 52 | XH161-177 | 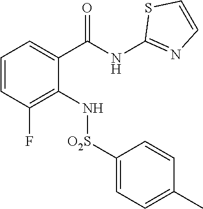 [SMILES: Cc1ccc(S(=O)(=O)Nc2c(F)cccc2C(=O)Nc2nccs2)cc1] | 3-fluoro-2-((4- methylphenyl) sulfonamido)- N-(thiazol-2- yl)benzamide |
| 53 | XH161-176 | 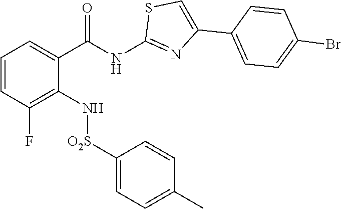 [SMILES: Cc1ccc(S(=O)(=O)Nc2c(F)cccc2C(=O)Nc2nc(-c3ccc(Br)cc3)cs2)cc1] | N-(4-(4- bromophenyl)thiazol- 2-yl)-3-fluoro-2-((4- methylphenyl) sulfonamido) benzamide |
| 54 | XH161-172 (MS1172) | 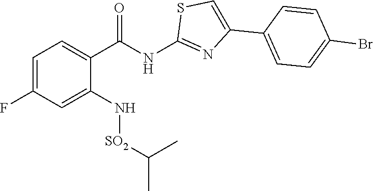 [SMILES: CC(C)S(=O)(=O)Nc1cc(F)ccc1C(=O)Nc1nc(-c2ccc(Br)cc2)cs1] | N-(4-(4- bromophenyl)thiazol- 2-yl)-4-fluoro-2-((1- methylethyl) sulfonamido) benzamide |
| 55 | XH161-173 | 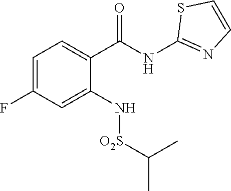 [SMILES: CC(C)S(=O)(=O)Nc1cc(F)ccc1C(=O)Nc1nccs1] | 4-fluoro-2-((1- methylethyl) sulfonamido)- N-(thiazol-2- yl)benzamide |
| 56 | XH168-72-1 | 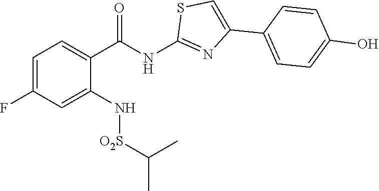 [SMILES: CC(C)S(=O)(=O)Nc1cc(F)ccc1C(=O)Nc1nc(-c2ccc(O)cc2)cs1] | 4-fluoro-N-(4-(4- hydroxyphenyl)thiazol- 2-yl)-2-((1- methylethyl)sulfonamido) benzamide |
| 57 | XH168-72-2 | 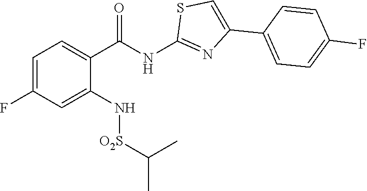 [SMILES: CC(C)S(=O)(=O)Nc1cc(F)ccc1C(=O)Nc1nc(-c2ccc(F)cc2)cs1] | 4-fluoro-N-(4-(4- fluorophenyl)thiazol-2- yl)-2-((1- methylethyl)sulfonamido) benzamide |
| 58 | XH168-72-3 | 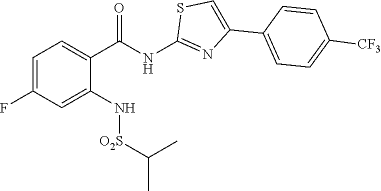 [SMILES: CC(C)S(=O)(=O)Nc1cc(F)ccc1C(=O)Nc1nc(-c2ccc(C(F)(F)F)cc2)cs1] | 4-fluoro-2-((1- methylethyl)sulfonamido)- N-(4-(4- (trifluoromethyl)phenyl) thiazol-2- yl)benzamide |
| 59 | XH168-79 | 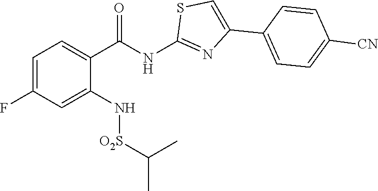 [SMILES: CC(C)S(=O)(=O)Nc1cc(F)ccc1C(=O)Nc1nc(-c2ccc(C#N)cc2)cs1] | N-(4-(4- cyanophenyl)thiazol-2- yl)-4-fluoro-2-((1- methylethyl)sulfonamido) benzamide |
| 60 | XH168-63 | 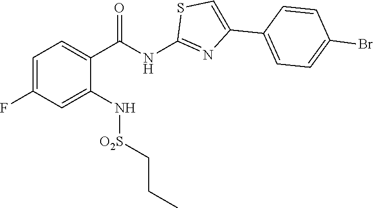 [SMILES: CCCS(=O)(=O)Nc1cc(F)ccc1C(=O)Nc1nc(-c2ccc(Br)cc2)cs1] | N-(4-(4- bromophenyl)thiazol- 2-yl)-4-fluoro-2- (propylsulfonamido) benzamide |
| 61 | XH168-76 | 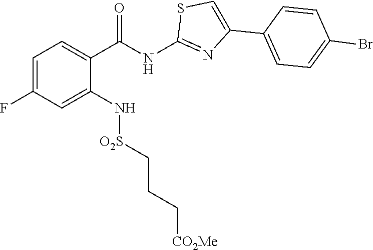 [SMILES: COC(=O)CCCS(=O)(=O)Nc1cc(F)ccc1C(=O)Nc1nc(-c2ccc(Br)cc2)cs1] | Methyl 4-(N-(2-((4-(4- bromophenyl)thiazol- 2-yl)carbamoyl)-5- fluorophenyl)sulfamoyl) butanoate |
| 62 | XH161-180 (MS1180) | 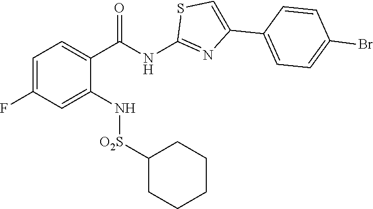 [SMILES: O=C(Nc1nc(-c2ccc(Br)cc2)cs1)c1ccc(F)cc1NS(=O)(=O)C1CCCCC1] | N-(4-(4- bromophenyl)thiazol- 2-yl)-2-(cyclo- hexanesulfonamido)- 4-fluorobenzamide |
| 63 | XH161-181 | 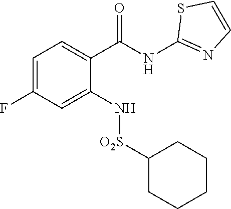 [SMILES: O=C(Nc1nccs1)c1ccc(F)cc1NS(=O)(=O)C1CCCCC1] | 2-(cyclo- hexanesulfonamido)- 4-fluoro-N- (thiazol-2- yl)benzamide |
| 64 | XH153-79-D | 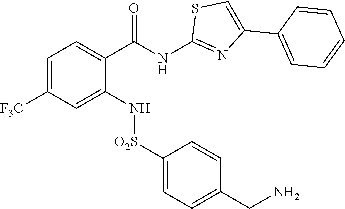 [SMILES: NCc1ccc(S(=O)(=O)Nc2cc(C(F)(F)F)ccc2C(=O)Nc2nc(-c3ccccc3)cs2)cc1] | 2-((4- (aminomethyl)phenyl) sulfonamido)-N-(4- phenylthiazol-2-yl)-4- (trifluoromethyl) benzamide |
| 65 | XH153-83 | 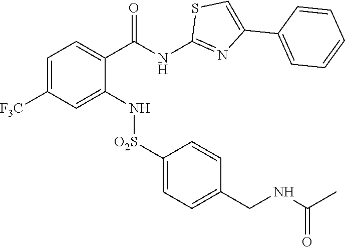 [SMILES: CC(=O)NCc1ccc(S(=O)(=O)Nc2cc(C(F)(F)F)ccc2C(=O)Nc2nc(-c3ccccc3)cs2)cc1] | 2-((4- (acetamidomethyl) phenyl)sulfonamido)-N- (4-phenylthiazol-2-yl)-4- (trifluoromethyl) benzamide |
| 66 | XH153-84-p1 | 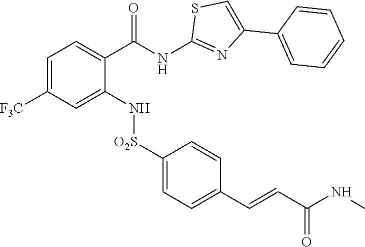 [SMILES: CNC(=O)/C=C/c1ccc(S(=O)(=O)Nc2cc(C(F)(F)F)ccc2C(=O)Nc2nc(-c3ccccc3)cs2)cc1] | (E)-2-((4-(3- (methylamino)-3- oxoprop-1-en-1- yl)phenyl)sulfonamido)- N-(4-phenylthiazol-2- yl)-4- (trifluoromethyl) benzamide |
| 67 | XH153-84-p2 | 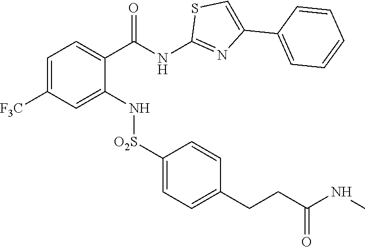 [SMILES: CNC(=O)CCc1ccc(S(=O)(=O)Nc2cc(C(F)(F)F)ccc2C(=O)Nc2nc(-c3ccccc3)cs2)cc1] | 2-((4-(3- (methylamino)-3- oxopropyl)phenyl) sulfonamido)-N-(4- phenylthiazol-2-yl)-4- (trifluoromethyl) benzamide |
| 68 | XH153-20 | 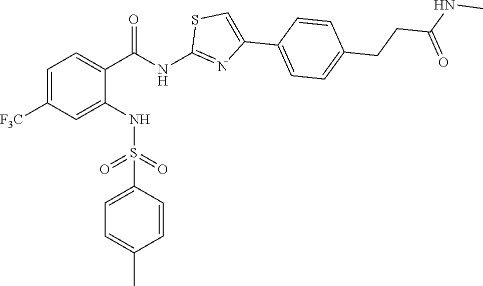 [SMILES: CNC(=O)CCc1ccc(-c2csc(NC(=O)c3ccc(C(F)(F)F)cc3NS(=O)(=O)c3ccc(C)cc3)n2)cc1] | N-(4-(4-(3- (methylamino)-3- oxopropyl)phenyl) thiazol-2-yl)-2-((4- methylphenyl) sulfonamido)-4- (trifluoromethyl) benzamide |
| 69 | XH181-103 | 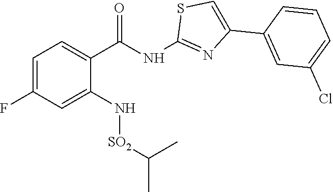 [SMILES: CC(C)S(=O)(=O)Nc1cc(F)ccc1C(=O)Nc1nc(-c2cccc(Cl)c2)cs1] | N-(4-(3- Chlorophenyl)thiazol-2- yl)-4-fluoro-2-((1- methylethyl)sulfonamido) benzamide |
| 70 | XH188-104 | 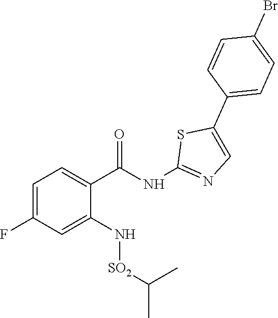 [SMILES: CC(C)S(=O)(=O)Nc1cc(F)ccc1C(=O)Nc1ncc(-c2ccc(Br)cc2)s1] | N-(5-(4- Bromophenyl)thiazol- 2-yl)-4-fluoro-2-((1- methylethyl)sulfonamido) benzamide |
| 71 | XH188-105 | 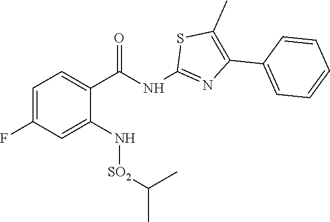 [SMILES: Cc1sc(NC(=O)c2ccc(F)cc2NS(=O)(=O)C(C)C)nc1-c1ccccc1] | 4-Fluoro-N-(5- methyl-4-phenylthiazol- 2-yl)-2-((1- methylethyl)sulfonamido) benzamide |
| 72 | XH188-111 | 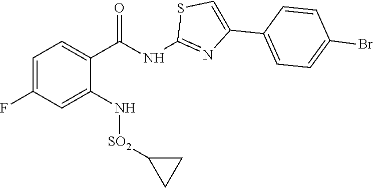 [SMILES: O=C(Nc1nc(-c2ccc(Br)cc2)cs1)c1ccc(F)cc1NS(=O)(=O)C1CC1] | N-(4-(4- Bromophenyl)thiazol-2- yl)-2-(cyclo- propanesulfonamido)- 4-fluorobenzamide |
| 73 | XH188-113 | 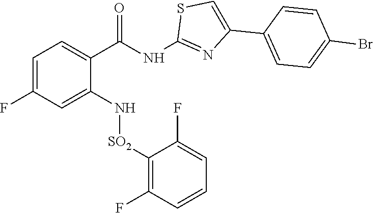 [SMILES: O=C(Nc1nc(-c2ccc(Br)cc2)cs1)c1ccc(F)cc1NS(=O)(=O)c1c(F)cccc1F] | N-(4-(4- Bromophenyl)thiazol-2- yl)-2-((2,6- difluorophenyl) sulfonamido)-4- fluorobenzamide |
| 74 | XH188-117 | 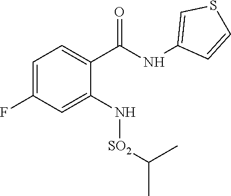 [SMILES: CC(C)S(=O)(=O)Nc1cc(F)ccc1C(=O)Nc1ccsc1] | 4-Fluoro-2-((1- methylethyl)sulfonamido)- N-(thiophen-3- yl)benzamide |
| 75 | XH188-119 | 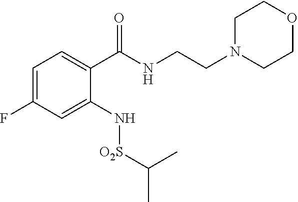 [SMILES: CC(C)S(=O)(=O)Nc1cc(F)ccc1C(=O)NCCN1CCOCC1] | 4-Fluoro-2-((1- methylethyl) sulfonamido)-N-(2- morpholinoethyl) benzamide |
| 76 | XH188-122 | 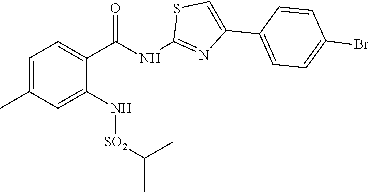 [SMILES: Cc1ccc(C(=O)Nc2nc(-c3ccc(Br)cc3)cs2)c(NS(=O)(=O)C(C)C)c1] | N-(4-(4- Bromophenyl)thiazol-2- yl)-4-methyl-2-((1- methylethyl)sulfonamido) benzamide |
| 77 | XH188-123 | 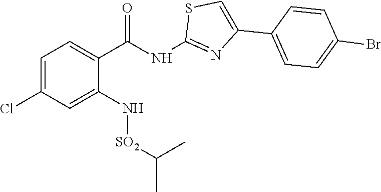 [SMILES: CC(C)S(=O)(=O)Nc1cc(Cl)ccc1C(=O)Nc1nc(-c2ccc(Br)cc2)cs1] | N-(4-(4- Bromophenyl)thiazol-2- yl)-4-chloro-2-((1- methylethyl)sulfonamido) benzamide |
| 78 | XH188-124 | 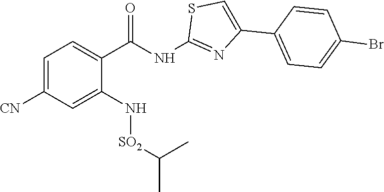 | N-(4-(4- Bromophenyl)thiazol-2- yl)-4-isocyano-2-((1- methylethyl)sulfonamido) benzamide |
| 79 | XH188-125 | 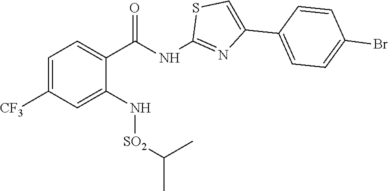 [SMILES: CC(C)S(=O)(=O)Nc1cc(C(F)(F)F)ccc1C(=O)Nc1nc(-c2ccc(Br)cc2)cs1] | N-(4-(4- Bromophenyl)thiazol-2- yl)-2-((1- methylethyl) sulfonamido)-4- (trifluoromethyl) benzamide |
| 80 | XH188-137 | 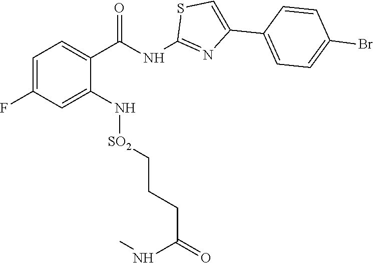 [SMILES: CNC(=O)CCCS(=O)(=O)Nc1cc(F)ccc1C(=O)Nc1nc(-c2ccc(Br)cc2)cs1] | N-(4-(4- Bromophenyl)thiazol-2- yl)-4-fluoro-2-((4- (methylamino)-4- oxobutyl)sulfonamido) benzamide |
| 81 | XH188-146 | 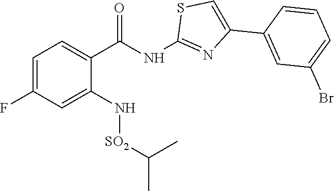 [SMILES: CC(C)S(=O)(=O)Nc1cc(F)ccc1C(=O)Nc1nc(-c2cccc(Br)c2)cs1] | N-(4-(3- Bromophenyl)thiazol-2- yl)-4-fluoro-2-((1- methylethyl)sulfonamido) benzamide |
| 82 | XH188-149 | 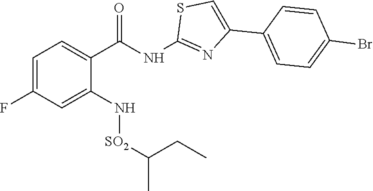 [SMILES: CCC(C)S(=O)(=O)Nc1cc(F)ccc1C(=O)Nc1nc(-c2ccc(Br)cc2)cs1] | N-(4-(4- bromophenyl)thiazol-2- yl)-4-fluoro-2-((1- methylpropyl)sulfonamido) benzamide |
| 83 | XH188-150 | 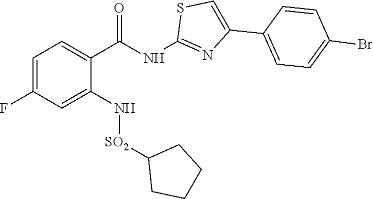 [SMILES: O=C(Nc1nc(-c2ccc(Br)cc2)cs1)c1ccc(F)cc1NS(=O)(=O)C1CCCC1] | N-(4-(4- Bromophenyl)thiazol-2- yl)-2- ((cyclopentyloxy)amino)- 4-fluorobenzamide |
| 84 | XH188-151 | 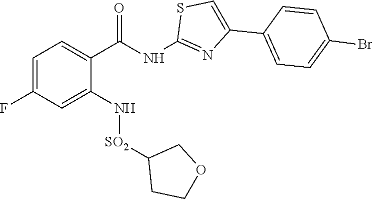 [SMILES: O=C(Nc1nc(-c2ccc(Br)cc2)cs1)c1ccc(F)cc1NS(=O)(=O)C1CCOC1] | N-(4-(4- bromophenyl)thiazol-2- yl)-4-fluoro-2- (((tetrahydrofuran-3- yl)oxy)amino)benzamide |
| 85 | XH188-170 | 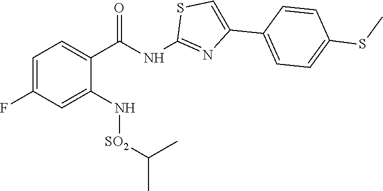 [SMILES: CSc1ccc(-c2csc(NC(=O)c3ccc(F)cc3NS(=O)(=O)C(C)C)n2)cc1] | 4-Fluoro-2-((1- methylethyl)sulfonamido)- N-(4-(4- (methylthio)phenyl) thiazol-2-yl)benzamide |
| 86 | XH188-171 | 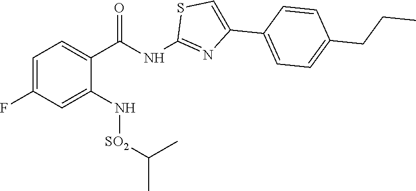 [SMILES: CCCc1ccc(-c2csc(NC(=O)c3ccc(F)cc3NS(=O)(=O)C(C)C)n2)cc1] | 4-Fluoro-2-((1- methylethyl)sulfonamido)- N-(4-(4- propylphenyl)thiazol-2- yl)benzamide |
| 87 | XH188-172 | 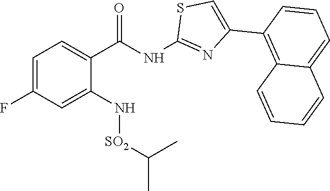 [SMILES: CC(C)S(=O)(=O)Nc1cc(F)ccc1C(=O)Nc1nc(-c2cccc3ccccc23)cs1] | 4-Fluoro-2-((1- methylethyl)sulfonamido)- N-(4-(naphthalen-1- yl)thiazol-2- yl)benzamide |
| 88 | XH188-173 | 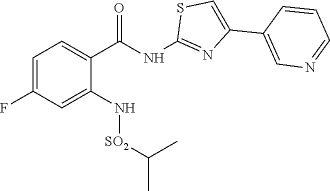 [SMILES: CC(C)S(=O)(=O)Nc1cc(F)ccc1C(=O)Nc1nc(-c2cccnc2)cs1] | 4-Fluoro-2-((1- methylethyl)sulfonamido)- N-(4-(pyridin-3- yl)thiazol-2- yl)benzamide |
| 89 | XH188-174 | 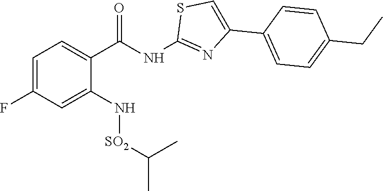 [SMILES: CCc1ccc(-c2csc(NC(=O)c3ccc(F)cc3NS(=O)(=O)C(C)C)n2)cc1] | N-(4-(4- Ethylphenyl)thiazol-2- yl)-4-fluoro-2-((1- methylethyl)sulfonamido) benzamide |
| 90 | XH188-175 | 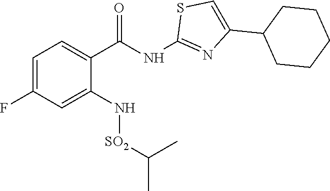 [SMILES: CC(C)S(=O)(=O)Nc1cc(F)ccc1C(=O)Nc1nc(C2CCCCC2)cs1] | N-(4- Cyclohexylthiazol-2-yl)- 4-fluoro-2-((1- methylethyl)sulfonamido) benzamide |
| 91 | XH188-176 | 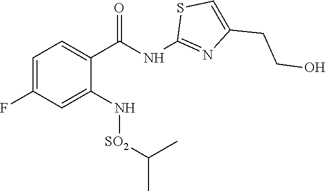 [SMILES: CC(C)S(=O)(=O)Nc1cc(F)ccc1C(=O)Nc1nc(CCO)cs1] | 4-Fluoro-N-(4-(2- hydroxyethyl)thiazol-2- yl)-2-((1- methylethyl)sulfonamido) benzamide |
| 92 | XH188-183 | 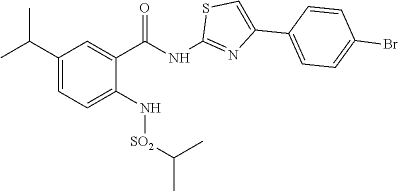 [SMILES: CC(C)c1ccc(NS(=O)(=O)C(C)C)c(C(=O)Nc2nc(-c3ccc(Br)cc3)cs2)c1] | N-(4-(4- bromophenyl)thiazol-2- yl)-5-isopropyl-2-((1- methylethyl)sulfonamido) benzamide |
| 93 | XH188-184 | 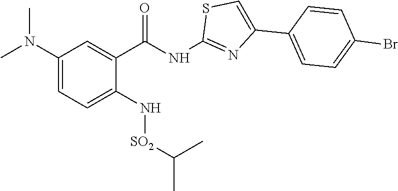 [SMILES: CC(C)S(=O)(=O)Nc1ccc(N(C)C)cc1C(=O)Nc1nc(-c2ccc(Br)cc2)cs1] | N-(4-(4- bromophenyl)thiazol-2- yl)-5-(dimethylamino)- 2-((1- methylethyl)sulfonamido) benzamide |
| 94 | XH188-185 | 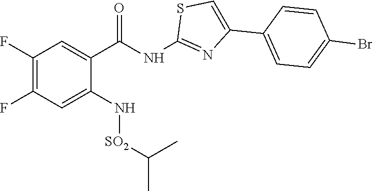 [SMILES: CC(C)S(=O)(=O)Nc1cc(F)c(F)cc1C(=O)Nc1nc(-c2ccc(Br)cc2)cs1] | N-(4-(4- Bromophenyl)thiazol-2- yl)-4,5-difluoro-2-((1- methylethyl)sulfonamido) benzamide |
| 95 | XH198-1 | 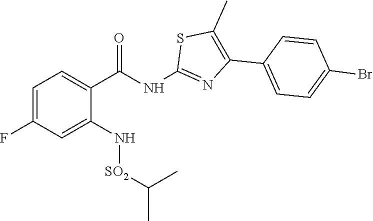 [SMILES: Cc1sc(NC(=O)c2ccc(F)cc2NS(=O)(=O)C(C)C)nc1-c1ccc(Br)cc1] | N-(4-(4- Bromophenyl)-5- methylthiazol-2-yl)-4- fluoro-2-((1- methylethyl)sulfonamido) benzamide |
| 96 | XH198-2 | 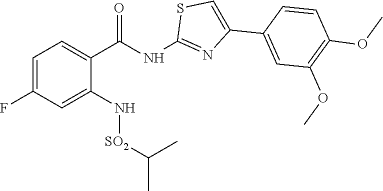 [SMILES: COc1ccc(-c2csc(NC(=O)c3ccc(F)cc3NS(=O)(=O)C(C)C)n2)cc1OC] | N-(4-(3,4- Dimethoxyphenyl)thiazol- 2-yl)-4-fluoro-2-((1- methylethyl)sulfonamido) benzamide |
| 97 | XH198-3 | 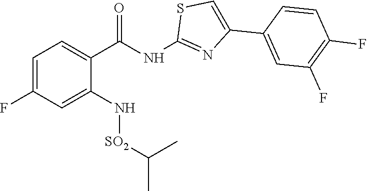 [SMILES: CC(C)S(=O)(=O)Nc1cc(F)ccc1C(=O)Nc1nc(-c2ccc(F)c(F)c2)cs1] | N-(4-(3,4- Dimethoxyphenyl)thiazol- 2-yl)-4-fluoro-2-((1- methylethyl)sulfonamido) benzamide |
| 98 | XH198-4 | 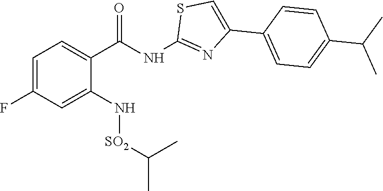 [SMILES: CC(C)c1ccc(-c2csc(NC(=O)c3ccc(F)cc3NS(=O)(=O)C(C)C)n2)cc1] | N-(4-(3,4- difluorophenyl)thiazol- 2-yl)-4-fluoro-2-((1- methylethyl)sulfonamido) benzamide |
| 99 | XH198-62 | 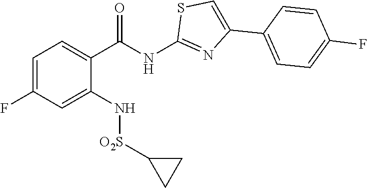 [SMILES: O=C(Nc1nc(-c2ccc(F)cc2)cs1)c1ccc(F)cc1NS(=O)(=O)C1CC1] | 2-(Cyclo- propanesulfonamido)- 4-fluoro-N-(4-(4- fluorophenyl)thiazol-2- yl)benzamide |
| 100 | XH193-63 | 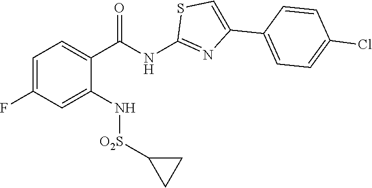 [SMILES: O=C(Nc1nc(-c2ccc(Cl)cc2)cs1)c1ccc(F)cc1NS(=O)(=O)C1CC1] | N-(4-(4- Chlorophenyl)thiazol-2- yl)-2-(cyclo- propanesulfonamido)- 4-fluorobenzamide |
| 101 | XH198-65 | 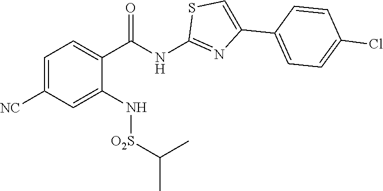 [SMILES: CC(C)S(=O)(=O)Nc1cc(C#N)ccc1C(=O)Nc1nc(-c2ccc(Cl)cc2)cs1] | N-(4-(4- Chlorophenyl)thiazol-2- yl)-4-cyano-2-((1- methylethyl)sulfonamido) benzamide |
| 102 | XH198-66 | 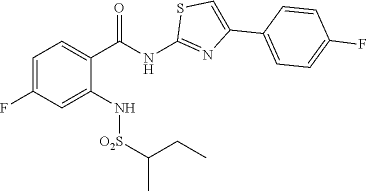 [SMILES: CCC(C)S(=O)(=O)Nc1cc(F)ccc1C(=O)Nc1nc(-c2ccc(F)cc2)cs1] | 4-Fluoro-N-(4-(4- fluorophenyl)thiazol-2- yl)-2-((1- methylpropyl)sulfonamido) benzamide |
| 103 | XH198-67 | 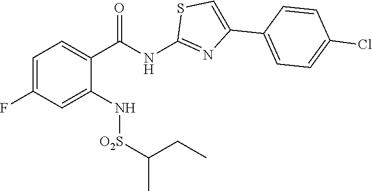 [SMILES: CCC(C)S(=O)(=O)Nc1cc(F)ccc1C(=O)Nc1nc(-c2ccc(Cl)cc2)cs1] | N-(4-(4- Chlorophenyl)thiazol-2- yl)-4-fluoro-2-((1- methylpropyl)sulfonamido) benzamide |
| 104 | XH198-70 | 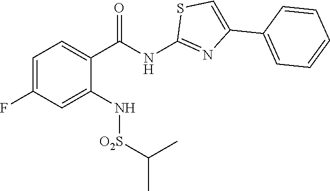 [SMILES: CC(C)S(=O)(=O)Nc1cc(F)ccc1C(=O)Nc1nc(-c2ccccc2)cs1] | 4-Fluoro-2-((1- methylethyl) sulfonamido)-N-(4- phenylthiazol-2- yl)benzamide |
| 105 | XH198-71 | 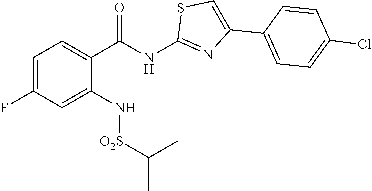 [SMILES: CC(C)S(=O)(=O)Nc1cc(F)ccc1C(=O)Nc1nc(-c2ccc(Cl)cc2)cs1] | N-(4-(4- Chlorophenyl)thiazol-2- yl)-4-fluoro-2-((1- methylethyl)sulfonamido) benzamide |
| 106 | XH188-135p1 | 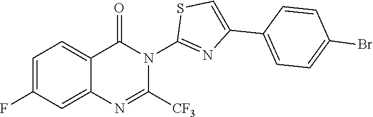 [SMILES: O=c1c2ccc(F)cc2nc(C(F)(F)F)n1-c1nc(-c2ccc(Br)cc2)cs1] | 3-(4-(4- Bromophenyl)thiazol-2- yl)-7-fluoro-2- (trifluoromethyl) quinazolin-4(3H)-one |
| 107 | XH188-135p2 | 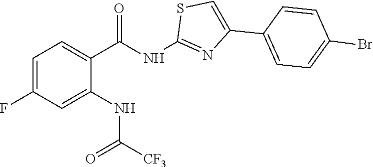 [SMILES: O=C(Nc1nc(-c2ccc(Br)cc2)cs1)c1ccc(F)cc1NC(=O)C(F)(F)F] | N-(4-(4- Bromophenyl)thiazol-2- yl)-4-fluoro-2-(2,2,2- trifluoroacetamido) benzamide |
| 108 | XH198-8 | 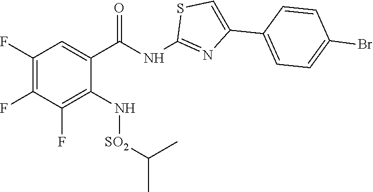 [SMILES: CC(C)S(=O)(=O)Nc1c(C(=O)Nc2nc(-c3ccc(Br)cc3)cs2)cc(F)c(F)c1F] | N-(4-(4- Bromophenyl)thiazol-2- yl)-3,4,5-trifluoro-2-((1- methylethyl)sulfonamido) benzamide |
| 109 | XH198-9 | 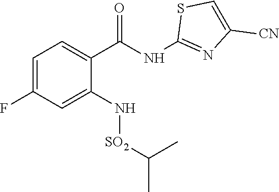 [SMILES: CC(C)S(=O)(=O)Nc1cc(F)ccc1C(=O)Nc1nc(C#N)cs1] | N-(4- Cyanothiazol-2-yl)-4- fluoro-2-((1- methylethyl)sulfonamido) benzamide |
| 110 | XH198-38P1 | 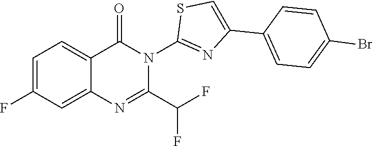 [SMILES: O=c1c2ccc(F)cc2nc(C(F)F)n1-c1nc(-c2ccc(Br)cc2)cs1] | 3-(4-(4- Bromophenyl)thiazol-2- yl)-2-(difluoromethyl)-7- fluoroquinazolin-4(3H)- one |
| 111 | XH198-38P2 | 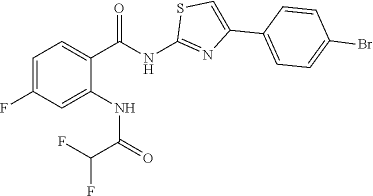 [SMILES: O=C(Nc1nc(-c2ccc(Br)cc2)cs1)c1ccc(F)cc1NC(=O)C(F)F] | N-(4-(4- Bromophenyl)thiazol-2- yl)-2-(2,2- difluoroacetamido)-4- fluorobenzamide |
| 112 | XH198-43P1 | 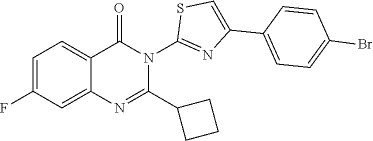 [SMILES: O=c1c2ccc(F)cc2nc(C2CCC2)n1-c1nc(-c2ccc(Br)cc2)cs1] | 3-(4-(4- Bromophenyl)thiazol-2- yl)-2-cyclobutyl-7- fluoroquinazolin-4(3H)- one (XH198- |
| 113 | XH198-43P2 | 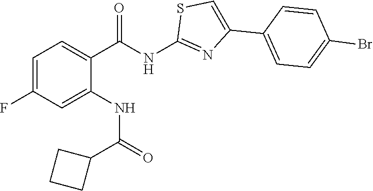 [SMILES: O=C(Nc1nc(-c2ccc(Br)cc2)cs1)c1ccc(F)cc1NC(=O)C1CCC1] | N-(4-(4- Bromophenyl)thiazol- 2-yl)-2- (cyclobutane- carboxamido)-4- fluorobenzamide |
| 114 | XH198-44 | 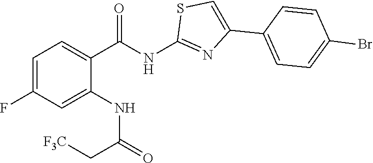 [SMILES: O=C(CC(F)(F)F)Nc1cc(F)ccc1C(=O)Nc1nc(-c2ccc(Br)cc2)cs1] | N-(4-(4- Bromophenyl)thiazol-2- yl)-4-fluoro-2-(3,3,3- trifluoropropanamido) benzamide |
| 115 | XH198-45 | 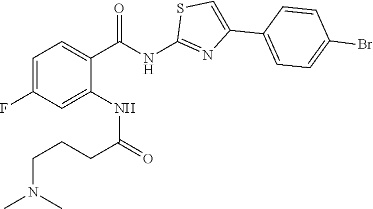 [SMILES: CN(C)CCCC(=O)Nc1cc(F)ccc1C(=O)Nc1nc(-c2ccc(Br)cc2)cs1] | N-(4-(4- bBromophenyl)thiazol- 2-yl)-2-(4- (dimethylamino) butanamido)-4- fluorobenzamide |
| 116 | XH198-46 | 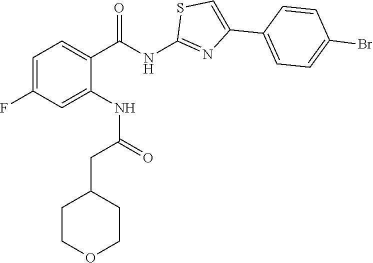 [SMILES: O=C(CC1CCOCC1)Nc1cc(F)ccc1C(=O)Nc1nc(-c2ccc(Br)cc2)cs1] | N-(4-(4- Bromophenyl)thiazol- 2-yl)-4-fluoro-2-(2- (tetrahydro-2H-pyran- 4-yl)acetamido) benzamide |
| 117 | XH198-48 | 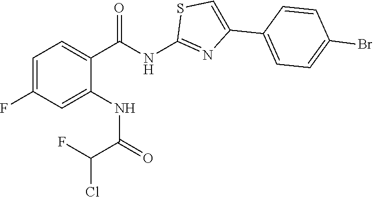 [SMILES: O=C(Nc1nc(-c2ccc(Br)cc2)cs1)c1ccc(F)cc1NC(=O)C(F)Cl] | N-(4-(4- Bromophenyl)thiazol-2- yl)-2-(2-chloro-2- fluoroacetamido)-4- fluorobenzamide |
| 118 | XH198-68P1 | 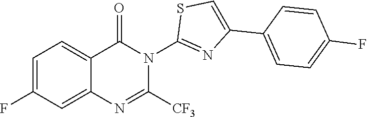 [SMILES: O=c1c2ccc(F)cc2nc(C(F)(F)F)n1-c1nc(-c2ccc(F)cc2)cs1] | 7-Fluoro-3-(4-(4- fluorophenyl)thiazol-2- yl)-2- (trifluoromethyl) quinazolin-4(3H)-one |
| 119 | XH198-68P2 | 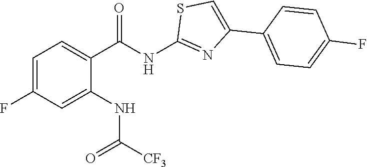 [SMILES: O=C(Nc1nc(-c2ccc(F)cc2)cs1)c1ccc(F)cc1NC(=O)C(F)(F)F] | 4-Fluoro-N-(4-(4- fluorophenyl)thiazol-2- yl)-2-(2,2,2- trifluoroacetamido) benzamide |
| 120 | XH198-69P1 | 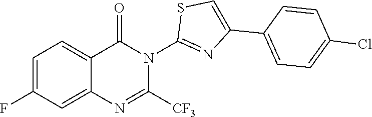 [SMILES: O=c1c2ccc(F)cc2nc(C(F)(F)F)n1-c1nc(-c2ccc(Cl)cc2)cs1] | 3-(4-(4- Chlorophenyl)thiazol-2- yl)-7-fluoro-2- (trifluoromethyl) quinazolin-4(3H)-one |
| 121 | XH198-69P2 | 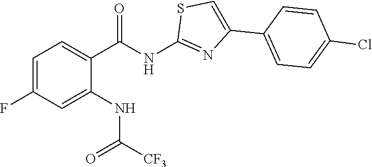 [SMILES: O=C(Nc1nc(-c2ccc(Cl)cc2)cs1)c1ccc(F)cc1NC(=O)C(F)(F)F] | N-(4-(4- chlorophenyl)thiazol-2- yl)-4-fluoro-2-(2,2,2- trifluoroacetamido) benzamide |
| 122 | XH208-34P1 | 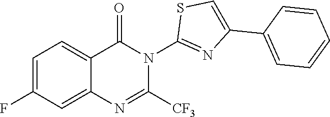 [SMILES: O=c1c2ccc(F)cc2nc(C(F)(F)F)n1-c1nc(-c2ccccc2)cs1] | 7-Fluoro-3-(4- phenylthiazol-2-yl)-2- (trifluoromethyl) quinazolin-4(3H)-one |
| 123 | XH208-34P2 | 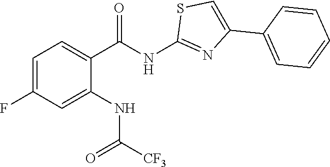 [SMILES: O=C(Nc1nc(-c2ccccc2)cs1)c1ccc(F)cc1NC(=O)C(F)(F)F] | 4-Fluoro-N-(4- phenylthiazol-2-yl)-2- (2,2,2- trifluoroacetamido) benzamide |
| 124 | XH208-39P1 | 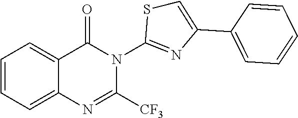 [SMILES: O=c1c2ccccc2nc(C(F)(F)F)n1-c1nc(-c2ccccc2)cs1] | 3-(4- Phenylthiazol-2-yl)-2- (trifluoromethyl) quinazolin-4(3H)-one |
| 125 | XH208-39P2 | 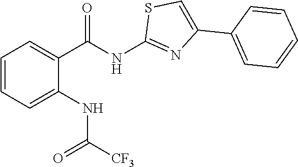 [SMILES: O=C(Nc1nc(-c2ccccc2)cs1)c1ccccc1NC(=O)C(F)(F)F] | N-(4- Phenylthiazol-2-yl)-2- (2,2,2- trifluoroacetamido) benzamide |
| 126 | XH208-40P1 | 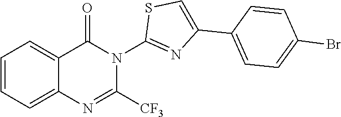 [SMILES: O=c1c2ccccc2nc(C(F)(F)F)n1-c1nc(-c2ccc(Br)cc2)cs1] | 3-(4-(4- Bromophenyl)thiazol- 2-yl)-2- (trifluoromethyl) quinazolin-4(3H)-one |
| 127 | XH208-40P2 | 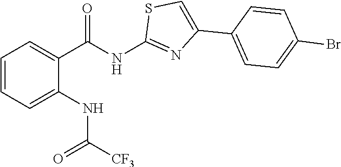 [SMILES: O=C(Nc1nc(-c2ccc(Br)cc2)cs1)c1ccccc1NC(=O)C(F)(F)F] | N-(4-(4- Bromophenyl)thiazol- 2-yl)-2-(2,2,2- trifluoroacetamido) benzamide |
| 128 | XH208-41P1 | 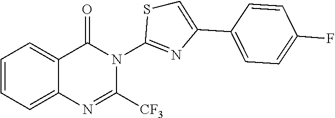 [SMILES: O=c1c2ccccc2nc(C(F)(F)F)n1-c1nc(-c2ccc(F)cc2)cs1] | 3-(4-(4- Fluorophenyl)thiazol-2- yl)-2- (trifluoromethyl) quinazolin-4(3H)-one |
| 129 | XH208-41P2 | 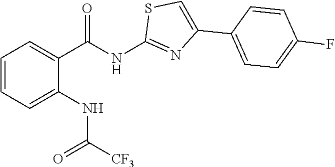 [SMILES: O=C(Nc1nc(-c2ccc(F)cc2)cs1)c1ccccc1NC(=O)C(F)(F)F] | N-(4-(4- Fluorophenyl)thiazol- 2-yl)-2-(2,2,2- trifluoroacetamido) benzamide |
| 130 | XH208-77P1 | 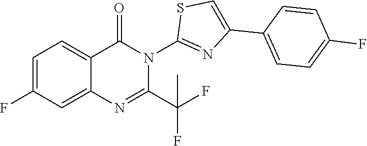 [SMILES: CC(F)(F)c1nc2cc(F)ccc2c(=O)n1-c1nc(-c2ccc(F)cc2)cs1] | 2-(1,1- Difluoroethyl)-7-fluoro- 3-(4-(4- fluorophenyl)thiazol-2- yl)quinazolin-4(3H)-one |
| 131 | XH208-77P2 | 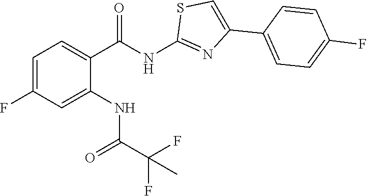 [SMILES: CC(F)(F)C(=O)Nc1cc(F)ccc1C(=O)Nc1nc(-c2ccc(F)cc2)cs1] | 2-(2,2- Difluoropropanamido)- 4-fluoro-N-(4-(4- fluorophenyl)thiazol-2- yl)benzamide |
| 132 | XH208-79P1 | 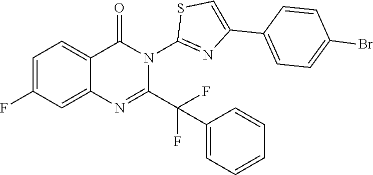 [SMILES: O=c1c2ccc(F)cc2nc(C(F)(F)c2ccccc2)n1-c1nc(-c2ccc(Br)cc2)cs1] | 3-(4-(4- Bromophenyl)thiazol-2- yl)-2-(difluoro (phenyl)methyl)- 7-fluoroquinazolin- 4(3H)-one |
| 133 | XH208-79P2 | 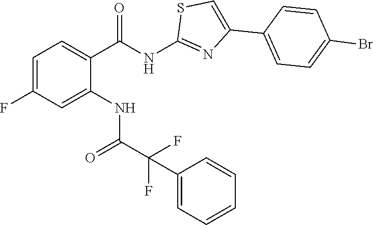 [SMILES: O=C(Nc1nc(-c2ccc(Br)cc2)cs1)c1ccc(F)cc1NC(=O)C(F)(F)c1ccccc1] | N-(4-(4- Bromophenyl)thiazol-2- yl)-2-(2,2-difluoro-2- phenylacetamido)-4- fluorobenzamide |
| 134 | XH208-80P1 | 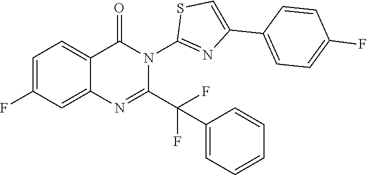 [SMILES: O=c1c2ccc(F)cc2nc(C(F)(F)c2ccccc2)n1-c1nc(-c2ccc(F)cc2)cs1] | 2-(Difluoro (phenyl)methyl)- 7-fluoro-3-(4-(4- fluorophenyl)thiazol-2- yl)quinazolin-4(3H)-one |
| 135 | XH208-80P2 | 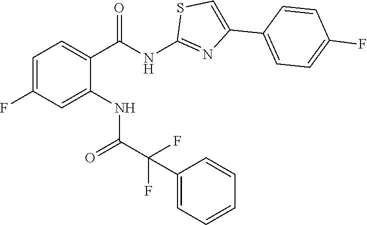 [SMILES: O=C(Nc1nc(-c2ccc(F)cc2)cs1)c1ccc(F)cc1NC(=O)C(F)(F)c1ccccc1] | 2-(2,2-Difluoro- 2-phenylacetamido)-4- fluoro-N-(4-(4- fluorophenyl)thiazol-2- yl)benzamide |
| 136 | XH208-81P1 | 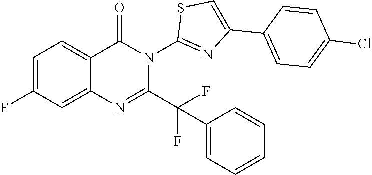 [SMILES: O=c1c2ccc(F)cc2nc(C(F)(F)c2ccccc2)n1-c1nc(-c2ccc(Cl)cc2)cs1] | 3-(4-(4- Chlorophenyl)thiazol-2- yl)-2-(difluoro (phenyl)methyl)- 7-fluoroquinazolin- 4(3H)-one |
| 137 | XH208-81P2 | 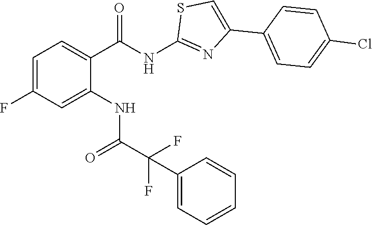 [SMILES: O=C(Nc1nc(-c2ccc(Cl)cc2)cs1)c1ccc(F)cc1NC(=O)C(F)(F)c1ccccc1] | N-(4-(4- Chlorophenyl)thiazol-2- yl)-2-(2,2-difluoro-2- phenylacetamido)-4- fluorobenzamide |
| 138 | XH208-85P1 | 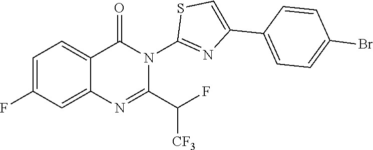 [SMILES: O=c1c2ccc(F)cc2nc(C(F)C(F)(F)F)n1-c1nc(-c2ccc(Br)cc2)cs1] | 3-(4-(4- Bromophenyl)thiazol-2- yl)-7-fluoro-2-(1,2,2,2- tetrafluoroethyl) quinazolin-4(3H)-one |
| 139 | XH208-85P2 | 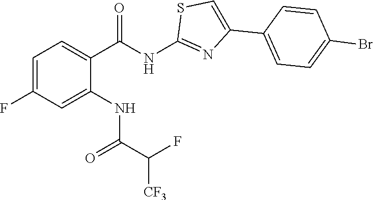 [SMILES: O=C(Nc1nc(-c2ccc(Br)cc2)cs1)c1ccc(F)cc1NC(=O)C(F)C(F)(F)F] | N-(4-(4- Bromophenyl)thiazol-2- yl)-4-fluoro-2-(2,3,3,3- tetrafluoropropanamido) benzamide |
| 140 | XH208-86P1 | 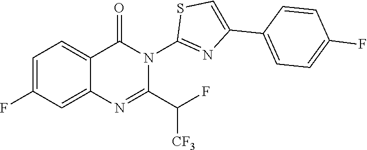 [SMILES: O=c1c2ccc(F)cc2nc(C(F)C(F)(F)F)n1-c1nc(-c2ccc(F)cc2)cs1] | 7-Fluoro-3-(4-(4- fluorophenyl)thiazol-2- yl)-2-(1,2,2,2- tetrafluoroethyl) quinazolin-4(3H)-one |
| 141 | XH208-86P2 | 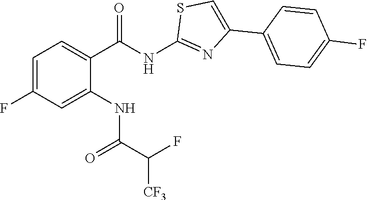 [SMILES: O=C(Nc1nc(-c2ccc(F)cc2)cs1)c1ccc(F)cc1NC(=O)C(F)C(F)(F)F] | 4-Fluoro-N-(4-(4- fluorophenyl)thiazol-2- yl)-2-(2,3,3,3- tetrafluoropropanamido) benzamide |
| 142 | XH208-87P1 | 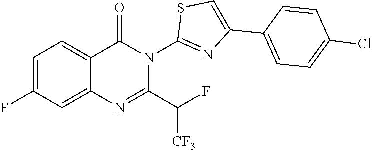 [SMILES: O=c1c2ccc(F)cc2nc(C(F)C(F)(F)F)n1-c1nc(-c2ccc(Cl)cc2)cs1] | 3-(4-(4- Chlorophenyl)thiazol-2- yl)-7-fluoro-2-(1,2,2,2- tetrafluoroethyl) quinazolin-4(3H)-one |
| 143 | XH208-87P2 | 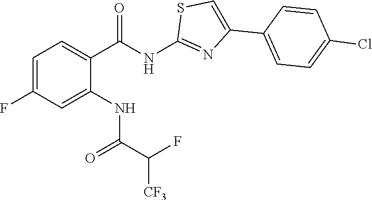 [SMILES: O=C(Nc1nc(-c2ccc(Cl)cc2)cs1)c1ccc(F)cc1NC(=O)C(F)C(F)(F)F] | N-(4-(4- Chlorophenyl)thiazol-2- yl)-4-fluoro-2-(2,3,3,3- tetrafluoropropanamido) benzamide |
| 144 | XH208-92P1 | 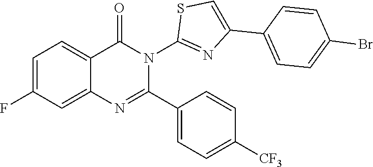 [SMILES: O=c1c2ccc(F)cc2nc(-c2ccc(C(F)(F)F)cc2)n1-c1nc(-c2ccc(Br)cc2)cs1] | 3-(4-(4- Bromophenyl)thiazol-2- yl)-7-fluoro-2-(4- (trifluoromethyl)phenyl) quinazolin-4(3H)-one |
| 145 | XH208-92P2 | 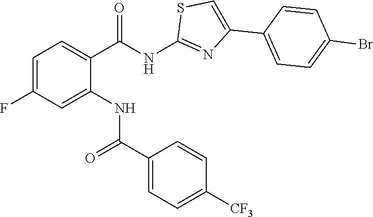 [SMILES: O=C(Nc1cc(F)ccc1C(=O)Nc1nc(-c2ccc(Br)cc2)cs1)c1ccc(C(F)(F)F)cc1] | N-(4-(4- Bromophenyl)thiazol-2- yl)-4-fluoro-2-(4- (trifluoromethyl) benzamido)benzamide |
| 146 | XH208-93P1 | 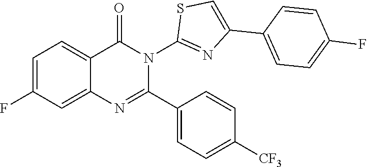 [SMILES: O=c1c2ccc(F)cc2nc(-c2ccc(C(F)(F)F)cc2)n1-c1nc(-c2ccc(F)cc2)cs1] | 7-Fluoro-3-(4-(4- fluorophenyl)thiazol-2- yl)-2-(4- (trifluoromethyl)phenyl) quinazolin-4(3H)-one |
| 147 | XH208-93P2 | 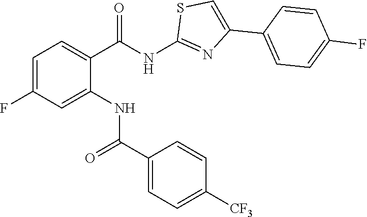 [SMILES: O=C(Nc1cc(F)ccc1C(=O)Nc1nc(-c2ccc(F)cc2)cs1)c1ccc(C(F)(F)F)cc1] | 4-Fluoro-N-(4-(4- fluorophenyl)thiazol-2- yl)-2-(4- (trifluoromethyl) benzamido)benzamide |
| 148 | XH208-94P1 | 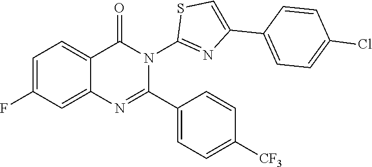 [SMILES: O=c1c2ccc(F)cc2nc(-c2ccc(C(F)(F)F)cc2)n1-c1nc(-c2ccc(Cl)cc2)cs1] | 3-(4-(4- Chlorophenyl)thiazol-2- yl)-7-fluoro-2-(4- (trifluoromethyl)phenyl) quinazolin-4(3H)-one |
| 149 | XH208-94P2 | 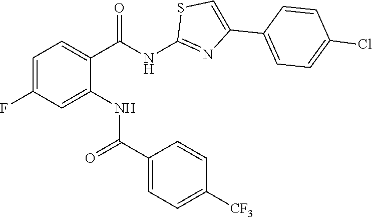 [SMILES: O=C(Nc1cc(F)ccc1C(=O)Nc1nc(-c2ccc(Cl)cc2)cs1)c1ccc(C(F)(F)F)cc1] | N-(4-(4- Chlorophenyl)thiazol-2- yl)-4-fluoro-2-(4- (trifluoromethyl) benzamido)benzamide |
| 150 | XH208-167p1 | 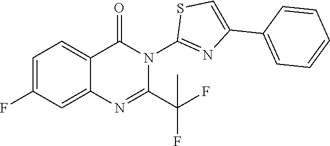 [SMILES: CC(F)(F)c1nc2cc(F)ccc2c(=O)n1-c1nc(-c2ccccc2)cs1] | 2-(1,1- Difluoroethyl)-7-fluoro- 3-(4-phenylthiazol-2- yl)quinazolin-4(3H)-one |
| 151 | XH208-167p2 | 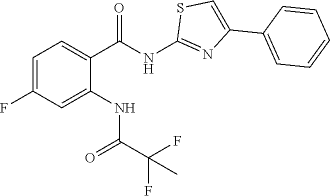 [SMILES: CC(F)(F)C(=O)Nc1cc(F)ccc1C(=O)Nc1nc(-c2ccccc2)cs1] | 2-(2,2- Difluoropropanamido)- 4-fluoro-N-(4- phenylthiazol-2- yl)benzamide |
| 152 | XH208-168p1 | 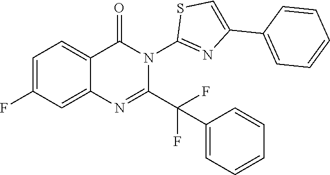 [SMILES: O=c1c2ccc(F)cc2nc(C(F)(F)c2ccccc2)n1-c1nc(-c2ccccc2)cs1] | 2-(Difluoro (phenyl)methyl)- 7-fluoro-3-(4- phenylthiazol-2- yl)quinazolin-4(3H)-one |
| 153 | XH208-168p2 | 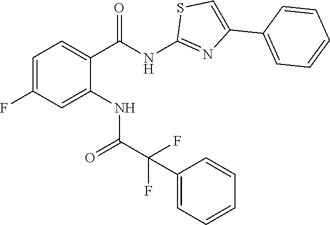 [SMILES: O=C(Nc1nc(-c2ccccc2)cs1)c1ccc(F)cc1NC(=O)C(F)(F)c1ccccc1] | 2-(2,2-Difluoro- 2-phenylacetamido)-4- fluoro-N-(4- phenylthiazol-2- yl)benzamide |
| 154 | XH216-30p1 | 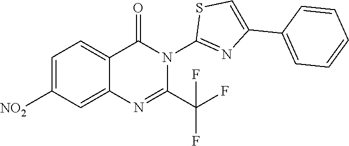 [SMILES: O=c1c2ccc([N+](=O)[O-])cc2nc(C(F)(F)F)n1-c1nc(-c2ccccc2)cs1] | 7-Nitro-3-(4- phenylthiazol-2-yl)-2- (trifluoromethyl) quinazolin-4(3H)-one |
| 155 | XH216-30p2 | 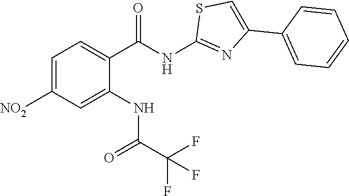 [SMILES: O=C(Nc1nc(-c2ccccc2)cs1)c1ccc([N+](=O)[O-])cc1NC(=O)C(F)(F)F] | 4-Nitro-N-(4- phenylthiazol-2-yl)-2- (2,2,2- trifluoroacetamido) benzamide |
| 156 | XH216-31p1 | 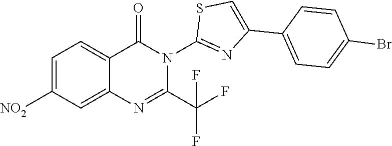 [SMILES: O=c1c2ccc([N+](=O)[O-])cc2nc(C(F)(F)F)n1-c1nc(-c2ccc(Br)cc2)cs1] | 3-(4-(4- Bromophenyl)thiazol-2- yl)-7-nitro-2- (trifluoromethyl) quinazolin-4(3H)-one |
| 157 | XH216-31p2 | 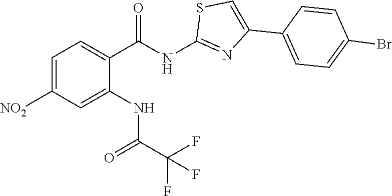 [SMILES: O=C(Nc1nc(-c2ccc(Br)cc2)cs1)c1ccc([N+](=O)[O-])cc1NC(=O)C(F)(F)F] | N-(4-(4- Bromo))phenyl)thiazol- 2-yl)-4-nitro-2-(2,2,2- trifluoroacetamido) benzamide |
| 158 | XH216-33p1 | 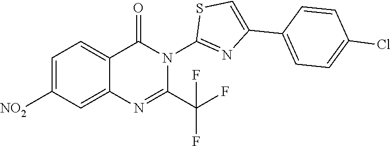 [SMILES: O=c1c2ccc([N+](=O)[O-])cc2nc(C(F)(F)F)n1-c1nc(-c2ccc(Cl)cc2)cs1] | 3-(4-(4- Chlorophenyl)thiazol-2- yl)-7-nitro-2- (trifluoromethyl) quinazolin-4(3H)-one |
| 159 | XH216-33p2 | 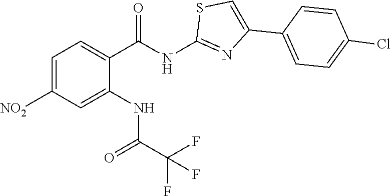 [SMILES: O=C(Nc1nc(-c2ccc(Cl)cc2)cs1)c1ccc([N+](=O)[O-])cc1NC(=O)C(F)(F)F] | N-(4-(4- Chlorophenyl)thiazol-2- yl)-4-nitro-2-(2,2,2- trifluoroacetamido) benzamide |
| 160 | XH216-57p1 | 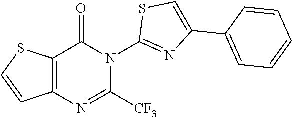 [SMILES: O=c1c2sccc2nc(C(F)(F)F)n1-c1nc(-c2ccccc2)cs1] | 3-(4- Phenylthiazol-2-yl)-2- (trifluoromethyl)thieno [3,2-d]pyrimidin-4(3H)- one |
| 161 | XH216-57p2 | 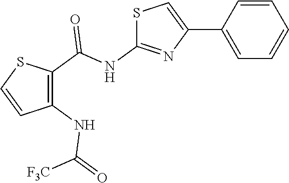 [SMILES: O=C(Nc1nc(-c2ccccc2)cs1)c1sccc1NC(=O)C(F)(F)F] | N-(4- Phenylthiazol-2-yl)-3- (2,2,2- trifluoroacetamido) thiophene-2- carboxamide |
| 162 | XH216-58p1 | 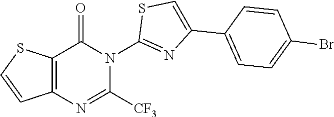 [SMILES: O=c1c2sccc2nc(C(F)(F)F)n1-c1nc(-c2ccc(Br)cc2)cs1] | 3-(4-(4- Bromophenyl)thiazol-2- yl)-2- (trifluoromethyl)thieno [3,2-d]pyrimidin-4(3H)- one |
| 163 | XH216-58p2 | 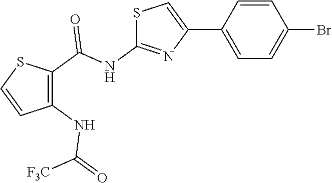 [SMILES: O=C(Nc1nc(-c2ccc(Br)cc2)cs1)c1sccc1NC(=O)C(F)(F)F] | N-(4-(4- Bromophenyl)thiazol-2- yl)-3-(2,2,2- trifluoroacetamido) thiophene-2- carboxamide |
| 164 | XH216-59p1 | 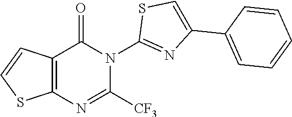 [SMILES: O=c1c2ccsc2nc(C(F)(F)F)n1-c1nc(-c2ccccc2)cs1] | 3-(4- Phenylthiazol-2-yl)-2- (trifluoromethyl)thieno [2,3-d]pyrimidin-4(3H)- one |
| 165 | XH216-59p2 | 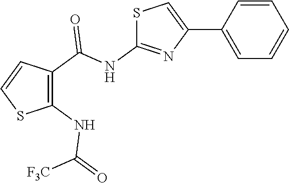 [SMILES: O=C(Nc1nc(-c2ccccc2)cs1)c1ccsc1NC(=O)C(F)(F)F] | N-(4- Phenylthiazol-2-yl)-2- (2,2,2- trifluoroacetamido) thiophene-3- carboxamide |
| 166 | XH216-60p1 | 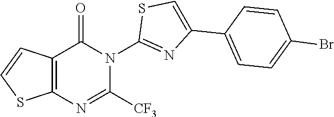 [SMILES: O=c1c2ccsc2nc(C(F)(F)F)n1-c1nc(-c2ccc(Br)cc2)cs1] | 3-(4-(4- Bromophenyl)thiazol-2- yl)-2- (trifluoromethyl)thieno [2,3-d]pyrimidin-4(3H)- one |
| 167 | XH216-60p2 | 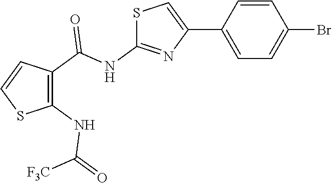 [SMILES: O=C(Nc1nc(-c2ccc(Br)cc2)cs1)c1ccsc1NC(=O)C(F)(F)F] | N-(4-(4- Bromophenyl)thiazol-2- yl)-2-(2,2,2- trifluoroacetamido) thiophene-3- carboxamide |
| 168 | XH216-76p1 | 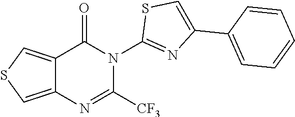 [SMILES: O=c1c2cscc2nc(C(F)(F)F)n1-c1nc(-c2ccccc2)cs1] | 3-(4- Phenylthiazol-2-yl)-2- (trifluoromethyl)thieno [3,4-d]pyrimidin-4(3H)- one |
| 169 | XH216-76p2 | 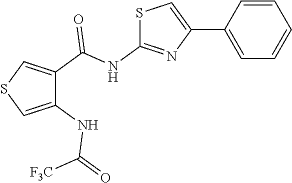 [SMILES: O=C(Nc1nc(-c2ccccc2)cs1)c1cscc1NC(=O)C(F)(F)F] | N-(4- Phenylthiazol-2-yl)-4- (2,2,2- trifluoroacetamido) thiophene-3- carboxamide |
| 170 | XH216-77p1 | 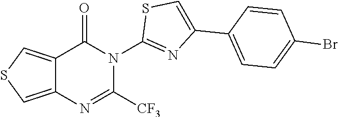 [SMILES: O=c1c2cscc2nc(C(F)(F)F)n1-c1nc(-c2ccc(Br)cc2)cs1] | 3-(4-(4- Bromophenyl)thiazol-2- yl)-2- (trifluoromethyl)thieno [3,4-d]pyrimidin-4(3H)- one |
| 171 | XH216-77p2 | 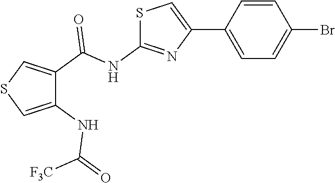 [SMILES: O=C(Nc1nc(-c2ccc(Br)cc2)cs1)c1cscc1NC(=O)C(F)(F)F] | N-(4-(4- Bromophenyl)thiazol-2- yl)-4-(2,2,2- trifluoroacetamido) thiophene-3- carboxamide |
| 172 | XH216-100P1 | 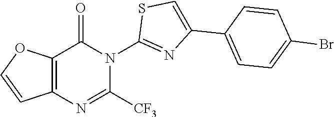 [SMILES: O=c1c2occc2nc(C(F)(F)F)n1-c1nc(-c2ccc(Br)cc2)cs1] | 3-(4-(4- Bromophenyl)thiazol-2- yl)-2- (trifluoromethyl)furo [3,2-d]pyrimidin-4(3H)- one |
| 173 | XH216-100P2 | 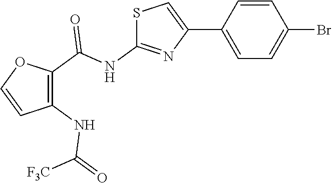 [SMILES: O=C(Nc1nc(-c2ccc(Br)cc2)cs1)c1occc1NC(=O)C(F)(F)F] | N-(4-(4- Bromophenyl)thiazol-2- yl)-3-(2,2,2- trifluoroacetamido) furan-2-carboxamide |
| 174 | XH216-118p2 | 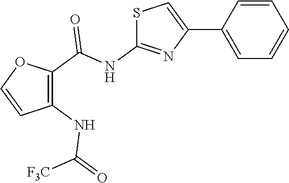 [SMILES: O=C(Nc1nc(-c2ccccc2)cs1)c1occc1NC(=O)C(F)(F)F] | N-(4- Phenylthiazol-2-yl)-3- (2,2,2- trifluoroacetamido) furan-2-carboxamide |
| 175 | XH216-119p2 | 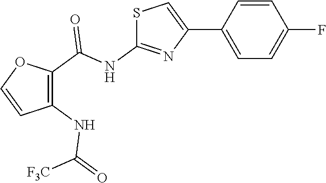 [SMILES: O=C(Nc1nc(-c2ccc(F)cc2)cs1)c1occc1NC(=O)C(F)(F)F] | N-(4-(4- Fluorophenyl)thiazol-2- yl)-3-(2,2,2- trifluoroacetamido) furan-2-carboxamide |
| 176 | XH216-125 | 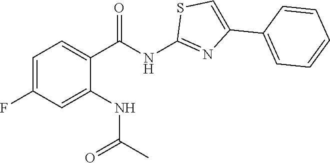 [SMILES: CC(=O)Nc1cc(F)ccc1C(=O)Nc1nc(-c2ccccc2)cs1] | 2-Acetamido-4- fluoro-N-(4- phenylthiazol-2- yl)benzamide |
| 177 | XH216-126 | 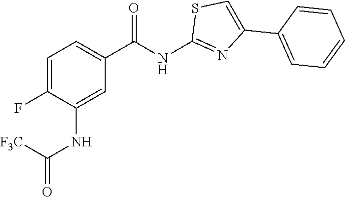 [SMILES: O=C(Nc1nc(-c2ccccc2)cs1)c1ccc(F)c(NC(=O)C(F)(F)F)c1] | 4-Fluoro-N-(4- phenylthiazol-2-yl)-3- (2,2,2- trifluoroacetamido) |
| 178 | XH216-127 | 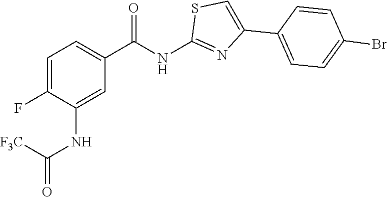 [SMILES: O=C(Nc1nc(-c2ccc(Br)cc2)cs1)c1ccc(F)c(NC(=O)C(F)(F)F)c1] | N-(4-(4- Bromophenyl)thiazol-2- yl)-4-fluoro-3-(2,2,2- trifluoroacetamido) benzamide |
| 179 | XH216-128 | 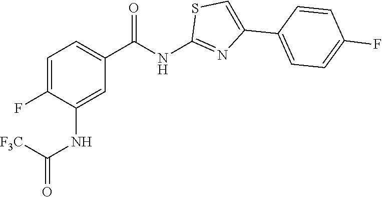 [SMILES: O=C(Nc1nc(-c2ccc(F)cc2)cs1)c1ccc(F)c(NC(=O)C(F)(F)F)c1] | 4-Fluoro-N-(4-(4- fluorophenyl)thiazol-2- yl)-3-(2,2,2- trifluoroacetamido) benzamide |
| 180 | XH216-129 | 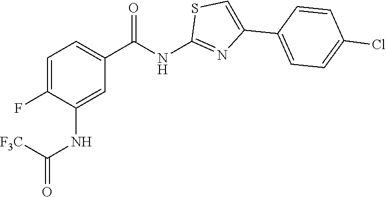 [SMILES: O=C(Nc1nc(-c2ccc(Cl)cc2)cs1)c1ccc(F)c(NC(=O)C(F)(F)F)c1] | N-(4-(4- Chlorophenyl)thiazol-2- yl)-4-fluoro-3-(2,2,2- trifluoroacetamido) benzamide |

### Chemistry General Procedures

All commercial chemical reagents and solvents were used for the reactions without further purification. Flash column chromatography was performed on Teledyne ISCO CombiFlash Rf+ instrument equipped with a 220/254/280 nm wavelength UV detector and a fraction collector. Normal phase column chromatography was conducted on silica gel columns with either hexane/ethyl acetate or dichloromethane/methanol as eluent. Reverse phase column chromatography was conducted on HP C18 RediSep Rf columns, and the gradient was set to 1000 of acetonitrile in H₂O containing 0.1% TFA progressing to 100% of acetonitrile. All final compounds were purified with preparative high-performance liquid chromatography (HPLC) on an Agilent Prep 1200 series with the UV detector set to 220/254 nm at a flow rate of 40 mL/min. Samples were injected onto a Phenomenex Luna 750×30 mm, 5 μm C18 column, and the gradient was set to 10% of acetonitrile in H₂O containing 0.1% TFA progressing to 100% of acetonitrile. All compounds assessed for biological activity have purity >95% as determined by an Agilent 1200 series system with DAD detector and a 2.1 mm×150 mm Zorbax 300SB-C18 5 m column for chromatography and high-resolution mass spectra (HRMS) that were acquired in positive ion mode using an Agilent G1969A API-TOF with an electrospray ionization (ESI) source. Samples (2 μL) were injected onto a C18 column at room temperature, and the flow rate was set to 0.4 mL/min with water containing 0.1% formic acid as solvent A and acetonitrile containing 0.1% formic acid as solvent B. Nuclear magnetic resonance (NMR) spectra were acquired on Bruker DRX 400 MHz for proton (¹H NMR) and 133 MHz for carbon (¹³C NMR). Chemical shifts for all compounds are reported in parts per million (ppm, 6). The format of chemical shift was reported as follows: chemical shift, multiplicity (s=singlet, d=doublet, t=triplet, q=quartet, m=multiplet), coupling constant (J values in Hz), and integration. All final compounds had >95% purity using the HPLC methods described above.

### EXAMPLES

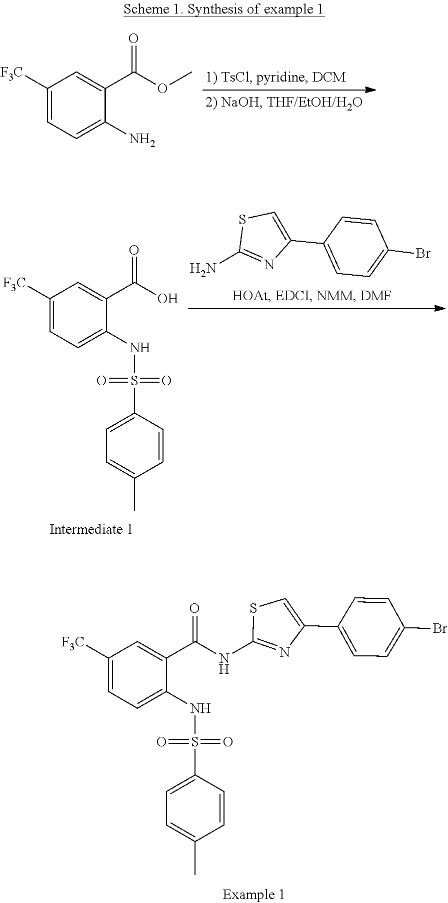 [SMILES: COC(=O)c1cc(C(F)(F)F)ccc1N ; Cc1ccc(S(=O)(=O)Nc2ccc(C(F)(F)F)cc2C(=O)O)cc1 ; Nc1nc(-c2ccc(Br)cc2)cs1 ; Cc1ccc(S(=O)(=O)Nc2ccc(C(F)(F)F)cc2C(=O)Nc2nc(-c3ccc(Br)cc3)cs2)cc1]

Example 1: N-(4-(4-bromophenyl)thiazol-2-yl)-2-((4-methylphenyl)sulfonamido)-5-(trifluoromethyl)benzamide (XH161-102). To a solution of methyl 2-amino-5-(trifluoromethyl)benzoate (438.3 g, 2 mmol) in DCM (2 mL) were added commercially available 4-methylbenzenesulfonyl chloride (419.4 g, 2.2 mmol, 1.1 equiv) and pyridine (0.49 mL, 6 mmol, 3.0 equiv). After being stirred for overnight at rt, the resulting mixture was purified by normal-phase column chromatography to afford crude product. To a solution of the crude product in THF/EtOH/H2O (6 mL/3 mL/3 mL) was added NaOH (240 mg, 6 mmol, 3.0 equiv) and the reaction was stirred at room temperature for 3 h. Then, the reaction mixture was acidified with 3 M HCl and extracted with ethyl acetate (EA, 3×10 mL). The combined organic layers were washed with brine solution (10 mL), dried over anhydrous Na₂SO₄, filtered and concentrated. The residue was purified by reverse-phase column chromatography (10%-100% acetonitrile/0.1% TFA in H₂O) to afford to yield intermediate 1 (510.8 mg, 71% yield).

To a solution of intermediate 1 (18 mg, 0.05 mmol) in DMF (0.5 mL) were added commercially available 4-(4-bromophenyl)thiazol-2-amine (12.8 mg, 0.05 mmol, 1.0 equiv), 1-ethyl-3-(3-dimethylaminopropyl)carbodiimide (EDCI, 14.3 mg, 0.075 mmol, 1.5 equiv), 1-hydroxy-7-azabenzo-triazole (HOAt, 10.2 mg, 0.075 mmol, 1.5 equiv), and N-methylmorpholine (NMM, 15.2 mg, 0.15 mmol, 3.0 equiv). After being stirred for overnight at rt, the crude reaction mixture was purified via prep-HPLC to yield title compound (4.9 mg, yield 16%). ¹H NMR (400 MHz, DMSO-d₆) δ ¹H NMR (400 MHz, DMSO-d₆) δ 13.04 (bs, 1H), 10.68 (bs, 1H), 8.27 (s, 1H), 7.90 (d, J=8.0 Hz, 2H), 7.86-7.83 (m, 2H), 7.74 (d, J=4.0 Hz, 2H), 7.65 (d, J=8.0 Hz, 2H), 7.57 (d, J=8.0 Hz, 1H), 7.32 (d, J=8.0 Hz, 2H), 2.28 (s, 3H). MS (ESI) [M+H]⁺=597.9.

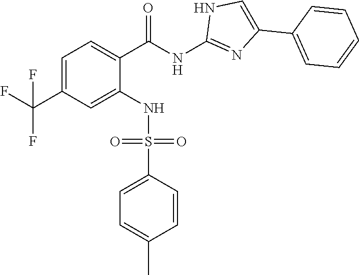 [SMILES: Cc1ccc(S(=O)(=O)Nc2cc(C(F)(F)F)ccc2C(=O)Nc2nc(-c3ccccc3)c[nH]2)cc1]

Example 2: 2-((4-methylphenyl)sulfonamido)-N-(4-phenyl-1H-imidazol-2-yl)-4-(trifluoromethyl)benzamide (XH153-7). Example 2 was synthesized following similar procedure for preparing example 1. (white solid, 2.1 mg, yield 30%) ¹H NMR (400 MHz, Methanol-d₄) δ 8.14 (d, J=8.0 Hz, 1H), 7.77 (s, 1H), 7.71 (d, J=8.0 Hz, 2H), 7.61 (d, J=8.0 Hz, 2H), 7.50-7.38 (m, 5H), 7.22 (d, J=8.0 Hz, 2H), 2.29 (s, 3H). MS (ESI) m/z 501.1 [M+H]⁺.

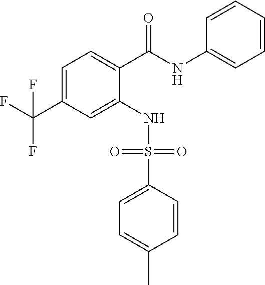 [SMILES: Cc1ccc(S(=O)(=O)Nc2cc(C(F)(F)F)ccc2C(=O)Nc2ccccc2)cc1]

Example 3: 2-((4-methylphenyl)sulfonamido)-N-phenyl-4-(trifluoromethyl)benzamide (XH153-10). Example 3 was synthesized following similar procedure for preparing example 1. (white solid, 5.8 mg, yield 48%) ¹H NMR (400 MHz, Methanol-d₄) δ 7.86-7.82 (m, 2H), 7.58-7.52 (m, 5H), 7.37 (t, J=8.0 Hz, 2H), 7.20-7.13 (m, 3H), 2.25 (s, 3H). MS (ESI) m/z 435.0 [M+H]⁺.

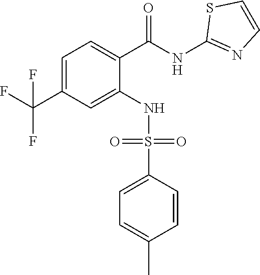 [SMILES: Cc1ccc(S(=O)(=O)Nc2cc(C(F)(F)F)ccc2C(=O)Nc2nccs2)cc1]

Example 4: 2-((4-methylphenyl)sulfonamido)-N-(thiazol-2-yl)-4-(trifluoromethyl)benzamide (xh153-74). Example 4 was synthesized following similar procedure for preparing example 1. (white solid, 16.6 mg, yield 67%) ¹H NMR (400 MHz, Chloroform-d) δ 10.95 (br, 2H), 8.03 (s, 2H), 7.70 (s, 2H), 7.44 (s, 1H), 7.26 (s, 1H), 7.18-7.15 (m, 3H), 2.29 (s, 3H). MS (ESI) m/z 442.1 [M+H]⁺.

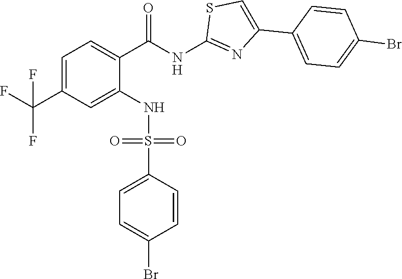 [SMILES: O=C(Nc1nc(-c2ccc(Br)cc2)cs1)c1ccc(C(F)(F)F)cc1NS(=O)(=O)c1ccc(Br)cc1]

Example 5: 2-((4-bromophenyl)sulfonamido)-N-(4-(4-bromophenyl)thiazol-2-yl)-4-(trifluoromethyl)benzamide (xh146-120). Example 5 was synthesized following similar procedure for preparing example 1. (yellow solid, 139 mg, yield 53%). ¹H NMR (400 MHz, Chloroform-d) δ 10.37 (bs, 1H), 8.04-8.01 (m, 1H), 7.74-7.66 (m, 5H), 7.57-7.50 (m, 4H), 7.45-7.43 (m, 2H), 7.27 (s, 1H). MS (ESI) m/z 661.9 [M+H]⁺.

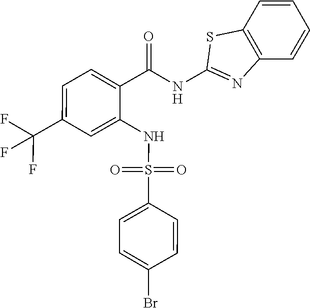 [SMILES: O=C(Nc1nc2ccccc2s1)c1ccc(C(F)(F)F)cc1NS(=O)(=O)c1ccc(Br)cc1]

Example 6: N-(benzo[d]thiazol-2-yl)-2-((4-methylphenyl)sulfonamido)-4-(trifluoromethyl)benzamide (xh146-123). Example 6 was synthesized following similar procedure for preparing example 1. (yellow solid, 128.1 mg, yield 58%). ¹H NMR (400 MHz, DMSO-d₆) δ 12.97 (s, 1H), 8.04-8.02 (m, 2H), 7.76-7.63 (m, 7H), 7.53-7.49 (m, 2H), 7.40-7.36 (m, 1H). MS (ESI) m/z 557.9 [M+H]⁻.

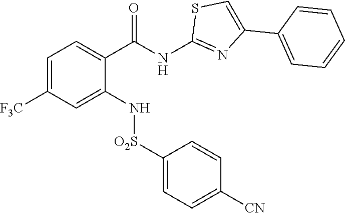 [SMILES: N#Cc1ccc(S(=O)(=O)Nc2cc(C(F)(F)F)ccc2C(=O)Nc2nc(-c3ccccc3)cs2)cc1]

Example 7: 2-((4-cyanophenyl)sulfonamido)-N-(4-phenylthiazol-2-yl)-4-(trifluoromethyl)benzamide (XH153-144). Example 7 was synthesized following similar procedure for preparing example 1. (white solid, 8.1 mg, yield 28%) ¹H NMR (400 MHz, Chloroform-d) δ 11.02 (br, 1H), 7.99 (d, J=8.0 Hz, 2H), 7.87 (s, 1H), 7.74-7.69 (m, 3H), 7.64 (d, J=8.0 Hz, 2H), 7.34-7.32 (m, 3H), 7.22-7.21 (m, 2H). MS (ESI) m/z 529.0 [M+H]⁺.

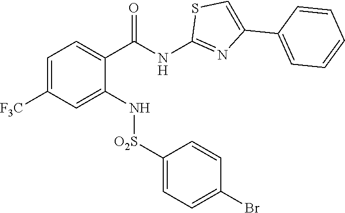 [SMILES: O=C(Nc1nc(-c2ccccc2)cs1)c1ccc(C(F)(F)F)cc1NS(=O)(=O)c1ccc(Br)cc1]

Example 8: 2-((4-bromophenyl)sulfonamido)-N-(4-phenylthiazol-2-yl)-4-(trifluoromethyl)benzamide (XH146-180). Example 8 was synthesized following similar procedure for preparing example 1. (yellow solid, 415 mg, yield 89%) ¹H NMR (400 MHz, Chloroform-d) δ 10.58 (s, 1H), 7.95 (s, 1H), 7.71-7.70 (m, 5H), 7.54 (d, J=8.0 Hz, 2H), 7.38 (t, J=8.0 Hz, 2H), 7.34-7.28 (m, 2H), 7.23 (s, 1H). MS (ESI) m/z 583.9 [M+H]⁺.

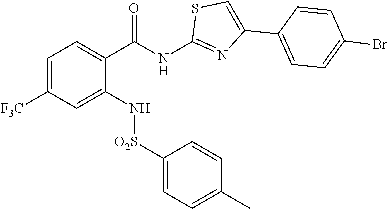 [SMILES: Cc1ccc(S(=O)(=O)Nc2cc(C(F)(F)F)ccc2C(=O)Nc2nc(-c3ccc(Br)cc3)cs2)cc1]

Example 9: N-(4-(4-bromophenyl)thiazol-2-yl)-2-((4-methylphenyl)sulfonamido)-4-(trifluoromethyl)benzamide (XH153-6). Example 9 was synthesized following similar procedure for preparing example 1. (yellow solid, 938 mg, yield 79%) ¹H NMR (400 MHz, CDCl₃) δ 10.09 (s, 1H), 7.72 (s, 1H), 7.53-7.47 (m, 2H), 7.44 (d, J=8.0 Hz, 1H), 7.39-7.36 (m, 2H), 7.28-7.25 (m, 2H), 7.06-7.02 (m, 2H), 6.96 (d, J=8.0 Hz, 2H), 2.09 (s, 3H). MS (ESI) m/z 598.0 [M+H]⁺.

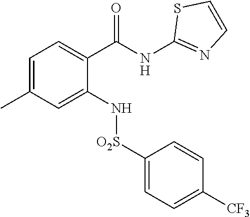 [SMILES: Cc1ccc(C(=O)Nc2nccs2)c(NS(=O)(=O)c2ccc(C(F)(F)F)cc2)c1]

Example 10: 4-Methyl-N-(thiazol-2-yl)-2-((4-(trifluoromethyl)phenyl)sulfonamido)benzamide (XH 153-168). Example 10 was synthesized following similar procedure for preparing example 1. (white solid, 10.5 mg, yield 42%)¹H NMR (400 MHz, Methanol-d₄) δ 7.83 (d, J=8.0 Hz, 2H), 7.70 (d, J=8.0 Hz, 1H), 7.63 (d, J=8.0 Hz, 2H), 7.46 (s, 2H), 7.15-7.09 (m, 2H), 2.38 (s, 3H). MS (ESI) m/z 442.4 [M+H]⁺.

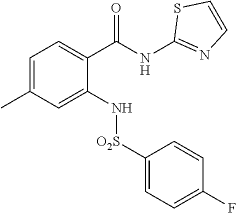 [SMILES: Cc1ccc(C(=O)Nc2nccs2)c(NS(=O)(=O)c2ccc(F)cc2)c1]

Example 11: 2-((4-Fluorophenyl)sulfonamido)-4-methyl-N-(thiazol-2-yl)benzamide (XH 153-157). Example 11 was synthesized following similar procedure for preparing example 1. (white solid, 10.3 mg, yield 40%) ¹H NMR (400 MHz, Methanol-d₄) δ 7.69-7.67 (m, 3H), 7.49-7.44 (m, 2H), 7.17 (s, 1H), 7.09-7.02 (m, 3H), 2.38 (s, 3H). MS (ESI) m/z 392.2 [M+H]⁺.

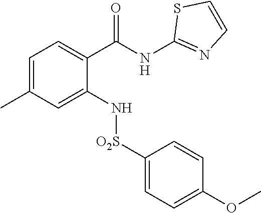

Example 12: 2-((4-methoxyphenyl)sulfonamido)-4-methyl-N-(thiazol-2-yl)benzamide (XH 153-148). Example 12 was synthesized following similar procedure for preparing example 1. (white solid, 8.4 mg, yield 34%) ¹H NMR (400 MHz, Chloroform-d) δ 11.82 (s, 1H), 9.98 (s, 1H), 7.67-7.65 (m, 2H), 7.58 (s, 1H), 7.52 (s, 1H), 6.98 (s, 2H), 6.89 (s, 1H), 6.65-6.64 (m, 2H), 3.61 (s, 3H), 2.42 (s, 3H). MS (ESI) m/z 404.1 [M+H]⁺.

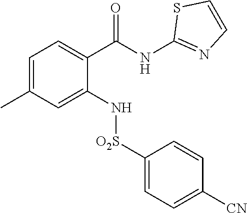 [SMILES: Cc1ccc(C(=O)Nc2nccs2)c(NS(=O)(=O)c2ccc(C#N)cc2)c1]

Example 13: 2-((4-cyanophenyl)sulfonamido)-4-methyl-N-(thiazol-2-yl)benzamide (XH 153-147). Example 13 was synthesized following similar procedure for preparing example 1. (white solid, 15.8 mg, yield 63%) ¹H NMR (400 MHz, Methanol-d₄) δ 7.76-7.74 (m, 2H), 7.67-7.62 (m, 3H), 7.49-7.45 (m, 2H), 7.17-7.11 (m, 2H), 2.39 (s, 3H). MS (ESI) m/z 399.3 [M+H]⁺.

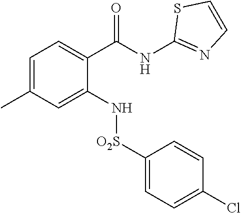 [SMILES: Cc1ccc(C(=O)Nc2nccs2)c(NS(=O)(=O)c2ccc(Cl)cc2)c1]

Example 14: 2-((4-chlorophenyl)sulfonamido)-4-methyl-N-(thiazol-2-yl)benzamide (XH 153-167). Example 14 was synthesized following similar procedure for preparing example 1. (white solid, 10.7 mg, yield 43%) ¹H NMR (400 MHz, Methanol-d₄) δ 7.69 (s, 1H), 7.60-7.59 (m, 2H), 7.48 (s, 1H), 7.44 (s, 1H), 7.30 (d, J=8.0 Hz, 2H), 7.16 (s, 1H), 7.09 (d, J=8.0 Hz, 1H), 2.38 (s, 3H). MS (ESI) m/z 408.5 [M+H]⁺.

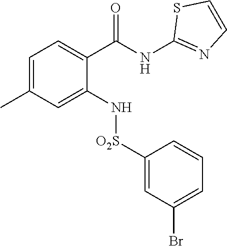 [SMILES: Cc1ccc(C(=O)Nc2nccs2)c(NS(=O)(=O)c2cccc(Br)c2)c1]

Example 15: 2-((3-bromophenyl)sulfonamido)-4-methyl-N-(thiazol-2-yl)benzamide (XH 153-145). Example 15 was synthesized following similar procedure for preparing example 1. (white solid, 9.8 mg, yield 40%) ¹H NMR (400 MHz, Methanol-d₄) δ 7.77 (s, 1H), 7.70 (d, J=8.0 Hz, 1H), 7.59 (d, J=8.0 Hz, 1H), 7.52 (d, J=8.0 Hz, 1H), 7.48 (s, 1H), 7.44 (s, 1H), 7.21 (t, J=8.0 Hz, 1H), 7.16 (s, 1H), 7.10 (d, J=8.0 Hz, 1H), 2.39 (s, 3H). MS (ESI) m/z 453.9 [M+H]⁺.

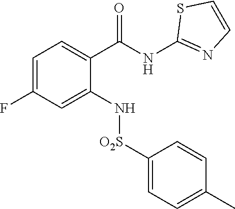 [SMILES: Cc1ccc(S(=O)(=O)Nc2cc(F)ccc2C(=O)Nc2nccs2)cc1]

Example 16: 4-fluoro-2-((4-methylphenyl)sulfonamido)-N-(thiazol-2-yl)benzamide (XH 153-182). Example 16 was synthesized following similar procedure for preparing example 1. (white solid, 18.3 mg, yield 72%) ¹H NMR (400 MHz, Chloroform-d) δ 7.72 (d, J=4.0 Hz, 3H), 7.48 (d, J=12.0 Hz, 1H), 7.12 (d, J=8.0 Hz, 2H), 7.03 (d, J=12.0 Hz, 2H), 6.82-6.77 (m, 1H), 2.25 (s, 3H). MS (ESI) m/z 392.6 [M+H]⁺.

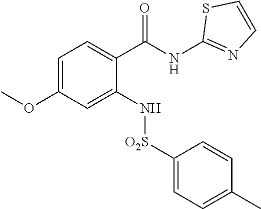

Example 17: 4-methoxy-2-((4-methylphenyl)sulfonamido)-N-(thiazol-2-yl)benzamide (XH153-155). Example 17 was synthesized following similar procedure for preparing example 1. (white solid, 20.2 mg, yield 81%) ¹H NMR (400 MHz, Methanol-d₄) δ 7.77 (d, J=8.0 Hz, 1H), 7.56 (d, J=8.0 Hz, 2H), 7.48 (s, 1H), 7.17-7.14 (m, 4H), 6.75 (d, J=12.0 Hz, 1H), 3.83 (s, 3H), 2.25 (s, 3H). MS (ESI) m/z 404.3 [M+H]⁺.

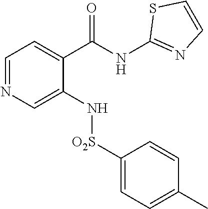 [SMILES: Cc1ccc(S(=O)(=O)Nc2cnccc2C(=O)Nc2nccs2)cc1]

Example 18: 3-((4-methylphenyl)sulfonamido)-N-(thiazol-2-yl)isonicotinamide (XH153-150). Example 18 was synthesized following similar procedure for preparing example 1. (yellow solid, 11.0 mg, yield 43%) ¹H NMR (400 MHz, Methanol-d₄) δ 8.85 (s, 1H), 8.43 (s, 1H), 8.07-8.05 (m, 1H), 7.54 (d, J=8.0 Hz, 2H), 7.48 (s, 1H), 7.16-7.12 (m, 3H), 2.23 (s, 3H). MS (ESI) m/z 375.2 [M+H]⁺.

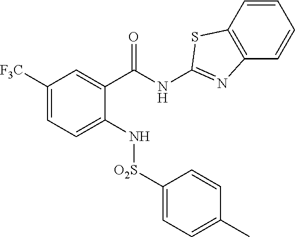 [SMILES: Cc1ccc(S(=O)(=O)Nc2ccc(C(F)(F)F)cc2C(=O)Nc2nc3ccccc3s2)cc1]

Example 19: N-(benzo[d]thiazol-2-yl)-2-((4-methylphenyl)sulfonamido)-5-(trifluoromethyl)benzamide (XH161-26). Example 19 was synthesized following similar procedure for preparing example 1. (white solid, 16.4 mg, yield 33%) ¹H NMR (400 MHz, Chloroform-d) δ 11.84 (s, 1H), 8.17 (s, 1H), 7.81-7.78 (m, 3H), 7.73-7.71 (m, 1H), 7.56 (dd, J=8.0, 4.0 Hz, 1H), 7.37-7.35 (m, 2H), 7.29-7.26 (m, 1H), 7.20 (d, J=8.0 Hz, 2H), 2.30 (s, 3H). MS (ESI) m/z 492.3 [M+H]⁺.

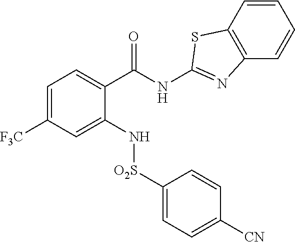 [SMILES: N#Cc1ccc(S(=O)(=O)Nc2cc(C(F)(F)F)ccc2C(=O)Nc2nc3ccccc3s2)cc1]

Example 20: N-(benzo[d]thiazol-2-yl)-2-((4-cyanophenyl)sulfonamido)-4-(trifluoromethyl)benzamide (XH161-2). Example 20 was synthesized following similar procedure for preparing example 1. (white solid, 9.4 mg, yield 19%) ¹H NMR (400 MHz, Chloroform-d) δ 8.21 (d, J=8.2 Hz, 1H), 7.96-7.94 (m, 3H), 7.83-7.81 (m, 1H), 7.70-7.68 (m, 2H), 7.62-7.60 (m, 1H), 7.55-7.51 (m, 1H), 7.44-7.39 (m, 2H). MS (ESI) m/z 503.3 [M+H]⁺.

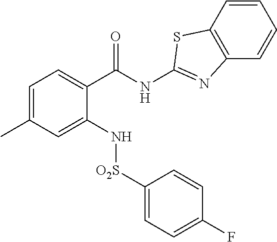 [SMILES: Cc1ccc(C(=O)Nc2nc3ccccc3s2)c(NS(=O)(=O)c2ccc(F)cc2)c1]

Example 21: N-(benzo[d]thiazol-2-yl)-2-((4-fluorophenyl)sulfonamido)-4-methylbenzamide (XH161-4). Example 21 was synthesized following similar procedure for preparing example 1. (white solid, 17.0 mg, yield 39%) 1H NMR (400 MHz, Chloroform-d) δ 10.52 (s, 1H), 7.89-7.84 (m, 3H), 7.57-7.55 (m, 2H), 7.36-7.33 (m, 1H), 7.30-7.26 (m, 1H), 7.13 (d, J=8.0 Hz, 1H), 7.03-6.89 (m, 2H), 6.76-6.73 (m, 1H), 2.32 (s, 3H). MS (ESI) m/z 442.3 [M+H]⁺.

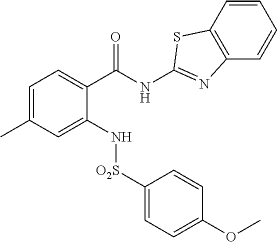 [SMILES: COc1ccc(S(=O)(=O)Nc2cc(C)ccc2C(=O)Nc2nc3ccccc3s2)cc1]

Example 22: N-(benzo[d]thiazol-2-yl)-2-((4-methoxyphenyl)sulfonamido)-4-methylbenzamide (XH161-5). Example 22 was synthesized following similar procedure for preparing example 1. (white solid, 18.8 mg, yield 41%) ¹H NMR (400 MHz, Chloroform-d) δ 10.37 (s, 1H), 7.87-7.84 (m, 1H), 7.73-7.71 (m, 2H), 7.58-7.55 (m, 2H), 7.37-7.35 (m, 3H), 6.83 (d, J=8.0 Hz, 1H), 6.76-6.74 (m, 2H), 3.62 (s, 3H), 2.35 (s, 3H). MS (ESI) m/z 454.9 [M+H]⁺.

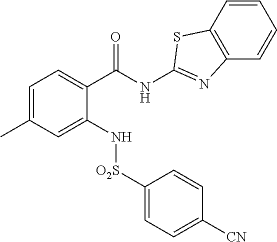 [SMILES: Cc1ccc(C(=O)Nc2nc3ccccc3s2)c(NS(=O)(=O)c2ccc(C#N)cc2)c1]

Example 23: N-(benzo[d]thiazol-2-yl)-2-((4-cyanophenyl)sulfonamido)-4-methylbenzamide (XH161-6). Example 23 was synthesized following similar procedure for preparing example 1. (white solid, 7.3 mg, yield 16%) ¹H NMR (400 MHz, Chloroform-d) δ 7.85 (d, J=8.0 Hz, 2H), 7.80 (d, J=8.0 Hz, 1H), 7.63 (d, J=8.0 Hz, 1H), 7.57 (d, J=12.0 Hz, 3H), 7.50 (s, 1H), 7.44-7.40 (m, 1H), 7.35-7.31 (m, 1H), 6.95-6.94 (m, 1H), 3.43 (s, 3H), 2.34 (s, 3H). MS (ESI) m/z 449.4[M+H]⁻.

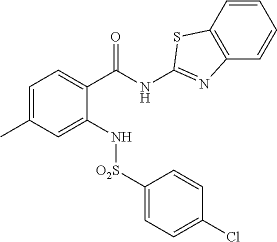 [SMILES: Cc1ccc(C(=O)Nc2nc3ccccc3s2)c(NS(=O)(=O)c2ccc(Cl)cc2)c1]

Example 24: N-(benzo[d]thiazol-2-yl)-2-((4-chlorophenyl)sulfonamido)-4-methylbenzamide (XH161-3). Example 24 was synthesized following similar procedure for preparing example 1. (white solid, 6.5 mg, yield 14%) ¹H NMR (400 MHz, Chloroform-d) δ 10.55 (bs, 1H), 7.82-7.80 (m, 1H), 7.75-7.72 (m, 2H), 7.51-7.48 (m, 2H), 7.32-7.22 (m, 4H), 7.13-7.11 (m, 1H), 6.73-6.70 (m, 1H), 2.29 (s, 3H). MS (ESI) m/z 459.1 [M+H]⁺.

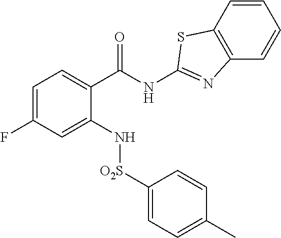 [SMILES: Cc1ccc(S(=O)(=O)Nc2cc(F)ccc2C(=O)Nc2nc3ccccc3s2)cc1]

Example 25: N-(benzo[d]thiazol-2-yl)-4-fluoro-2-((4-methylphenyl)sulfonamido)benzamide (XH161-19). Example 25 was synthesized following similar procedure for preparing example 1. (white solid, 15.4 mg, yield 35%) ¹H NMR (400 MHz, Chloroform-d) δ 11.25 (s, 1H), 7.84-7.82 (m, 1H), 7.77 (d, J=8.0 Hz, 3H), 7.46 (dd, J=8.0, 4.0 Hz, 1H), 7.37-7.29 (m, 2H), 7.19-7.17 (m 3H), 6.60-6.56 (m, 1H), 2.28 (s, 3H). MS (ESI) m/z 442.3 [M+H]⁺.

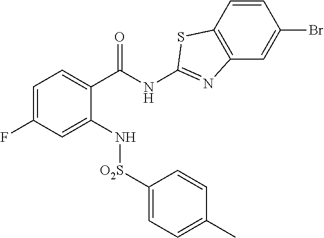 [SMILES: Cc1ccc(S(=O)(=O)Nc2cc(F)ccc2C(=O)Nc2nc3cc(Br)ccc3s2)cc1]

Example 26: N-(5-bromobenzo[d]thiazol-2-yl)-4-fluoro-2-((4-methylphenyl)sulfonamido)benzamide (XH161-37). Example 26 was synthesized following similar procedure for preparing example 1. (white solid, 8.4 mg, yield 16%) ¹H NMR (400 MHz, Chloroform-d) δ 10.72 (s, 1H), 7.76-7.67 (m, 4H), 7.53-7.47 (m, 3H), 7.17 (d, J=8.0 Hz, 2H), 6.75-6.70 (m, 1H), 2.28 (s, 3H). MS (ESI) m/z 521.9 [M+H]⁺.

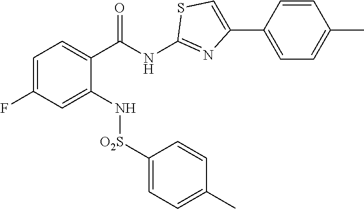 [SMILES: Cc1ccc(-c2csc(NC(=O)c3ccc(F)cc3NS(=O)(=O)c3ccc(C)cc3)n2)cc1]

Example 27: 4-fluoro-2-((4-methylphenyl)sulfonamido)-N-(4-(p-tolyl)thiazol-2-yl)benzamide (XH161-32). Example 27 was synthesized following similar procedure for preparing example 1. (white solid, 20.8 mg, yield 43%) ¹H NMR (400 MHz, Chloroform-d) δ 10.87 (bs, 1H), 7.74 (d, J=8.0 Hz, 2H), 7.60-7.55 (m, 3H), 7.37-7.34 (m, 1H), 7.21-7.11 (m, 5H), 6.63-6.58 (m, 1H), 2.35-2.32 (m, 6H). MS (ESI) m/z 482.2 [M+H]⁺.

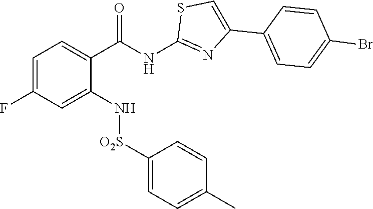 [SMILES: Cc1ccc(S(=O)(=O)Nc2cc(F)ccc2C(=O)Nc2nc(-c3ccc(Br)cc3)cs2)cc1]

Example 28: N-(4-(4-bromophenyl)thiazol-2-yl)-4-fluoro-2-((4-methylphenyl)sulfonamido)benzamide (XH161-33). Example 28 was synthesized following similar procedure for preparing example 1. (white solid, 20.3 mg, yield 37%) ¹H NMR (400 MHz, Chloroform-d) δ 10.59 (s, 1H), 9.71 (s, 1H), 7.73 (d, J=8.0 Hz, 2H), 7.65 (d, J=12.0 Hz, 2H), 7.58-7.50 (m, 3H), 7.47-7.44 (m, 1H), 7.23-7.18 (m, 3H), 6.80-6.74 (m, 1H), 2.31 (s, 3H). MS (ESI) m/z 547.4 [M+H]⁺.

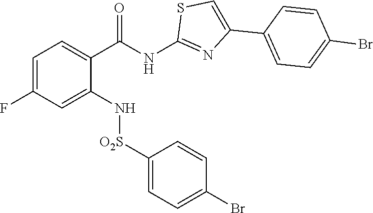 [SMILES: O=C(Nc1nc(-c2ccc(Br)cc2)cs1)c1ccc(F)cc1NS(=O)(=O)c1ccc(Br)cc1]

Example 29: 2-((4-bromophenyl)sulfonamido)-N-(4-(4-bromophenyl)thiazol-2-yl)-4-fluorobenzamide (XH161-25). Example 29 was synthesized following similar procedure for preparing example 1. (white solid, 13.8 mg, yield 43%) ¹H NMR (400 MHz, Chloroform-d) δ 10.72 (bs, 1H), 7.73-7.71 (m, 2H), 7.65 (d, J=8.0 Hz, 3H), 7.56-7.53 (m, 4H), 7.49-7.46 (m, 1H), 7.26-7.23 (m, 2H), 6.86-6.81 (m, 1H). MS (ESI) m/z 612.5 [M+H]⁺.

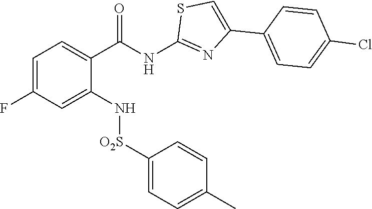 [SMILES: Cc1ccc(S(=O)(=O)Nc2cc(F)ccc2C(=O)Nc2nc(-c3ccc(Cl)cc3)cs2)cc1]

Example 30: N-(4-(4-chlorophenyl)thiazol-2-yl)-4-fluoro-2-((4-methylphenyl)sulfonamido)benzamide (XH161-31). Example 30 was synthesized following similar procedure for preparing example 1. (white solid, 21.0 mg, yield 42%) ¹H NMR (400 MHz, Chloroform-d) δ 10.60 (bs, 1H), 9.89 (bs, 1H), 7.74 (d, J=8.0 Hz, 2H), 7.69 (d, J=12.0 Hz, 2H), 7.56-7.52 (m, 1H), 7.45-7.42 (m, 1H), 7.34 (d, J=8.0 Hz, 2H), 7.21-7.18 (m, 3H), 6.75-6.71 (m, 1H), 2.31 (s, 3H). MS (ESI) m/z 502.3 [M+H]⁺.

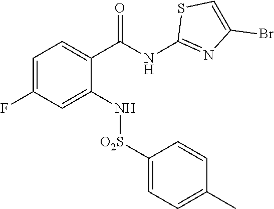 [SMILES: Cc1ccc(S(=O)(=O)Nc2cc(F)ccc2C(=O)Nc2nc(Br)cs2)cc1]

Example 31: N-(4-bromothiazol-2-yl)-4-fluoro-2-((4-methylphenyl)sulfonamido)benzamide (XH161-36). Example 31 was synthesized following similar procedure for preparing example 1. (white solid, 5.0 mg, yield 21%) ¹H NMR (400 MHz, Methanol-d₄) δ 7.77 (t, J=8.0 Hz, 1H), 7.52 (d, J=8.0 Hz, 2H), 7.36 (d, J=12.0 Hz, 1H), 7.24-7.12 (m, 3H), 7.00 (s, 1H), 2.27 (s, 3H). MS (ESI) m/z 471.4 [M+H]⁺.

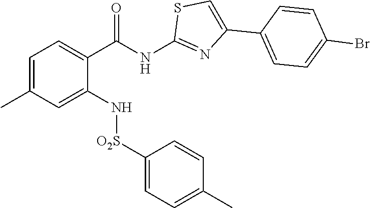 [SMILES: Cc1ccc(S(=O)(=O)Nc2cc(C)ccc2C(=O)Nc2nc(-c3ccc(Br)cc3)cs2)cc1]

Example 32: N-(4-(4-bromophenyl)thiazol-2-yl)-4-methyl-2-((4-methylphenyl)sulfonamido)benzamide (XH161-94). Example 32 was synthesized following similar procedure for preparing example 1. (white solid, 3.3 mg, yield 12%) ¹H NMR (400 MHz, Chloroform-d) δ 10.08 (s, 1H), 9.24 (s, 1H), 7.69 (d, J=8.0 Hz, 2H), 7.62 (d, J=8.0 Hz, 2H), 7.57-7.54 (m, 3H), 7.42 (d, J=8.0 Hz, 1H), 7.26-7.24 (m, 2H), 7.09 (d, J=8.0 Hz, 2H), 6.99 (d, J=8.0 Hz, 1H), 2.40 (s, 3H), 2.24 (s, 3H). MS (ESI) m/z 543.8 [M+H]⁺.

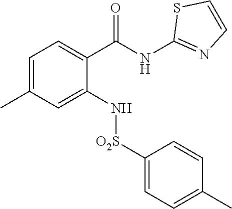

Example 33: 4-methyl-2-((4-methylphenyl)sulfonamido)-N-(thiazol-2-yl)benzamide (XH161-98). Example 33 was synthesized following similar procedure for preparing example 1. (white solid, 11.0 mg, yield 57%) ¹H NMR (400 MHz, Methanol-d₄) δ 7.65 (d, J=8.0 Hz, 1H), 7.48-7.44 (m, 4H), 7.17 (d, J=4.0 Hz, 1H), 7.07-7.06 (m, 3H), 2.37 (s, 3H), 2.22 (s, 3H). MS (ESI) m/z 388.5 [M+H]⁺.

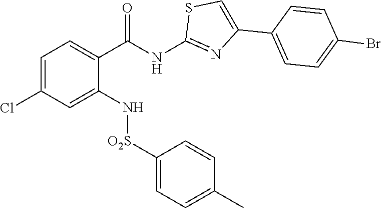 [SMILES: Cc1ccc(S(=O)(=O)Nc2cc(Cl)ccc2C(=O)Nc2nc(-c3ccc(Br)cc3)cs2)cc1]

Example 34: N-(4-(4-bromophenyl)thiazol-2-yl)-4-chloro-2-((4-methylphenyl)sulfonamido)benzamide (XH161-95). Example 34 was synthesized following similar procedure for preparing example 1. (white solid, 3.5 mg, yield 12%) ¹H NMR (400 MHz, Chloroform-d) δ 10.35 (bs, 1H), 7.78 (s, 1H), 7.68 (t, J=8.0 Hz, 4H), 7.56-7.51 (m, 3H), 7.26-7.24 (m, 2H), 7.17-7.12 (m, 3H), 2.29 (s, 3H). MS (ESI) m/z 563.6 [M+H]⁺.

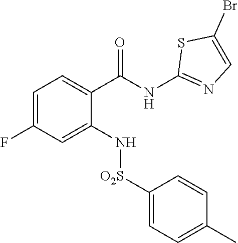 [SMILES: Cc1ccc(S(=O)(=O)Nc2cc(F)ccc2C(=O)Nc2ncc(Br)s2)cc1]

Example 35: N-(5-bromothiazol-2-yl)-4-fluoro-2-((4-methylphenyl)sulfonamido)benzamide (XH161-35). Example 35 was synthesized following similar procedure for preparing example 1. (white solid, 7.1 mg, yield 30%) ¹H NMR (400 MHz, Chloroform-d) δ 11.41-10.46 (m, 2H), 7.72 (d, J=8.0 Hz, 2H), 7.65 (dd, J=8.0, 4.0 Hz, 1H), 7.50 (dd, J=12.0, 4.0 Hz, 1H), 7.16 (d, J=8.0 Hz, 2H), 6.91 (s, 1H), 6.84-6.79 (m, 1H), 2.28 (s, 3H). MS (ESI) m/z 471.8 [M+H]⁺.

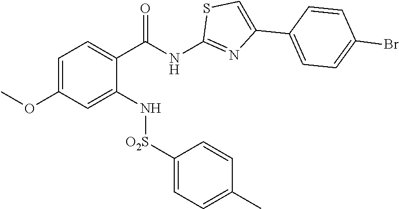 [SMILES: COc1ccc(C(=O)Nc2nc(-c3ccc(Br)cc3)cs2)c(NS(=O)(=O)c2ccc(C)cc2)c1]

Example 36: N-(4-(4-bromophenyl)thiazol-2-yl)-4-methoxy-2-((4-methylphenyl)sulfonamido)benzamide (XH161-96). Example 36 was synthesized following similar procedure for preparing example 1. (white solid, 3.4 mg, yield 12%) ¹H NMR (400 MHz, Chloroform-d) δ 10.66 (bs, 1H), 9.38 (bs, 1H), 7.72-7.67 (m, 4H), 7.55-7.48 (m, 3H), 7.21 (s, 1H), 7.26 (s, 1H), 7.16-7.15 (m 2H), 6.65-6.62 (m, 1H), 3.85 (s, 3H), 2.29 (s, 3H). MS (ESI) m/z 559.3 [M+H]⁺.

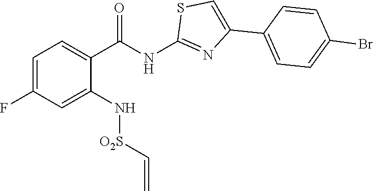 [SMILES: C=CS(=O)(=O)Nc1cc(F)ccc1C(=O)Nc1nc(-c2ccc(Br)cc2)cs1]

Example 37: N-(4-(4-bromophenyl)thiazol-2-yl)-4-fluoro-2-(vinylsulfonamido)benzamide (XH161-94). Example 37 was synthesized following similar procedure for preparing example 1. (white solid, 3.3 mg, yield 12%) ¹H NMR (400 MHz, Methanol-d₄) δ 8.06 (t, J=8.0 Hz, 1H), 7.86 (d, J=8.0 Hz, 2H), 7.57-7.51 (m, 3H), 7.38 (d, J=12.0 Hz, 1H), 7.01 (t, J=8.0 Hz, 1H), 6.79 (dd, J=16.0, 8.0 Hz, 1H), 6.33-6.29 (m, 1H), 6.07 (d, J=8.0 Hz, 1H). MS (ESI) m/z 543.8 [M+H]⁺.

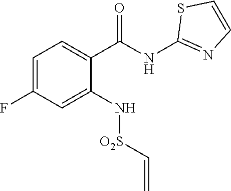 [SMILES: C=CS(=O)(=O)Nc1cc(F)ccc1C(=O)Nc1nccs1]

Example 38: 4-fluoro-N-(thiazol-2-yl)-2-(vinylsulfonamido)benzamide (XH161-135).

Example 38 was synthesized following similar procedure for preparing example 1. (white solid, 3.6 mg, yield 27%) ¹H NMR (400 MHz, Methanol-d₄) δ 8.14 (t, J=8.0 Hz, 1H), 7.47-7.46 (m, 1H), 7.35 (d, J=12.0 Hz, 1H), 7.13-7.12 (m, 1H), 6.95 (t, J=8.0 Hz, 1H), 6.75 (dd, J=16.0, 8.0 Hz, 1H), 6.31-6.27 (m, 1H), 6.06-6.03 (d, J=10.0 Hz, 1H). MS (ESI) m/z 328.4 [M+H]⁺.

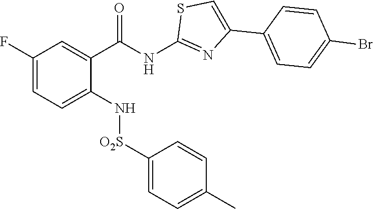 [SMILES: Cc1ccc(S(=O)(=O)Nc2ccc(F)cc2C(=O)Nc2nc(-c3ccc(Br)cc3)cs2)cc1]

Example 39: N-(4-(4-bromophenyl)thiazol-2-yl)-5-fluoro-2-((4-methylphenyl)sulfonamido)benzamide (XH161-132). Example 39 was synthesized following similar procedure for preparing example 1. (white solid, 6.0 mg, yield 17%) ¹H NMR (400 MHz, DMSO-d₆) δ 12.61 (s, 1H), 9.87 (s, 1H), 7.90 (d, J=8.0 Hz, 2H), 7.82 (s, 1H), 7.66 (d, J=8.0 Hz, 3H), 7.49 (d, J=8.0 Hz, 2H), 7.40 (t, J=8.0 Hz, 1H), 7.29 (s, 1H), 7.18 (d, J=8.0 Hz, 2H), 2.20 (s, 3H). MS (ESI) m/z 547.7 [M+H]⁺.

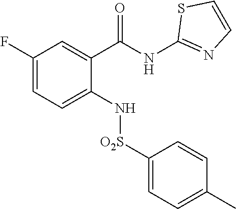 [SMILES: Cc1ccc(S(=O)(=O)Nc2ccc(F)cc2C(=O)Nc2nccs2)cc1]

Example 40: 5-fluoro-2-((4-methylphenyl)sulfonamido)-N-(thiazol-2-yl)benzamide (XH161-133). Example 40 was synthesized following similar procedure for preparing example 1. (white solid, 7.9 mg, yield 31%) ¹H NMR (400 MHz, Chloroform-d) δ 9.67 (bs, 1H), 7.77-7.73 (m, 1H), 7.57-7.54 (m, 2H), 7.40-7.38 (m, 1H), 7.30-7.27 (m, 1H), 7.08-7.07 (m, 1H), 6.98-6.96 (m, 3H), 2.15 (s, 3H). MS (ESI) m/z 392.3 [M+H]⁺.

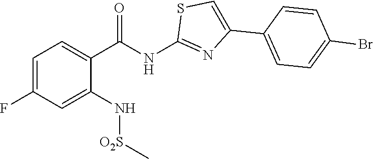 [SMILES: CS(=O)(=O)Nc1cc(F)ccc1C(=O)Nc1nc(-c2ccc(Br)cc2)cs1]

Example 41: N-(4-(4-bromophenyl)thiazol-2-yl)-4-fluoro-2-(methylsulfonamido)benzamide (XH161-101). Example 41 was synthesized following similar procedure for preparing example 1. (white solid, 2.0 mg, yield 9%) ¹H NMR (400 MHz, Chloroform-d) δ 7.87-7.83 (m, 1H), 7.70-7.67 (m, 2H), 7.61-7.56 (m, 3H), 7.52-7.50 (m, 1H), 7.22 (s, 1H), 3.14 (s, 3H).

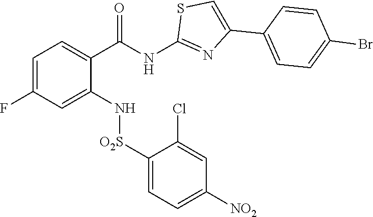 [SMILES: O=C(Nc1nc(-c2ccc(Br)cc2)cs1)c1ccc(F)cc1NS(=O)(=O)c1ccc([N+](=O)[O-])cc1Cl]

Example 42: N-(4-(4-bromophenyl)thiazol-2-yl)-2-((2-chloro-4-nitrophenyl)sulfonamido)-4-fluorobenzamide (XH161-136). Example 43 was synthesized following similar procedure for preparing example 1. (yellow solid, 6.9 mg, yield 22%) ¹H NMR (400 MHz, Chloroform-d) δ 11.67 (bs, 1H), 8.43-8.40 (m, 1H), 8.30-8.24 (m, 2H), 7.76-7.72 (m, 1H), 7.65 (d, J=8.0 Hz, 2H), 7.54 (d, J=8.0 Hz, 2H), 7.39 (d, J=12.0 Hz, 1H), 7.24 (s, 1H), 6.82 (t, J=8.0 Hz, 1H). MS (ESI) m/z 613.3 [M+H]⁺.

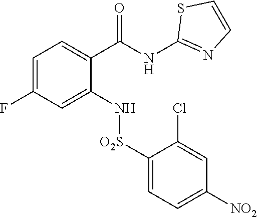 [SMILES: O=C(Nc1nccs1)c1ccc(F)cc1NS(=O)(=O)c1ccc([N+](=O)[O-])cc1Cl]

Example 43: 2-((2-chloro-4-nitrophenyl)sulfonamido)-4-fluoro-N-(thiazol-2-yl)benzamide (XH161-137). Example 44 was synthesized following similar procedure for preparing example 1. (white solid, 10.4 mg, yield 45%) ¹H NMR (400 MHz, Chloroform-d) δ 8.42 (d, J=8.0 Hz, 1H), 8.28-8.25 (m, 2H), 8.05 (dd, J=8.0, 4.0 Hz, 1H), 7.47 (d, J=4.0 Hz, 1H), 7.40 (d, J=12.0 Hz, 1H), 7.14 (d, J=4.0 Hz, 1H), 6.89 (t, J=8.0 Hz, 1H). MS (ESI) m/z 457.6 [M+H]⁻.

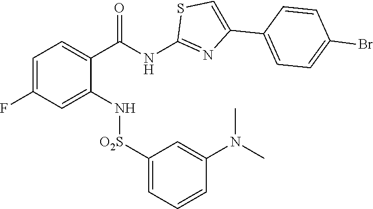 [SMILES: CN(C)c1cccc(S(=O)(=O)Nc2cc(F)ccc2C(=O)Nc2nc(-c3ccc(Br)cc3)cs2)c1]

Example 44: N-(4-(4-bromophenyl)thiazol-2-yl)-2-((3-(dimethylamino)phenyl)sulfonamido)-4-fluorobenzamide (XH161-169). Example 45 was synthesized following similar procedure for preparing example 1. (white solid, 4.5 mg, yield 26%) ¹H NMR (400 MHz, Methanol-d₄) δ 7.88-7.79 (m, 3H), 7.57-7.52 (m, 3H), 7.42 (d, J=8.0 Hz, 1H), 7.15-7.11 (m, 1H), 7.01 (t, J=8.0 Hz, 1H), 6.92 (s, 1H), 6.81-6.75 (m, 2H), 2.75 (s, 6H).

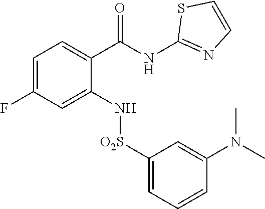 [SMILES: CN(C)c1cccc(S(=O)(=O)Nc2cc(F)ccc2C(=O)Nc2nccs2)c1]

Example 46: 2-((3-(dimethylamino)phenyl)sulfonamido)-4-fluoro-N-(thiazol-2-yl)benzamide (XH161-170). Example 46 was synthesized following similar procedure for preparing example 1. (white solid, 2.8 mg, yield 28%) ¹H NMR (400 MHz, Methanol-d₄) δ 7.93 (t, J=8.0 Hz, 1H), 7.48-7.46 (m, 1H), 7.41-7.37 (m, 1H), 7.16-7.11 (m, 2H), 6.95-6.88 (m, 3H), 6.80-6.77 (m, 1H), 2.78 (s, 6H). MS (ESI) m/z 421.4 [M+H]⁺.

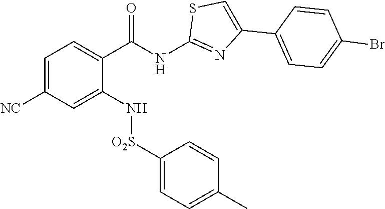 [SMILES: Cc1ccc(S(=O)(=O)Nc2cc(C#N)ccc2C(=O)Nc2nc(-c3ccc(Br)cc3)cs2)cc1]

Example 46: N-(4-(4-bromophenyl)thiazol-2-yl)-4-cyano-2-((4-methylphenyl)sulfonamido)benzamide (XH161-153). Example 47 was synthesized following similar procedure for preparing example 1. (white solid, 5.6 mg, yield 16%) ¹H NMR (400 MHz, Chloroform-d) δ 8.01 (s, 1H), 7.78-7.75 (m, 1H), 7.70-7.66 (m, 4H), 7.59-7.56 (m, 2H), 7.44-7.42 (m, 1H), 7.26-7.24 (m, 1H), 7.19-7.16 (m, 2H), 2.30 (s, 3H).

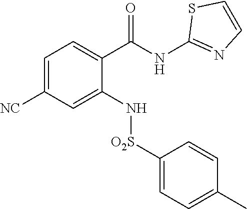 [SMILES: Cc1ccc(S(=O)(=O)Nc2cc(C#N)ccc2C(=O)Nc2nccs2)cc1]

Example 47: 4-cyano-2-((4-methylphenyl)sulfonamido)-N-(thiazol-2-yl)benzamide (XH161-154). Example 48 was synthesized following similar procedure for preparing example 1. (white solid, 4.7 mg, yield 19%) ¹H NMR (400 MHz, Chloroform-d) δ 11.38 (br, 2H), 7.96-7.93 (m, 2H), 7.71 (d, J=8.0 Hz, 2H), 7.35 (d, J=8.0 Hz, 1H), 7.14 (d, J=8.0 Hz, 2H), 7.03 (s, 2H), 2.28 (s, 3H). MS (ESI) m/z 399.8 [M+H]⁺.

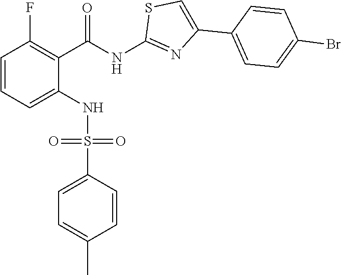 [SMILES: Cc1ccc(S(=O)(=O)Nc2cccc(F)c2C(=O)Nc2nc(-c3ccc(Br)cc3)cs2)cc1]

Example 48: N-(4-(4-bromophenyl)thiazol-2-yl)-2-fluoro-6-((4-methylphenyl)sulfonamido)benzamide (XH168-77). Example 49 was synthesized following similar procedure for preparing example 1. (white solid, 3.9 mg, yield 14%) ¹H NMR (400 MHz, Methanol-d₄) δ 7.87 (d, J=8.0 Hz, 2H), 7.57-7.51 (m, 4H), 7.46 (d, J=8.0 Hz, 2H), 7.41 (d, J=8.0 Hz, 1H), 7.11 (t, J=8.0 Hz, 1H), 7.01 (d, J=8.0 Hz, 2H), 2.10 (s, 1H). MS (ESI) m/z 547.8 [M+H]⁺.

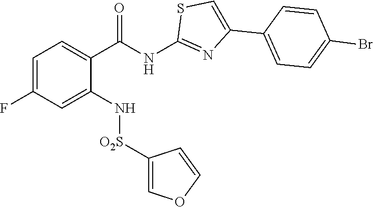 [SMILES: O=C(Nc1nc(-c2ccc(Br)cc2)cs1)c1ccc(F)cc1NS(=O)(=O)c1ccoc1]

Example 49: N-(4-(4-bromophenyl)thiazol-2-yl)-4-fluoro-2-(furan-3-sulfonamido)benzamide (XH181-20) Example 50 was synthesized following similar procedure for preparing example 1. (white solid, 3.2 mg, yield 12%) ¹H NMR (400 MHz, Chloroform-d) δ 10.89 (s, 1H), 9.79 (s, 1H), 7.99 (s, 1H), 7.71-7.66 (m, 3H), 7.54 (d, J=8.0 Hz, 3H), 7.39 (s, 1H), 7.23 (s, 1H), 6.87 (t, J=8.0 Hz, 1H), 6.63 (s, 1H). MS (ESI) m/z 523.9 [M+H]⁺.

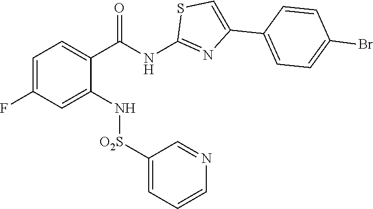 [SMILES: O=C(Nc1nc(-c2ccc(Br)cc2)cs1)c1ccc(F)cc1NS(=O)(=O)c1cccnc1]

Example 50: N-(4-(4-bromophenyl)thiazol-2-yl)-4-fluoro-2-(pyridine-3-sulfonamido)benzamide (XH181-38) Example 51 was synthesized following similar procedure for preparing example 1. (white solid, 5.8 mg, yield 15%) ¹H NMR (400 MHz, DMSO-d₆) δ 12.79 (br, 1H), 10.68 (br, 1H), 8.96 (s, 1H), 8.76 (s, 1H), 8.18 (d, J=8.0 Hz, 1H), 7.93-7.88 (m, 3H), 7.78 (s, 1H), 7.65 (d, J=8.0 Hz, 2H), 7.56 (s, 1H), 7.12 (d, J=12.0 Hz, 1H). MS (ESI) m/z 535.1 [M+H]⁺.

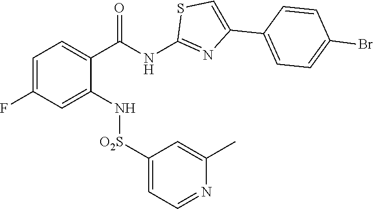 [SMILES: Cc1cc(S(=O)(=O)Nc2cc(F)ccc2C(=O)Nc2nc(-c3ccc(Br)cc3)cs2)ccn1]

Example 51: N-(4-(4-bromophenyl)thiazol-2-yl)-4-fluoro-2-((2-methylpyridine)-4-sulfonamido)benzamide (XH181-39) Example 52 was synthesized following similar procedure for preparing example 1. (white solid, 4.2 mg, yield 13%) ¹H NMR (400 MHz, Methanol-d₄) δ 8.71 (s, 1H), 8.02 (d, J=8.0 Hz, 1H), 7.7-7.83 (m, 3H), 7.57-7.53 (m, 3H), 7.43 (d, J=8.0 Hz, 1H), 7.29 (d, J=8.0 Hz, 1H), 7.10 (t, J=8.0 Hz, 1H),

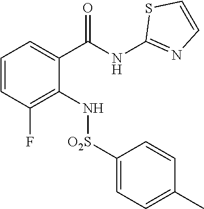 [SMILES: Cc1ccc(S(=O)(=O)Nc2c(F)cccc2C(=O)Nc2nccs2)cc1]

Example 52: 3-fluoro-2-((4-methylphenyl)sulfonamido)-N-(thiazol-2-yl)benzamide (XH161-177). Example 53 was synthesized following similar procedure for preparing example 1. (white solid, 5.6 mg, yield 45%) ¹H NMR (400 MHz, Methanol-d₄) δ 7.50-7.48 (m, 2H), 7.44-7.41 (m, 3H), 7.36-7.31 (m, 1H), 7.18 (s, 1H), 7.06 (d, J=8.0 Hz, 2H), 2.21 (s, 3H). MS (ESI) m/z 392.1 [M+H]⁺.

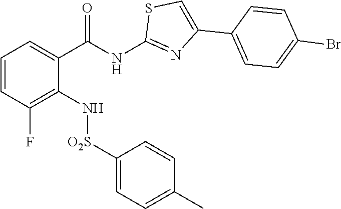 [SMILES: Cc1ccc(S(=O)(=O)Nc2c(F)cccc2C(=O)Nc2nc(-c3ccc(Br)cc3)cs2)cc1]

Example 53: N-(4-(4-bromophenyl)thiazol-2-yl)-3-fluoro-2-((4-methylphenyl)sulfonamido)benzamide (XH161-176). Example 54 was synthesized following similar procedure for preparing example 1. (white solid, 2.8 mg, yield 18%) ¹H NMR (400 MHz, Methanol-d₄) δ 7.87 (d, J=8.0 Hz, 2H), 7.56 (d, J=8.0 Hz, 2H), 7.50 (s, 1H), 7.45-7.35 (m, 5H), 7.03 (d, J=8.0 Hz, 2H), 2.10 (s, 3H). MS (ESI) m/z 547.6 [M+H]⁺.

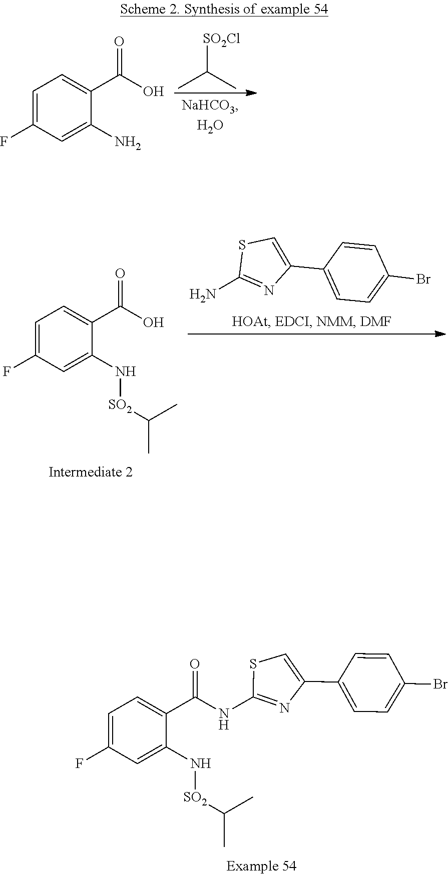

Example 54: N-(4-(4-bromophenyl)thiazol-2-yl)-4-fluoro-2-((1-methylethyl)sulfonamido)benzamide (XH161-172) To a solution of methyl 2-amino-4-fluorobenzoic acid (100 mg, 0.64 mmol) and NaHCO₃ (107.5 mg, 1.28 mmol, 2 equiv) in H₂O (5 mL) was added commercially available propane-2-sulfonyl chloride (109.5 mg, 0.768 mmol, 1.2 equiv) under 0° C. in ice bath. Then the reaction was warmed to room temperature and stirred for overnight at rt. The resulting mixture was purified by reverse-phase column chromatography to afford intermediate 2 (72.5 mg, 43% yield).

To a solution of intermediate 1 (20 mg, 0.77 mmol) in DMF (0.5 mL) were added commercially available 4-(4-bromophenyl)thiazol-2-amine (19.6 mg, 0.077 mmol, 1.0 equiv), 1-ethyl-3-(3-dimethylaminopropyl)carbodiimide (EDCI, 22.1 mg, 0.1155 mmol, 1.5 equiv), 1-hydroxy-7-azabenzo-triazole (HOAt, 15.7 mg, 0.1155 mmol, 1.5 equiv), and N-methylmorpholine (NMM, 23.3 mg, 0.231 mmol, 3.0 equiv). After being stirred for overnight at rt, the crude reaction mixture was purified via prep-HPLC to yield the title compound (5.83 mg, yield 15%). ¹H NMR (400 MHz, DMSO-d₆) δ 12.96 (s, 1H), 10.41 (s, 1H), 8.13 (t, J=8.0 Hz, 1H), 7.90 (d, J=8.0 Hz, 2H), 7.82 (s, 1H), 7.65 (d, J=8.0 Hz, 2H), 7.40 (d, J=12.0 Hz, 1H), 7.10 (d, J=12.0 Hz, 1H), 3.56-3.51 (m, 1H), 1.28-1.27 (m, 6H). MS (ESI) m/z 498.8 [M+H]⁺.

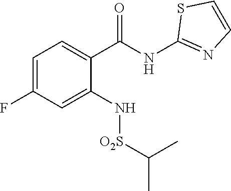 [SMILES: CC(C)S(=O)(=O)Nc1cc(F)ccc1C(=O)Nc1nccs1]

Example 55: 4-fluoro-2-((1-methylethyl)sulfonamido)-N-(thiazol-2-yl)benzamide (XH161-173). Example 55 was synthesized following similar procedure for preparing example 54. (white solid, 15.1 mg, yield 57%) ¹H NMR (400 MHz, Methanol-d₄) δ 8.17 (t, J=8.0 Hz, 1H), 7.53-7.46 (m, 2H), 7.13-7.12 (m, 1H), 6.93 (t, J=8.0 Hz, 1H), 3.45-3.34 (m, 1H), 1.33-1.31 (m, 6H). MS (ESI) m/z 344.1 [M+H]⁺.

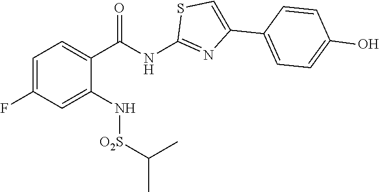 [SMILES: CC(C)S(=O)(=O)Nc1cc(F)ccc1C(=O)Nc1nc(-c2ccc(O)cc2)cs1]

Example 56: 4-fluoro-N-(4-(4-hydroxyphenyl)thiazol-2-yl)-2-((1-methylethyl)sulfonamido)benzamide (XH168-72-1) Example 56 was synthesized following similar procedure for preparing example 54. (white solid, 7.4 mg, yield 42%) ¹H NMR (400 MHz, Methanol-d₄) δ 8.39 (t, J=8.0 Hz, 1H), 7.78 (d, J=8.0 Hz, 2H), 7.59 (d, J=8.0 Hz, 1H), 7.40 (d, J=8.0 Hz, 2H), 7.08 (s, 1H), 7.00 (t, J=8.0 Hz, 1H), 3.53-3.45 (m, 1H), 1.36-1.35 (m, 6H). MS (ESI) m/z 436.5 [M+H]⁺.

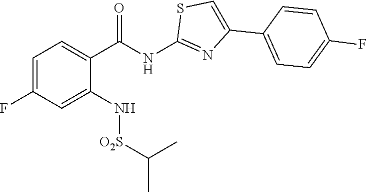 [SMILES: CC(C)S(=O)(=O)Nc1cc(F)ccc1C(=O)Nc1nc(-c2ccc(F)cc2)cs1]

Example 57: 4-fluoro-N-(4-(4-fluorophenyl)thiazol-2-yl)-2-((1-methylethyl)sulfonamido)benzamide (XH168-72-2) Example 57 was synthesized following similar procedure for preparing example 54. (white solid, 2.4 mg, yield 14%) ¹H NMR (400 MHz, Methanol-d₄) δ 8.08 (t, J=8.0 Hz, 1H), 7.95 (t, J=8.0 Hz, 1H), 7.56 (d, J=8.0 Hz, 1H), 7.41 (s, 1H), 7.14 (t, J=8.0 Hz, 2H), 6.99 (t, J=8.0 Hz, 1H), 3.46-3.39 (m, 1H), 1.35-1.34 (m, 6H). MS (ESI) m/z 439.1 [M+H]⁺.

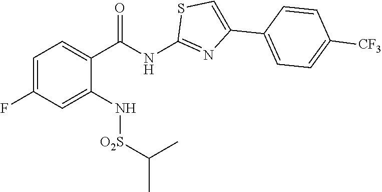 [SMILES: CC(C)S(=O)(=O)Nc1cc(F)ccc1C(=O)Nc1nc(-c2ccc(C(F)(F)F)cc2)cs1]

Example 58: 4-fluoro-2-((1-methylethyl)sulfonamido)-N-(4-(4-(trifluoromethyl)phenyl)thiazol-2-yl)benzamide (XH168-72-3) Example 58 was synthesized following similar procedure for preparing example 55. (white solid, 2.4 mg, yield 12%) ¹H NMR (400 MHz, Methanol-d₄) δ 8.13 (d, J=8.0 Hz, 2H), 8.08 (t, J=8.0 Hz, 2H), 7.70 (d, J=8.0 Hz, 2H), 7.66 (s, 1H), 7.57 (d, J=12.0 Hz, 1H), 7.00 (t, J=8.0 Hz, 1H), 3.47-3.40 (m, 1H), 1.35-1.34 (m, 6H). MS (ESI) m/z 488.7 [M+H]⁺.

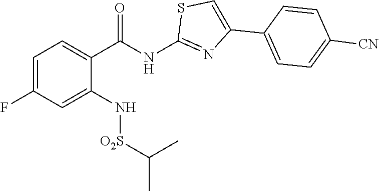 [SMILES: CC(C)S(=O)(=O)Nc1cc(F)ccc1C(=O)Nc1nc(-c2ccc(C#N)cc2)cs1]

Example 59: N-(4-(4-cyanophenyl)thiazol-2-yl)-4-fluoro-2-((1-methylethyl)sulfonamido)benzamide (XH168-79) Example 60 was synthesized following similar procedure for preparing example 54. (white solid, 3.7 mg, yield 21%) ¹H NMR (400 MHz, Methanol-d₄) δ 8.13 (d, J=8.0 Hz, 2H), 8.07 (t, J=8.0 Hz, 1H), 7.78-7.73 (m, 3H), 7.57 (d, J=12.0 Hz, 1H), 7.01 (t, J=8.0 Hz, 1H), 3.47-3.44 (m, 1H), 1.35-1.34 (m, 6H). MS (ESI) m/z 445.3 [M+H]⁺.

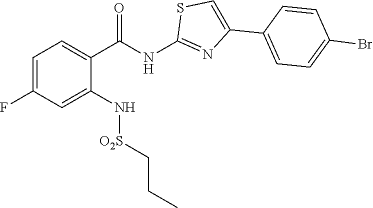 [SMILES: CCCS(=O)(=O)Nc1cc(F)ccc1C(=O)Nc1nc(-c2ccc(Br)cc2)cs1]

Example 60: N-(4-(4-bromophenyl)thiazol-2-yl)-4-fluoro-2-(propylsulfonamido)benzamide (XH168-63) Example 60 was synthesized following similar procedure for preparing example 54. (white solid, 7.8 mg, yield 16%) ¹H NMR (400 MHz, Chloroform-d) δ 10.49-10.13 (br, 2H), 7.49 (t, J=8.0 Hz, 1H), 7.43-7.41 (m, 2H), 7.35-7.30 (m, 3H), 7.06 (s, 1H), 7.01 (s, 1H), 6.56 (t, J=8.0 Hz, 1H), 2.99 (t, J=8.0 Hz, 2H), 1.72 (q, J=8.0 Hz, 2H), 0.85 (t, J=8.0 Hz, 3H). MS (ESI) m/z 499.1 [M+H]⁺.

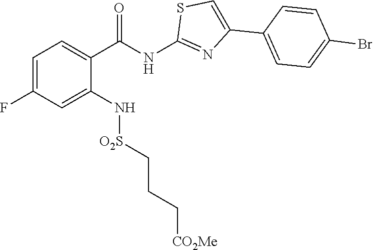 [SMILES: COC(=O)CCCS(=O)(=O)Nc1cc(F)ccc1C(=O)Nc1nc(-c2ccc(Br)cc2)cs1]

Example 61: Methyl 4-(N-(2-((4-(4-bromophenyl)thiazol-2-yl)carbamoyl)-5-fluorophenyl)sulfamoyl)butanoate (XH168-76) Example 61 was synthesized following similar procedure for preparing example 54. (white solid, 6.6 mg, yield 24%) ¹H NMR (400 MHz, Methanol-d₄) δ 8.07 (t, 0.1=8.0 Hz, 1H), 7.85 (d, J=8.0 Hz, 2H), 7.54 (d, J=8.0 Hz, 2H), 7.51-7.49 (m, 2H), 7.01 (t, J=8.0 Hz, 1H), 3.54 (s, 3H), 3.33-3.30 (m, 2H), 2.44 (t, J=8.0 Hz, 2H), 2.07-2.00 (m, 2H). MS (ESI) m/z 558.0 [M+H]⁺.

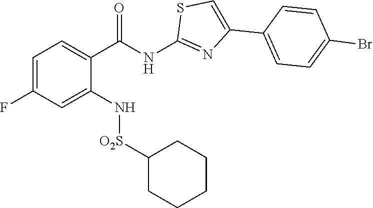 [SMILES: O=C(Nc1nc(-c2ccc(Br)cc2)cs1)c1ccc(F)cc1NS(=O)(=O)C1CCCCC1]

Example 62: N-(4-(4-bromophenyl)thiazol-2-yl)-2-(cyclohexanesulfonamide)-4-fluorobenzamide (XH161-180). Example 62 was synthesized following similar procedure for preparing example 54. (white solid, 4.8 mg, yield 13%) ¹H NMR (400 MHz, Methanol-d₄) δ 8.05 (t, J=8.0 Hz, 1H), 7.87-7.85 (m, 2H), 7.56-7.50 (m, 4H), 7.00 (t, J=8.0 Hz, 1H), 3.18-3.12 (m, 1H), 2.13-2.09 (m, 2H), 1.82-1.79 (m, 2H), 1.65-1.61 (m, 1H), 1.56-1.47 (m, 2H), 1.30-1.13 (m, 3H). MS (ESI) m/z 538.9 [M+H]⁺.

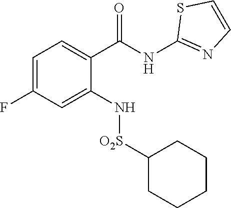 [SMILES: O=C(Nc1nccs1)c1ccc(F)cc1NS(=O)(=O)C1CCCCC1]

Example 63: 2-(cyclohexanesulfonamide)-4-fluoro-N-(thiazol-2-yl)benzamide (XH161-181).

Example 63 was synthesized following similar procedure for preparing example 54. (white solid, 14.3 mg, yield 57%) ¹H NMR (400 MHz, Methanol-d₄) δ 8.14 (t, J=8.0 Hz, 1H), 7.53-7.47 (m, 2H), 7.14-7.13 (m, 1H), 6.97-6.91 (m, 1H), 3.12 (t, J=12.0 Hz, 1H), 2.11-2.08 (m, 2H), 1.81-1.77 (m, 2H), 1.64-1.61 (m, 1H), 1.51 (q, J=12.0 Hz, 2H), 1.28-1.12 (m, 3H). MS (ESI) m/z 384.9 [M+H]⁺.

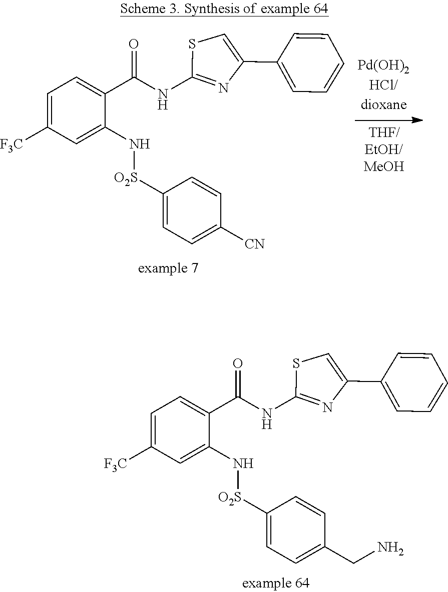 [SMILES: N#Cc1ccc(S(=O)(=O)Nc2cc(C(F)(F)F)ccc2C(=O)Nc2nc(-c3ccccc3)cs2)cc1 ; NCc1ccc(S(=O)(=O)Nc2cc(C(F)(F)F)ccc2C(=O)Nc2nc(-c3ccccc3)cs2)cc1]

### Example 64: 2-((4-(aminomethyl)phenyl)sulfonamido)-N-(4-phenylthiazol-2-yl)-4-(trifluoromethyl)benzamide (XH153-79-D)

To a solution of example 7 (200 mg, 0.38 mmol) in THF/EtOH/MeOH (1.2 mL/4.8 mL/1.2 mL) were added commercially available Pd(OH)₂ (79.4 mg, 0.038 mmol, 0.1 equiv) and HCl/dioxane (4 M, 0.38 mL). After being stirred for overnight at 50° C., the crude reaction mixture was purified via prep-HPLC to yield title compound (33.8 mg, yield 17%). ¹H NMR (400 MHz, Chloroform-d) δ 11.59 (br, 1H), 8.94 (br, 3H), 8.00 (s, 1H), 7.81 (d, J=8.0 Hz, 1H), 7.75 (d, J=8.0 Hz, 2H), 7.60 (d, J=8.0 Hz, 1H), 7.53-7.43 (m, 3H), 7.37 (d, J=8.0 Hz, 2H), 7.29 (s, 1H), 6.97 (d, J=8.0 Hz, 2H), 3.69 (s, 2H). MS (ESI) m/z 533.1 [M+H]⁺.

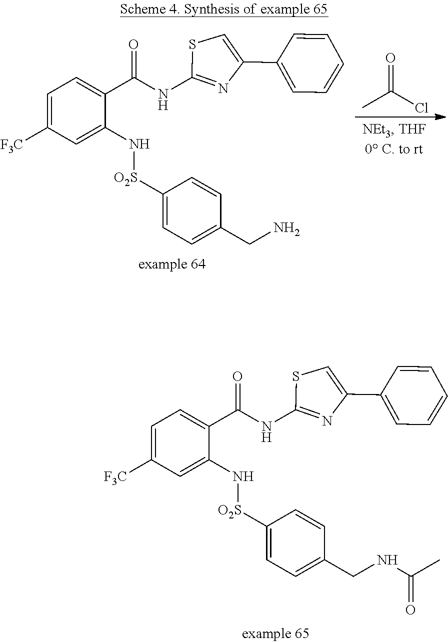

Example 65: 2-((4-(acetamidomethyl)phenyl)sulfonamido)-N-(4-phenylthiazol-2-yl)-4-(trifluoromethyl)benzamide (XH153-83). To a solution of Example 64 (7.7 mg, 0.014 mmol) in THF (5 mL) were added NEt₃ (1.8 mg, 0.016 mmol, 1.3 equiv) and acetyl chloride (1.3 mg, 0.0168 mmol, 1.2 equiv) under 0° C. in ice bath. Then the reaction mixture was warmed to room temperature and stirred at rt for 1 h. The resulting crude mixture was purified via prep-HPLC to yield the title compound (4.2 mg, 52% yield). ¹H NMR (400 MHz, Methanol-d₄) δ 7.94-7.89 (m, 4H), 7.58-7.56 (m, 3H), 7.49 (s, 1H), 7.42 (t, J=8.0 Hz, 2H), 7.33 (t, J=8.0 Hz, 1H), 7.18 (d, J=8.0 Hz, 2H), 4.19 (s, 2H), 1.90 (s, 3H). MS (ESI) m/z 575.2 [M+H]⁺.

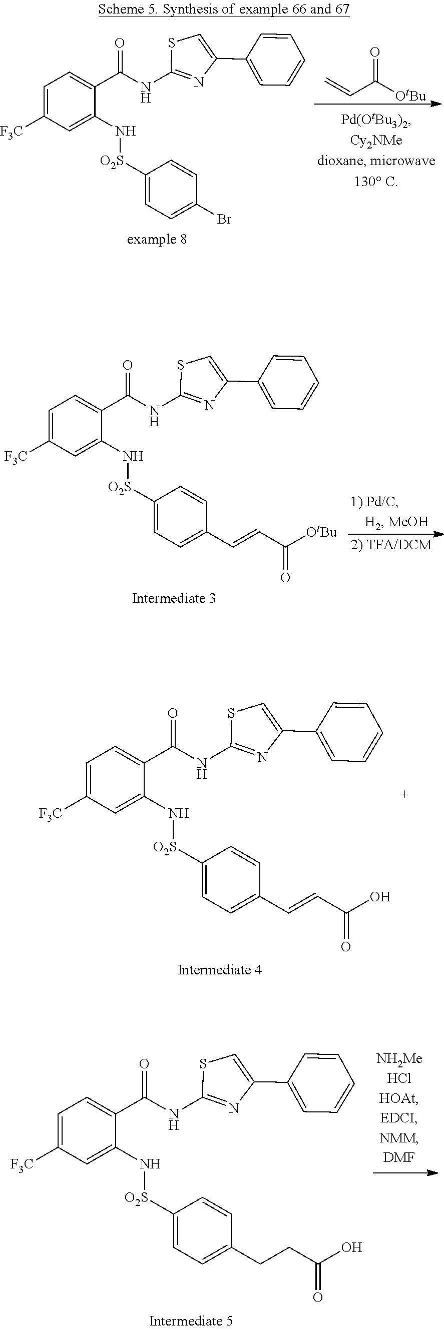

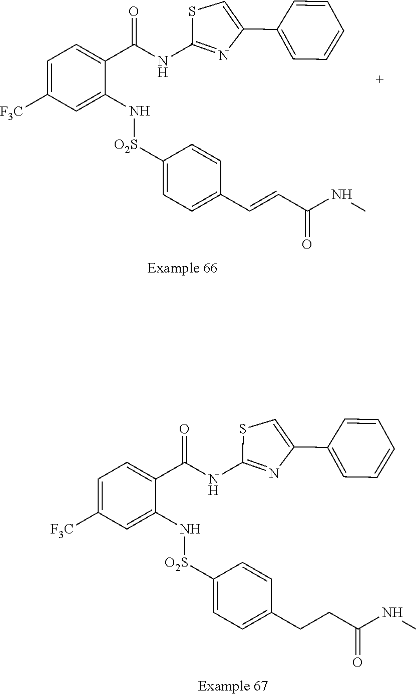 [SMILES: CNC(=O)/C=C/c1ccc(S(=O)(=O)Nc2cc(C(F)(F)F)ccc2C(=O)Nc2nc(-c3ccccc3)cs2)cc1 ; CNC(=O)CCc1ccc(S(=O)(=O)Nc2cc(C(F)(F)F)ccc2C(=O)Nc2nc(-c3ccccc3)cs2)cc1]

To a solution of Example 8 (200 mg, 0.34 mmol) in dioxane (2 mL) were added tert-butyl acrylate (87.2 mg, 0.68 mmol, 2 equiv), N-cyclohexyl-N-methylcyclohexanamine (Cy₂NMe, 6.6 mg, 0.034 mmol, 0.1 equiv) and bis(tri-tert-butylphosphine)palladium (Pd(PtBu₃)₂ 1.3 mg, 0.0168 mmol, 1.2 equiv) under Ar. Then the reaction mixture was stirred under microwave irradiation for 3 h at 130° C. The resulting crude mixture was purified by reverse-phase column chromatography to yield intermediate 3.

To a solution of intermediate 3 (110 mg, 0.17 mmol) in MeOH (5 mL) was added Pd/C (10 mg). The reaction mixture was vacuumed and recharged with H₂ three times. After the reaction was stirred at rt for overnight, the reaction mixture was filtered with celite pad and the celite was washed with MeOH (10 mL×3). The solvent in the filtrate was removed. The resulting crude product was re-dissolved in DCM (0.5 mL). To the resulting solution was added TFA (0.5 mL). The reaction mixture was stirred at rt 1 h. The resulting crude mixtures were purified by reverse-phase column chromatography to yield intermediate 4 and intermediate 5 (XH153-18).

To a solution of the mixture of intermediate 4 and intermediate 5 (10 mg, 0.017 mmol) in DMF (0.5 mL) were added commercially available Methylamine hydrochloride (1.1 mg, 0.017 mmol, 1.0 equiv), 1-ethyl-3-(3-dimethylaminopropyl)carbodiimide (EDCI, 4.9 mg, 0.0255 mmol, 1.5 equiv), 1-hydroxy-7-azabenzo-triazole (HOAt, 3.5 mg, 0.0255 mmol, 1.5 equiv), and N-methylmorpholine (NMM, 5.2 mg, 0.051 mmol, 3.0 equiv). After being stirred for overnight at rt, the resulting crude mixtures were purified via prep-HPLC to yield the title compounds, Example 66 (1.8 mg, yield 18%) and Example 67 (5.3 mg, yield 58%).

Example 66: (E)-2-((4-(3-(methylamino)-3-oxoprop-1-en-1-yl)phenyl)sulfonamido)-N-(4-phenylthiazol-2-yl)-4-(trifluoromethyl)benzamide (XH153-84-p1) ¹H NMR (400 MHz, Methanol-d₄) δ 7.89-7.86 (m, 4H), 7.64 (s, 1H), 7.57-7.55 (m, 2H), 7.47-7.37 (m, 5H), 7.30-7.26 (m, 2H), 6.28 (d, J=16.0 Hz, 1H), 2.60 (s, 3H). MS (ESI) m/z 587.1 [M+H]⁺.

Example 67: 2-((4-(3-(methylamino)-3-oxopropyl)phenyl)sulfonamido)-N-(4-phenylthiazol-2-yl)-4-(trifluoromethyl)benzamide (XH153-84-p2). ¹H NMR (400 MHz, Methanol-d₄) δ 7.93-7.88 (m, 4H), 7.59 (d, J=8.0 Hz, 1H), 7.51-7.49 (m, 3H), 7.41 (t, J=8.0 Hz, 2H), 7.32 (t, J=8.0 Hz, 1H), 7.11 (d, J=8.0 Hz, 2H), 2.74 (t, J=8.0 Hz, 2H), 2.51 (s, 3H), 2.25 (t, J=8.0 Hz, 2H). MS (ESI) m/z 589.2 [M+H]⁺.

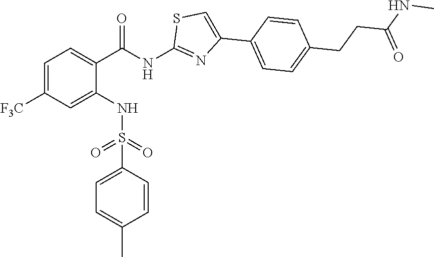 [SMILES: CNC(=O)CCc1ccc(-c2csc(NC(=O)c3ccc(C(F)(F)F)cc3NS(=O)(=O)c3ccc(C)cc3)n2)cc1]

Example 68: N-(4-(4-(3-(methylamino)-3-oxopropyl)phenyl)thiazol-2-yl)-2-((4-methylphenyl)sulfonamido)-4-(trifluoromethyl)benzamide (XH153-20). Example 68 was synthesized following similar procedure for preparing example 67. (white solid, 7.0 mg, yield 68%) ¹H NMR (400 MHz, Methanol-d₄) δ 7.87-7.84 (m, 4H), 7.58 (s, 1H), 7.46 (d, J=8.0 Hz, 3H), 7.27 (d, J=8.0 Hz, 2H), 7.07 (d, J=8.0 Hz, 2H), 2.94 (t, J=8.0 Hz, 2H), 2.67 (s, 3H), 2.49 (t, J=8.0 Hz, 2H), 2.16 (s, 3H). MS (ESI) m/z 603.9 [M+H]⁺.

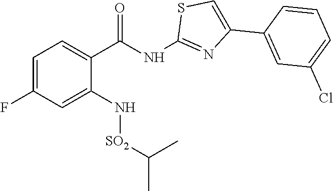 [SMILES: CC(C)S(=O)(=O)Nc1cc(F)ccc1C(=O)Nc1nc(-c2cccc(Cl)c2)cs1]

Example 69: N-(4-(3-chlorophenyl)thiazol-2-yl)-4-fluoro-2-((1-methylethyl)sulfonamido)benzamide (XH188-103) Example 69 was synthesized following similar procedure for preparing example 54. ¹H NMR (400 MHz, Methanol-d₄) δ 8.08 (t, J=8.0 Hz, 1H), 7.99 (s, 1H), 7.86 (d, J=8.0 Hz, 1H), 7.58-7.55 (m, 2H), 7.39 (t, J=8.0 Hz, 1H), 7.32-7.30 (m, 1H), 7.03-6.98 (m, 1H), 3.46-3.40 (m, 1H), 1.35-1.34 (m, 6H). MS (ESI) m/z 454.1 [M+H]⁺.

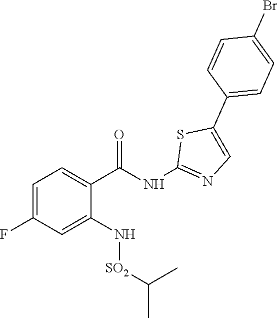 [SMILES: CC(C)S(=O)(=O)Nc1cc(F)ccc1C(=O)Nc1ncc(-c2ccc(Br)cc2)s1]

Example 70: N-(5-(4-bromophenyl)thiazol-2-yl)-4-fluoro-2-((1-methylethyl)sulfonamido)benzamide (XH188-104)) Example 70 was synthesized following similar procedure for preparing example 54. ¹H NMR (400 MHz, Chloroform-d) δ 10.93 (s, 1H), 7.94-7.90 (m, 1H), 7.64 (dd, J=11.2, 2.4 Hz, 1H), 7.61-7.54 (m, 2H), 7.40 (t, J=9.6 Hz, 3H), 6.87-6.82 (m, 1H), 3.41-3.37 (m, 1H), 1.45-1.44 (m, 6H). MS (ESI) m/z 499.9 [M+H]⁺.

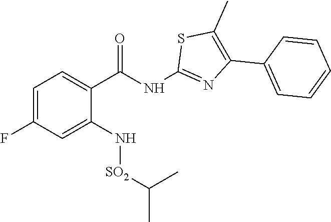 [SMILES: Cc1sc(NC(=O)c2ccc(F)cc2NS(=O)(=O)C(C)C)nc1-c1ccccc1]

Example 71: 4-Fluoro-N-(5-methyl-4-phenylthiazol-2-yl)-2-((1-methylethyl)sulfonamido)benzamide (XH188-105) Example 71 was synthesized following similar procedure for preparing example 54. ¹H NMR (400 MHz, Methanol-d₄) δ 8.18-8.14 (m, 1H), 7.63-7.60 (m, 2H), 7.52 (t, J=11.2 Hz, 2.8 Hz, 1H), 7.47 (t, J=7.2 Hz, 1H), 7.41-7.37 (t, J=7.4 Hz, 1H), 6.96-6.91 (m, 1H), 3.44-3.37 (m, 1H), 2.47 (s, 3H), 1.34-1.32 (m, 6H). MS (ESI) m/z 434.2 [M+H]⁺.

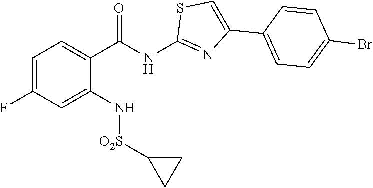 [SMILES: O=C(Nc1nc(-c2ccc(Br)cc2)cs1)c1ccc(F)cc1NS(=O)(=O)C1CC1]

Example 72: N-(4-(4-bromophenyl)thiazol-2-yl)-2-(cyclopropanesulfonamido)-4-fluorobenzamide (XH188-111) Example 72 was synthesized following similar procedure for preparing example 54. ¹H NMR (400 MHz, Chloroform-d) δ 10.60 (s, 1H), 7.70 (dd, J=8.8, 5.6 Hz, 1H), 7.62 (d, J=8.8 Hz, 2H), 7.58 (dd, J=10.8, 2.4 Hz, 1H), 7.51 (d, J=8.4 Hz, 2H), 7.22 (s, 1H), 6.81-6.76 (m, 1H), 2.64-2.57 (m, 1H), 1.34-1.30 (m, 2H), 1.06-1.01 (m, 2H). (white solid, yield 24%). MS (ESI) m/z 497.3 [M+H]⁺.

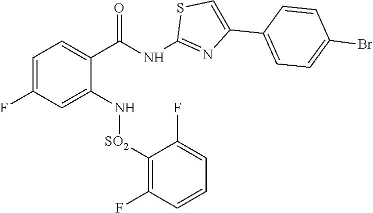 [SMILES: O=C(Nc1nc(-c2ccc(Br)cc2)cs1)c1ccc(F)cc1NS(=O)(=O)c1c(F)cccc1F]

Example 73: N-(4-(4-bromophenyl)thiazol-2-yl)-2-((2,6-difluorophenyl)sulfonamido)-4-fluorobenzamide (XH188-113) Example 73 was synthesized following similar procedure for preparing example 1. ¹H NMR (400 MHz, Chloroform-d) δ 7.88-7.85 (m, 1H), 7.62-7.61 (m, 2H), 7.55-7.47 (m, 4H), 7.19 (s, 1H), 7.00 (t, J=9.2 Hz, 2H), 6.84-6.79 (m, 1H). MS (ESI) m/z 469.9 [M+H]⁺.

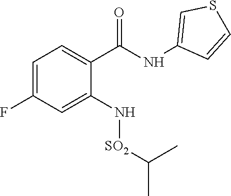 [SMILES: CC(C)S(=O)(=O)Nc1cc(F)ccc1C(=O)Nc1ccsc1]

Example 74: 4-Fluoro-2-((1-methylethyl)sulfonamido)-N-(thiophen-3-yl)benzamide (XH188-117) Example 74 was synthesized following similar procedure for preparing example 54. ¹H NMR (400 MHz, Chloroform-d) δ 10.79 (s, 1H), 8.34 (s, 1H), 7.67-7.62 (m, 2H), 7.54 (dd, J=10.8, 2.4 Hz, 1H), 7.30 (dd, J=5.2, 3.2 Hz, 1H), 7.12 (dd, J=5.2, 1.2 Hz, 1H), 6.84-6.79 (m, 1H), 3.38-3.32 (m, 1H), 1.42-1.40 (m, 6H). MS (ESI) m/z 343.1 [M+H]⁺.

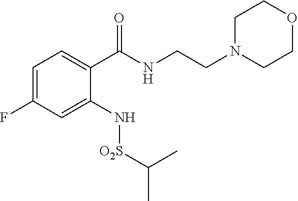 [SMILES: CC(C)S(=O)(=O)Nc1cc(F)ccc1C(=O)NCCN1CCOCC1]

Example 75: 4-Fluoro-2-((1-methylethyl)sulfonamido)-N-(2-morpholinoethyl)benzamide (XH188-119) Example 75 was synthesized following similar procedure for preparing example 54. ¹H NMR (400 MHz, Methanol-d₄) δ 7.81 (dd, J=8.8, 6.0 Hz, 1H), 7.45 (dd, J=10.8, 2.4 Hz, 1H), 6.95-6.91 (m, 1H), 4.10-4.07 (m, 2H), 3.79-3.65 (m, 6H), 3.42-3.38 (m, 3H), 3.25-3.19 (m, 2H), 1.35-1.33 (m, 6H). MS (ESI) m/z 374.4 [M+H]⁺.

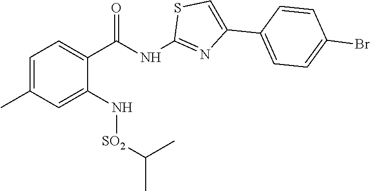 [SMILES: Cc1ccc(C(=O)Nc2nc(-c3ccc(Br)cc3)cs2)c(NS(=O)(=O)C(C)C)c1]

Example 76: N-(4-(4-Bromophenyl)thiazol-2-yl)-4-methyl-2-((1-methylethyl)sulfonamido)benzamide (XH188-122) Example 76 was synthesized following similar procedure for preparing example 54. ¹H NMR (400 MHz, Chloroform-d) δ 10.42 (s, 1H), 10.18 (s, 1H), 7.65-7.63 (m, 3H), 7.54-7.49 (m, 3H), 7.21 (s, 1H), 6.88 (dd, J=8.4, 1.6 Hz, 1H), 3.39-3.30 (m, 1H), 2.39 (s, 3H), 1.42-1.41 (m, 6H). MS (ESI) m/z 495.9 [M+H]⁺.

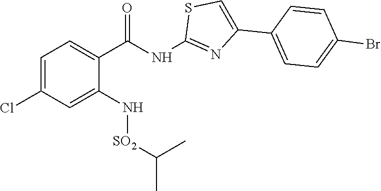 [SMILES: CC(C)S(=O)(=O)Nc1cc(Cl)ccc1C(=O)Nc1nc(-c2ccc(Br)cc2)cs1]

Example 77: N-(4-(4-Bromophenyl)thiazol-2-yl)-4-chloro-2-((1-methylethyl)sulfonamido)benzamide (XH188-123) Example 77 was synthesized following similar procedure for preparing example 54. ¹H NMR (400 MHz, Chloroform-d) δ 10.51 (bs, 2H), 7.86 (d, J=2.0 Hz, 1H), 7.63-7.58 (m, 3H), 7.52-7.50 (m, 2H), 7.22 (s, 1H), 7.03 (dd, J=8.4, 2.0 Hz, 1H), 3.42-3.35 (m, 1H), 1.45-1.43 (m, 6H). MS (ESI) m/z 515.9 [M+H]⁺.

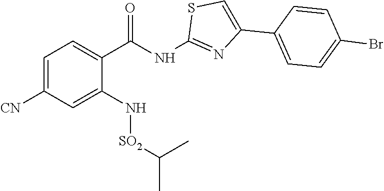

Example 78: N-(4-(4-Bromophenyl)thiazol-2-yl)-4-isocyano-2-((1-methylethyl)sulfonamido)benzamide (XH188-124) Example 78 was synthesized following similar procedure for preparing example 54. ¹H NMR (400 MHz, Chloroform-d) δ 10.68 (s, 1H), 8.13 (s, 1H), 7.88 (d, J=8.0 Hz, 1H), 7.61 (d, J=8.4 Hz, 2H), 7.55 (d, J=8.4 Hz, 2H), 7.36 (d, J=8.4, 1H), 7.22 (s, 1H), 3.41-3.35 (m, 1H), 1.46-1.44 (m, 6H). MS (ESI) m/z 506.9 [M+H]⁺.

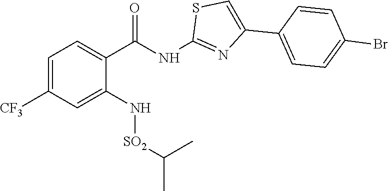 [SMILES: CC(C)S(=O)(=O)Nc1cc(C(F)(F)F)ccc1C(=O)Nc1nc(-c2ccc(Br)cc2)cs1]

Example 79: N-(4-(4-Bromophenyl)thiazol-2-yl)-2-((1-methylethyl)sulfonamido)-4-(trifluoromethyl)benzamide (XH188-125) Example 79 was synthesized following similar procedure for preparing example 54. ¹H NMR (400 MHz, Chloroform-d) δ 10.45 (s, 1H), 8.04 (s, 1H), 7.72 (d, J=8.4 Hz, 1H), 7.53 (d, J=8.4 Hz, 2H), 7.45 (d, J=8.4 Hz, 2H), 7.23 (s, 1H), 7.18 (d, J=8.4 Hz, 1H), 3.39-3.32 (m, 1H), 1.45-1.44 (m, 6H). MS (ESI) m/z 549.9 [M+H]⁺.

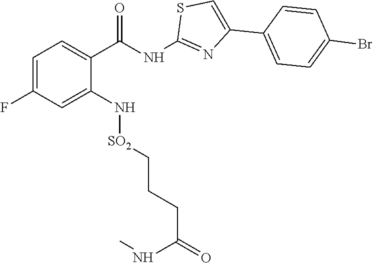 [SMILES: CNC(=O)CCCS(=O)(=O)Nc1cc(F)ccc1C(=O)Nc1nc(-c2ccc(Br)cc2)cs1]

Example 80: N-(4-(4-Bromophenyl)thiazol-2-yl)-4-fluoro-2-((4-(methylamino)-4-oxobutyl)sulfonamido)benzamide (XH188-137) Example 80 was synthesized following similar procedure for preparing example 54. ¹H NMR (400 MHz, Methanol-d₄) δ 8.08 (t, J=7.6 Hz, 1H), 7.87 (d, J=8.4 Hz, 2H), 7.57-7.49 (m, 4H), 7.06-7.01 (m, 1H), 3.54 (s, 3H), 3.39-3.33 (m, 2H), 2.45 (t, J=7.2 Hz, 2H), 2.08-2.00 (m, 2H). MS (ESI) m/z 555.9 [M+H]⁺.

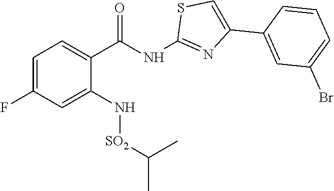 [SMILES: CC(C)S(=O)(=O)Nc1cc(F)ccc1C(=O)Nc1nc(-c2cccc(Br)c2)cs1]

Example 81: N-(4-(3-Bromophenyl)thiazol-2-yl)-4-fluoro-2-((1-methylethyl)sulfonamido)benzamide (xh188-146) Example 82 was synthesized following similar procedure for preparing example 54. ¹H NMR (400 MHz, Chloroform-d) δ 10.59 (s, 1H), 7.88-7.87 (m, 1H), 7.65-7.61 (m, 21H), 7.58-7.55 (m, 1H), 7.46-7.44 (m, 1H), 7.29-7.24 (m, 2H), 6.70-6.66 (m, 1H), 3.45-3.34 (m, 1H), 1.48-1.47 (m, 6H). MS (ESI) m/z 498.1 [M+H]⁻.

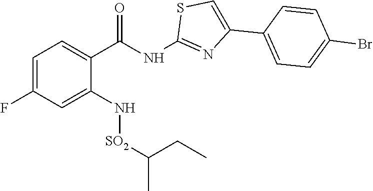 [SMILES: CCC(C)S(=O)(=O)Nc1cc(F)ccc1C(=O)Nc1nc(-c2ccc(Br)cc2)cs1]

Example 82: N-(4-(4-Bromophenyl)thiazol-2-yl)-4-fluoro-2-((1-methylpropyl)sulfonamido)benzamide (XH188-149) Example 83 was synthesized following similar procedure for preparing example 54. ¹H NMR (400 MHz, Chloroform-d) δ 10.64 (s, 1H), 7.75 (dd, J=9.2, 6.0 Hz, 1H), 7.65-7.59 (m, 3H), 7.55 (d, J=8.8 Hz, 2H), 7.22 (s, 1H), 6.82-6.78 (m, 1H), 3.18-3.10 (m, 1H), 2.16-2.06 (m, 1H), 1.72-1.61 (m, 1H), 1.41 (d, J=6.8 Hz, 3H), 1.03 (t, 0.1=7.6 Hz, 3H). MS (ESI) m/z 549.9 [M+H]⁺.

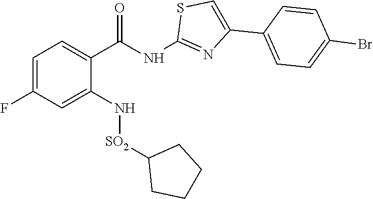 [SMILES: O=C(Nc1nc(-c2ccc(Br)cc2)cs1)c1ccc(F)cc1NS(=O)(=O)C1CCCC1]

Example 83: N-(4-(4-Bromophenyl)thiazol-2-yl)-2-((cyclopentyloxy)amino)-4-fluorobenzamide (XH188-150) Example 84 was synthesized following similar procedure for preparing example 54. H NMR (400 MHz, Chloroform-d) δ 10.64 (s, 1H), 7.73 (dd, J=8.8, 5.6 Hz, 1H), 7.69-7.62 (m, 3H), 7.53 (d, J=8.4 Hz, 2H), 7.22 (s, 1H), 6.83-6.78 (m, 1H), 3.68-3.60 (m, 1H), 2.17-2.08 (m, 2H), 2.01-1.93 (m, 3H), 1.88-1.79 (m, 2H), 1.66-1.60 (m, 2H). MS (ESI) m/z 524.0 [M+H]⁺.

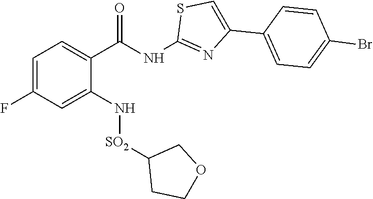 [SMILES: O=C(Nc1nc(-c2ccc(Br)cc2)cs1)c1ccc(F)cc1NS(=O)(=O)C1CCOC1]

Example 84: N-(4-(4-Bromophenyl)thiazol-2-yl)-4-fluoro-2-(((tetrahydrofuran-3-yl)oxy)amino)benzamide (XH188-151) Example 85 was synthesized following similar procedure for preparing example 54. ¹H NMR (400 MHz, Chloroform-d) δ 10.78 (s, 1H), 7.76 (dd, J=8.8, 5.6 Hz, 1H), 7.69-7.66 (m, 3H), 7.55 (d, J=8.4 Hz, 2H), 7.23 (s, 1H), 6.92-6.87 (m, 1H), 4.19-4.16 (m, 1H), 3.97-3.90 (m, 3H), 3.76 (q, J=7.6 Hz, 1H), 2.46-2.38 (m, 1H), 2.30-2.21 (m, 1H). MS (ESI) m/z 525.9 [M+H]⁺.

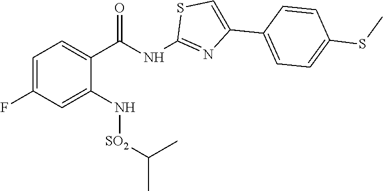 [SMILES: CSc1ccc(-c2csc(NC(=O)c3ccc(F)cc3NS(=O)(=O)C(C)C)n2)cc1]

Example 85: 4-Fluoro-2-((1-methylethyl)sulfonamido)-N-(4-(4-(methylthio)phenyl)thiazol-2-yl)benzamide (XH188-170) Example 86 was synthesized following similar procedure for preparing example 54. ¹H NMR (400 MHz, Chloroform-d) δ 10.76 (s, 1H), 7.66 (dd, J=9.2, 6.0 Hz, 1H), 7.61 (d, J=8.0 Hz, 2H), 7.54 (dd, J=11.2, 2.4 Hz, 1H), 7.18 (d, J=8.0 Hz, 2H), 7.14 (m, 1H), 6.68-6.63 (m, 1H), 3.39-3.32 (m, 1H), 2.59 (t, J=7.6 Hz, 2H), 1.68-1.59 (m, 2H), 1.43-1.41 (m, 6H), 0.94 (t, J=7.2 Hz, 3H). MS (ESI) m/z 466.1 [M+H]⁺.

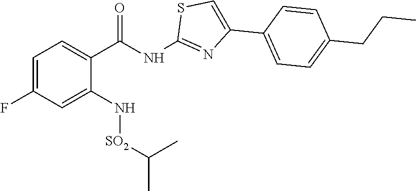 [SMILES: CCCc1ccc(-c2csc(NC(=O)c3ccc(F)cc3NS(=O)(=O)C(C)C)n2)cc1]

Example 86: 4-Fluoro-2-((1-methylethyl)sulfonamido)-N-(4-(4-propylphenyl)thiazol-2-yl)benzamide (xh188-171) Example 87 was synthesized following similar procedure for preparing example 54. ¹H NMR (400 MHz, Chloroform-d) δ 10.76 (s, 1H), 7.66 (dd, J=9.2, 6.0 Hz, 1H), 7.61 (d, J=8.0 Hz, 2H), 7.54 (dd, J=11.2, 2.4 Hz, 1H), 7.18 (d, J=8.0 Hz, 2H), 7.14 (m, 1H), 6.68-6.63 (m, 1H), 3.39-3.32 (m, 1H), 2.59 (t, J=7.6 Hz, 2H), 1.68-1.59 (m, 2H), 1.43-1.41 (m, 6H), 0.94 (t, J=7.2 Hz, 3H). MS (ESI) m/z 462.1 [M+H]⁺.

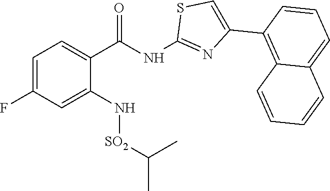 [SMILES: CC(C)S(=O)(=O)Nc1cc(F)ccc1C(=O)Nc1nc(-c2cccc3ccccc23)cs1]

Example 87: 4-Fluoro-2-((1-methylethyl)sulfonamido)-N-(4-(naphthalen-1-yl)thiazol-2-yl)benzamide (xh188-172) Example 88 was synthesized following similar procedure for preparing example 54. ¹H NMR (400 MHz, Chloroform-d) δ 10.43 (s, 1H), 8.04 (d, J=8.4 Hz, 1H), 7.86 (d, J=8.4 Hz, 1H), 7.77 (d, J=8.4 Hz, 1H), 7.50-7.43 (m, 2H), 7.37-7.28 (m, 3H), 7.23-7.20 (m, 2H), 6.26-6.21 (m, 1H), 3.33-3.26 (m, 1H), 1.42-1.41 (m, 6H). MS (ESI) m/z 470.1 [M+H]⁺.

Example 88-107 were synthesized following similar procedure for preparing example 54.

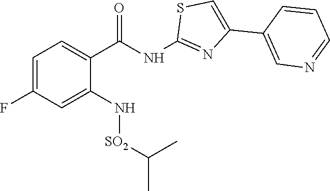 [SMILES: CC(C)S(=O)(=O)Nc1cc(F)ccc1C(=O)Nc1nc(-c2cccnc2)cs1]

Example 88: 4-Fluoro-2-((1-methylethyl)sulfonamido)-N-(4-(pyridin-3-yl)thiazol-2-yl)benzamide (XH188-173) ¹H NMR (400 MHz, Methanol-d₄) δ 9.33 (s, 1H), 8.93 (d, J=8.0 Hz, 1H), 8.70 (s, 1H), 8.10-8.06 (m, 1H), 7.99-7.95 (m, 2H), 7.56 (dd, J=11.2, 2.4 Hz, 1H), 7.04-6.99 (m, 1H), 3.48-3.40 (m, 1H), 1.36-1.34 (m, 6H). MS (ESI) m/z 421.1 [M+H]⁺.

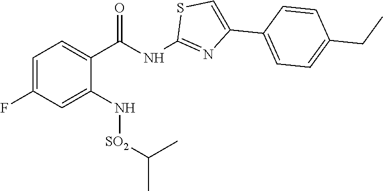 [SMILES: CCc1ccc(-c2csc(NC(=O)c3ccc(F)cc3NS(=O)(=O)C(C)C)n2)cc1]

Example 89: N-(4-(4-ethylphenyl)thiazol-2-yl)-4-fluoro-2-((1-methylethyl)sulfonamido)benzamide (XH188-174) ¹H NMR (400 MHz, Chloroform-d) δ 10.78 (s, 1H), 7.68 (dd, J=8.8, 6.0 Hz, 1H), 7.60 (d, J=8.0 Hz, 2H), 7.53 (dd, J=11.1, 2.4 Hz, 1H), 7.20 (d, J=7.6 Hz, 2H), 7.13 (s, 1H), 6.73-6.63 (m, 1H), 3.39-3.32 (m, 1H), 2.66 (q, J=7.6 Hz, 2H). MS (ESI) m/z 448.1 [M+H]⁺.

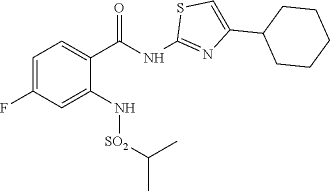 [SMILES: CC(C)S(=O)(=O)Nc1cc(F)ccc1C(=O)Nc1nc(C2CCCCC2)cs1]

Example 90: N-(4-Cyclohexylthiazol-2-yl)-4-fluoro-2-((1-methylethyl)sulfonamido)benzamide (XH188-175) ¹H NMR (400 MHz, Chloroform-d) δ 11.80 (bs, 1H), 8.02 (dd, J=8.4, 6.0 Hz, 1H), 7.56 (dd, J=11.2, 2.8 Hz, 1H), 6.84-6.79 (m, 1H), 6.45 (s, 1H), 3.40-3.33 (m, 1H), 2.58-2.53 (m, 1H), 2.04-1.99 (m, 2H), 1.86-1.82 (m, 2H), 1.77-1.73 (m, 1H), 1.40-1.31 (m, 9H), 1.28-1.22 (m, 2H). MS (ESI) m/z 426.1 [M+H]⁺.

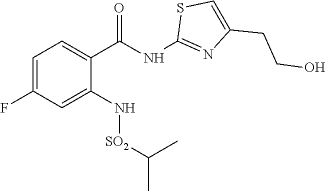 [SMILES: CC(C)S(=O)(=O)Nc1cc(F)ccc1C(=O)Nc1nc(CCO)cs1]

Example 91: 4-Fluoro-N-(4-(2-hydroxyethyl)thiazol-2-yl)-2-((1-methylethyl)sulfonamido)benzamide (XH188-176) ¹H NMR (400 MHz, Chloroform-d) δ 10.59 (bs, 1H), 8.23 (dd, J=9.2, 6.0 Hz, 1H), 7.63 (dd, J=10.8, 2.4 Hz, 1H), 6.97-6.92 (m, 1H), 6.89 (s, 1H), 3.99 (t, J=6.0 Hz, 2H), 3.43-3.33 (m, 1H), 3.05 (t, J=5.6 Hz, 2H), 1.42-1.40 (m, 6H). MS (ESI) m/z 388.1 [M+H]⁺.

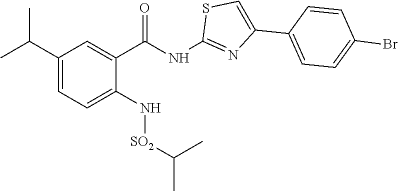 [SMILES: CC(C)c1ccc(NS(=O)(=O)C(C)C)c(C(=O)Nc2nc(-c3ccc(Br)cc3)cs2)c1]

Example 92: N-(4-(4-Bromophenyl)thiazol-2-yl)-5-isopropyl-2-((1-methylethyl)sulfonamido)benzamide (XH188-183) ¹H NMR (400 MHz, Chloroform-d) δ 10.14 (bs, 1H), 9.85 (bs, 1H), 7.80 (d, J=8.8 Hz, 1H), 7.69 (d, J=8.4 Hz, 2H), 7.56-7.52 (m, 3H), 7.42 (d, J=8.8 Hz, 1H), 7.23 (s, 1H), 3.38-3.31 (m, 1H), 2.96-2.89 (m, 1H), 1.41-1.40 (m, 6H), 1.29-1.27 (d, J=6.9 Hz, 6H). MS (ESI) m/z 522.0 [M+H]⁺.

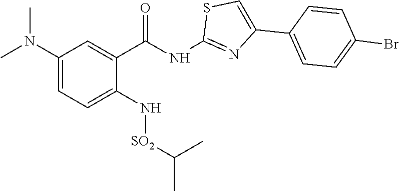 [SMILES: CC(C)S(=O)(=O)Nc1ccc(N(C)C)cc1C(=O)Nc1nc(-c2ccc(Br)cc2)cs1]

Example 93: N-(4-(4-Bromophenyl)thiazol-2-yl)-5-(dimethylamino)-2-((1-methylethyl)sulfonamido)benzamide (XH188-184) ¹H NMR (400 MHz, Chloroform-d) δ 9.96 (bs, 1H), 8.00 (s, 1H), 7.88 (d, J=9.2 Hz, 1H), 7.68-7.62 (m, 4H), 7.49 (d, J=9.2 Hz, 1H), 7.22 (s, 1H), 3.37-3.30 (m, 1H), 3.17 (s, 6H), 1.41-1.39 (m, 6H). MS (ESI) m/z 523.1 [M+H]⁺.

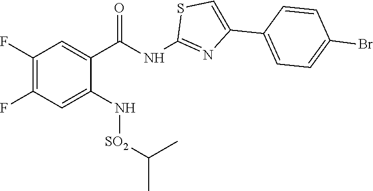 [SMILES: CC(C)S(=O)(=O)Nc1cc(F)c(F)cc1C(=O)Nc1nc(-c2ccc(Br)cc2)cs1]

Example 94: N-(4-(4-Bromophenyl)thiazol-2-yl)-4,5-difluoro-2-((1-methylethyl)sulfonamido)benzamide (XH188-185). ¹H NMR (400 MHz, Chloroform-d) δ 10.46 (bs, 1H), 7.68 (dd, J=12.0, 6.8 Hz, 1H), 7.54 (d, J=8.4 Hz, 2H), 7.47-7.38 (m, 3H), 7.24 (s, 1H), 3.36-3.29 (m, 1H), 1.43-1.42 (m, 6H). MS (ESI) m/z 515.9 [M+H]⁺.

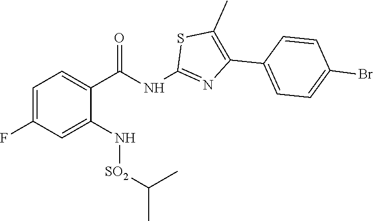 [SMILES: Cc1sc(NC(=O)c2ccc(F)cc2NS(=O)(=O)C(C)C)nc1-c1ccc(Br)cc1]

Example 95: N-(4-(4-Bromophenyl)-5-methylthiazol-2-yl)-4-fluoro-2-((1-methylethyl)sulfonamido)benzamide (XH198-1) ¹H NMR (400 MHz, Chloroform-d) δ 10.75 (bs, 1H), 7.68 (dd, J=8.8, 6.0 Hz, 1H), 7.57 (dd, J=10.8, 2.4 Hz, 1H), 7.49 (d, J=8.4 Hz, 2H), 7.33 (d, J=8.4 Hz, 2H), 6.70-6.65 (m, 1H), 3.39-3.32 (m, 1H), 2.50 (s, 3H), 1.43-1.42 (m, 6H). MS (ESI) m/z 512.0 [M+H]⁺.

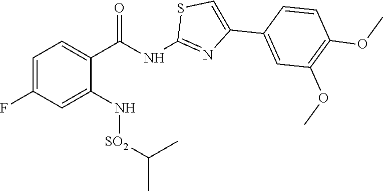 [SMILES: COc1ccc(-c2csc(NC(=O)c3ccc(F)cc3NS(=O)(=O)C(C)C)n2)cc1OC]

Example 96: N-(4-(3,4-Dimethoxyphenyl)thiazol-2-yl)-4-fluoro-2-((1-methylethyl)sulfonamido)benzamide (XH198-2) ¹H NMR (400 MHz, Chloroform-d) δ 10.60 (s, 1H), 8.04 (dd, J=9.6, 6.4 Hz, 1H), 7.62 (dd, J=11.8, 2.4 Hz, 1H), 7.38 (s, 1H), 7.30 (d, J=8.4 Hz, 1H), 7.09 (s, 1H), 6.93-6.85 (m, 2H), 3.98 (s, 3H), 3.93 (s, 3H), 3.42-3.35 (m, 1H), 1.44-1.42 (m, 6H). MS (ESI) m/z 480.1 [M+H]⁻.

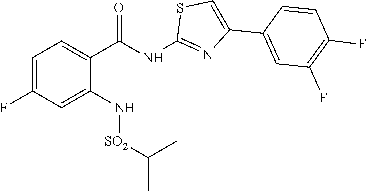 [SMILES: CC(C)S(=O)(=O)Nc1cc(F)ccc1C(=O)Nc1nc(-c2ccc(F)c(F)c2)cs1]

Example 97: N-(4-(3,4-Dimethoxyphenyl)thiazol-2-yl)-4-fluoro-2-((1-methylethyl)sulfonamido)benzamide (XH198-3) ¹H NMR (400 MHz, Chloroform-d) δ 10.64 (bs, 1H), 9.76 (bs, 1H), 7.75 (dd, J=8.8, 5.8 Hz, 1H), 7.67-7.61 (m, 2H), 7.55-7.51 (m, 1H), 7.24-7.18 (m, 2H), 6.89-6.84 (m, 1H), 3.42-3.36 (m, 1H), 1.45-1.43 (m, 6H). MS (ESI) m/z 456.0 [M+H]⁺.

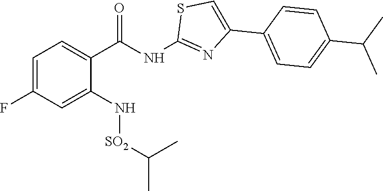 [SMILES: CC(C)c1ccc(-c2csc(NC(=O)c3ccc(F)cc3NS(=O)(=O)C(C)C)n2)cc1]

Example 98: N-(4-(3,4-Difluorophenyl)thiazol-2-yl)-4-fluoro-2-((1-methylethyl)sulfonamido)benzamide (Xh198-4) ¹H NMR (400 MHz, Chloroform-d) δ 10.90 (bs, 1H), 7.81-7.74 (m, 3H), 7.66 (dd, J=11.2, 2.8 Hz, 1H), 7.38-7.34 (m, 3H), 6.80-6.76 (m, 1H), 3.51-3.44 (m, 1H), 3.07-2.98 (m, 1H), 1.55-1.53 (m, 6H), 1.38-1.37 (m, 6H). MS (ESI) m/z 462.1 [M+H]⁺.

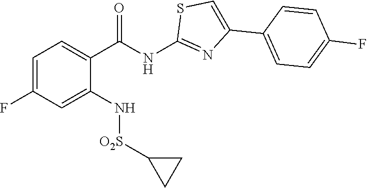 [SMILES: O=C(Nc1nc(-c2ccc(F)cc2)cs1)c1ccc(F)cc1NS(=O)(=O)C1CC1]

Example 99: 2-(Cyclopropanesulfonamido)-4-fluoro-N-(4-(4-fluorophenyl)thiazol-2-yl)benzamide (XH198-62) ¹H NMR (400 MHz, Chloroform-d) δ 10.61 (s, 1H), 7.76 (t, J=9.2 Hz, 1H), 7.71 (t, J=8.4 Hz, 2H), 7.55 (d, J=10.8 Hz, 1H), 7.14 (s, 1H), 7.10 (t, J=8.4 Hz, 2H), 6.78 (t, J=8.4 Hz, 2H), 2.63-2.57 (m, 1H), 1.34-1.30 (m, 2H), 1.06-1.01 (m, 2H). MS (ESI) m/z 436.0 [M+H]⁺.

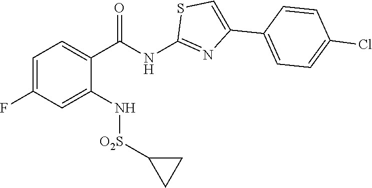 [SMILES: O=C(Nc1nc(-c2ccc(Cl)cc2)cs1)c1ccc(F)cc1NS(=O)(=O)C1CC1]

Example 100: N-(4-(4-Chlorophenyl)thiazol-2-yl)-2-(cyclopropanesulfonamido)-4-fluorobenzamide (XH198-63) ¹H NMR (400 MHz, Chloroform-d) δ 10.67 (s, 1H), 7.82 (t, J=8.4 Hz, 1H), 7.73 (d, J=8.4 Hz, 2H), 7.62 (d, J=10.8 Hz, 1H), 7.40 (d, J=8.0 Hz, 2H), 7.21 (s, 1H), 6.89 (t, J=9.2 Hz, 1H), 2.64-2.57 (m, 1H), 1.33-1.30 (m, 2H), 1.04-1.02 (m, 2H). MS (ESI) m/z 452.0 [M+H]⁺.

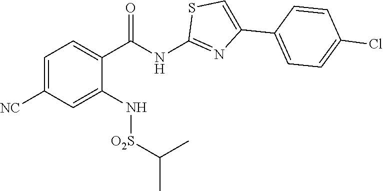 [SMILES: CC(C)S(=O)(=O)Nc1cc(C#N)ccc1C(=O)Nc1nc(-c2ccc(Cl)cc2)cs1]

Example 101: N-(4-(4-Chlorophenyl)thiazol-2-yl)-4-cyano-2-((1-methylethyl)sulfonamido)benzamide (XH198-65) ¹H NMR (400 MHz, Chloroform-d) δ 10.87 (s, 1H), 8.12 (s, 1H), 8.00 (d, J=8.4 Hz, 1H), 7.67 (d, J=8.4 Hz, 2H), 7.42-7.38 (m, 3H), 7.18 (s, 1H), 3.42-3.33 (m, 1H), 1.45-1.44 (m, 6H). MS (ESI) m/z 461.0 [M+H]⁺.

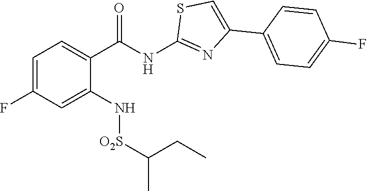 [SMILES: CCC(C)S(=O)(=O)Nc1cc(F)ccc1C(=O)Nc1nc(-c2ccc(F)cc2)cs1]

Example 102: 4-Fluoro-N-(4-(4-fluorophenyl)thiazol-2-yl)-2-((1-methylpropyl)sulfonamido)benzamide (XH198-66) ¹H NMR (400 MHz, Chloroform-d) δ 10.64 (s, 1H), 7.73-7.67 (m, 3H), 7.54 (d, J=10.8 Hz, 1H), 7.13 (s, 1H), 7.07 (t, J=8.4 Hz, 2H), 6.70 (t, J=6.8 Hz, 1H), 3.17-3.08 (m, 1H), 2.15-2.04 (m, 1H), 1.72-1.60 (m, 1H), 1.40 (d, J=6.8 Hz, 3H), 1.03 (t, J=7.2 Hz, 3H). MS (ESI) m/z 452.1 [M+H]⁺.

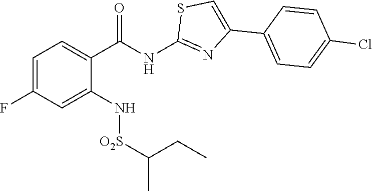 [SMILES: CCC(C)S(=O)(=O)Nc1cc(F)ccc1C(=O)Nc1nc(-c2ccc(Cl)cc2)cs1]

Example 103: N-(4-(4-Chlorophenyl)thiazol-2-yl)-4-fluoro-2-((1-methylpropyl)sulfonamido)benzamide (XH198-67) ¹H NMR (400 MHz, Chloroform-d) δ 10.63 (s, 1H), 7.70-6.64 (m, 3H), 7.55 (d, J=11.2 Hz, 1H), 7.34 (d, J=8.4 Hz, 2H), 7.19 (s, 1H), 6.69 (t, J=6.4 Hz, 2H), 3.18-3.09 (m, 1H), 2.15-2.04 (m, 1H), 1.72-1.61 (m, 1H), 1.41 (d, J=6.8 Hz, 3H), 1.03 (t, J=7.6 Hz, 3H). MS (ESI) m/z 468.1 [M+H]⁺.

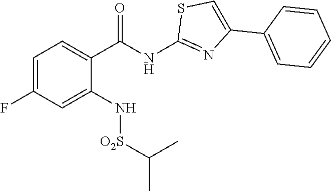 [SMILES: CC(C)S(=O)(=O)Nc1cc(F)ccc1C(=O)Nc1nc(-c2ccccc2)cs1]

Example 104: 4-Fluoro-2-((1-methylethyl)sulfonamido)-N-(4-phenylthiazol-2-yl)benzamide (XH198-70) ¹H NMR (400 MHz, Chloroform-d) δ 10.65 (s, 1H), 7.68-7.63 (m, 3H), 7.49 (d, J=10.0 Hz, 1H), 7.38-7.30 (m, 3H), 7.19 (s, 1H), 6.61 (t, J=7.6 Hz, 1H), 3.40-3.29 (m, 1H), 1.43-1.42 (m, 6H). MS (ESI) m/z 420.1 [M+H]⁺.

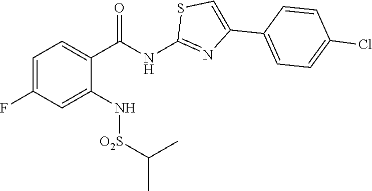 [SMILES: CC(C)S(=O)(=O)Nc1cc(F)ccc1C(=O)Nc1nc(-c2ccc(Cl)cc2)cs1]

Example 105: N-(4-(4-Chlorophenyl)thiazol-2-yl)-4-fluoro-2-((1-methylethyl)sulfonamido)benzamide (XH198-71) ¹H NMR (400 MHz, Chloroform-d) δ 10.60 (s, 1H), 7.63 (d, J=8.4 Hz, 3H), 7.55 (d, J=11.2 Hz, 1H), 7.32 (d, J=8.0 Hz, 2H), 7.19 (s, 1H), 6.65 (d, J=9.6 Hz, 1H), 3.41-3.31 m, 1H), 1.44-1.42 (m, 6H). MS (ESI) m/z 454.0 [M+H]⁺.

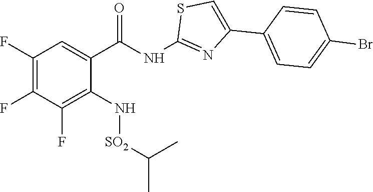 [SMILES: CC(C)S(=O)(=O)Nc1c(C(=O)Nc2nc(-c3ccc(Br)cc3)cs2)cc(F)c(F)c1F]

Example 106: N-(4-(4-Bromophenyl)thiazol-2-yl)-3,4,5-trifluoro-2-((1-methylethyl)sulfonamido)benzamide (XH198-8). ¹H NMR (400 MHz, Chloroform-d) δ 7.59 (d, J=8.4 Hz, 2H), 7.53-7.44 (m, 3H), 7.18 (s, 1H), 3.60-3.53 (m, 1H), 1.52-1.51 (m, 6H). MS (ESI) m/z 433.9 [M+H]⁺.

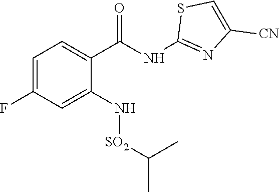 [SMILES: CC(C)S(=O)(=O)Nc1cc(F)ccc1C(=O)Nc1nc(C#N)cs1]

Example 107: N-(4-Cyanothiazol-2-yl)-4-fluoro-2-((1-methylethyl)sulfonamido)benzamide (XH198-9) ¹H NMR (400 MHz, Methanol-d₄) δ 8.09 (s, 1H), 8.04 (dd, J=9.2, 6.4 Hz, 1H), 7.56 (dd, J=11.2, 2.4 Hz, 1H), 7.00 (td, J=8.0, 2.8 Hz, 1H), 3.48-3.41 (m, 1H), 1.35-1.33 (m, 6H). MS (ESI) m/z 369.1 [M+H]⁺.

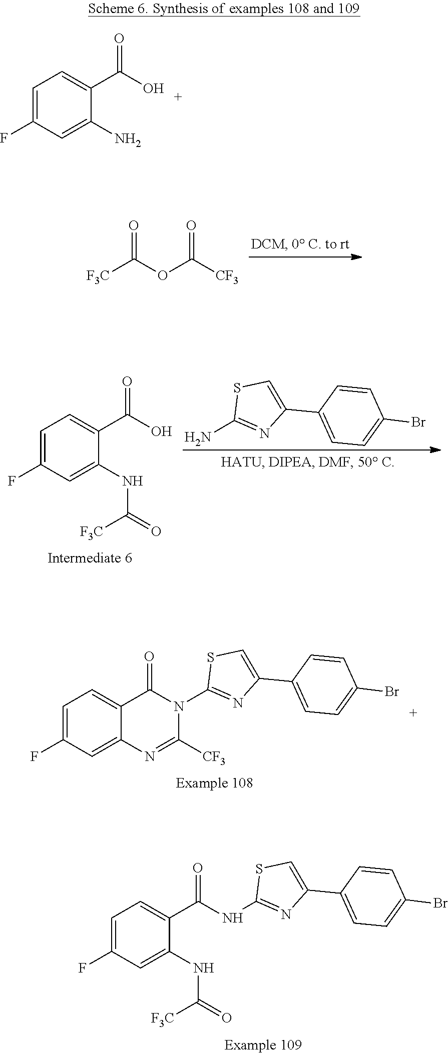 [SMILES: Nc1cc(F)ccc1C(=O)O ; O=C(OC(=O)C(F)(F)F)C(F)(F)F ; O=C(O)c1ccc(F)cc1NC(=O)C(F)(F)F ; Nc1nc(-c2ccc(Br)cc2)cs1 ; O=c1c2ccc(F)cc2nc(C(F)(F)F)n1-c1nc(-c2ccc(Br)cc2)cs1 ; O=C(Nc1nc(-c2ccc(Br)cc2)cs1)c1ccc(F)cc1NC(=O)C(F)(F)F]

To a solution of 2-amino-4-fluorobenzoic acid (55.1 mg, 1 mmol) in DCM (5 mL) was added commercially available 2,2,2-trifluoroacetic anhydride (221 mg, 1.05 mmol, 1.05 equiv) under 0° C., then move to room temperature. After being stirred for additional 30 min at rt, the resulting mixture was purified by reverse-phase ISCO to afford to yield intermediate 6 (143 mg, 57% yield).

To a solution of intermediate 6 (50 mg, 0.2 mmol) in DMF (0.5 mL) were added commercially available 4-(4-bromophenyl)thiazol-2-amine (51 mg, 0.2 mmol, 1.0 equiv), 0-(7-Azabenzotriazol-1-yl)-N,N,N,N-tetramethyluronium hexafluorophosphate (HATU, 91.2 mg, 0.24 mmol, 1.2 equiv), and diisopropylethylamine (DIPEA, 104 μL, 0.6 mmol, 3 equiv). After being stirred at 50° C. overnight, the crude reaction mixture was purified via prep-HPLC to yield example 108 (24 mg, yield 27%) and example 109 (4.3 mg, yield 4%)

Example 108: 3-(4-(4-Bromophenyl)thiazol-2-yl)-7-fluoro-2-(trifluoromethyl)quinazolin-4(3H)-one (XH188-135P1) ¹H NMR (400 MHz, Chloroform-d) δ 8.38 (dd, J=9.2, 4 Hz, 1H), 7.77-7.74 (m, 3H), 7.60-7.55 (m, 3H), 7.41 (td, J=8.4, 2.4 Hz, 1H). (white solid, yield 34%). MS (ESI) m/z 471.9 [M+H]⁺.

Example 109: N-(4-(4-Bromophenyl)thiazol-2-yl)-4-fluoro-2-(2,2,2-trifluoroacetamido)benzamide (XH188-135P2) ¹H NMR (400 MHz, Chloroform-d) δ 12.41 (bs, 1H), 8.52 (dd, J=10.8, 2.8 Hz, 1H), 7.82 (dd, J=9.2, 6 Hz, 1H), 7.65 (d, J=8.4 Hz, 2H), 7.54 (d, J=8.4 Hz, 2H), 7.24 (s, 1H), 7.00-6.96 (m, 1H). MS (ESI) m/z 489.9 [M+H]⁺.

Examples 110-180 were synthesized following the same procedure for preparing examples 108 and 109.

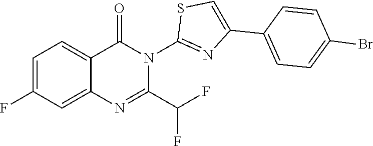 [SMILES: O=c1c2ccc(F)cc2nc(C(F)F)n1-c1nc(-c2ccc(Br)cc2)cs1]

Example 110: 3-(4-(4-Bromophenyl)thiazol-2-yl)-2-(difluoromethyl)-7-fluoroquinazolin-4(3H)-one (XH198-38p1) ¹H NMR (400 MHz, Chloroform-d) δ 8.41 (dd, J=8.8, 5.6 Hz, 1H), 7.77 (d, J=8.2 Hz, 2H), 7.60 (d, J=8.2 Hz, 2H), 7.39 (td, J=8.4, 2.4 Hz, 1H), 6.90 (t, J_{FH}=53.3 Hz, 1H). MS (ESI) m/z 452.0 [M+H]⁺.

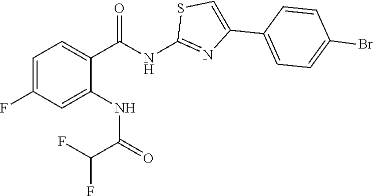 [SMILES: O=C(Nc1nc(-c2ccc(Br)cc2)cs1)c1ccc(F)cc1NC(=O)C(F)F]

Example 111: N-(4-(4-Bromophenyl)thiazol-2-yl)-2-(2,2-difluoroacetamide)-4-fluorobenzamide (XH198-38P2) ¹H NMR (400 MHz, Chloroform-d) δ 12.42 (s, 1H), 8.51 (dd, J=11.2, 2.6 Hz, 1H), 8.18 (dd, J=8.9, 6.2 Hz, 1H), 7.72 (d, J=7.6 Hz, 2H), 7.54 (d, J=8.3 Hz, 2H), 6.95-6.87 (m, 1H), 6.60 (s, 1H), 6.01 (t, J_{FH}=54.3 Hz, 1H). MS (ESI) m/z 470.0 [M+H]⁺.

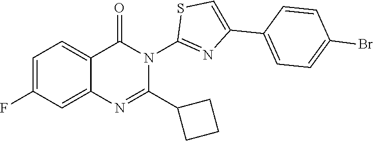 [SMILES: O=c1c2ccc(F)cc2nc(C2CCC2)n1-c1nc(-c2ccc(Br)cc2)cs1]

Example 112: 3-(4-(4-Bromophenyl)thiazol-2-yl)-2-cyclobutyl-7-fluoroquinazolin-4(3H)-one (XH198-43P1) ¹H NMR (400 MHz, Chloroform-d) δ 7.78 (dd, J=8.8, 6.0 Hz, 1H), 7.69 (d, J=8.4 Hz, 2H), 7.53 (d, J=8.4 Hz, 2H), 7.29-7.27 (m, 1H), 7.16 (s, 1H), 6.91 (dd, J=8.4, 2.4 Hz, 1H), 3.56-3.47 (m, 1H), 2.43-2.34 (m, 2H), 2.31-2.21 (m, 2H), 2.10-2.03 (m, 2H). MS (ESI) m/z 456.0 [M+H]⁺.

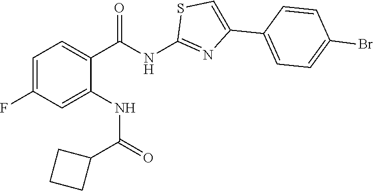 [SMILES: O=C(Nc1nc(-c2ccc(Br)cc2)cs1)c1ccc(F)cc1NC(=O)C1CCC1]

Example 113: N-(4-(4-Bromophenyl)thiazol-2-yl)-2-(cyclobutanecarboxamido)-4-fluorobenzamide (XH198-43P2) ¹H NMR (400 MHz, Chloroform-d) δ 11.02 (s, 1H), 8.64 (dd, J=11.6, 2.4 Hz, 1H), 7.79 (dd, J=8.8, 6.0 Hz, 1H), 7.68 (d, J=8.4 Hz, 2H), 7.57 (d, J=8.4 Hz, 2H), 7.21 (s, 1H), 6.87 (dd, J=7.2, 2.0 Hz, 1H), 3.34-3.25 (m, 1H), 2.47-2.24 (m, 4H), 2.08-1.98 (m, 2H). MS (ESI) m/z 474.0 [M+H]⁻.

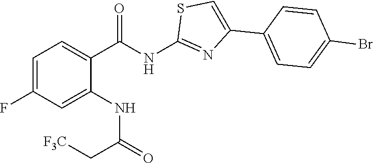 [SMILES: O=C(CC(F)(F)F)Nc1cc(F)ccc1C(=O)Nc1nc(-c2ccc(Br)cc2)cs1]

Example 114: N-(4-(4-Bromophenyl)thiazol-2-yl)-4-fluoro-2-(3,3,3-trifluoropropanamido)benzamide (XH198-44). ¹H NMR (400 MHz, Chloroform-d) δ 11.46 (s, 1H), 8.52 (dd, J=11.6, 2.8 Hz, 1H), 7.72 (dd, J=8.8, 6.0 Hz, 1H), 7.63 (d, J=8.0 Hz, 2H), 7.52 (d, J=8.0 Hz, 2H), 7.23 (s, 1H), 6.85 (dd, J=7.3, 2.4 Hz, 1H), 3.38-3.31 (m, 2H). MS (ESI) m/z 502.3 [M+H]⁺.

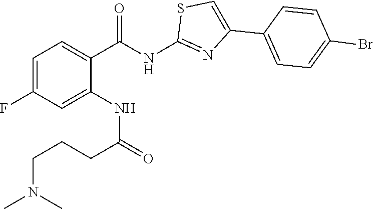 [SMILES: CN(C)CCCC(=O)Nc1cc(F)ccc1C(=O)Nc1nc(-c2ccc(Br)cc2)cs1]

Example 115: N-(4-(4-Bromophenyl)thiazol-2-yl)-2-(4-(dimethylamino)butanamido)-4-fluorobenzamide (XH198-45) ¹H NMR (400 MHz, Chloroform-d) δ 12.42 (s, 1H), 11.18 (s, 1H), 8.47 (dd, J=11.6, 2.4 Hz, 1H), 8.04 (dd, J=8.8, 6.0 Hz, 1H), 7.66-7.61 (m, 4H), 7.21 (s, 1H), 6.98-6.93 (m, 1H), 3.49 (s, 6H), 3.21 (s, 2H), 2.72-2.69 (m, 2H), 2.22 (s, 2H). MS (ESI) m/z 505.1 [M+H]⁺.

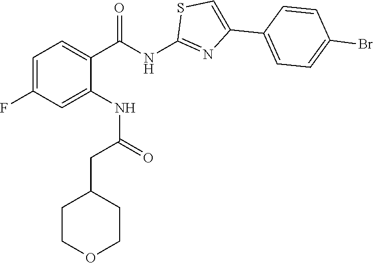 [SMILES: O=C(CC1CCOCC1)Nc1cc(F)ccc1C(=O)Nc1nc(-c2ccc(Br)cc2)cs1]

Example 116: N-(4-(4-Bromophenyl)thiazol-2-yl)-4-fluoro-2-(2-(tetrahydro-2H-pyran-4-yl)acetamido)benzamide (XH198-46) ¹H NMR (400 MHz, Chloroform-d) δ 11.07 (s, 1H), 8.59 (dd, J=11.6, 2.4 Hz, 1H), 8.04 (dd, J=8.8, 5.6 Hz, 1H), 7.62 (s, 4H), 7.19 (s, 1H), 6.92 (td, J=9.2, 8.4, 2.6 Hz, 1H), 3.99 (dd, J=11.6, 4.4 Hz, 2H), 3.46 (td, J=12.4, 2.4 Hz, 2H), 2.42 (d, J=7.2 Hz, 2H), 2.25-2.14 (m, 1H), 1.73 (d, J=12.0 Hz, 2H), 1.44 (qd, J=12.0, 4.0 Hz, 2H). MS (ESI) m/z 518.1 [M+H]⁺.

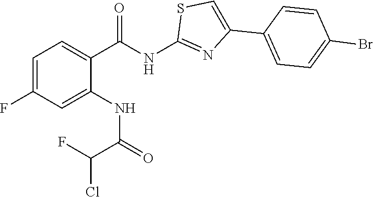 [SMILES: O=C(Nc1nc(-c2ccc(Br)cc2)cs1)c1ccc(F)cc1NC(=O)C(F)Cl]

Example 117: N-(4-(4-Bromophenyl)thiazol-2-yl)-2-(2-chloro-2-fluoroacetamido)-4-fluorobenzamide (XH198-48) ¹H NMR (400 MHz, Chloroform-d) δ 12.10 (s, 1H), 8.53 (dd, J=11.2, 3.2 Hz, 1H), 7.79 (dd, J=9.2, 6.0 Hz, 1H), 7.63 (d, J=8.4 Hz, 2H), 7.53 (d, J=8.4 Hz, 2H), 7.23 (s, 1H), 6.92 (t, J=6.8 Hz, 2H), 6.46 (d, J_{FH}=50.7 Hz, 1H). MS (ESI) m/z 485.3 [M+H]⁺.

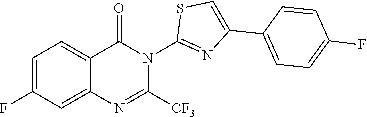 [SMILES: O=c1c2ccc(F)cc2nc(C(F)(F)F)n1-c1nc(-c2ccc(F)cc2)cs1]

Example 118: 7-Fluoro-3-(4-(4-fluorophenyl)thiazol-2-yl)-2-(trifluoromethyl)quinazolin-4(3H)-one (XH198-68P1) ¹H NMR (400 MHz, Chloroform-d) δ 8.38 (dd, J=8.8, 6.0 Hz, 1H), 7.87-7.83 (m, 2H), 7.70 (s, 1H), 7.58 (dd, J=8.2, 2.4 Hz, 1H), 7.41 (td, J=8.4, 2.4 Hz, 1H), 7.13 (t, J=8.4 Hz, 2H). MS (ESI) m/z 410.0 [M+H]⁺.

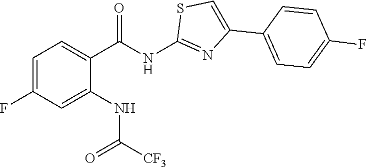 [SMILES: O=C(Nc1nc(-c2ccc(F)cc2)cs1)c1ccc(F)cc1NC(=O)C(F)(F)F]

Example 119: 4-Fluoro-N-(4-(4-fluorophenyl)thiazol-2-yl)-2-(2,2,2-trifluoroacetamido)benzamide (XH198-68P2) ¹H NMR (400 MHz, Chloroform-d) δ 12.46 (s, 1H), 8.51 (dd, J=10.8, 2.8 Hz, 1H), 7.87 (dd, J=8.2, 6.0 Hz, 1H), 7.75 (dd, J=8.8, 5.6 Hz, 2H), 7.17 (s, 1H), 7.11 (t, J=8.4 Hz, 2H), 7.02-6.97 (m, 1H). MS (ESI) m/z 428.0 [M+H]⁺.

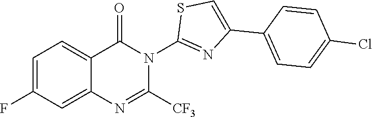 [SMILES: O=c1c2ccc(F)cc2nc(C(F)(F)F)n1-c1nc(-c2ccc(Cl)cc2)cs1]

Example 120: 3-(4-(4-Chlorophenyl)thiazol-2-yl)-7-fluoro-2-(trifluoromethyl)quinazolin-4(3H)-one (XH198-69P1) ¹H NMR (400 MHz, Chloroform-d) δ 8.38 (dd, J=8.4, 5.6 Hz, 1H), 7.81 (d, J=8.4 Hz, 2H), 7.75 (s, 1H), 7.58 (dd, J=8.8, 2.4 Hz, 1H), 7.43-7.39 (m, 3H). MS (ESI) m/z 426.0 [M+H]⁺.

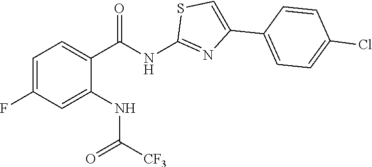 [SMILES: O=C(Nc1nc(-c2ccc(Cl)cc2)cs1)c1ccc(F)cc1NC(=O)C(F)(F)F]

Example 121: N-(4-(4-chlorophenyl)thiazol-2-yl)-4-fluoro-2-(2,2,2-trifluoroacetamido)benzamide (XH198-69P2) ¹H NMR (400 MHz, Chloroform-d) δ 12.44 (s, 1H), 8.53 (dd, J=10.8, 2.4 Hz, 1H), 7.95 (dd, J=8.4, 5.6 Hz, 1H), 7.75-7.71 (m, 2H), 7.42-7.40 (m, 3H), 7.23 (s, 1H), 7.05-7.00 (m, 1H). MS (ESI) m/z 444.0 [M+H]⁺.

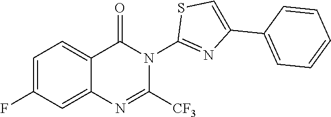 [SMILES: O=c1c2ccc(F)cc2nc(C(F)(F)F)n1-c1nc(-c2ccccc2)cs1]

Example 122: 7-Fluoro-3-(4-phenylthiazol-2-yl)-2-(trifluoromethyl)quinazolin-4(3H)-one (XH208-34p1) ¹H NMR (400 MHz, Chloroform-d) δ 8.42-8.32 (m, 1H), 7.93-7.84 (m, 2H), 7.76 (s, 1H), 7.58 (d, J=8.0 Hz, 1H), 7.50-7.32 (m, 4H). MS (ESI) m/z 392.0 [M+H]⁺.

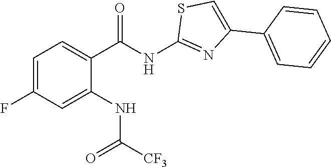 [SMILES: O=C(Nc1nc(-c2ccccc2)cs1)c1ccc(F)cc1NC(=O)C(F)(F)F]

Example 123: 4-Fluoro-N-(4-phenylthiazol-2-yl)-2-(2,2,2-trifluoroacetamido)benzamide (XH208-34P2) ¹H NMR (400 MHz, Chloroform-d) δ 12.39 (s, 1H), 8.57-8.46 (m, 1H), 8.24-8.14 (m, 1H), 7.82-7.74 (m, 2H), 7.54-7.43 (m, 3H), 7.22 (s, 1H), 7.13-7.05 (m, 1H). MS (ESI) m/z 410.1 [M+H]⁺.

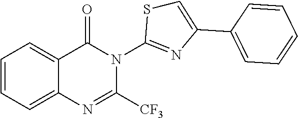 [SMILES: O=c1c2ccccc2nc(C(F)(F)F)n1-c1nc(-c2ccccc2)cs1]

Example 124: 3-(4-Phenylthiazol-2-yl)-2-(trifluoromethyl)quinazolin-4(3H)-one (XH208-39P1) ¹H NMR (400 MHz, Chloroform-d) δ 8.36 (d, J=8.0 Hz, 1H), 7.99-7.84 (m, 4H), 7.75 (s, 1H), 7.73-7.66 (m, 1H), 7.50-7.41 (m, 2H), 7.40-7.34 (m, 1H). MS (ESI) m/z 374.0 [M+H]⁺.

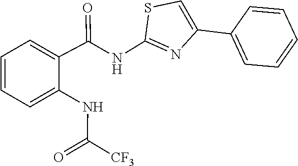 [SMILES: O=C(Nc1nc(-c2ccccc2)cs1)c1ccccc1NC(=O)C(F)(F)F]

Example 125: N-(4-Phenylthiazol-2-yl)-2-(2,2,2-trifluoroacetamido)benzamide (XH208-39P2) ¹H NMR (400 MHz, Chloroform-d) δ 12.15 (s, 1H), 8.69 (d, J=8.0 Hz, 1H), 7.98-7.91 (m, 2H), 7.82-7.75 (m, 2H), 7.72-7.67 (m, 2H), 7.43 (s, 2H), 7.22 (s, 1H). MS (ESI) m/z 392.0 [M+H]⁺.

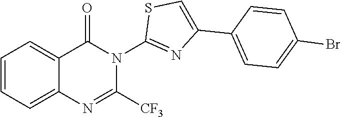 [SMILES: O=c1c2ccccc2nc(C(F)(F)F)n1-c1nc(-c2ccc(Br)cc2)cs1]

Example 126: 3-(4-(4-Bromophenyl)thiazol-2-yl)-2-(trifluoromethyl)quinazolin-4(3H)-one (XH208-40P1) ¹H NMR (400 MHz, Chloroform-d) δ 8.36 (d, J=8.0 Hz, 1H), 7.99-7.85 (m, 2H), 7.81-7.64 (m, 4H), 7.61-7.51 (m, 2H). MS (ESI) m/z 452.0 [M+H]⁺.

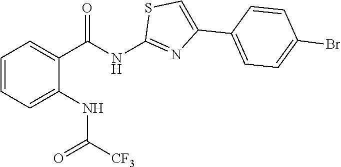 [SMILES: O=C(Nc1nc(-c2ccc(Br)cc2)cs1)c1ccccc1NC(=O)C(F)(F)F]

Example 127: N-(4-(4-Bromophenyl)thiazol-2-yl)-2-(2,2,2-trifluoroacetamido)benzamide (XH208-40P2) ¹H NMR (400 MHz, Chloroform-d) δ 12.17 (s, 1H), 8.80 (s, 1H), 8.71-8.65 (m, 1H), 8.21-8.14 (m, 1H), 7.67 (d, J=8.0 Hz, 1H), 7.64-7.54 (m, 4H), 7.31-7.27 (m, 1H). MS (ESI) m/z 472.0 [M+H]⁺.

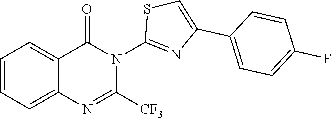 [SMILES: O=c1c2ccccc2nc(C(F)(F)F)n1-c1nc(-c2ccc(F)cc2)cs1]

Example 128: 3-(4-(4-Fluorophenyl)thiazol-2-yl)-2-(trifluoromethyl)quinazolin-4(3H)-one (XH208-41P1) ¹H NMR (400 MHz, Chloroform-d) δ 8.36 (d, J=8.0 Hz, 1H), 7.95-7.91 (m, 2H), 7.89-7.83 (m, 2H), 7.74-7.64 (m, 2H), 7.12 (d, J=8.8 Hz, 2H). MS (ESI) m/z 392.0 [M+H]⁺.

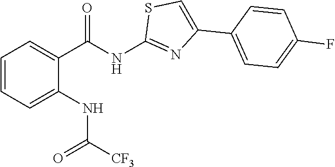 [SMILES: O=C(Nc1nc(-c2ccc(F)cc2)cs1)c1ccccc1NC(=O)C(F)(F)F]

Example 129: N-(4-(4-Fluorophenyl)thiazol-2-yl)-2-(2,2,2-trifluoroacetamido)benzamide (XH208-41P2) ¹H NMR (400 MHz, Chloroform-d) δ 12.13 (s, 1H), 8.66 (d, J=8.0 Hz, 1H), 7.90-7.86 (m, 1H), 7.82-7.77 (m, 1H), 7.76-7.70 (m, 1H), 7.68-7.59 (m, 1H), 7.31 (t, J=8.0 Hz, 1H), 7.14-7.01 (m, 3H). MS (ESI) m/z 410.0 [M+H]⁺.

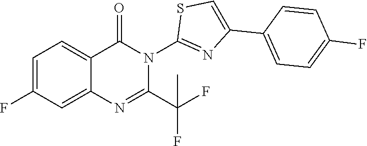 [SMILES: CC(F)(F)c1nc2cc(F)ccc2c(=O)n1-c1nc(-c2ccc(F)cc2)cs1]

Example 130: 2-(1,1-Difluoroethyl)-7-fluoro-3-(4-(4-fluorophenyl)thiazol-2-yl)quinazolin-4(3H)-one (XH208-77P1) ¹H NMR (400 MHz, Chloroform-d) δ 8.41-8.20 (m, 1H), 7.94-7.79 (m, 2H), 7.67 (s, 1H), 7.49 (d, J=9.0 Hz, 1H), 7.39-7.28 (m, 1H), 7.11 (d, J=8.0 Hz, 2H), 2.13 (t, J=19.0 Hz, 3H). MS (ESI) m/z 406.0 [M+H]⁺.

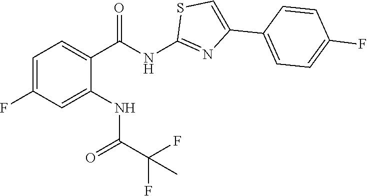 [SMILES: CC(F)(F)C(=O)Nc1cc(F)ccc1C(=O)Nc1nc(-c2ccc(F)cc2)cs1]

Example 131: 2-(2,2-Difluoropropanamide)-4-fluoro-N-(4-(4-fluorophenyl)thiazol-2-yl)benzamide (XH208-77P2) ¹H NMR (400 MHz, Chloroform-d) δ 12.06 (s, 1H), 8.60-8.49 (m, 1H), 7.88-7.80 (m, 2H), 7.79-7.69 (m, 2H), 7.16-7.07 (m, 3H), 7.a00-6.88 (m, 1H), 1.93 (t, J=19.0 Hz, 3H). MS (ESI) m/z 424.1 [M+H]⁺.

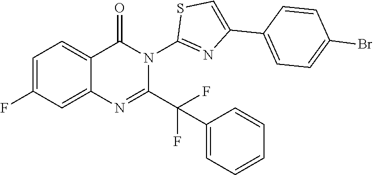 [SMILES: O=c1c2ccc(F)cc2nc(C(F)(F)c2ccccc2)n1-c1nc(-c2ccc(Br)cc2)cs1]

Example 132: 3-(4-(4-Bromophenyl)thiazol-2-yl)-2-(difluoro(phenyl)methyl)-7-fluoroquinazolin-4(3H)-one (XH208-79P1) ¹H NMR (400 MHz, Chloroform-d) δ 8.39-8.26 (m, 1H), 7.66 (s, 1H), 7.61-7.54 (m, 1H), 7.52-7.29 (m, 10H). MS (ESI) m/z 528.1 [M+H]⁺.

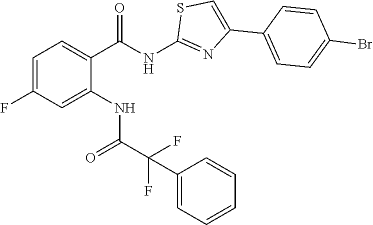 [SMILES: O=C(Nc1nc(-c2ccc(Br)cc2)cs1)c1ccc(F)cc1NC(=O)C(F)(F)c1ccccc1]

Example 133: N-(4-(4-Bromophenyl)thiazol-2-yl)-2-(2,2-difluoro-2-phenylacetamido)-4-fluorobenzamide (XH208-79P2) ¹H NMR (400 MHz, Chloroform-d) δ 12.26 (s, 1H), 8.55-8.48 (m, 1H), 7.79-7.70 (m, 3H), 7.69-7.60 (m, 2H), 7.55-7.47 (m, 5H), 7.25 (s, 1H), 6.94-6.84 (m, 1H). MS (ESI) m/z 546.1 [M+H]⁺.

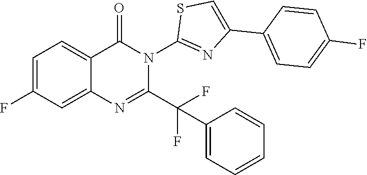 [SMILES: O=c1c2ccc(F)cc2nc(C(F)(F)c2ccccc2)n1-c1nc(-c2ccc(F)cc2)cs1]

Example 134: 2-(Difluoro(phenyl)methyl)-7-fluoro-3-(4-(4-fluorophenyl)thiazol-2-yl)quinazolin-4(3H)-one (XH208-80P1) ¹H NMR (400 MHz, Chloroform-d) δ 8.37-8.26 (m, 1H), 7.63-7.49 (m, 4H), 7.46-7.28 (m, 6H), 7.10-7.00 (m, 2H). MS (ESI) m/z 468.1 [M+H]⁺.

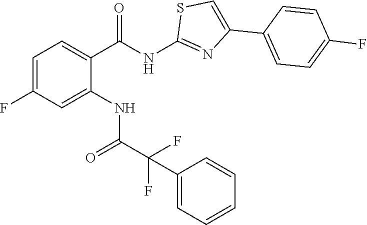 [SMILES: O=C(Nc1nc(-c2ccc(F)cc2)cs1)c1ccc(F)cc1NC(=O)C(F)(F)c1ccccc1]

Example 135: 2-(2,2-Difluoro-2-phenylacetamido)-4-fluoro-N-(4-(4-fluorophenyl)thiazol-2-yl)benzamide (XH208-80P2) ¹H NMR (400 MHz, Chloroform-d) δ 12.25 (s, 1H), 8.50 (d, J=10.2 Hz, 1H), 7.86-7.67 (m, 5H), 7.54-7.42 (m, 3H), 7.17 (s, 1H), 7.09 (t, J=8.8 Hz, 2H), 6.97-6.84 (m, 1H). MS (ESI) m/z 486.1 [M+H]⁺.

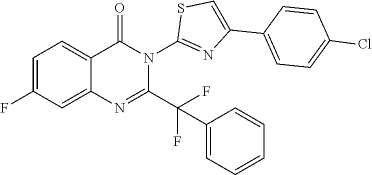 [SMILES: O=c1c2ccc(F)cc2nc(C(F)(F)c2ccccc2)n1-c1nc(-c2ccc(Cl)cc2)cs1]

Example 136: 3-(4-(4-Chlorophenyl)thiazol-2-yl)-2-(difluoro(phenyl)methyl)-7-fluoroquinazolin-4(3H)-one (XH208-81P1) ¹H NMR (400 MHz, Chloroform-d) δ 8.38-8.24 (m, 1H), 7.68-7.63 (m, 1H), 7.61-7.55 (m, 1H), 7.54-7.48 (m, 2H), 7.44-7.29 (m, 8H). MS (ESI) m/z 484.1 [M+H]⁺.

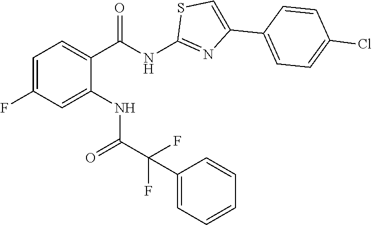 [SMILES: O=C(Nc1nc(-c2ccc(Cl)cc2)cs1)c1ccc(F)cc1NC(=O)C(F)(F)c1ccccc1]

Example 137: N-(4-(4-Chlorophenyl)thiazol-2-yl)-2-(2,2-difluoro-2-phenylacetamido)-4-fluorobenzamide (XH208-81P2) ¹H NMR (400 MHz, Chloroform-d) δ 12.24 (s, 1H), 8.50 (d, J=11.2 Hz, 1H), 7.82-7.67 (m, 5H), 7.58-7.46 (m, 3H), 7.42-7.33 (m, 2H), 7.23 (s, 1H), 6.93-6.80 (m, 1H). MS (ESI) m/z 502.1 [M+H]⁺.

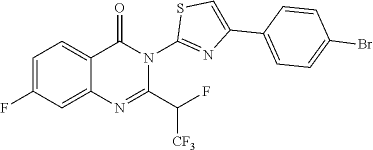 [SMILES: O=c1c2ccc(F)cc2nc(C(F)C(F)(F)F)n1-c1nc(-c2ccc(Br)cc2)cs1]

Example 138: 3-(4-(4-Bromophenyl)thiazol-2-yl)-7-fluoro-2-(1,2,2,2-tetrafluoroethyl)quinazolin-4(3H)-one (XH208-85P1) ¹H NMR (400 MHz, Chloroform-d) δ 8.38 (t, J=8.0 Hz, 1H), 7.82-7.67 (m, 3H), 7.63-7.50 (m, 3H), 7.37 (t, J=8.0 Hz, 1H), 6.34-6.09 (m, 1H). MS (ESI) m/z 502.0 [M+H]⁻.

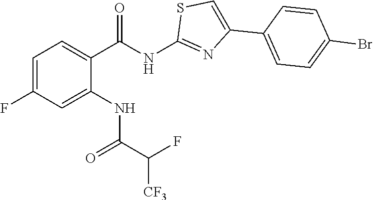 [SMILES: O=C(Nc1nc(-c2ccc(Br)cc2)cs1)c1ccc(F)cc1NC(=O)C(F)C(F)(F)F]

Example 139: N-(4-(4-Bromophenyl)thiazol-2-yl)-4-fluoro-2-(2,3,3,3-tetrafluoropropanamide)benzamide (XH208-85P2) ¹H NMR (400 MHz, Chloroform-d) δ 11.99 (s, 1H), 8.86 (d, J=12.0 Hz, 1H), 8.29-8.08 (m, 1H), 7.80-7.64 (m, 2H), 7.63-7.49 (m, 3H), 7.31 (s, 1H), 6.96-6.79 (m, 1H). MS (ESI) m/z 520.0 [M+H]⁺.

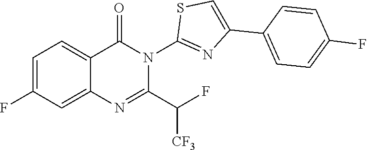 [SMILES: O=c1c2ccc(F)cc2nc(C(F)C(F)(F)F)n1-c1nc(-c2ccc(F)cc2)cs1]

Example 140: 7-Fluoro-3-(4-(4-fluorophenyl)thiazol-2-yl)-2-(1,2,2,2-tetrafluoroethyl)quinazolin-4(3H)-one (XH208-86P1) ¹H NMR (400 MHz, Chloroform-d) δ 8.43-8.32 (m, 1H), 7.91-7.81 (m, 2H), 7.69 (s, 1H), 7.61-7.52 (m, 1H), 7.42-7.32 (m, 1H), 7.15 (t, J=6.8 Hz, 2H), 6.35-6.12 (m, 1H). MS (ESI) m/z 442.0 [M+H]⁺.

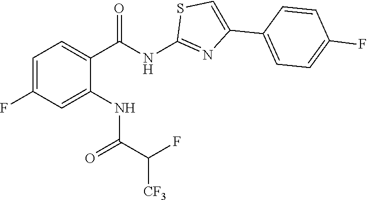 [SMILES: O=C(Nc1nc(-c2ccc(F)cc2)cs1)c1ccc(F)cc1NC(=O)C(F)C(F)(F)F]

Example 141: 4-Fluoro-N-(4-(4-fluorophenyl)thiazol-2-yl)-2-(2,3,3,3-tetrafluoropropanamide)benzamide (XH208-86P2) ¹H NMR (400 MHz, Chloroform-d) δ 11.98 (s, 1H), 8.87 (d, J=12.0 Hz, 1H), 8.25-8.16 (m, 1H), 7.88-7.78 (m, 2H), 7.74-7.65 (m, 1H), 7.19-7.07 (m, 3H), 6.88 (t, J=8.0 Hz, 1H). MS (ESI) m/z 460.0 [M+H]⁺.

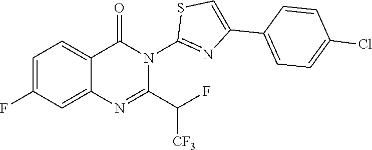 [SMILES: O=c1c2ccc(F)cc2nc(C(F)C(F)(F)F)n1-c1nc(-c2ccc(Cl)cc2)cs1]

Example 142: 3-(4-(4-Chlorophenyl)thiazol-2-yl)-7-fluoro-2-(1,2,2,2-tetrafluoroethyl)quinazolin-4(3H)-one (XH208-87P1) ¹H NMR (400 MHz, Chloroform-d) δ 8.45-8.31 (m, 1H), 7.81 (d, J=8.8 Hz, 2H), 7.74 (s, 1H), 7.56 (d, J=8.8 Hz, 1H), 7.47-7.33 (m, 3H), 6.39-6.12 (m, 1H). MS (ESI) m/z 458.0 [M+H]⁺.

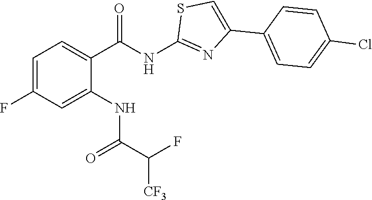 [SMILES: O=C(Nc1nc(-c2ccc(Cl)cc2)cs1)c1ccc(F)cc1NC(=O)C(F)C(F)(F)F]

Example 143: N-(4-(4-Chlorophenyl)thiazol-2-yl)-4-fluoro-2-(2,3,3,3-tetrafluoropropanamido)benzamide (XH208-87P2) ¹H NMR (400 MHz, Chloroform-d) δ 11.96 (s, 1H), 8.86 (d, J=12.0 Hz, 1H), 8.23-8.15 (m, 1H), 7.82-7.72 (m, 2H), 7.69-7.59 (m, 1H), 7.51-7.37 (m, 4H), 7.30 (s, 1H), 6.93-6.83 (m, 1H). MS (ESI) m/z 476.0 [M+H]⁺.

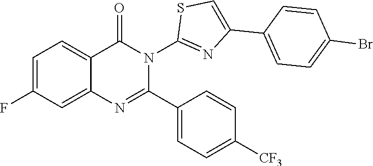 [SMILES: O=c1c2ccc(F)cc2nc(-c2ccc(C(F)(F)F)cc2)n1-c1nc(-c2ccc(Br)cc2)cs1]

Example 144: 3-(4-(4-Bromophenyl)thiazol-2-yl)-7-fluoro-2-(4-(trifluoromethyl)phenyl)quinazolin-4(3H)-one (XH208-92P1) ¹H NMR (400 MHz, Chloroform-d) δ 8.45-8.35 (m, 1H), 7.65 (d, J=8.4 Hz, 2H), 7.59 (d, J=8.4 Hz, 2H), 7.54-7.44 (m, 4H), 7.41-7.36 (m, 2H), 7.36-7.28 (m, 1H). MS (ESI) m/z 546.0 [M+H]⁺.

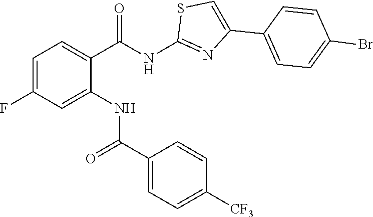 [SMILES: O=C(Nc1cc(F)ccc1C(=O)Nc1nc(-c2ccc(Br)cc2)cs1)c1ccc(C(F)(F)F)cc1]

Example 145: N-(4-(4-Bromophenyl)thiazol-2-yl)-4-fluoro-2-(4-(trifluoromethyl)benzamido)benzamide (XH208-92P2) ¹H NMR (400 MHz, Chloroform-d) δ 12.19 (s, 1H), 8.78 (d, J=11.2 Hz, 1H), 8.17 (d, J=8.0 Hz, 2H), 8.08-7.99 (m, 1H), 7.84 (d, J=8.0 Hz, 2H), 7.74-7.56 (m, 4H), 7.21 (s, 1H), 7.07-6.93 (m, 1H). MS (ESI) m/z 564.1 [M+H]⁺.

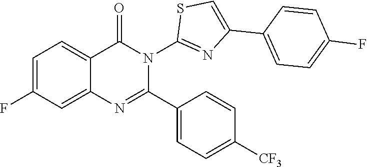 [SMILES: O=c1c2ccc(F)cc2nc(-c2ccc(C(F)(F)F)cc2)n1-c1nc(-c2ccc(F)cc2)cs1]

Example 146: 7-Fluoro-3-(4-(4-fluorophenyl)thiazol-2-yl)-2-(4-(trifluoromethyl)phenyl)quinazolin-4(3H)-one (XH208-93P1) ¹H NMR (400 MHz, Chloroform-d) δ 8.46-8.36 (m, 1H), 7.87-7.75 (m, 1H), 7.66 (d, J=8.0 Hz, 2H), 7.59 (d, J=8.0 Hz, 2H), 7.53-7.41 (m, 4H), 7.36-7.29 (m, 1H), 7.08-6.97 (m, 1H). MS (ESI) m/z 486.1 [M+H]⁺.

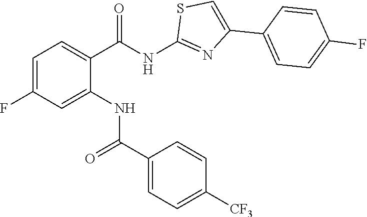 [SMILES: O=C(Nc1cc(F)ccc1C(=O)Nc1nc(-c2ccc(F)cc2)cs1)c1ccc(C(F)(F)F)cc1]

Example 147: 4-Fluoro-N-(4-(4-fluorophenyl)thiazol-2-yl)-2-(4-(trifluoromethyl)benzamido)benzamide (XH208-93P2) ¹H NMR (400 MHz, Chloroform-d) δ 12.18 (s, 1H), 8.73 (d, J=12.0 Hz, 1H), 8.17 (d, J=8.0 Hz, 2H), 7.93-7.77 (m, 3H), 7.77-7.60 (m, 2H), 7.19-7.05 (m, 3H), 6.93-6.82 (m, 1H). MS (ESI) m/z 504.1 [M+H]⁺.

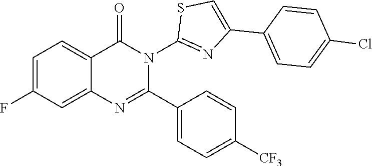 [SMILES: O=c1c2ccc(F)cc2nc(-c2ccc(C(F)(F)F)cc2)n1-c1nc(-c2ccc(Cl)cc2)cs1]

Example 148: 3-(4-(4-Chlorophenyl)thiazol-2-yl)-7-fluoro-2-(4-(trifluoromethyl)phenyl)quinazolin-4(3H)-one (XH208-94P1) ¹H NMR (400 MHz, Chloroform-d) δ 8.45-8.36 (m, 1H), 7.65 (d, J=8.0 Hz, 2H), 7.59 (d, J=8.0 Hz, 2H), 7.53-7.47 (m, 2H), 7.47-7.41 (m, 2H), 7.37-7.28 (m, 3H). MS (ESI) m/z 502.0 [M+H]⁺.

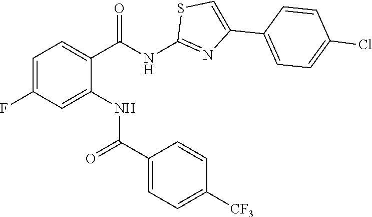 [SMILES: O=C(Nc1cc(F)ccc1C(=O)Nc1nc(-c2ccc(Cl)cc2)cs1)c1ccc(C(F)(F)F)cc1]

Example 149: N-(4-(4-Chlorophenyl)thiazol-2-yl)-4-fluoro-2-(4-(trifluoromethyl)benzamido)benzamide (XH208-94P2) ¹H NMR (400 MHz, Chloroform-d) δ 12.25 (s, 1H), 8.77 (d, J=11.6 Hz, 1H), 8.18 (d, J=8.0 Hz, 2H), 7.90-7.66 (m, 5H), 7.39 (d, J=8.4 Hz, 2H), 7.23 (s, 1H), 6.98-6.88 (m, 1H). MS (ESI) m/z 520.1 [M+H]⁺.

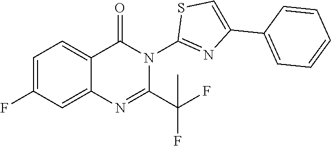 [SMILES: CC(F)(F)c1nc2cc(F)ccc2c(=O)n1-c1nc(-c2ccccc2)cs1]

Example 150: 2-(1,1-Difluoroethyl)-7-fluoro-3-(4-phenylthiazol-2-yl)quinazolin-4(3H)-one (XH208-167p1) ¹H NMR (400 MHz, Chloroform-d) δ 8.41-8.31 (m, 1H), 7.93-7.81 (m, 2H), 7.76-7.72 (m, 1H), 7.52-7.46 (m, 1H), 7.46-7.40 (m, 2H), 7.40-7.33 (m, 2H), 2.13 (d, J=20.0 Hz, 3H). MS (ESI) m/z 388.1 [M+H]⁺.

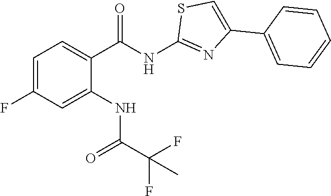 [SMILES: CC(F)(F)C(=O)Nc1cc(F)ccc1C(=O)Nc1nc(-c2ccccc2)cs1]

Example 151: 2-(2,2-Difluoropropanamide)-4-fluoro-N-(4-phenylthiazol-2-yl)benzamide (XH208-167p2) ¹H NMR (400 MHz, Chloroform-d) δ 11.98 (s, 1H), 8.57 (d, J=8.0 Hz, 1H), 8.20 (t, J=8.0 Hz, 1H), 7.82-7.67 (m, 2H), 7.55-7.47 (m, 3H), 7.19 (s, 1H), 7.10-6.99 (m, 1H), 1.93 (d, J=20.0 Hz, 3H). MS (ESI) m/z 406.1 [M+H]⁺.

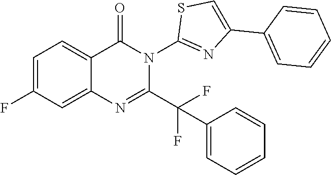 [SMILES: O=c1c2ccc(F)cc2nc(C(F)(F)c2ccccc2)n1-c1nc(-c2ccccc2)cs1]

Example 152: 2-(Difluoro(phenyl)methyl)-7-fluoro-3-(4-phenylthiazol-2-yl)quinazolin-4(3H)-one (XH208-168p1) ¹H NMR (400 MHz, Chloroform-d) δ 11.98 (s, 1H), 8.57 (d, J=8.0 Hz, 1H), 8.20 (t, J=8.0 Hz, 1H), 7.82-7.67 (m, 2H), 7.55-7.47 (m, 3H), 7.19 (s, 1H), 7.10-6.99 (m, 1H), 1.93 (d, J=20.0 Hz, 3H). MS (ESI) m/z 450.1 [M+H]⁺.

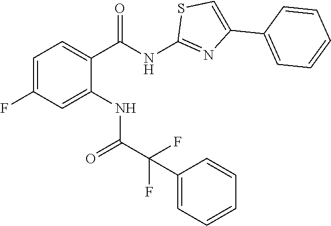 [SMILES: O=C(Nc1nc(-c2ccccc2)cs1)c1ccc(F)cc1NC(=O)C(F)(F)c1ccccc1]

Example 153: 2-(2,2-Difluoro-2-phenylacetamido)-4-fluoro-N-(4-phenylthiazol-2-yl)benzamide (XH208-168p2) ¹H NMR (400 MHz, Chloroform-d) δ 12.19 (s, 1H), 8.47 (d, J=8.0 Hz, 1H), 7.87 (d, J=8.0 Hz, 1H), 7.78-7.67 (m, 4H), 7.59-7.46 (m, 3H), 7.45-7.32 (m, 3H), 7.22 (s, 1H), 6.88 (d, J=8.0 Hz, 1H). MS (ESI) m/z 468.1 [M+H]⁺.

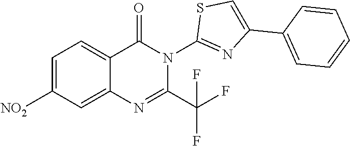 [SMILES: O=c1c2ccc([N+](=O)[O-])cc2nc(C(F)(F)F)n1-c1nc(-c2ccccc2)cs1]

Example 154: 7-Nitro-3-(4-phenylthiazol-2-yl)-2-(trifluoromethyl)quinazolin-4(3H)-one (XH216-30p1) ¹H NMR (400 MHz, Chloroform-d) δ 8.77 (s, 1H), 8.55 (d, J=8.0 Hz, 1H), 8.46 (d, J=8.0 Hz, 1H), 7.88 (d, J=8.0 Hz, 2H), 7.80 (s, 1H), 7.53-7.36 (m, 3H). MS (ESI) m/z 419.0 [M+H]⁺.

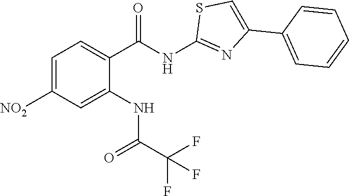 [SMILES: O=C(Nc1nc(-c2ccccc2)cs1)c1ccc([N+](=O)[O-])cc1NC(=O)C(F)(F)F]

Example 155: 4-Nitro-N-(4-phenylthiazol-2-yl)-2-(2,2,2-trifluoroacetamido)benzamide (XH216-30p2) ¹H NMR (400 MHz, Chloroform-d) δ 12.15 (s, 1H), 9.32 (s, 1H), 7.81 (d, J=8.0 Hz, 2H), 7.62-7.47 (m, 2H), 7.32-7.28 (m, 4H). MS (ESI) m/z 437.1 [M+H]⁺.

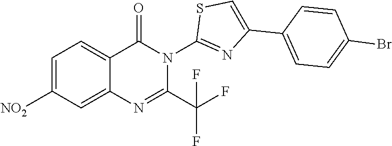 [SMILES: O=c1c2ccc([N+](=O)[O-])cc2nc(C(F)(F)F)n1-c1nc(-c2ccc(Br)cc2)cs1]

Example 156: 3-(4-(4-Bromophenyl)thiazol-2-yl)-7-nitro-2-(trifluoromethyl)quinazolin-4(3H)-one (XH216-31p1) ¹H NMR (400 MHz, Chloroform-d) δ 8.78 (s, 1H), 8.55 (d, J=8.0 Hz, 1H), 8.47 (d, J=8.0 Hz, 1H), 7.80 (s, 1H), 7.75 (d, J=8.0 Hz, 2H), 7.58 (d, J=8.0 Hz, 2H). MS (ESI) m/z 496.9 [M+H]⁺.

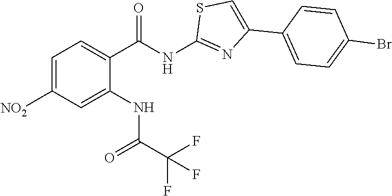 [SMILES: O=C(Nc1nc(-c2ccc(Br)cc2)cs1)c1ccc([N+](=O)[O-])cc1NC(=O)C(F)(F)F]

Example 157: N-(4-(4-Bromo))phenyl)thiazol-2-yl)-4-nitro-2-(2,2,2-trifluoroacetamido)benzamide (XH216-31p2) ¹H NMR (400 MHz, Chloroform-d) δ 12.33 (s, 1H), 9.53 (s, 1H), 8.13-7.93 (m, 2H), 7.58 (d, J=8.0 Hz, 2H), 7.52 (d, J=8.0 Hz, 2H). MS (ESI) m/z 514.9 [M+H]⁺.

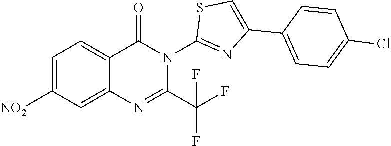 [SMILES: O=c1c2ccc([N+](=O)[O-])cc2nc(C(F)(F)F)n1-c1nc(-c2ccc(Cl)cc2)cs1]

Example 158: 3-(4-(4-Chlorophenyl)thiazol-2-yl)-7-nitro-2-(trifluoromethyl)quinazolin-4(3H)-one (XH216-33p1) ¹H NMR (400 MHz, Chloroform-d) δ 8.80-8.73 (m, 1H), 8.54 (d, J=8.0 Hz, 1H), 8.47 (d, J=8.0 Hz, 1H), 7.86-7.74 (m, 3H), 7.46-7.38 (m, 2H). MS (ESI) m/z 453.0 [M+H]⁺.

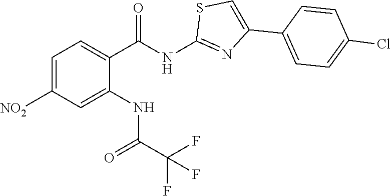 [SMILES: O=C(Nc1nc(-c2ccc(Cl)cc2)cs1)c1ccc([N+](=O)[O-])cc1NC(=O)C(F)(F)F]

Example 159: N-(4-(4-Chlorophenyl)thiazol-2-yl)-4-nitro-2-(2,2,2-trifluoroacetamido)benzamide (XH216-33p2) ¹H NMR (400 MHz, Chloroform-d) δ 12.19 (s, 1H), 9.54 (s, 1H), 8.32 (d, J=8.0 Hz, 1H), 8.19 (d, J=8.0 Hz, 1H), 7.72-7.56 (m, 2H), 7.53-7.41 (m, 2H), 7.23 (s, 1H). MS (ESI) m/z 471.0 [M+H]⁺.

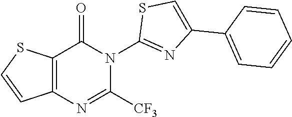 [SMILES: O=c1c2sccc2nc(C(F)(F)F)n1-c1nc(-c2ccccc2)cs1]

Example 160: 3-(4-Phenylthiazol-2-yl)-2-(trifluoromethyl)thieno[3,2-d]pyrimidin-4(3H)-one (XH216-57p1) ¹H NMR (400 MHz, Chloroform-d) δ 7.99 (d, J=5.4 Hz, 1H), 7.88 (d, J=7.8 Hz, 2H), 7.77 (s, 1H), 7.58-7.51 (m, 1H), 7.49-7.40 (m, 2H), 7.40-7.34 (m, 1H). MS (ESI) m/z 380.0 [M+H]⁺.

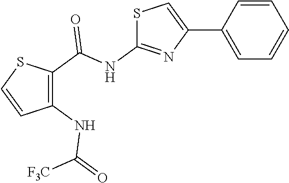 [SMILES: O=C(Nc1nc(-c2ccccc2)cs1)c1sccc1NC(=O)C(F)(F)F]

Example 161: N-(4-Phenylthiazol-2-yl)-3-(2,2,2-trifluoroacetamido)thiophene-2-carboxamide (XH216-57p2) ¹H NMR (400 MHz, Chloroform-d) δ 11.13 (s, 1H), 8.13-8.04 (m, 1H), 7.70-7.58 (m, 5H), 6.59-6.47 (m, 2H). MS (ESI) m/z 398.0 [M+H]⁺.

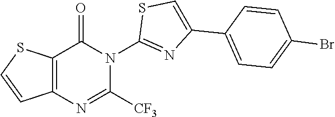 [SMILES: O=c1c2sccc2nc(C(F)(F)F)n1-c1nc(-c2ccc(Br)cc2)cs1]

Example 162: 3-(4-(4-Bromophenyl)thiazol-2-yl)-2-(trifluoromethyl)thieno[3,2-d]pyrimidin-4(3H)-one (XH216-58p1) ¹H NMR (400 MHz, Chloroform-d) δ 8.04-7.93 (m, 1H), 7.82-7.70 (m, 3H), 7.60-7.53 (m, 3H). MS (ESI) m/z 457.9 [M+H]⁺.

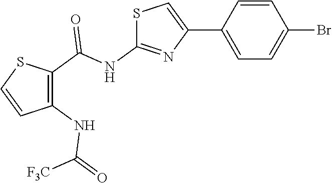 [SMILES: O=C(Nc1nc(-c2ccc(Br)cc2)cs1)c1sccc1NC(=O)C(F)(F)F]

Example 163: N-(4-(4-Bromophenyl)thiazol-2-yl)-3-(2,2,2-trifluoroacetamido)thiophene-2-carboxamide (XH216-58p2) ¹H NMR (400 MHz, Chloroform-d) δ 11.03 (s, 1H), 8.96 (s, 2H), 8.16-8.08 (m, 1H), 7.71-7.68 (m, 2H), 6.57 (s, 2H). MS (ESI) m/z 475.0 [M+H]⁺.

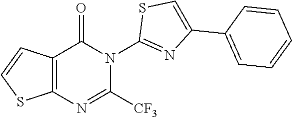 [SMILES: O=c1c2ccsc2nc(C(F)(F)F)n1-c1nc(-c2ccccc2)cs1]

Example 164: 3-(4-Phenylthiazol-2-yl)-2-(trifluoromethyl)thieno[2,3-d]pyrimidin-4(3H)-one (XH216-59p1) ¹H NMR (400 MHz, Chloroform-d) δ 7.86-7.76 (m, 2H), 7.70 (s, 1H), 7.58-7.52 (m, 1H), 7.52-7.46 (m, 1H), 7.41-7.36 (m, 2H), 7.34-7.27 (m, 1H). MS (ESI) m/z 380.0 [M+H]⁺.

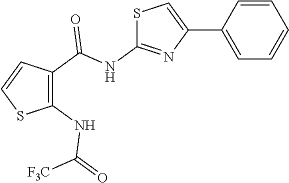 [SMILES: O=C(Nc1nc(-c2ccccc2)cs1)c1ccsc1NC(=O)C(F)(F)F]

Example 165: N-(4-Phenylthiazol-2-yl)-2-(2,2,2-trifluoroacetamido)thiophene-3-carboxamide (XH216-59p2) ¹H NMR (400 MHz, Chloroform-d) δ 12.48 (s, 1H), 7.81-7.63 (m, 2H), 7.56-7.47 (m, 1H), 7.46-7.28 (m, 3H), 7.13 (s, 1H), 7.08-6.96 (m, 1H). MS (ESI) m/z 398.0 [M+H]⁺.

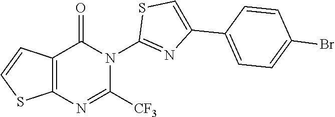 [SMILES: O=c1c2ccsc2nc(C(F)(F)F)n1-c1nc(-c2ccc(Br)cc2)cs1]

Example 166: 3-(4-(4-Bromophenyl)thiazol-2-yl)-2-(trifluoromethyl)thieno[2,3-d]pyrimidin-4(3H)-one (XH216-60p1) ¹H NMR (400 MHz, Chloroform-d) δ 7.80-7.69 (m, 2H), 7.67-7.47 (m, 4H), 7.43-7.26 (m, 1H). MS (ESI) m/z 457.9 [M+H]⁺.

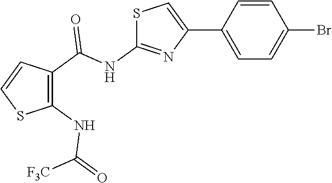 [SMILES: O=C(Nc1nc(-c2ccc(Br)cc2)cs1)c1ccsc1NC(=O)C(F)(F)F]

Example 167: N-(4-(4-Bromophenyl)thiazol-2-yl)-2-(2,2,2-trifluoroacetamido)thiophene-3-carboxamide (XH216-60p2) ¹H NMR (400 MHz, Chloroform-d) δ 12.55 (s, 1H), 7.74-7.64 (m, 2H), 7.64-7.54 (m, 2H), 7.50-7.40 (m, 1H), 7.21 (s, 1H), 7.15-7.04 (m, 1H). MS (ESI) m/z 475.9 [M+H]⁺.

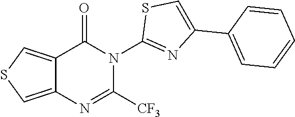 [SMILES: O=c1c2cscc2nc(C(F)(F)F)n1-c1nc(-c2ccccc2)cs1]

Example 168: 3-(4-Phenylthiazol-2-yl)-2-(trifluoromethyl)thieno[3,4-d]pyrimidin-4(3H)-one (XH216-76p1) ¹H NMR (400 MHz, Chloroform-d) δ 8.51-8.40 (m, 1H), 7.94-7.84 (m, 3H), 7.74 (s, 1H), 7.46-7.41 (m, 2H), 7.41-7.34 (m, 1H). MS (ESI) m/z 380.0 [M+H]⁺.

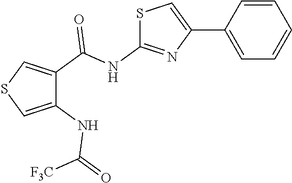 [SMILES: O=C(Nc1nc(-c2ccccc2)cs1)c1cscc1NC(=O)C(F)(F)F]

Example 169: N-(4-Phenylthiazol-2-yl)-4-(2,2,2-trifluoroacetamido)thiophene-3-carboxamide (XH216-76p2) ¹H NMR (400 MHz, Chloroform-d) δ 11.42 (s, 1H), 8.21-8.04 (m, 2H), 7.83-7.67 (m, 2H), 7.49-7.30 (m, 3H), 7.21 (s, 1H). MS (ESI) m/z 398.0 [M+H]⁺.

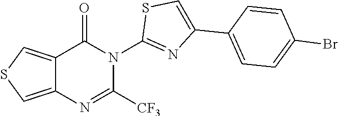 [SMILES: O=c1c2cscc2nc(C(F)(F)F)n1-c1nc(-c2ccc(Br)cc2)cs1]

Example 170: 3-(4-(4-Bromophenyl)thiazol-2-yl)-2-(trifluoromethyl)thieno[3,4-d]pyrimidin-4(3H)-one (XH216-77p1) ¹H NMR (400 MHz, Chloroform-d) δ 8.51-8.37 (m, 1H), 7.98-7.87 (m, 1H), 7.81-7.71 (m, 3H), 7.59-7.53 (m, 2H). MS (ESI) m/z 457.9 [M+H]⁺.

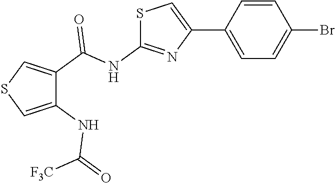 [SMILES: O=C(Nc1nc(-c2ccc(Br)cc2)cs1)c1cscc1NC(=O)C(F)(F)F]

Example 171: N-(4-(4-Bromophenyl)thiazol-2-yl)-4-(2,2,2-trifluoroacetamido)thiophene-3-carboxamide (XH216-77p2) ¹H NMR (400 MHz, Chloroform-d) δ 11.45 (s, 1H), 8.25 (s, 1H), 8.18 (s, 1H), 7.68 (d, J=8.0 Hz, 2H), 7.58 (d, J=8.0 Hz, 2H), 7.23 (s, 1H). MS (ESI) m/z 475.9 [M+H]⁺.

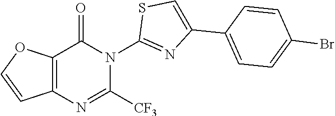 [SMILES: O=c1c2occc2nc(C(F)(F)F)n1-c1nc(-c2ccc(Br)cc2)cs1]

Example 172: 3-(4-(4-Bromophenyl)thiazol-2-yl)-2-(trifluoromethyl)furo[3,2-d]pyrimidin-4(3H)-one (XH216-100P1) ¹H NMR (400 MHz, Chloroform-d) δ 7.97 (d, J=2.1 Hz, 1H), 7.78 (s, 1H), 7.75 (s, 1H), 7.73 (s, 1H), 7.61-7.53 (m, 2H), 7.02 (d, J=2.1 Hz, 1H). MS (ESI) m/z 441.9 [M+H]⁺.

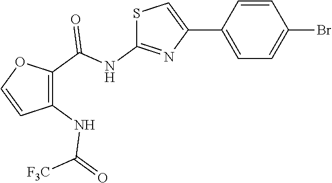 [SMILES: O=C(Nc1nc(-c2ccc(Br)cc2)cs1)c1occc1NC(=O)C(F)(F)F]

Example 173: N-(4-(4-Bromophenyl)thiazol-2-yl)-3-(2,2,2-trifluoroacetamido)furan-2-carboxamide (XH216-100P2) ¹H NMR (400 MHz, Chloroform-d) δ 10.24 (s, 1H), 7.77-7.64 (m, 2H), 7.58-7.52 (m, 3H), 7.49 (s, 1H), 7.22 (s, 1H). MS (ESI) m/z 459.9 [M+H]⁺.

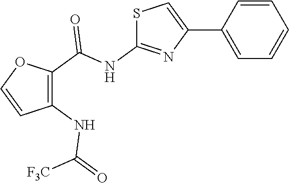 [SMILES: O=C(Nc1nc(-c2ccccc2)cs1)c1occc1NC(=O)C(F)(F)F]

Example 174: N-(4-Phenylthiazol-2-yl)-3-(2,2,2-trifluoroacetamido)furan-2-carboxamide (XH216-118p2) ¹H NMR (400 MHz, Methanol-d₄) δ 7.83 (d, J=8.0 Hz, 2H), 7.38 (d, J=8.0 Hz, 3H), 7.29 (d, J=8.0 Hz, 1H), 7.21 (s, 1H). MS (ESI) m/z 382.1 [M+H]⁺.

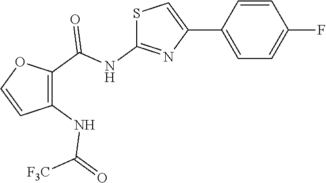 [SMILES: O=C(Nc1nc(-c2ccc(F)cc2)cs1)c1occc1NC(=O)C(F)(F)F]

Example 175: N-(4-(4-Fluorophenyl)thiazol-2-yl)-3-(2,2,2-trifluoroacetamido)furan-2-carboxamide (XH216-119p2) ¹H NMR (400 MHz, Methanol-d₄) δ 8.01-7.88 (m, 2H), 7.77 (s, 1H), 7.41 (s, 1H), 7.38 (s, 1H), 7.13 (d, J=8.0 Hz, 2H). MS (ESI) m/z 400.0 [M+H]⁺.

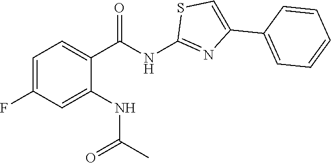 [SMILES: CC(=O)Nc1cc(F)ccc1C(=O)Nc1nc(-c2ccccc2)cs1]

Example 176: 2-Acetamido-4-fluoro-N-(4-phenylthiazol-2-yl)benzamide (XH216-125) ¹H NMR (400 MHz, Methanol-d₄) δ 8.22 (d, J=12.0 Hz, 1H), 8.04-7.87 (m, 3H), 7.48-7.34 (m, 3H), 7.34-7.22 (m, 1H), 7.03-6.91 (m, 1H), 2.22 (s, 3H). MS (ESI) m/z 356.1 [M+H]⁺.

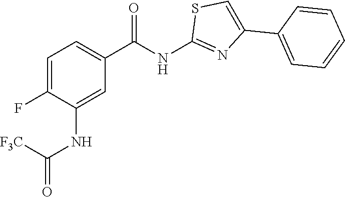 [SMILES: O=C(Nc1nc(-c2ccccc2)cs1)c1ccc(F)c(NC(=O)C(F)(F)F)c1]

Example 177: 4-Fluoro-N-(4-phenylthiazol-2-yl)-3-(2,2,2-trifluoroacetamido)benzamide (XH216-126) ¹H NMR (400 MHz, Chloroform-d) δ 8.87 (d, J=8.0 Hz, 1H), 8.09 (s, 1H), 7.96-7.85 (m, 1H), 7.78-7.68 (m, 2H), 7.44-7.36 (m, 2H), 7.33 (s, 1H), 7.18 (s, 1H). MS (ESI) m/z 410.1 [M+H]⁺.

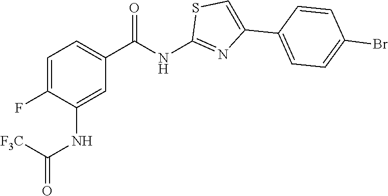 [SMILES: O=C(Nc1nc(-c2ccc(Br)cc2)cs1)c1ccc(F)c(NC(=O)C(F)(F)F)c1]

Example 178: N-(4-(4-Bromophenyl)thiazol-2-yl)-4-fluoro-3-(2,2,2-trifluoroacetamido)benzamide (XH216-127) ¹H NMR (400 MHz, Chloroform-d) δ 8.95-8.78 (m, 1H), 8.15 (s, 1H), 7.99-7.88 (m, 1H), 7.73-7.62 (m, 2H), 7.57-7.44 (m, 2H), 7.40-7.31 (m, 1H), 7.21 (s, 1H). MS (ESI) m/z 488.0 [M+H]⁺.

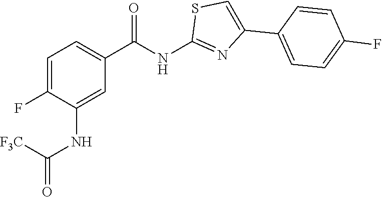 [SMILES: O=C(Nc1nc(-c2ccc(F)cc2)cs1)c1ccc(F)c(NC(=O)C(F)(F)F)c1]

Example 179: 4-Fluoro-N-(4-(4-fluorophenyl)thiazol-2-yl)-3-(2,2,2-trifluoroacetamido)benzamide (XH216-128) ¹H NMR (400 MHz, Methanol-d₄) δ 8.36-8.30 (m, 1H), 8.09-8.00 (m, 1H), 8.00-7.92 (m, 2H), 7.49-7.35 (m, 2H), 7.18-7.05 (m, 2H). MS (ESI) m/z 428.0 [M+H]⁺.

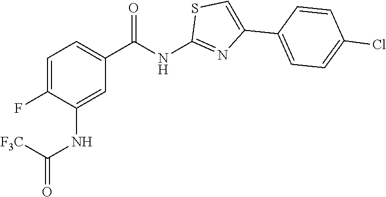 [SMILES: O=C(Nc1nc(-c2ccc(Cl)cc2)cs1)c1ccc(F)c(NC(=O)C(F)(F)F)c1]

Example 180: N-(4-(4-Chlorophenyl)thiazol-2-yl)-4-fluoro-3-(2,2,2-trifluoroacetamido)benzamide (XH216-129) ¹H NMR (400 MHz, Methanol-d₄) δ 8.37-8.26 (m, 1H), 8.14-7.99 (m, 1H), 7.93 (d, J=12.0 Hz, 2H), 7.50-7.37 (m, 4H). MS (ESI) m/z 444.0 [M+H]⁺.

TABLE 2
Additional compounds that can be made according to the above methods.

| Example | | |
|---|---|---|
| No. | Structure | Name |
| 181 | 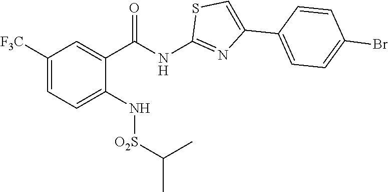 [SMILES: CC(C)S(=O)(=O)Nc1ccc(C(F)(F)F)cc1C(=O)Nc1nc(-c2ccc(Br)cc2)cs1] | N-(4-(4- bromophenyl)thiazol-2- yl)-2-((1- methylethyl) sulfonamido)-5- (trifluoromethyl) benzamide |
| 182 | 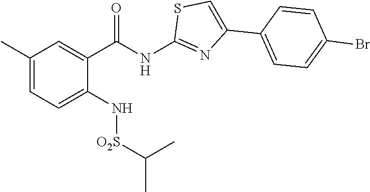 [SMILES: Cc1ccc(NS(=O)(=O)C(C)C)c(C(=O)Nc2nc(-c3ccc(Br)cc3)cs2)c1] | N-(4-(4- bromophenyl)thiazol-2- yl)-5-methyl-2-((1- methylethyl)sulfonamido) benzamide |
| 183 | 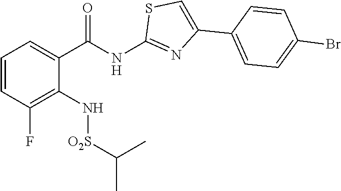 [SMILES: CC(C)S(=O)(=O)Nc1c(F)cccc1C(=O)Nc1nc(-c2ccc(Br)cc2)cs1] | N-(4-(4- bromophenyl)thiazol-2- yl)-3-fluoro-2-((1- methylethyl)sulfonamido) benzamide |
| 184 | 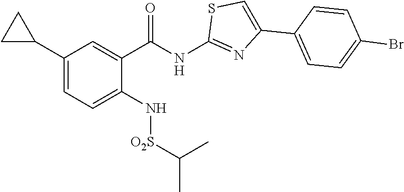 [SMILES: CC(C)S(=O)(=O)Nc1ccc(C2CC2)cc1C(=O)Nc1nc(-c2ccc(Br)cc2)cs1] | N-(4-(4- bromophenyl)thiazol-2- yl)-5-cyclopropyl-2-((1- methylethyl)sulfonamido) benzamide |
| 185 | 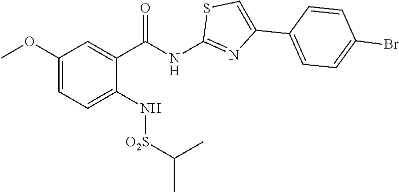 [SMILES: COc1ccc(NS(=O)(=O)C(C)C)c(C(=O)Nc2nc(-c3ccc(Br)cc3)cs2)c1] | N-(4-(4- bromophenyl)thiazol-2- yl)-5-methoxy-2-((1- methylethyl)sulfonamido) benzamide |
| 186 | 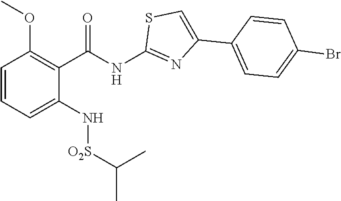 [SMILES: COc1cccc(NS(=O)(=O)C(C)C)c1C(=O)Nc1nc(-c2ccc(Br)cc2)cs1] | N-(4-(4- bromophenyl)thiazol-2- yl)-2-methoxy-6-((1- methylethyl)sulfonamido) benzamide |
| 187 | 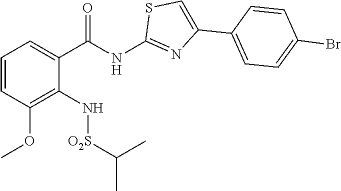 [SMILES: COc1cccc(C(=O)Nc2nc(-c3ccc(Br)cc3)cs2)c1NS(=O)(=O)C(C)C] | N-(4-(4- bromophenyl)thiazol-2- yl)-3-methoxy-2-((1- methylethyl)sulfonamido) benzamide |
| 188 | 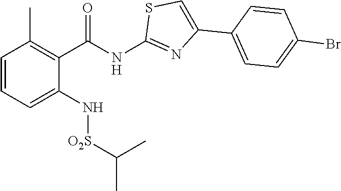 [SMILES: Cc1cccc(NS(=O)(=O)C(C)C)c1C(=O)Nc1nc(-c2ccc(Br)cc2)cs1] | N-(4-(4- bromophenyl)thiazol-2- yl)-2-methyl-6-((1- methylethyl)sulfonamido) benzamide |
| 189 | 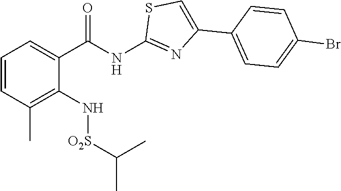 [SMILES: Cc1cccc(C(=O)Nc2nc(-c3ccc(Br)cc3)cs2)c1NS(=O)(=O)C(C)C] | N-(4-(4- bromophenyl)thiazol-2- yl)-3-methyl-2-((1- methylethyl)sulfonamido) benzamide |
| 190 | 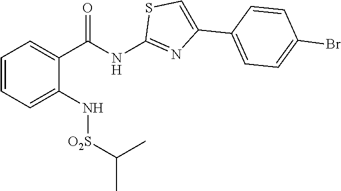 [SMILES: CC(C)S(=O)(=O)Nc1ccccc1C(=O)Nc1nc(-c2ccc(Br)cc2)cs1] | N-(4-(4- bromophenyl)thiazol-2- yl)-2-((1- methylethyl)sulfonamido) benzamide |
| 191 | 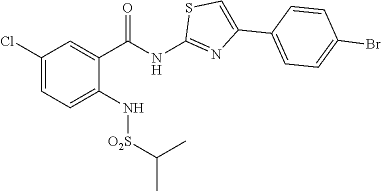 [SMILES: CC(C)S(=O)(=O)Nc1ccc(Cl)cc1C(=O)Nc1nc(-c2ccc(Br)cc2)cs1] | N-(4-(4- bromophenyl)thiazol-2- yl)-5-chloro-2-((1- methylethyl)sulfonamido) benzamide |
| 192 | 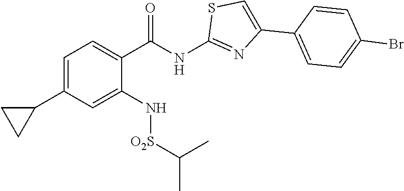 [SMILES: CC(C)S(=O)(=O)Nc1cc(C2CC2)ccc1C(=O)Nc1nc(-c2ccc(Br)cc2)cs1] | N-(4-(4- bromophenyl)thiazol-2- yl)-4-cyclopropyl-2-((1- methylethyl)sulfonamido) benzamide |
| 193 | 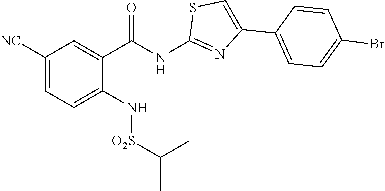 [SMILES: CC(C)S(=O)(=O)Nc1ccc(C#N)cc1C(=O)Nc1nc(-c2ccc(Br)cc2)cs1] | N-(4-(4- bromophenyl)thiazol-2- yl)-5-cyano-2-((1- methylethyl)sulfonamido) benzamide |
| 194 | 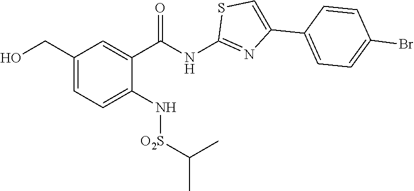 [SMILES: CC(C)S(=O)(=O)Nc1ccc(CO)cc1C(=O)Nc1nc(-c2ccc(Br)cc2)cs1] | N-(4-(4- bromophenyl)thiazol-2- yl)-5-(hydroxymethyl)- 2-((1- methylethyl)sulfonamido) benzamide |
| 195 | 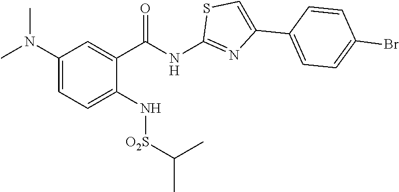 [SMILES: CC(C)S(=O)(=O)Nc1ccc(N(C)C)cc1C(=O)Nc1nc(-c2ccc(Br)cc2)cs1] | N-(4-(4- bromophenyl)thiazol-2- yl)-5-(dimethylamino)- 2-((1- methylethyl)sulfonamido) benzamide |
| 196 | 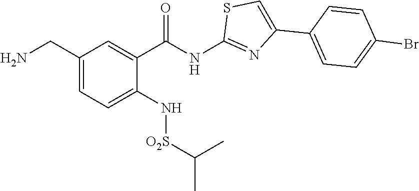 [SMILES: CC(C)S(=O)(=O)Nc1ccc(CN)cc1C(=O)Nc1nc(-c2ccc(Br)cc2)cs1] | 5-(aminomethyl)-N-(4- (4- bromophenyl)thiazol-2- yl)-2-((1- methylethyl)sulfonamido) benzamide |
| 197 | 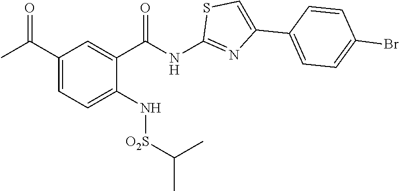 [SMILES: CC(=O)c1ccc(NS(=O)(=O)C(C)C)c(C(=O)Nc2nc(-c3ccc(Br)cc3)cs2)c1] | 5-acetyl-N-(4-(4- bromophenyl)thiazol-2- yl)-2-((1- methylethyl)sulfonamido) benzamide |
| 198 | 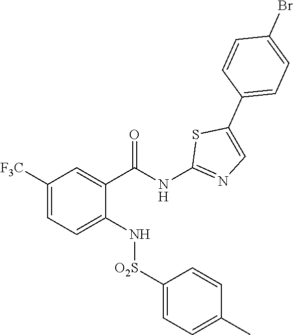 [SMILES: Cc1ccc(S(=O)(=O)Nc2ccc(C(F)(F)F)cc2C(=O)Nc2ncc(-c3ccc(Br)cc3)s2)cc1] | N-(5-(4- bromophenyl)thiazol-2- yl)-2-((4- methylphenyl) sulfonamido)-5- (trifluoromethyl) benzamide |
| 199 | 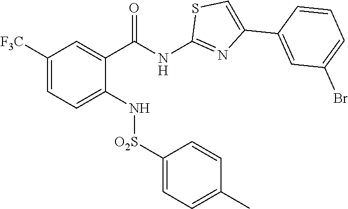 [SMILES: Cc1ccc(S(=O)(=O)Nc2ccc(C(F)(F)F)cc2C(=O)Nc2nc(-c3cccc(Br)c3)cs2)cc1] | N-(4-(3- bromophenyl)thiazol-2- yl)-2-((4- methylphenyl) sulfonamido)-5- (trifluoromethyl) benzamide |
| 200 | 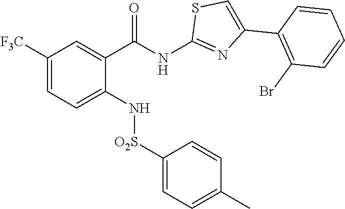 [SMILES: Cc1ccc(S(=O)(=O)Nc2ccc(C(F)(F)F)cc2C(=O)Nc2nc(-c3ccccc3Br)cs2)cc1] | N-(4-(2- bromophenyl)thiazol-2- yl)-2-((4- methylphenyl) sulfonamido)-5- (trifluoromethyl) benzamide |
| 201 | 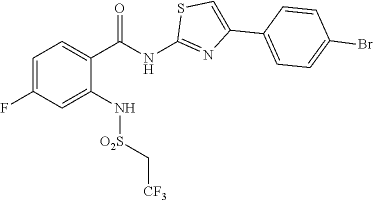 [SMILES: O=C(Nc1nc(-c2ccc(Br)cc2)cs1)c1ccc(F)cc1NS(=O)(=O)CC(F)(F)F] | N-(4-(4- bromophenyl)thiazol-2- yl)-4-fluoro-2-((2,2,2- trifluoroethyl) sulfonamido)benzamide |
| 202 | 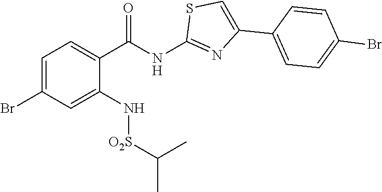 [SMILES: CC(C)S(=O)(=O)Nc1cc(Br)ccc1C(=O)Nc1nc(-c2ccc(Br)cc2)cs1] | 4-bromo-N-(4-(4- bromophenyl)thiazol-2- yl)-2-((1- methylethyl) sulfonamido)benzamide |
| 203 | 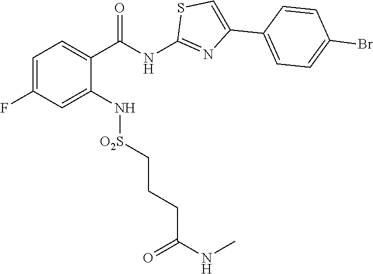 [SMILES: CNC(=O)CCCS(=O)(=O)Nc1cc(F)ccc1C(=O)Nc1nc(-c2ccc(Br)cc2)cs1] | N-(4-(4- bromophenyl)thiazol-2- yl)-4-fluoro-2-((4- (methylamino)-4- oxobutyl)sulfonamido) benzamide |
| 204 | 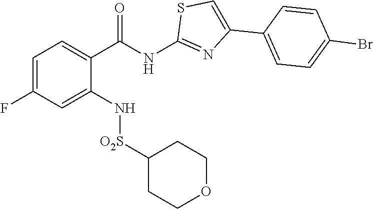 [SMILES: O=C(Nc1nc(-c2ccc(Br)cc2)cs1)c1ccc(F)cc1NS(=O)(=O)C1CCOCC1] | N-(4-(4- bromophenyl)thiazol-2- yl)-4-fluoro-2- ((tetrahydro-2H- pyran)-4- sulfonamido)benzamide |
| 205 | 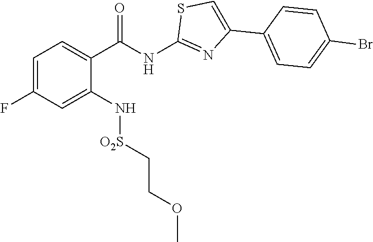 [SMILES: COCCS(=O)(=O)Nc1cc(F)ccc1C(=O)Nc1nc(-c2ccc(Br)cc2)cs1] | N-(4-(4- bromophenyl)thiazol-2- yl)-4-fluoro-2-((2- methoxyethyl)sulfonamido) benzamide |
| 206 | 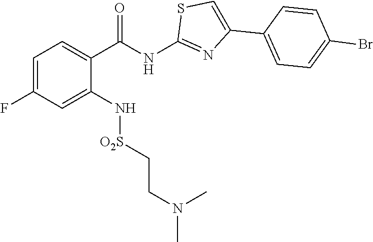 [SMILES: CN(C)CCS(=O)(=O)Nc1cc(F)ccc1C(=O)Nc1nc(-c2ccc(Br)cc2)cs1] | N-(4-(4- bromophenyl)thiazol-2- yl)-2-((2- (dimethylamino)ethyl) sulfonamido)-4- fluorobenzamide |
| 207 | 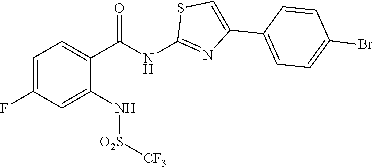 [SMILES: O=C(Nc1nc(-c2ccc(Br)cc2)cs1)c1ccc(F)cc1NS(=O)(=O)C(F)(F)F] | N-(4-(4- bromophenyl)thiazol-2- yl)-4-fluoro-2- ((trifluoromethyl) sulfonamido)benzamide |
| 208 | 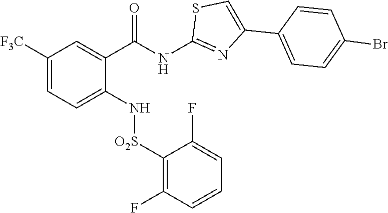 [SMILES: O=C(Nc1nc(-c2ccc(Br)cc2)cs1)c1cc(C(F)(F)F)ccc1NS(=O)(=O)c1c(F)cccc1F] | N-(4-(4- bromophenyl)thiazol-2- yl)-2-((2,6- difluorophenyl) sulfonamido)-5- (trifluoromethyl) benzamide |

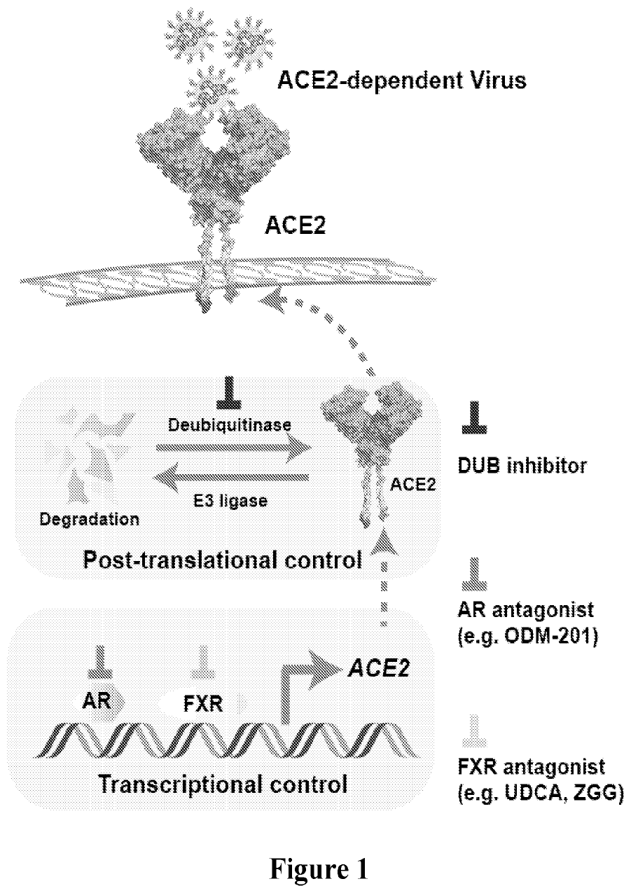

Example 209. (FIG. 1) A schematic model showing the regulation of ACE2 expression on transcriptional and post-translational levels, as well as strategies targeting ACE2 expression modulation against viral infection.

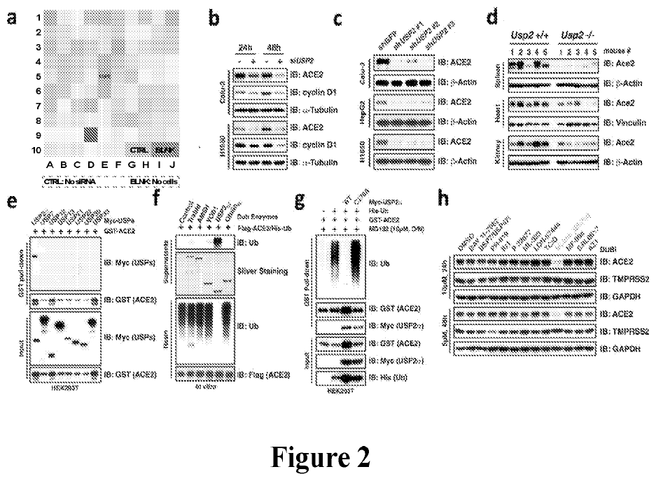

Example 210. (FIG. 2) Identification of USP2 as a physiological deubiquitinase of ACE2. (a) A heatmap showing the results of RNAi-based deubiquitinating enzyme screening, pinpointing USP2 as the leading candidate. An arrayed human ON-TARGETplus siRNA library against deubiquitinating enzyme (horizon) was transfected into HepG2-ACE2-Luc cells seeded in six-well plates using Lipofectamine™ RNAiMAX with reverse transfection. All procedures were following manufacturer's instructions. ACE2-Luc signals were measured at 48 hours post-transfection. (b) siRNA-based silencing of USP2 led to ACE2 protein reduction in Calu-3 and H1650 cells. (c) shRNA-mediated USP2 knock-down led to ACE2 protein reduction. shRNA against USP2 were purchased from Sigma and lentiviral particles were packaged, following infecting indicated cell lines to silence USP2. (d) Immunoblot examination of Ace2 protein abundance in indicated tissues derived from Usp2 knockout and its WT counterpart mice. (e) Immunoprecipitation assay showing that ACE2 specifically interacts with USP2a in cells. (f) In vitro deubiquitination assay showed that USP2 serves as the deubiquitinase of ACE2, governing its deubiquitination. Flag-tagged ACE2 and His-tagged Ubiquitin were co-transfected into HEK293 cells, cells were treated with MG132 (10 μM) for overnight before being harvested. Flag-ACE2 were immunoprecipitated and co-incubated with indicated deubiquitinating enzymes following manufacturer's instruction (R & D Systems™ K-400). (g) The catalytically inactive mutant form of USP2a (C276A) abolished its ability of deubiquitination of ACE2. Plasmids expressing indicated proteins were transfected into HEK293 cells with PEI, and cells were treated with MG132 (10 μM) for overnight before being harvested and analyzed. (h) Immunoblot analysis of ACE2 protein abundance following indicated deubiquitinase inhibitor treatment, showing that USP2 inhibition led to dramatic reduction of ACE2 in HepG2 cells.

Example 211. (FIG. 3) Screening of USP2 inhibitors using USP2 enzymatic activity assay. (a-c) Selected USP2 inhibitors were tested in USP2 activity assay at 5 M. The inhibition effect was compared between selected USP2 inhibitor and the reported USP2 inhibitor ML364.

Example 212. (FIG. 4) IC₅₀ determination of selected USP2 inhibitors using USP2 enzymatic activity assays.

Example 213. (FIG. 5) Screening of selected USP2 inhibitors in HepG2 cells using immunoblotting experiments. (a,b) The effect of selected USP2 inhibitors (5 M) on modulating the protein levels of ACE2 and cyclin D1 in HepG2 cells. (c) MS102 showed enhanced efficacy on reducing ACE2 protein levels than ML364 in HepG2 cells. (d) The effect of selected USP2 inhibitors (1 and 5 μM) on modulating the protein levels of ACE2 and cyclin D1 in HepG2 cells. (e-f) The effect of selected USP2 inhibitors (5 M) on modulating the protein levels of ACE2 and cyclin D1 in HepG2 cells. (g-j) The effect of selected USP2 inhibitors at indicated concentration on modulating the protein levels of cyclin D1 in SUM159 cells.

Example 214. (FIG. 6) Selected USP2 inhibitors concentration-dependently reduce ACE2 and cyclin D1 protein level in HepG2 cells.

Example 215. (FIG. 7) MS102 showing improved efficacy in antivirus infection with less toxicity. (a) Pseudotype entry of indicated viral strains following DMSO, ML364 and MS102 treatment in HepG2 cells. Cells were pre-incubated with indicated compounds at 5 μM for 24 hours, following being infected with corresponding pseudotype particles for additional 48 hours. The P values were calculated using Student's t-test (two-sided). ***P<0.001, n=3. (b) Cytopathic effect assay evaluating the activity of indicated compounds at inhibiting authentic hCoV-NL63 and hCoV-OC43 viruses. (c) Summary of the parameters of indicated compounds against corresponding viral strains. EC₅₀: the concentration for 50% maximal effect, TC₅₀: 50% of toxic concentration, TI: therapeutic index=TC₅₀/EC₅₀.

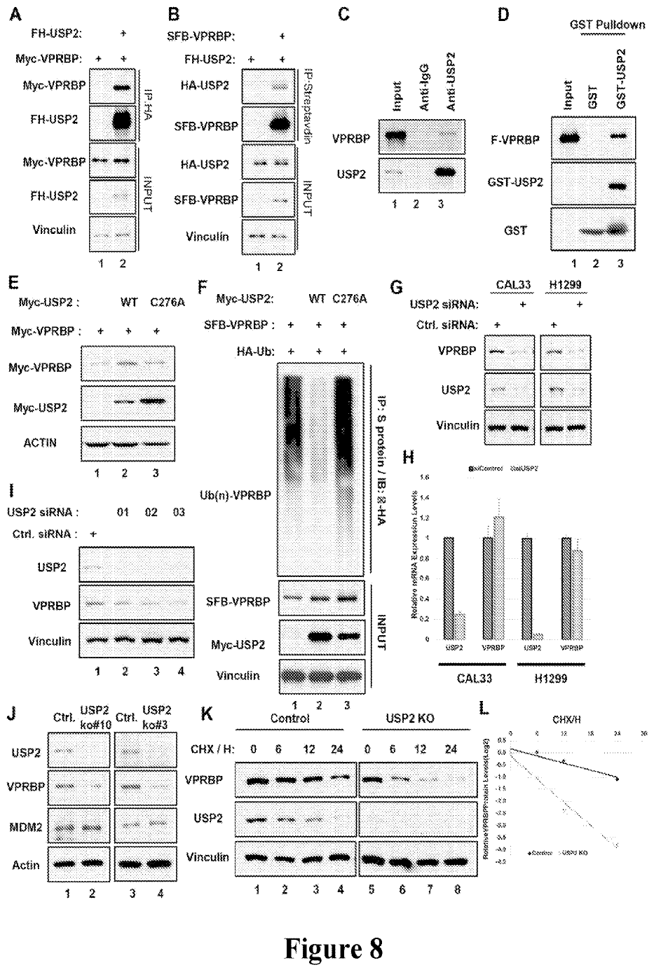

Example 216. (FIG. 8) Identification of VPRBP as a bonafide substrate of USP2. (a) Western blot analysis for VPRBP after immunoprecipitation (IP) of FH-USP2, with HA beads, from H1299 cells transfected with Myc-VPRBP alone or with FH-USP2. (b) Western blot analysis for USP2 after immunoprecipitation (IP) of SFB-VPRBP, with streptavidin beads, from H1299 cells transfected with FH-USP2 alone or with SFB-VPRBP. (c) Western blot analysis for endogenous VPRBP after immunoprecipitation of endogenous USP2 and control IgG in A375 cells. (d) Western blot analysis of F-VPRBP pulled down by GST or GST-USP2 protein in in vitro GST-pull down assay. (e) Western blot analysis of Myc-VPRBP in H1299 whole cell extracts transfected with Myc-VPRBP alone, or plus Myc-USP2 wildtype (WT) or C276A. (f) Western blot analysis of ubiquitinated VPRBP (Ub(n)-VPRBP) after immunoprecipitation of SFB-VPRBP by S protein beads under denaturing condition in H1299 cells transfected with indicated constructs. (g) & (h), Cal33 and H1299 cells were transfected with control or USP2 siRNA for 96 h. Whole cell lysates were subjected to SDS-PAGE followed by western blot analysis (g). Total RNA was extracted for cDNA synthesis and QPCR analysis (h). ***p<0.001; **p<0.01; ns p>0.05. (i) Western blot analysis of VPRBP in H1299 cells transfected with control or USP2 siRNA oligos. (j) Western blot analysis of VPRBP and MDM2 in H1299 control or USP2 knockout (KO) cells. (k) & (l), H1299 control and USP2 knockout (KO) cells were treated with 100 μg/ml cycloheximide (CHX) for indicated time. Whole cell extracts were subjected to SDS-PAGE followed by western blot analysis (k). VPRBP protein abundance was quantified with Image J software (l).

Example 217. (FIG. 9) Screening of selected USP2 inhibitors using cell viability assays. H460 cells were treated with 10 M of indicated compounds for 48 h, cell viabilities were indicated by the images of cell morphology.

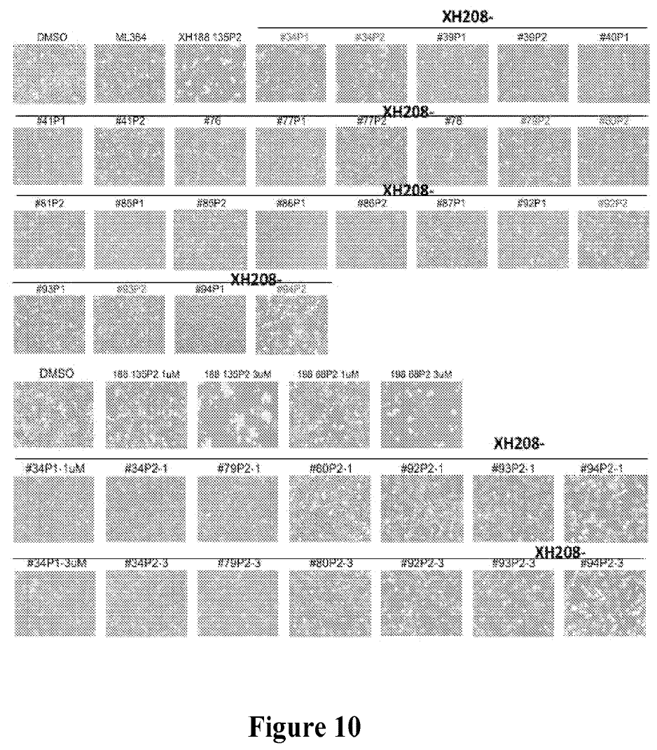

Example 218. (FIG. 10) Screening of selected USP2 inhibitors using cell viability assays. A375 cells were treated with indicated concentration of indicated compounds for 48 h, cell viabilities were indicated by the images of cell morphology.

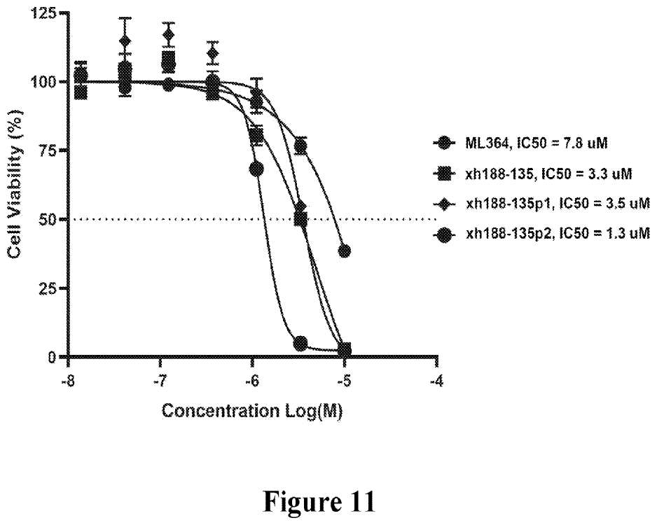

Example 219. (FIG. 11) GI₅₀ determination of selected compounds in A375 cells after 48 hours treatment.

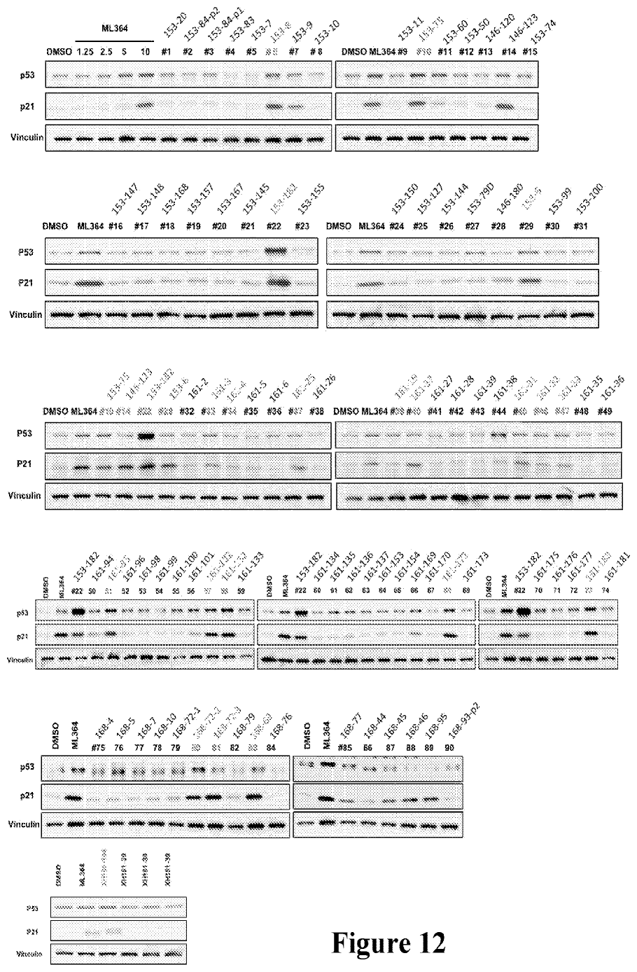

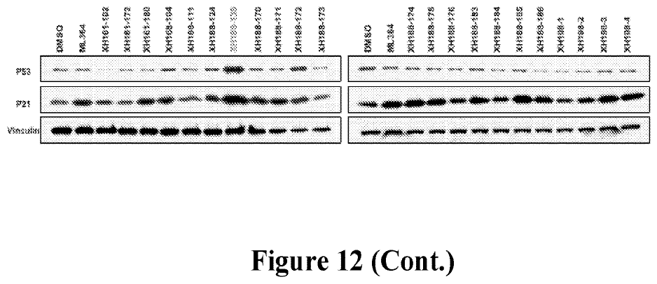

Example 220. (FIG. 12) Screening of selected USP2 inhibitors in H460 cells using immunoblotting experiments. The effect of selected USP2 inhibitors (10 μM, 48 h treatment time) on modulating the protein levels of p53 and p21 in H460 cells.

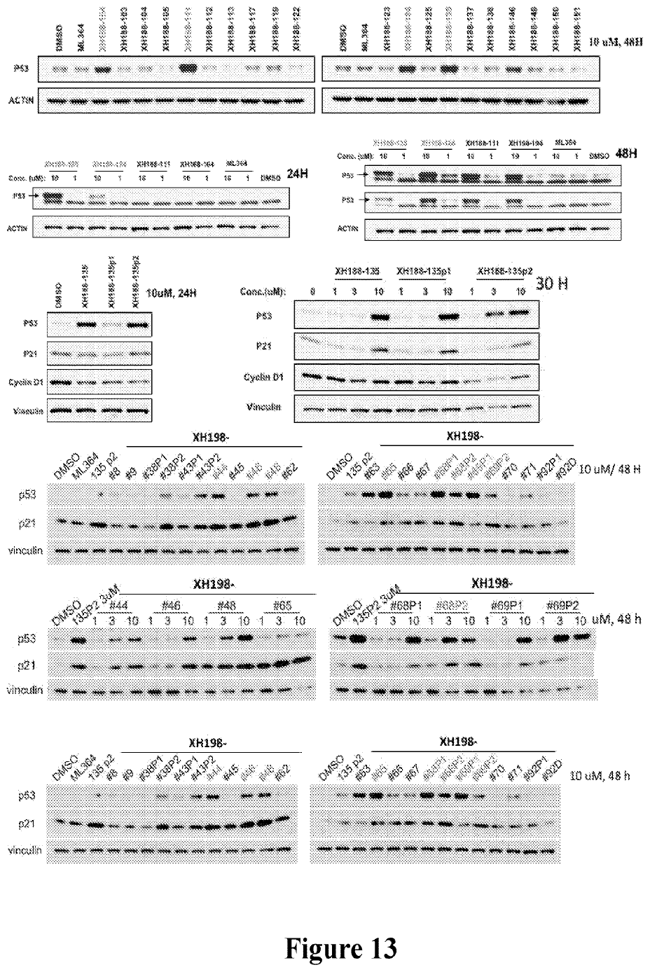

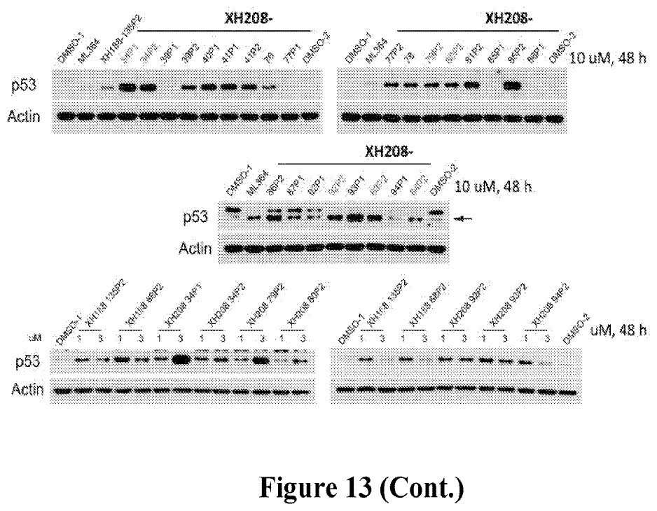

Example 221. (FIG. 13) Screening of selected USP2 inhibitors in A375 cells using immunoblotting experiments. The effect of selected USP2 inhibitors with indicated concentration after indicated treatment time on modulating the protein levels of p53 and p21 in A375 cells.

Example 222. (FIG. 14) PK results of selected compounds. (a) Plasma concentrations of MS102 and ML346 over 24 h after i.p. injection at 50 mg/kg dose. (b) Plasma concentrations of MS102 over 24 h using different administration routes: i.v. (5 mg/kg), i.p. (50 mg/kg) and p.o. (50 mg/kg) by. (c) Plasma concentrations of XH161-102, XH161-172, XH161-180 and XH153-182 over 8 h at 5 mg/kg dose by i.p. injection. (d) Plasma concentrations of XH161-172 over 24 h using different administration routes: i.v. (1 mg/kg) and p.o. (10 mg/kg). (e) Plasma concentrations of XH161-180 over 24 h using different administration routes: i.v. (1 mg/kg) and p.o. (10 mg/kg).

### Biology

### Antibodies

Following antibodies were used for co-IP assay and western blot analysis: anti-USP2 (Abgent Cat #AP2131c, RRID:AB_2212429); anti-VPRBP (Bethyl Cat #A301-888A, RRID:AB_1524107); anti-VPRBP (Santacruz Biotechnology Cat #sc-376850, RRID:AB_2905506); anti-HA (Roche Cat #11867431001, RRID:AB_390919): anti-IRF1 (Santa Cruz Biotechnology Cat #sc-74530, RRID:AB_2126826); anti-p53 (Santa Cruz Biotechnology Cat #sc-126, RRID:AB_628082); anti-mouse p53(Leica Biosystems Cat #NCL-L-p53-CM5p, RRID:AB_2895247); anti-PUMA (Santa Cruz Biotechnology Cat #sc-28226, RRID:AB 2064827); anti-p21 (Santa Cruz Biotechnology Cat #sc-53870, RRID:AB 785026); anti-Actin (Sigma-Aldrich Cat #A5441, RRID:AB_476744); anti-Flag (Sigma-Aldrich Cat #F3165, RRID:AB_259529); anti-vinculin(Sigma-Aldrich Cat #V9131, RRID:AB_477629). ACE2 (ab15348), TMPRSS2 (ab10913), GAPDH (sc-47724), cyclin B1 (sc-245), LC3B (#3868), oa-Tubulin (#2144S), R-Actin (#3700), cyclin D1 (sc-718), anti-His tag (A190-114A), K48-Ub (4289S), Ubiquitin (10201-2-AP), anti-Myc tag (#2278S), anti-GST tag (#2622), GFP (ab290), and SARS-CoV-2 Spike Protein (#99423S).

### Cell Lines and Cell Culture

All cells were incubated in a humidified atmosphere at 37° C. and 5% CO₂. H1299, H1650, H460, HepG2 and HEK293T were purchased from ATCC (American Type Culture Collection). Calu-3 cell line was a kid gift by the P. Liu lab at University of North Carolina at Chapel Hill. H1299, H1650, H460, HepG2 and HEK293T were cultured in RPMI-1640 medium. The other cells were cultured in DMEM medium. All cell culture media were supplemented with 10% fetal bovine serum (catalog number: 10437028) and 1% penicillin/streptomycin (catalog number: 151401122).

### Cell Viability Assays

0.3 million of H460 cells were seeded in 6-well plate. Next day, cells were treated with 10 uM of drugs for 48 h. DMSO served as negative control. Images of cell morphology were obtained with Olympus IX51 microscope, indicating the cell viability upon treatment.

### Deubiquitinase Screening

The human ON-TARGETplus siRNA library targeting human deubiquitinating enzymes (cat #G-104705-01, Horizon Discovery) was used to perform the deubiquitinase screen. Reverse transfection was performed with Lipofectamine RNAiMAX (cat #13778075, Invitrogen) following the manufacturer's instructions. Briefly, 6 μmol of RNAi duplex were diluted in 100 μl of serum-free Opti-MEM medium. Then, 1 μl of Lipofectamine RNAiMAX was added into each well containing the diluted RNAi molecules. Mixed gently and incubated at room temperature for 20 min. HepG2_ACE2_Luc cells were diluted with antibiotic-free DMEM medium to a concentration of 100,000 cells per ml and 500 μl of the above cell-containing medium was transferred into each well of the 24-well cell culture plates. Cells were lysed and luminescence was measured 48 hours post-transfection.

### Establishment of P53 or USP2 Knockout Cells by CRISPR Technology

EMT6 cells were transfected with pCW-CAS9 and pLKO empty vector or pLKO-P53 crispr gRNA by using Lipofectamine 3000. pLKO empty vector and pCW-CAS9 constructs are gifts from Dr. Laura Pasqualucci. pLKO-P53 crispr gRNA were generated by inserting P53 crispr gRNA (target sequence: ACCATCGGAGCAGCGCTCA) into pLKO empty vector. 1 μg/ml of doxycycline (Sigma, Cat #D9891) was used to induce CAS9 expression. At 48 h post-transfection, cells were selected with 2 μg/ml puromycin (Sigma, Cat #P9620) for 3 days to get the control and P53-null pools, which were then used to grow single clones. Single clones were firstly determined by anti-P53 (cm-5) western blot. Next, potential P53-null clones were treated with MDM2 antagonist RG7388 (Medchem Express, Cat #HY-15676) to stabilize P53 followed by western blot to determine the protein level of P53 and its downstream targets such as P21 and PUMA. Clones that have undetectable P53 and no change of P53 downstream targets upon RG7388 treatment were defined as P53-null clones. Finally, P53-null clones were further identified by genomic DNA sequencing to confirm the DNA editing by using P53 specific primers (F: TGATCGTTACTCGGCTTGTC; R: GTCTGCCTGTCTTCCAGATAC).

H1299 control and USP2 knockout cells were generated by using similar method with control double nickase plasmid (Santacruz biotechnology, sc-437281) and USP2 double nickase plasmid (Santacruz biotechnology, sc-411243-NIC). USP2 knockout clones were first identified with anti-USP2 antibody by western blot analysis and further confirmed by genomic DNA sequencing using USP2 specific primers(F: GAGTCTTTGAATGGCCAG GA; R: CTGTCCAGCTTCTGGGTTAG).

### In Vitro Deubiquitination Assay

The in vitro de-ubiquitination assay was performed based on the UbiCREST Deubiquitinase Enzyme set (K-400, R & D Systems), following the manufacturer's instructions. The simplified workflow is as follows: transfection of HEK293T cells with Flag-tagged ACE2 and His-tagged ubiquitin to get ubiquitinated ACE2 proteins. Then, treatment of transfected cells with MG132 in a final concentration of 10 μM for overnight before performing immunoprecipitation as described above to get purified ACE2-ubiquitin complex and remove free ubiquitin and DUBs. Enzymatic treatment of the ubiquitinated ACE2 proteins with the DUBs was performed as indicated. SDS-PAGE separation of digested products, which were then analyzed by ubiquitin and target protein, respectively. The silver staining was performed with the Pierece™ Silver Stain Kit (24612) following the manufacturer's instructions.

### DUB Enzymatic Activity Assay

The USP2 (E-504-050, R & D Systems) and SARS-CoV-2 PLpro (100735, BPS Bioscience) deubiquitinase activity was determined with the DUB Activity Assay Kit (701490, Cayman), slightly modified based on the manufacturer's instructions. Briefly, the inhibitors were diluted as indicated with 40 μL of 1×DUB assay buffer, followed by adding 5 μL of DUB proteins. The plate was covered and incubated with the DUB-inhibitor mix at 37° C. for 30 minutes. Then, the plate cover was removed, and the reactions were initiated by quickly adding 5 μL of Ubiquitin-AMC to all wells being used. The fluorescence was read in an Infinite M1000 microplate reader (Tecan), with an excitation wavelength of 360 nm and emission wavelength of 460 nm. The DUB activity was calculated using the formulation provided by the kit.

### Luciferase Assay

H1650 or HepG2 cells were cultured in 96-well or 24-well plates and treated with compounds for 1 hour, 24 hours or 48 hours prior to be exposed to pseudotyped viral strains for additional 48 hours as indicated in corresponding figure legends. Cells were lysed at room temperature for 30 min by adding Gly-gly buffer (25 mM Gly-gly, pH 7.8; 15 mM MgSO₄; 4 mM EGTA, pH 7.8; 1 mM DTT and 1% Triton X-100). 150 μl each well for 6-well plate or 50 μl each well for 96-well plates. 25 μl of lysates per well were aliquoted and transferred to white flat 96-well plates accordingly, followed by adding 25 μl of the luciferase assay mixture (20 mM Gly-gly, pH 7.8; 12 mM MgSO₄; 3 mM EGTA, pH 7.8; 0.2 mM potassium phosphate, pH 7.8; 2 mM ATP; 1.5 mM DTT and 1.25 mg/ml firefly luciferin). Fluorescence intensity was measured by using Epoch Microplate Spectrophotometer (BioTek).

### Immunoblot and Immunoprecipitation

### Method 1.

The detailed procedure for performing immunoblot and immunoprecipitation assays was described in our previous work (Dang et al., 2021). In brief, cells were lysed in protease and phosphatase inhibitor-contained EBC buffer (50 mM Tris-HCl, pH 7.4; 120 mM NaCl; 0.5% NP-40). Protein concentrations were determined, and an equal amount of protein samples were harvested, followed by adding 5×SDS loading buffer (312 mM Tris-HCl, 10% SDS, 25 mM P-mercaptoethanol, 50% glycerol, and 0.05% bromophenol blue) and boiling for 10 min. SDS-PAGE electrophoresis, wet electroblotting, milk blocking, primary and secondary antibody incubation, and ECL development were conducted sequentially.

For immunoprecipitation analysis, cell lysates were incubated with Glutathione Sepharose 4B resin, or HA-/Flag-conjugated agarose beads for 4 h at 4° C., respectively. The recovered immunoprecipitates were washed four times with NETN buffer (20 mM Tris, pH 8.0; 100 mM NaCl; 0.5 mM EDTA and 0.5% NP-40), resolved by SDS-PAGE and immunoblotted with indicated antibodies.

### Method 2:

Whole cell extracts were prepared with Flag lysis buffer (50 mM Tris-HCl, pH 8.0, 137 mM NaCl, 1 mM NaF, 1 mM NaVO₃, 1% Triton X-100, 0.2% sarkosyl, 0.5 mM DTT, 0.5 mM PMSF and 10% glycerol) containing fresh-added protease inhibitors. For cytosolic and nuclear fractions, the cell pellet was firstly incubated with Harvest buffer (10 mM Hepes (pH 8.0), 50 mM NaCl, 0.5 M sucrose, 0.1 mM EDTA, and 0.25% Triton X-100) containing fresh-added protease inhibitors for 5 min on ice followed by centrifugation @120 g for 10 min. The supernatant was cytosolic fraction. After wash twice with buffer A (10 mM Hepes (pH 8.0), 10 mM KCl, 0.1 mM EDTA, and 0.1 mM EGTA), nuclear pellet was lyzed with BC100 (50 mM Tris-HCl, pH 8.0, 100 mM NaCl, 0.2% Triton X-100, and 10% glycerol) containing fresh-added protease inhibitors for 30 min on ice to get the nuclear fraction. Protein concentration was measured with the protein assay dye reagent (Bio-Rad, Cat #5000006) as manufacturer's user guide. 20-60 μg total proteins were loaded to and separated in SDS-PAGE precast gels, then transferred to nitrocellulose membrane. After incubation with primary antibodies for overnight at 4° C., HRP-conjugated secondary antibodies were used, and western blot signals were detected on autoradiographic films after incubating with ECL substrate.

For co-immunoprecipitation assay, cells were lyzed and mild sonicated in BC100 buffer containing fresh-added proteinase inhibitors. Whole cell extracts were incubated with anti-Flag M2 beads (Sigma, Cat #A2220), S protein agarose beads (Sigma, Cat #69704) or streptavidin Sepharose™ beads (GE healthcare, Cat #17511301) overnight at 4° C. The next day, after wash with BC100 buffer, immunoprecipitates were eluted with flag peptide (Sigma, Cat #F3290), glycine-HCL, pH2.5 or 2 mg/ml biotin (Sigma, Cat #B4501), pH8.0 respectively. Ubiquitination assays were performed under denaturing condition. Briefly, whole cell extracts were supplemented with SDS (final concentration: 1%) and boiled for 5 min to denature proteins. After that, whole cell extracts were 1:10 diluted with cell lysis buffer to lower SDS concentration to 0.1%. Agarose beads were then added for immunoprecipitation.

### Pharmacokinetic Determination

Pharmacokinetic studies for ML364, MS102, MS1172, MS1180 and XH153-182 were performed by Sai Life Sciences Limited (India). Experiments were design with 3 mice/time point/compound. Animals were administered intraperitoneally, intravenously as slow bolus injection through tail vein, or through oral route with solution formulation of indicated compound at indicated dose. The formulation vehicles were 10% v/v NMP, 5% v/v Solutol HS-15 and 85% v/v Normal saline for ML364 and 10% v/v NMP, 10% Solutol HS-15 and 80% Normal saline for MS102, MS 1172, MS 1180 and XH153-182. Blood samples (approximately 60 μL) were collected under light isoflurane anesthesia (Surgivet®) from a set of three mice at different time points. Immediately after blood collection, plasma was harvested by centrifugation at 4000 rpm, 10 min at 4° C., and samples were stored at −70±10° C. until bioanalysis. All samples were processed for analysis by protein precipitation method and analyzed with the fit-for-purpose LC-MS/MS method (LLOQ=1.01 ng/mL). The plasma pharmacokinetic parameters were estimated using the non-compartmental analysis tool of Phoenix® WinNonlin software (Ver 8.0).

### Plasmids and Reagents

ACE2 cDNA ORF clone (HG10108-CF), TMPRSS2 cDNA ORF clone (HG13070), SARS-CoV-2 NSP3 gene ORF cDNA clone (VG40593-UT), SARS coronavirus (strain WH20) PLpro gene ORF cDNA clone (VG40524-CF) and MERS-CoV PLpro gene ORF cDNA clone (VG40522-CF) were purchased from Sino Biological US Inc. Spike pseudotyping plasmids of Delta variant (B.1.617.2), Alpha variant (B.1.1.7), Beta variant (B.1.351), Gamma variant (P.1) and Omicron (B.1.1.529) were purchased from InvivoGen. pMD2.G (#12259), psPAX2 (#12260) and pLenti-CMV-GFP (#17448) were purchased from Addgene. shRNA bacterial clone oligos against ACE2 (TRCN0000046693, TRCN0000046694, TRCN0000046695) and USP2 (TRCN0000007277, TRCN0000007278, TRCN0000011080) were purchased from Sigma Aldrich Inc. Myc-tagged USP2c WT and C276A constructs were kind gifts from Dr. Wei Gu at Columbia University Irving Medical Center. pCG1-SARS-2-S-HA plasmid was a kind gift by the Stefan Pöhlmann's Lab at German Primate Center, Germany. Spike pseudotyping plasmids of MERS, SARS-CoV-1, WIV1, SHC014, Rs4231, Rs4084, LYRa11, Rs7327, hCoV-229E and Khosta-2 were provided by Dr. Michael Letko at Washington State University. All of those homemade constructs have been validated by sanger sequencing. MG-132 (S2619), Chloroquine (S6999), and Cycloheximide (S7418) were purchased from Selleckchem. Dub inhibitors used in this study were purchased from MedChemExpress. siRNA molecules targeting USP2 were purchased from Qiagen (cat #1027416).

shRNA and siRNA

VPRBP siRNA smartpool (Dharmacon, Cat #L-021119-01-0005); p53 siRNA smartpool (Dharmacon, Cat #L-003329-00-0005); Control siRNA smartpool (Dharmacon, Cat #D-001810-10-50); CUL4A siRNA smartpool (Dharmacon, Cat #L-012610-00-0005); CUL4B siRNA smartpool (Dharmacon, Cat #L-017965-00-0005); DDB1 siRNA smartpool (Dharmacon, Cat #L-012890-00-0005); USP2 siRNA #1-3 (Qiagen, Cat #1027416_5; Cat #1027416_6; Cat #1027416_8) VPRBP shRNA #1-4 (Milliporesigma, Cat #TRCN0000265223; Cat #TRCN0000265224; Cat #TRCN0000251843; Cat #TRCN0000251844).

### Transfection and Lentivirus 1 Transduction.

Transfection of constructs and siRNA oligos was performed with lipofectamine 3000 reagents (Thermofisher scientific, Cat #L3000150) as manufacture's user guide. For lentivirus packaging, HEK293T cells were transfected with shVPRBP-pLKO, Δ8.9 and pCMV-VSVG constructs. At 48 h post-transfection, media containing viruses was harvested and filtered through 0.45 μm syringe filter. Before adding to cells, viruses were concentrated by using Lenti-X Concentrator (Clotech, Cat #631321) as manufacturer's manual and resuspended with complete growth media. EMT6 cells were transduced with viruses overnight and kept growing for 3 days to get shVPRBP pool cells. The VPRBP knockdown efficiency was determined by QPCR or western blot analysis.

### XTT Cytoprotection Assay

For XTT cytoprotection assay against hCoV-NL63 and hCoV-OC43 viral strains, the service was provided by ImQuest BioSciences Inc. Briefly, LLC-MK2 or BHK21 cells were incubated at 37° C./5% CO₂ with serially diluted compound prior to the addition of a known titer of CoV strain NL63 or OC43, respectively. The cultures were then incubated for 6 days at 37° C./5% CO₂. Following the incubation, the cells were stained with the tetrazolium dye XTT and read at 450/650 nm on a spectrophotometer to evaluate cellular viability. Efficacy and toxicity values were calculated using linear regression analysis. The EC₅₀ and TC₅₀ were generated by the service provider using XLfit 4 in conjunction with Xcel and 4-parameter curve.

### GST-Pulldown Assay

GST-fused proteins and F-VPRBP proteins were expressed and purified in E. coli Rosetta (DE3) competent cells (Milliporesigma, Cat #70954-4) and HEK293T cells respectively. After incubation of GST or GST-fused protein and F-VPRBP with GST resin overnight at 4° C., beads were washed 5 times with BC100 buffer, then boiled with SDS loading buffer. Precipitates were subjected to western blot analysis and Ponceau S staining.

As used herein, in case of discrepancy between the structure and chemical name provided for a particular compound, the structure shall control.

### REFERENCES

Altun, M., Kramer, H. B., Willems, L. I., McDermott, J. L., Leach, C. A., Goldenberg, S. J., Kumar, K. G., Konietzny, R., Fischer, R., Kogan, E., et al. (2011). Activity-based chemical proteomics accelerates inhibitor development for deubiquitylating enzymes. Chem. Biol. 18, 1401-1412.10.1016/j.chembiol.2011.08.018.
Chuang, S. J., Cheng, S. C., Tang, H. C., Sun, C. Y., and Chou, C. Y. (2018). 6-Thioguanine is a noncompetitive and slow binding inhibitor of human deubiquitinating protease USP2. Sci. Rep. 8, 3102.10.1038/s41598-018-21476-w.
Dang, F., Nie, L., Zhou, J., Shimizu, K., Chu, C., Wu, Z., Fassl, A., Ke, S., Wang, Y., Zhang, J., et al. (2021). Inhibition of CK1 epsilon potentiates the therapeutic efficacy of CDK4/6 inhibitor in breast cancer. Nat Commun 12, 5386.10.1038/s41467-021-25700-6.
Davis, M. I., Pragani, R., Fox, J. T., Shen, M., Parmar, K., Gaudiano, E. F., Liu, L., Tanega, C., McGee, L., Hall, M. D., et al. (2016). Small molecule inhibition of the ubiquitin-specific protease USP2 accelerates cyclin D1 degradation and leads to cell cycle arrest in colorectal cancer and mantle cell lymphoma models. J. Biol. Chem. 291, 24628-24640.10.1074/jbc.M116.738567.
Frappier, L., and Verrijzer, C. P. (2011). Gene expression control by protein deubiquitinases. Curr. Opin. Genet. Dev. 21, 207-213.10.1016/j.gde.2011.02.005.
Komander, D., Clague, M. J., and Urbe, S. (2009). Breaking the chains: structure and function of the deubiquitinases. Nat Rev Mol Cell Biol 10, 550-563.10.1038/nrm2731.
Magiera, K., Tomala, M., Kubica, K., De Cesare, V., Trost, M., Zieba, B. J., Kachamakova-Trojanowska, N., Les, M., Dubin, G., Holak, T. A., and Skalniak, L. (2017). Lithocholic acid hydroxyamide destabilizes cyclin D1 and induces G(0)/G(1) arrest by inhibiting deubiquitinase USP2a. Cell Chem. Biol. 24, 458-470.e418.10.1016/j.chembiol.2017.03.002.
Nicholson, B., Leach, C. A., Goldenberg, S. J., Francis, D. M., Kodrasov, M. P., Tian, X., Shanks, J., Sterner, D. E., Bernal, A., Mattern, M. R., et al. (2008). Characterization of ubiquitin and ubiquitin-like-protein isopeptidase activities. Protein Sci. 17, 1035-1043.10.1110/ps.083450408.
Ohayon, S., Refua, M., Hendler, A., Aharoni, A., and Brik, A. (2015). Harnessing the oxidation susceptibility of deubiquitinases for inhibition with small molecules. Angew. Chem. Int. Ed. 54, 599-603. https://doi.org/10.1002/anie.201408411.
Tomala, M. D., Magiera-Mularz, K., Kubica, K., Krzanik, S., Zieba, B., Musielak, B., Pustula, M., Popowicz, G. M., Sattler, M., Dubin, G., et al. (2018). Identification of small-molecule inhibitors of USP2a. Eur. J. Med. Chem. 150, 261-267.10.1016/j.ejmech.2018.03.009.
Vamisetti, G. B., Meledin, R., Gopinath, P., and Brik, A. (2019). Halogen substituents in the isoquinoline scaffold switches the selectivity of inhibition between USP2 and USP7. ChemBioChem 20, 282-286. https://doi.org/10.1002/cbic.201800612.

1. A USP2 inhibitor, comprising a compound of Formula (I): 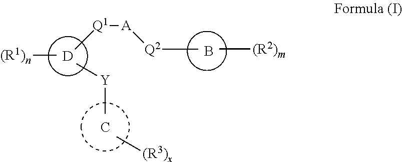
wherein
A is C═O, CH₂ or absent;
Y is a bond, or a bivalent moiety selected from —N(R⁴)—, —N(R⁴)—C₁-C₃ alkylene-, —N(R⁴)—S(O)₂—, —N(R⁴)—C(O)—, —S(O)₂— N(R⁴)—, or —C(O)—N(R⁴)—;
Ring B is a ring structure selected from C₃-C₁₂ cycloalkyl, 3-12 membered heterocyclic, C₆-C₁₀ aryl, or 5-10 membered heteroaryl;
Ring C is absent, or a ring structure selected from C₆-C₁₀ aryl, or 5-10 membered heteroaryl;
Ring D is a ring structure selected from C₅-C₁₀ aryl, 5-10 membered heteroaryl or 3-12 membered heterocyclic;
Q¹ and Q² are independently selected from absent, O, NR⁵, C₁-C₃ alkylene, C₂-C₆ alkenylene, or C₂-C₆ alkynylene, where each said C₁-C₃ alkylene, C₂-C₆ alkenylene, or C₂-C₆ alkynylene is optionally substituted with one or more of halogen, cyano, hydroxyl, oxo, C₁-C₆ alkyl, C₃-C₆ cycloalkyl, C₁-C₆ alkoxyl, or C₃-C₆ cycloalkoxy;
each R¹, each R², and each R³ are independently selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, two adjacent R², or two adjacent R³, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
each R⁴, each R⁵, each R⁶ and each R⁷ are independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
each R⁸ and each R⁹ are independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-Cia aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form a 4-8 membered heterocyclic; and
n is 0, 1, 2, 3, or 4;
m is 0, 1, 2, 3, 4, 5, 6, 7, 8, or 9; and
x is 0, 1, 2, 3, 4, 5, 6, 7, 8, or 9; and
pharmaceutically acceptable salts or tautomers thereof.

2. The USP2 inhibitor of claim 1 wherein the compound is a compound of Formula (I-A): 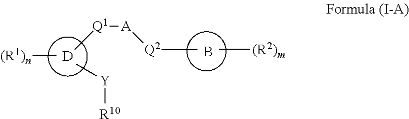
wherein
R¹⁰ is selected from H, OR⁶, NR⁶R⁷, C₁-C₆ alkyl, C₁-C₆ heteroalkyl, C₂-C₆ alkenyl, C₂-C₆ alkynyl, C₃-C₁₂ cycloalkyl, or 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, or 3-12 membered heterocyclic is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; and
A is C═O, CH₂ or absent;
Y is a bond, or a bivalent moiety selected from —N(R⁴)—, —N(R⁴)—C₁-C₃ alkylene-, —N(R⁴)—S(O)₂—, —N(R⁴)—C(O)—, —S(O)₂— N(R⁴)—, or —C(O)—N(R⁴)—;
Ring B is a ring structure selected from C₃-C₁₂ cycloalkyl, 3-12 membered heterocyclic, C₆-C₁₀ aryl, or 5-10 membered heteroaryl;
Ring D is a ring structure selected from C₅-C₁₀ aryl, 5-10 membered heteroaryl or 3-12 membered heterocyclic;
Q¹ and Q² are independently selected from absent, O, NR⁵, C₁-C₃ alkylene, C₂-C₆ alkenylene, or C₂-C₆ alkynylene, where each said C₁-C₃ alkylene, C₂-C₆ alkenylene, or C₂-C₆ alkynylene is optionally substituted with one or more of halogen, cyano, hydroxyl, oxo, C₁-C₆ alkyl, C₃-C₆ cycloalkyl, C₁-C₆ alkoxyl, or C₃-C₆ cycloalkoxy;
R¹ and R² are each independently selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, two adjacent R², or two adjacent R³, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R⁴ and R⁵ are independently selected from H or C₁-C₆ alkyl;
each of R⁶ and R⁷ are independently selected from H, C₁-C₆ alkyl, C₁-C₆ heteroalkyl, C₂-C₆alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
each R⁸ and each R⁹ are independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-Cia aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form a 4-8 membered heterocyclic; and
n is 0, 1, 2, 3, or 4;
m is 0, 1, 2, 3, 4, 5, 6, 7, 8, or 9; and
pharmaceutically acceptable salts or tautomers thereof.

3. The USP2 inhibitor of claim 2, wherein the compound is a compound of Formula (I-A-1): 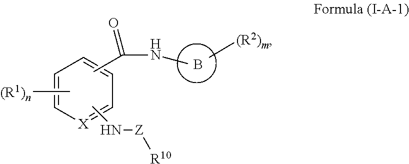
wherein
X is CH or N;
Z is C═O or SO₂;
Ring B is a ring structure selected from C₃-C₁₂ cycloalkyl, 3-12 membered heterocyclic, C₆-C₁₀ aryl, or 5-10 membered heteroaryl;
R¹, R², and R³ are independently selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, two adjacent R², or two adjacent R³, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R⁶ and R⁷ are each independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
R⁸ and R⁹ are each independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-C₁₀ aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form 4-8 membered heterocyclic;
R¹⁰ is selected from H, OR⁶, NR⁶R⁷, C₁-C₆ alkyl, C₁-C₆ heteroalkyl, C₂-C₆ alkenyl, C₂-C₆ alkynyl, C₃-C₁₂ cycloalkyl, or 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, or 3-12 membered heterocyclic is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; and
n is 0, 1, 2, 3, or 4;
m is 0, 1, 2, 3, 4, 5, 6, 7, 8, or 9; and
pharmaceutically acceptable salts or tautomers thereof.

4. The USP2 inhibitor of claim 3, wherein the compound is a compound of Formula (I-A-2): 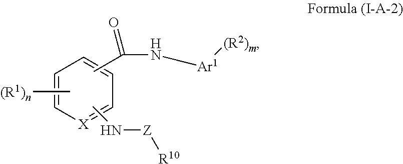
wherein
Ar¹ is selected from C₆-C₁₀ aryl or 5-10 membered heteroaryl;
X is CH or N;
Z is C═O or SO₂;
R¹, R², and R³ are independently selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, two adjacent R², or two adjacent R³, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R⁶ and R⁷ are each independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
R⁸ and R⁹ are each independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-C₁₀ aryl, or 5-10 membered heteroaryl is optionally substituted with halogen, cyano, oxo, C₁-C₆ alkyl, C₁—C₆ heteroalkyl, C₃-C₁₂ cycloalkyl, or 3-12 membered heterocyclic; or R⁸ and R⁹, together with the atoms to which they are attached, optionally form 4-8 membered heterocyclic;
R¹⁰ is selected from H, OR⁶, NR⁶R⁷, C₁-C₆ alkyl, C₁-C₆ heteroalkyl, C₂-C₆ alkenyl, C₂-C₆ alkynyl, C₃-C₁₂ cycloalkyl, or 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, or 3-12 membered heterocyclic is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; and
n is 0, 1, 2, 3, or 4;
m is 0, 1, 2, 3, 4, 5, 6, 7, 8, or 9; and
pharmaceutically acceptable salts or tautomers thereof.

5. The compound of claim 4, wherein Ar¹ is selected from the group consisting of

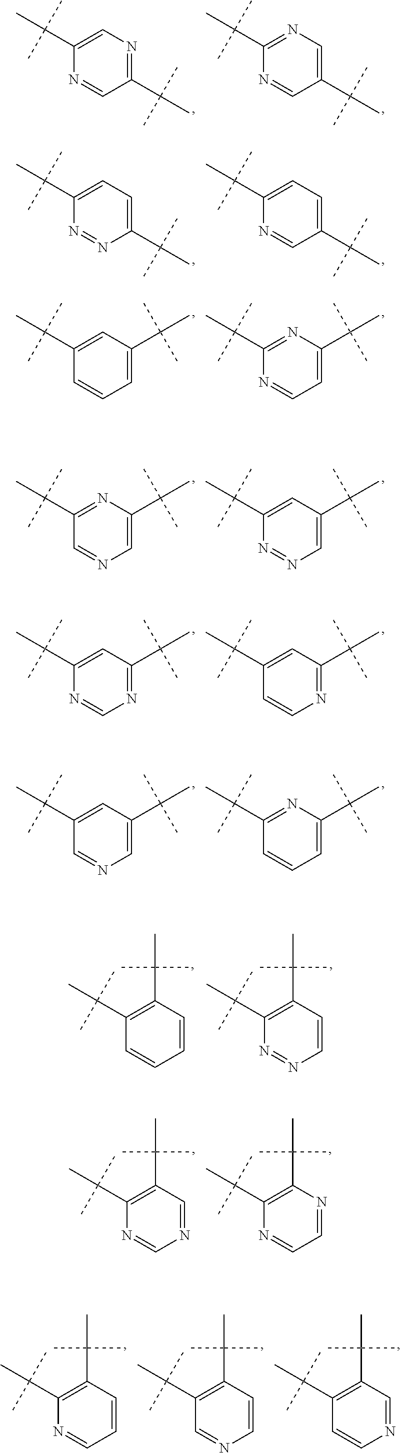

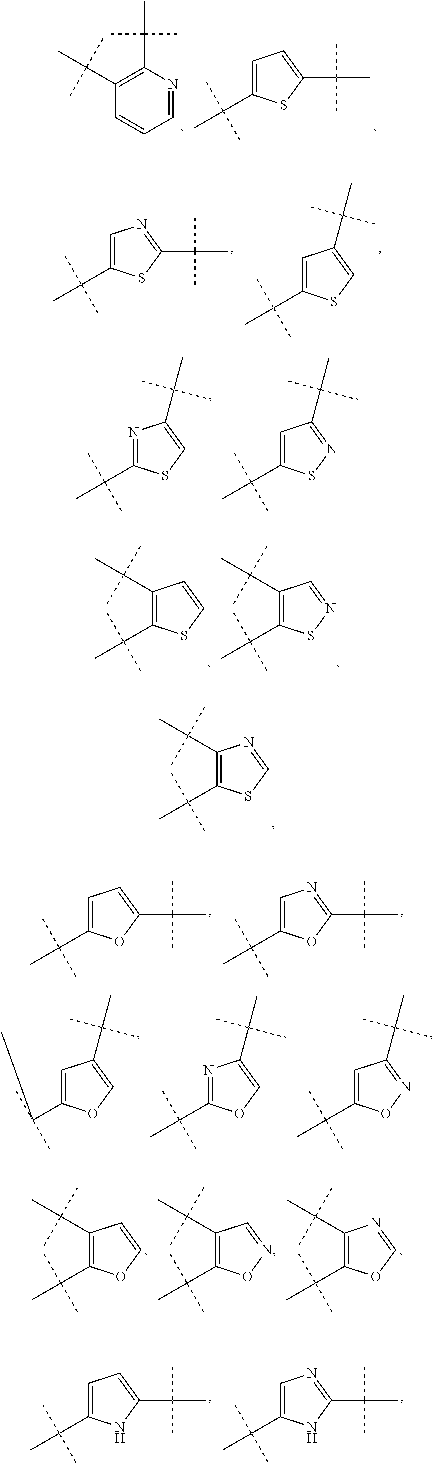

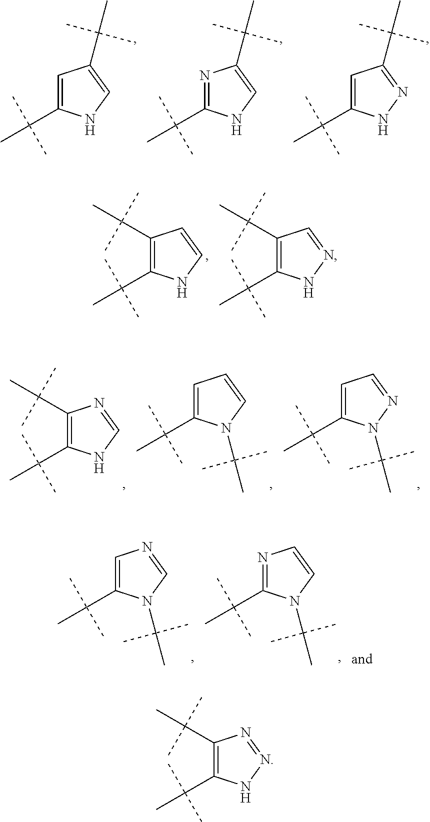

6. The USP2 inhibitor of claim 4, wherein the compound is a compound of Formulae (I-A-3), (I-A-4) or (I-A-5): 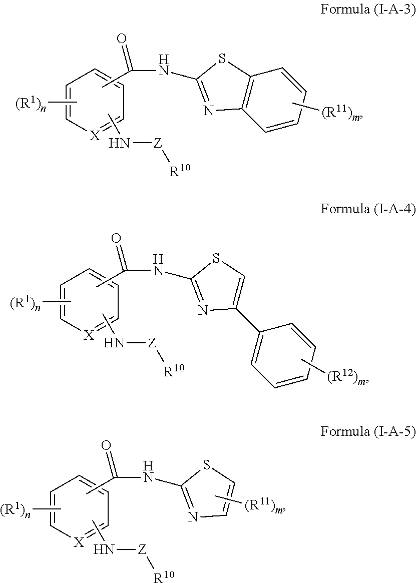
wherein
each R¹¹ is independently selected from H, halogen, cyano, OR⁶, NR⁶R⁷, C(O)OR⁸, C(O)NR⁸R⁹, S(O)₂NR⁸R⁹, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
each R¹² is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹², together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
X is CH or N;
Z is C═O or SO₂;
R¹ is selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R⁶ and R⁷ are each independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
R⁸ and R⁹ are each independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-C₁₀ aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form 4-8 membered heterocyclic;
R¹⁰ is selected from H, OR⁶, NR⁶R⁷, C₁-C₆ alkyl, C₁-C₆ heteroalkyl, C₂-C₆ alkenyl, C₂-C₆ alkynyl, C₃-C₁₂ cycloalkyl, or 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, or 3-12 membered heterocyclic is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; and
n is 0, 1, 2, 3, or 4;
m is 0, 1, 2, 3, 4, 5, 6, 7, 8, or 9; and
pharmaceutically acceptable salts or tautomers thereof.

7. The USP2 inhibitor of claim 6, wherein the compound is a compound of Formulae (I-A-6), (I-A-7) or (I-A-8): 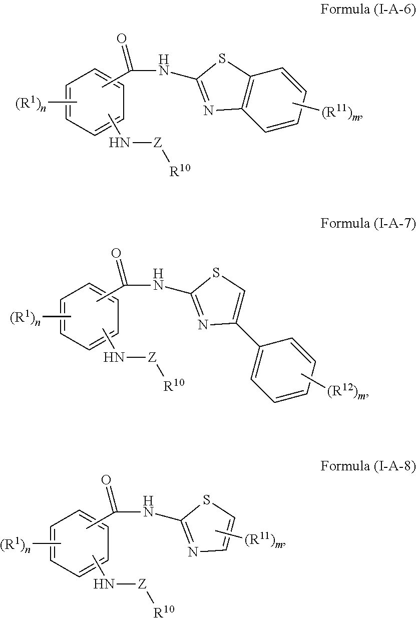
wherein
Z is C═O or SO₂;
each R¹¹ is independently selected from H, halogen, cyano, OR⁶, NR⁶R⁷, C(O)OR⁸, C(O)NR⁸R⁹, S(O)₂NR⁸R⁹, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
each R¹² is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹², together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R¹ is selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R⁶ and R⁷ are each independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
R⁸ and R⁹ are each independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-C₁₀ aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form 4-8 membered heterocyclic;
R¹⁰ is selected from H, OR⁶, NR⁶R⁷, C₁-C₆ alkyl, C₁-C₆ heteroalkyl, C₂-C₆ alkenyl, C₂-C₆ alkynyl, C₃-C₁₂ cycloalkyl, or 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, or 3-12 membered heterocyclic is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; and
n is 0, 1, 2, 3, or 4;
m is 0, 1, 2, 3, 4, 5, 6, 7, 8, or 9; and
pharmaceutically acceptable salts or tautomers thereof.

8. The USP2 inhibitor of claim 7, wherein the compound is a compound of Formulae (I-A-9), (I-A-10) or (I-A-11): 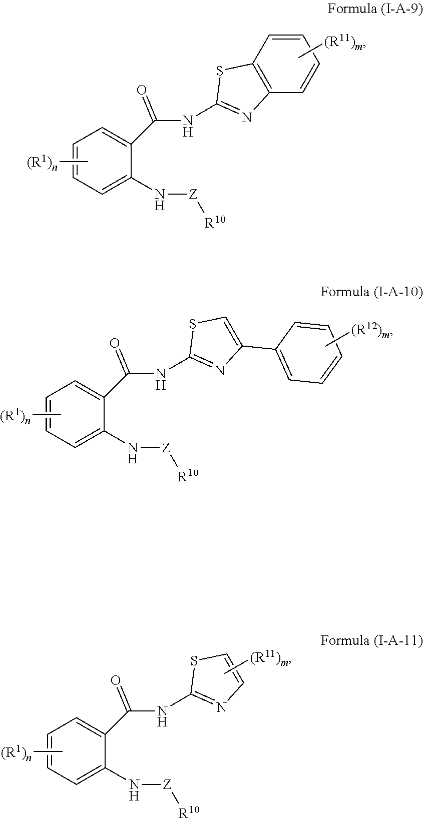
wherein
Z is C═O or SO₂;
each R¹¹ is independently selected from H, halogen, cyano, OR⁶, N⁶R⁷, C(O)OR⁸, C(O)NR⁸R⁹, S(O)₂NR⁸R⁹, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
each R¹² is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹², together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R¹ is selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R⁶ and R⁷ are each independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
R⁸ and R⁹ are each independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-C₁₀ aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form 4-8 membered heterocyclic;
R¹⁰ is selected from H, OR⁶, NR⁶R⁷, C₁-C₆ alkyl, C₁-C₆ heteroalkyl, C₂-C₆ alkenyl, C₂-C₆ alkynyl, C₃-C₁₂ cycloalkyl, or 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, or 3-12 membered heterocyclic is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; and
n is 0, 1, 2, 3, or 4;
m is 0, 1, 2, 3, 4, 5, 6, 7, 8, or 9; and
pharmaceutically acceptable salts or tautomers thereof.

9. The USP2 inhibitor of claim 7, wherein the compound is a compound of Formulae (I-A-12), (I-A-13), (I-A-14), (I-A-15), (I-A-16) or (I-A-17): 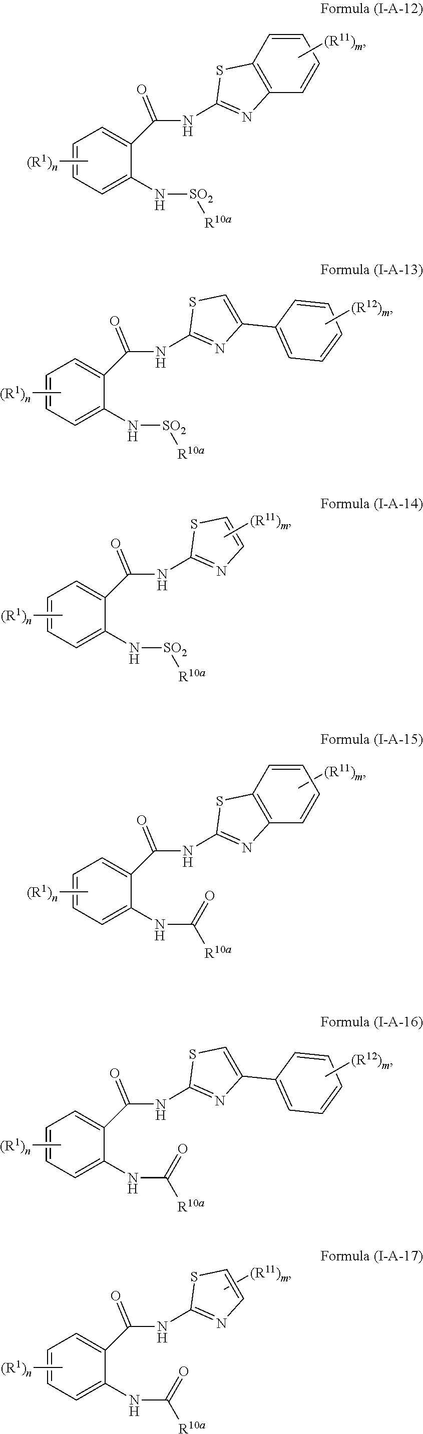
wherein
R^{10a} is selected from H, C₁-C₆ alkyl, C₂-C₆ alkenyl, C₃-C₁₂ cycloalkyl, or 3-12 membered heterocyclic, where each said C₁-C₆ alkyl, C₂-C₆ alkenyl, C₃-C₁₂ cycloalkyl, or 3-12 membered heterocyclic is optionally substituted with halogen, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl.
each R¹¹ is independently selected from H, halogen, cyano, OR⁶, NR⁶R⁷, C(O)OR⁸, C(O)NR⁸R⁹, S(O)₂NR⁸R⁹, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
each R¹² is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹², together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R¹ is selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R⁶ and R⁷ are each independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
R⁸ and R⁹ are each independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-C₁₀ aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form 4-8 membered heterocyclic; and
n is 0, 1, 2, 3, or 4;
m is 0, 1, 2, 3, 4, 5, 6, 7, 8, or 9; and
pharmaceutically acceptable salts or tautomers thereof.

10. A USP2 inhibitor, comprising a compound of Formulae (I-B1), (I-B2) or (I-B3): 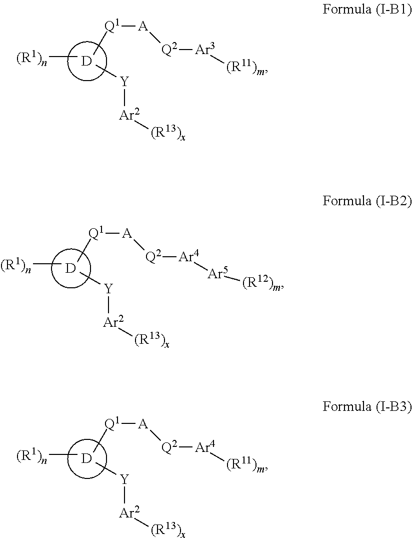
wherein
Ar² is selected from C₆-C₁₀ aryl, or 5-10 membered heteroaryl;
Ar³ is selected from 7-10 membered bicyclic heteroaryl;
Ar⁴ is selected from 5-6 membered monocyclic heteroaryl;
Ar⁵ is selected from C₆-C₁₀ aryl, or 5-10 membered heteroaryl;
each R¹³ is independently selected from H, halogen, cyano, OR⁶, N⁶R⁷, C(O)OR⁶, C(O)NR⁶R⁷, S(O)₂N⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹³, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
A is C═O, CH₂ or absent;
Ring D is a ring structure selected from C₅-C₁₀ aryl, 5-10 membered heteroaryl or 3-12 membered heterocyclic;
Y is a bond, or a bivalent moiety selected from —N(R⁴)—, —N(R⁴)—C₁-C₃ alkylene-, —N(R⁴)—S(O)₂—, —N(R⁴)—C(O)—, —S(O)₂— N(R⁴)—, or —C(O)—N(R⁴)—;
Q¹ and Q² are independently selected from absent, O, NR⁵, C₁-C₃ alkylene, C₂-C₆ alkenylene, or C₂-C₆ alkynylene, where each said C₁-C₃ alkylene, C₂-C₆ alkenylene, or C₂-C₆ alkynylene is optionally substituted with one or more of halogen, cyano, hydroxyl, oxo, C₁-C₆ alkyl, C₃-C₆ cycloalkyl, C₁-C₆ alkoxyl, or C₃-C₆ cycloalkoxy;
R¹ is selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R⁶ and R⁷ are each independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
R⁸ and R⁹ are each independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-C₁₀ aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form 4-8 membered heterocyclic;
each R¹¹ is independently selected from H, halogen, cyano, OR⁶, NR⁶R⁷, C(O)OR⁸, C(O)NR⁸R⁹, S(O)₂NR⁸R⁹, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
each R¹² is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹², together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
n is 0, 1, 2, 3, or 4;
m is 0, 1, 2, 3, 4, 5, 6, 7, 8, or 9; and
x is 0, 1, 2, 3, 4, 5, 6, 7, 8, or 9; and
pharmaceutically acceptable salts or tautomers thereof.

11. The USP2 inhibitor of claim 10, wherein the compound is a compound of Formulae (I-B1), (I-B2) or (I-B3-1): 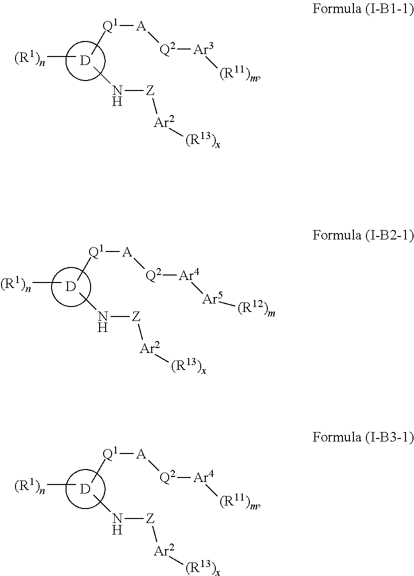
wherein
Ar² is selected from C₆-C₁₀ aryl, or 5-10 membered heteroaryl;
Ar³ is selected from 7-10 membered bicyclic heteroaryl;
Ar⁴ is selected from 5-6 membered monocyclic heteroaryl;
Ar⁵ is selected from C₆-C₁₀ aryl, or 5-10 membered heteroaryl;
Ring D is a ring structure selected from C₅-C₁₀ aryl, 5-10 membered heteroaryl or 3-12 membered heterocyclic;
each R¹³ is independently selected from H, halogen, cyano, OR⁶, N⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹³, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
A is C═O, CH₂ or absent;
Z is C═O or SO₂;
Q¹ and Q² are independently selected from absent, O, NR⁵, C₁-C₃ alkylene, C₂-C₆ alkenylene, or C₂-C₆ alkynylene, where each said C₁-C₃ alkylene, C₂-C₆ alkenylene, or C₂-C₆ alkynylene is optionally substituted with one or more of halogen, cyano, hydroxyl, oxo, C₁-C₆ alkyl, C₃-C₆ cycloalkyl, C₁-C₆ alkoxyl, or C₃-C₆ cycloalkoxy;
R¹ is selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R⁶ and R⁷ are each independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
R⁸ and R⁹ are each independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-C₁₀ aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form 4-8 membered heterocyclic;
each R¹¹ is independently selected from H, halogen, cyano, OR⁶, NR⁶R⁷, C(O)OR⁸, C(O)NR⁸R⁹, S(O)₂NR⁸R⁹, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
each R¹² is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹², together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
n is 0, 1, 2, 3, or 4;
m is 0, 1, 2, 3, 4, 5, 6, 7, 8, or 9; and
x is 0, 1, 2, 3, 4, 5, 6, 7, 8, or 9; and
pharmaceutically acceptable salts or tautomers thereof.

12. The USP2 inhibitor of claim 11, wherein the compound is a compound of Formulae (I-B1-2), (I-B2-2) or (I-B3-2): 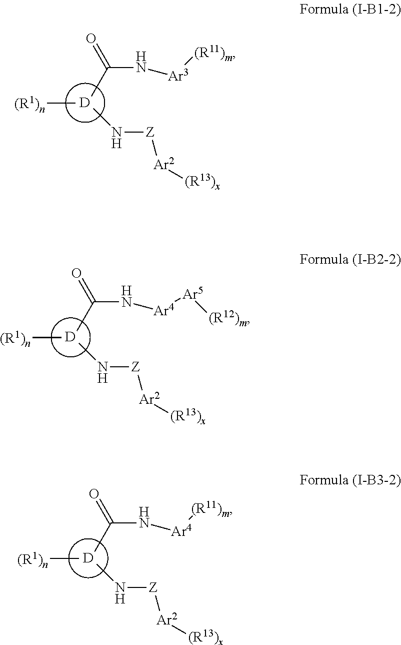
wherein
Ar² is selected from C₆-C₁₀ aryl, or 5-10 membered heteroaryl;
Ar³ is selected from 7-10 membered bicyclic heteroaryl;
Ar⁴ is selected from 5-6 membered monocyclic heteroaryl;
Ar⁵ is selected from C₆-C₁₀ aryl, or 5-10 membered heteroaryl;
Ring D is a ring structure selected from C₅-C₁₀ aryl, 5-10 membered heteroaryl or 3-12 membered heterocyclic;
each R¹³ is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹³, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
Z is C═O or SO₂;
R¹ is selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R⁶ and R⁷ are each independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
R⁸ and R⁹ are each independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-C₁₀ aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form 4-8 membered heterocyclic;
each R¹¹ is independently selected from H, halogen, cyano, OR⁶, NR⁶R⁷, C(O)OR⁸, C(O)NR⁸R⁹, S(O)₂NR⁸R⁹, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
each R¹² is independently selected from H, halogen, cyano, OR⁶, N⁶R⁷, C(O)OR⁶, C(O)NR⁶R⁷, S(O)₂N⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹², together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
n is 0, 1, 2, 3, or 4;
m is 0, 1, 2, 3, 4, 5, 6, 7, 8, or 9; and
x is 0, 1, 2, 3, 4, 5, 6, 7, 8, or 9; and
pharmaceutically acceptable salts or tautomers thereof.

13. The USP2 inhibitor of claim 12, wherein the compound is a compound of Formulae (I-B1-3), (I-B2-3) or (I-B3-3): 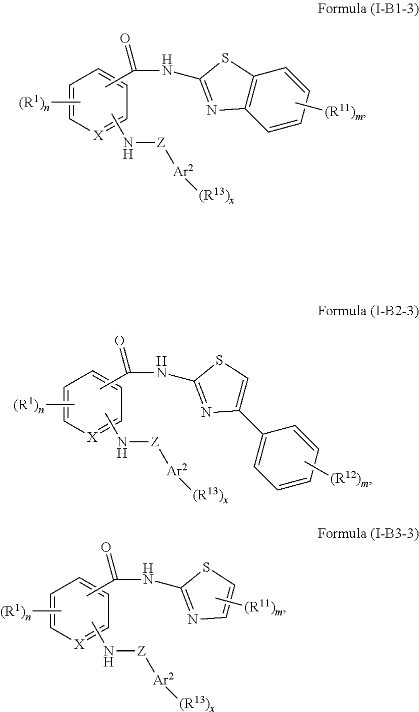
wherein
Ar² is selected from C₆-C₁₀ aryl, or 5-10 membered heteroaryl;
each R¹³ is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹³, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
X is CH or N;
Z is C═O or SO₂;
R¹ is selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R⁶ and R⁷ are each independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
R⁸ and R⁹ are each independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-C₁₀ aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form 4-8 membered heterocyclic;
each R¹¹ is independently selected from H, halogen, cyano, OR⁶, NR⁶R⁷, C(O)OR⁸, C(O)NR⁸R⁹, S(O)₂NR⁸R⁹, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
each R¹² is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹², together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
n is 0, 1, 2, 3, or 4;
m is 0, 1, 2, 3, 4, 5, 6, 7, 8, or 9; and
x is 0, 1, 2, 3, 4, 5, 6, 7, 8, or 9; and
pharmaceutically acceptable salts or tautomers thereof.

14. The USP2 inhibitor of claim 13, wherein the compound is a compound of Formulae (I-B1-4), (I-B2-4) or (I-B3-4): 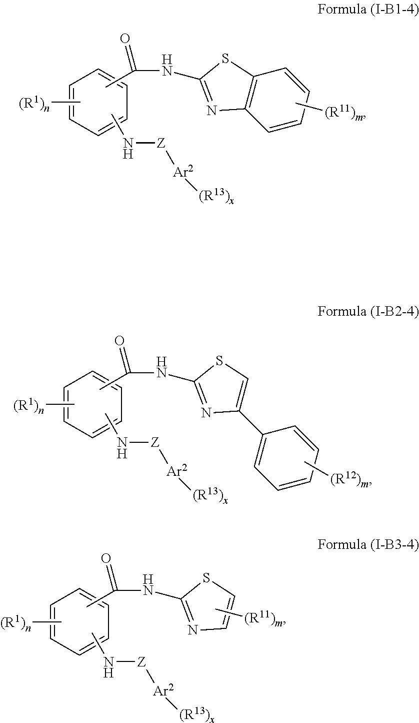
wherein
Ar² is selected from C₆-C₁₀ aryl, or 5-10 membered heteroaryl;
Z is C═O or SO₂;
each R¹³ is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹³, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R¹ is selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R⁶ and R⁷ are each independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
R⁸ and R⁹ are each independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-C₁₀ aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form 4-8 membered heterocyclic;
each R¹¹ is independently selected from H, halogen, cyano, OR⁶, NR⁶R⁷, C(O)OR⁸, C(O)NR⁸R⁹, S(O)₂NR⁸R⁹, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
each R¹² is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹², together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
n is 0, 1, 2, 3, or 4;
m is 0, 1, 2, 3, 4, 5, 6, 7, 8, or 9; and
x is 0, 1, 2, 3, 4, 5, 6, 7, 8, or 9; and
pharmaceutically acceptable salts or tautomers thereof.

15. The USP2 inhibitor of claim 14, wherein the compound is a compound of Formulae (I-B1-5), (I-B2-5) or (I-B3-5): 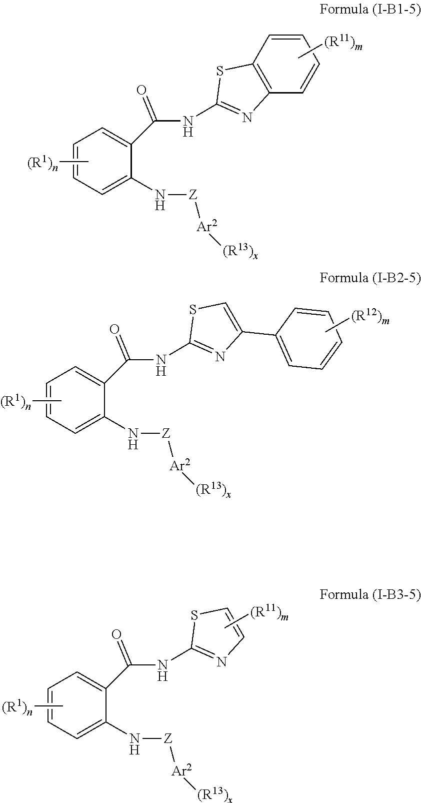
wherein
Ar² is selected from C₆-C₁₀ aryl, or 5-10 membered heteroaryl;
Z is C═O or SO₂;
each R¹³ is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹³, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R¹ is selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R⁶ and R⁷ are each independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
R⁸ and R⁹ are each independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-C₁₀ aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form 4-8 membered heterocyclic;
each R¹¹ is independently selected from H, halogen, cyano, OR⁶, NR⁶R⁷, C(O)OR⁸, C(O)NR⁸R⁹, S(O)₂NR⁸R⁹, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
each R¹² is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹², together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
n is 0, 1, 2, 3, or 4;
m is 0, 1, 2, 3, 4, 5, 6, 7, 8, or 9; and
x is 0, 1, 2, 3, 4, 5, 6, 7, 8, or 9; and
pharmaceutically acceptable salts or tautomers thereof.

16. The USP2 inhibitor of claim 15, wherein the compound is a compound of Formulae (I-B2-6) or (I-B2-7): 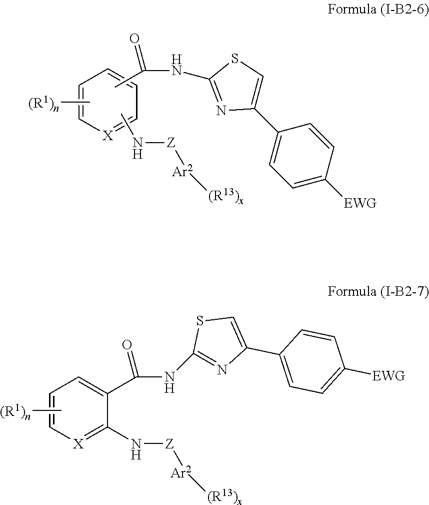
wherein
EWG is an electron withdrawing group;
X is CH or N;
Z is C═O or SO₂;
Ar² is selected from C₆-C₁₀ aryl, or 5-10 membered heteroaryl;
each R¹³ is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹³, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R¹ is selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R⁶ and R⁷ are each independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
R⁸ and R⁹ are each independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-C₁₀ aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form 4-8 membered heterocyclic;
n is 0, 1, 2, 3, or 4;
m is 0, 1, 2, 3, 4, 5, 6, 7, 8, or 9; and
x is 0, 1, 2, 3, 4, 5, 6, 7, 8, or 9; and
pharmaceutically acceptable salts or tautomers thereof.

17. A compound according to claim 16, wherein the EWG is selected from the group consisting of halogen, cyano, C(O)OR⁶, C(O)NR⁶R⁷, S(O)₂NR⁶R⁷, and C₁-C₆ haloalkyl.

18. The USP2 inhibitor of claim 16, wherein the compound is a compound of Formulae (I-B2-8), (I-B2-9), (I-B2-10), (I-B2-11), (I-B2-12), (I-B2-13), (I-B2-14), (I-B2-15), (I-B2-16) or (I-B2-17): 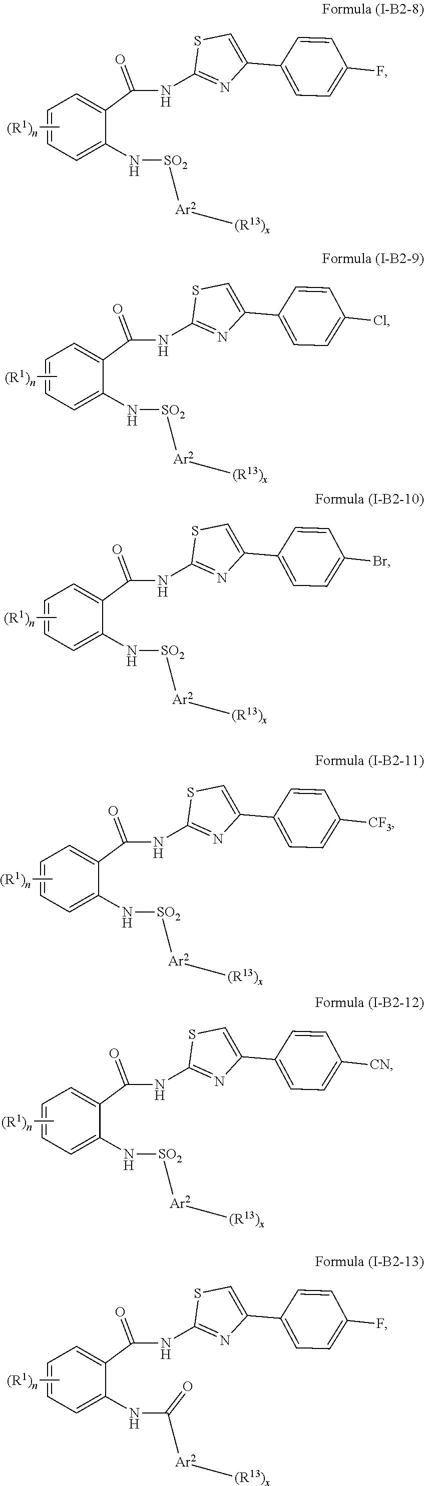 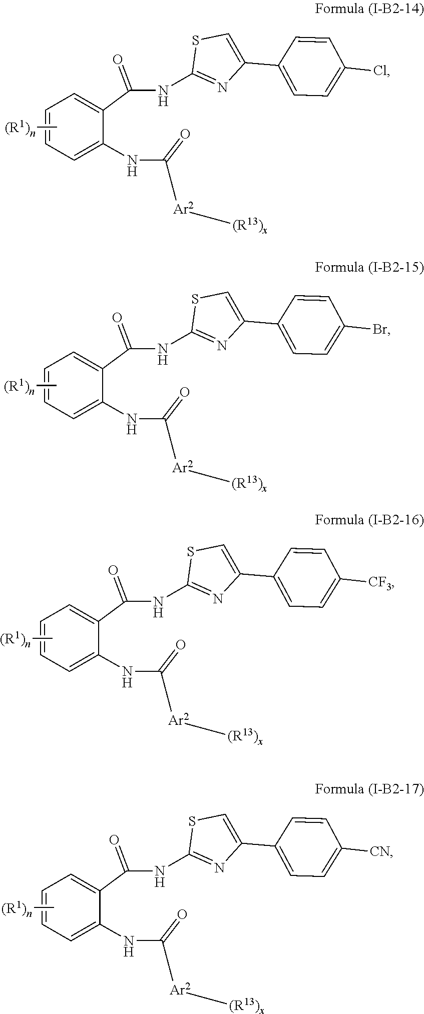
wherein
Ar² is selected from C₆-C₁₀ aryl, or 5-10 membered heteroaryl;
each R¹³ is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹³, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R¹ is selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R⁶ and R⁷ are each independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
R⁸ and R⁹ are each independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-C₁₀ aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form 4-8 membered heterocyclic;
n is 0, 1, 2, 3, or 4;
m is 0, 1, 2, 3, 4, 5, 6, 7, 8, or 9; and
x is 0, 1, 2, 3, 4, 5, 6, 7, 8, or 9; and
pharmaceutically acceptable salts or tautomers thereof.

19. A USP2 inhibitor, comprising a compound of Formulae (I-C1), (I-C2) or (I-C3). 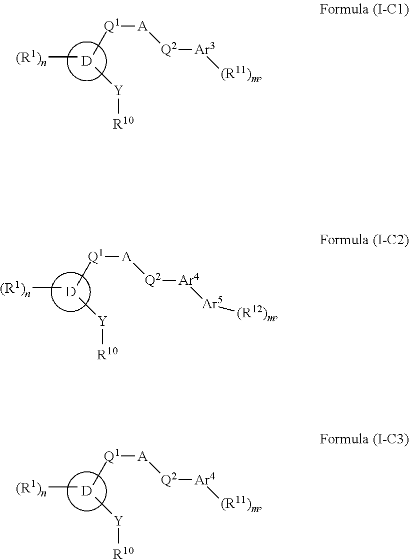
wherein
A is C═O, CH₂ or absent;
Y is a bond, or a bivalent moiety selected from —N(R⁴)—, —N(R⁴)—C₁-C₃ alkylene-, —N(R⁴)—S(O)₂—, —N(R⁴)—C(O)—, —S(O)₂— N(R⁴)—, or —C(O)—N(R⁴)—;
Ring D is a ring structure selected from C₅-C₁₀ aryl, 5-10 membered heteroaryl or 3-12 membered heterocyclic;
Ar² is selected from C₆-C₁₀ aryl, or 5-10 membered heteroaryl;
Ar³ is selected from 7-10 membered bicyclic heteroaryl;
Ar⁴ is selected from 5-6 membered monocyclic heteroaryl;
Ar⁵ is selected from C₆-C₁₀ aryl, or 5-10 membered heteroaryl;
Q¹ and Q² are independently selected from absent, O, NR⁵, C₁-C₃ alkylene, C₂-C₆ alkenylene, or C₂-C₆ alkynylene, where each said C₁-C₃ alkylene, C₂-C₆ alkenylene, or C₂-C₆ alkynylene is optionally substituted with one or more of halogen, cyano, hydroxyl, oxo, C₁-C₆ alkyl, C₃-C₆ cycloalkyl, C₁-C₆ alkoxyl, or C₃-C₆ cycloalkoxy;
R¹ is independently selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹ together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
each R⁴, each R⁵, each R⁶ and each R⁷ are independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
each R⁸ and each R⁹ are independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-Cia aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form a 4-8 membered heterocyclic;
R¹⁰ is selected from H, OR⁶, NR⁶R⁷, C₁-C₆ alkyl, C₁-C₆ heteroalkyl, C₂-C₆ alkenyl, C₂-C₆ alkynyl, C₃-C₁₂ cycloalkyl, or 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, or 3-12 membered heterocyclic is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl;
each R¹¹ is independently selected from H, halogen, cyano, OR⁶, NR⁶R⁷, C(O)OR⁸, C(O)NR⁸R⁹, S(O)₂NR⁸R⁹, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
and
n is 0, 1, 2, 3, or 4;
m is 0, 1, 2, 3, 4, 5, 6, 7, 8, or 9; and
pharmaceutically acceptable salts or tautomers thereof.

20. A USP2 inhibitor, comprising a compound of Formulae (I-C1-1), (I-C₂-1), (I-C3-1), (I-C1-2), (I-C2-2) or (I-C3-2): 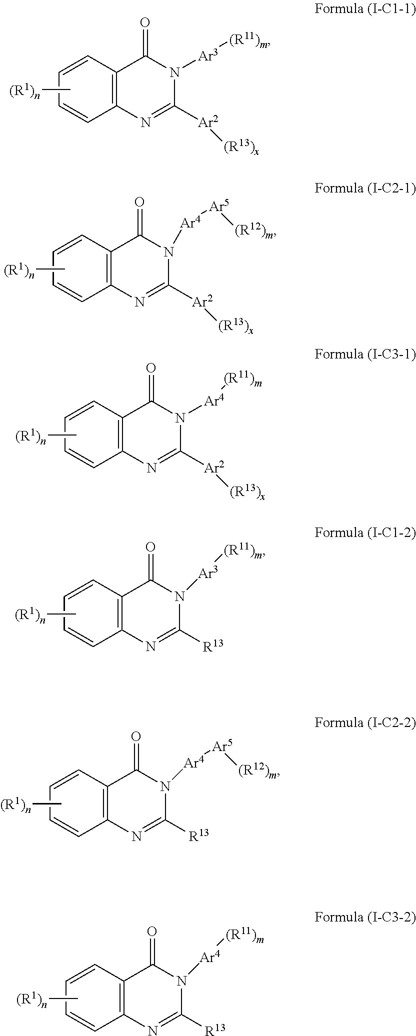
wherein
Ar² is selected from C₆-C₁₀ aryl, or 5-10 membered heteroaryl;
Ar³ is selected from 7-10 membered bicyclic heteroaryl;
Ar⁴ is selected from 5-6 membered monocyclic heteroaryl;
Ar⁵ is selected from C₆-C₁₀ aryl, or 5-10 membered heteroaryl;
R¹ is independently selected from H, halogen, cyano, oxo, OR⁶, N⁶R⁷, C(O)OR⁶, C(O)NR⁶R⁷, S(O)₂N⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹ together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
each R¹¹ is independently selected from H, halogen, cyano, OR⁶, NR⁶R⁷, C(O)OR⁸, C(O)NR⁸R⁹, S(O)₂NR⁸R⁹, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
each R¹² is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹², together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R⁶ and R⁷ are each independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
R⁸ and R⁹ are each independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-C₁₀ aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form 4-8 membered heterocyclic;
each R¹³ is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹³, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
n is 0, 1, 2, 3, or 4;
m is 0, 1, 2, 3, 4, 5, 6, 7, 8, or 9;
x is 0, 1, 2, 3, 4, 5, 6, 7, 8, or 9; and
pharmaceutically acceptable salts or tautomers thereof.

21. The USP2 inhibitor of claim 20, wherein the compound is a compound of Formulae (I-C2-3), or (I-C2-4): 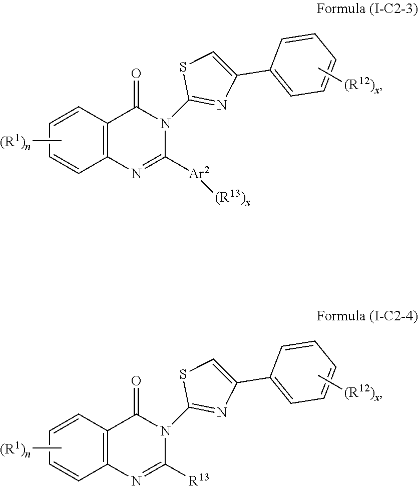
wherein,
Ar² is selected from C₆-C₁₀ aryl, or 5-10 membered heteroaryl;
R¹ is independently selected from H, halogen, cyano, oxo, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹ together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
each R¹² is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹², together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
R⁶ and R⁷ are each independently selected from H, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C₁-C₆ alkyl, C₁-C₆ heteroalkyl, C₃-C₁₂ cycloalkyl, or 3-12 membered heterocyclic; or R⁶ and R⁷, together with the atoms to which they are attached, optionally form 4-8 membered heterocyclic;
R⁸ and R⁹ are each independently selected from H, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₃-C₁₂ cycloalkyl, 3-12 membered heterocyclic, C₆-C₁₀ aryl, or 5-10 membered heteroaryl is optionally substituted with halogen, cyano, oxo, C₁-C₆ alkyl, C₁-C₆ heteroalkyl, C₃-C₁₂ cycloalkyl, or 3-12 membered heterocyclic; or R⁸ and R⁹, together with the atoms to which they are attached, optionally form 4-8 membered heterocyclic;
each R¹³ is independently selected from H, halogen, cyano, OR⁶, NR⁶R⁷, C(O)OR⁶, C(O)NR⁶R⁷, S(O)₂NR⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹³, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
n is 0, 1, 2, 3, or 4;
x is 0, 1, 2, 3, 4, 5, 6, 7, 8, or 9; and
pharmaceutically acceptable salts or tautomers thereof.

22. The USP2 inhibitor of claim 21, wherein the compound is a compound of Formulae (I-C2-5) or (I-C2-6): 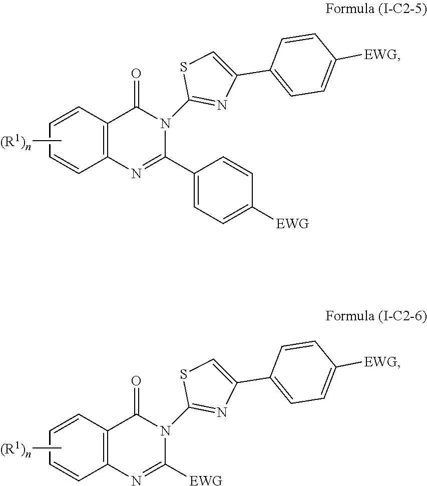
wherein
EWG is an electron withdrawing group;
R¹ is selected from H, halogen, cyano, oxo, OR⁶, NR⁶R⁷, C(O)OR⁶, C(O)NR⁶R⁷, S(O)₂N⁶R⁷, C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl, where each said C₁-C₆ alkyl, C₁-C₆ heteroalkyl, C₂-C₆ alkenyl, C₂-C₆ alkynyl, C₃-C₁₂ cycloalkyl, 3-12 membered heterocyclic, C₆-C₁₀ aryl, or 5-10 membered heteroaryl is optionally substituted with halogen, cyano, oxo, OR⁸, NR⁸R⁹, C(O)OR⁸, C(O)NR⁸R⁹, C₁-C₆ alkyl, C₁-C₆ heteroalkyl, C₃-C₁₂ cycloalkyl, 3-12 membered heterocyclic, C₆-C₁₀ aryl, or 5-10 membered heteroaryl; or two adjacent R¹, together with the atoms to which they are attached, optionally form C₄-C₆ cycloalkyl or 5-7 membered heterocyclic, where each said C₃-C₆ cycloalkyl or 5-7 membered heterocyclic is optionally substituted with C₁-C₆ alkyl;
n is 0, 1, 2, 3, or 4; and
pharmaceutically acceptable salts or tautomers thereof.

23. (canceled)

24. A USP2 inhibitor, comprising a compound selected from the group consisting of:

| | | |
|---|---|---|
| XH153-182 | 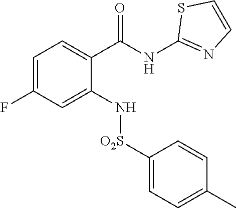 [SMILES: Cc1ccc(S(=O)(=O)Nc2cc(F)ccc2C(=O)Nc2nccs2)cc1] | 4-fluoro-2-((4- methylphenyl) sulfonamido)- N-(thiazol-2- yl)benzamide |
| XH161-172 (MS1172) | 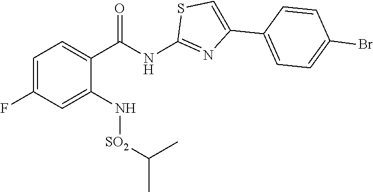 [SMILES: CC(C)S(=O)(=O)Nc1cc(F)ccc1C(=O)Nc1nc(-c2ccc(Br)cc2)cs1] | N-(4-(4- bromophenyl)thiazol-2- yl)-4-fluoro-2-((1- methylethyl) sulfonamido)benzamide |
| XH161-180 (MS1180) | 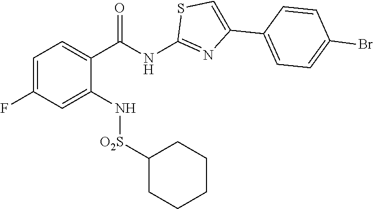 [SMILES: O=C(Nc1nc(-c2ccc(Br)cc2)cs1)c1ccc(F)cc1NS(=O)(=O)C1CCCCC1] | N-(4-(4- bromophenyl)thiazol-2- yl)-2- (cyclohexane- sulfonamido)- 4-fluorobenzamide |
| XH208-93P2 | 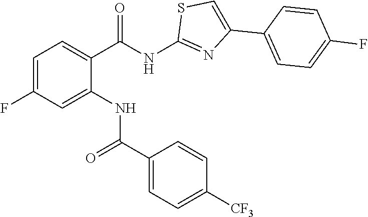 [SMILES: O=C(Nc1cc(F)ccc1C(=O)Nc1nc(-c2ccc(F)cc2)cs1)c1ccc(C(F)(F)F)cc1] | 4-Fluoro-N-(4-(4- fluorophenyl)thiazol-2- yl)-2-(4- (trifluoromethyl) benzamido) benzamide |
| XH208-94P2 | 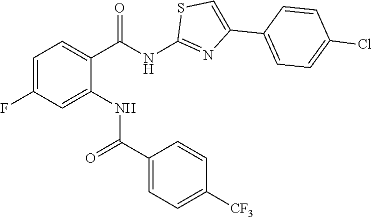 [SMILES: O=C(Nc1cc(F)ccc1C(=O)Nc1nc(-c2ccc(Cl)cc2)cs1)c1ccc(C(F)(F)F)cc1] | N-(4-(4- Chlorophenyl)thiazol-2- yl)-4-fluoro-2-(4- (trifluoromethyl) benzamido) benzamide |
| XH198-68P2 | 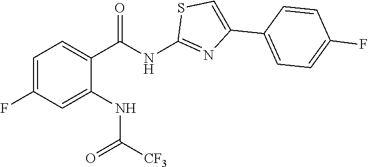 [SMILES: O=C(Nc1nc(-c2ccc(F)cc2)cs1)c1ccc(F)cc1NC(=O)C(F)(F)F] | 4-Fluoro-N-(4-(4- fluorophenyl)thiazol-2- yl)-2-(2,2,2- trifluoroacetamido) benzamide |
| XH188-135p1 | 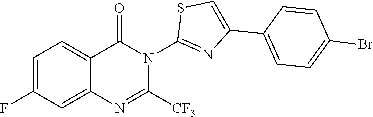 [SMILES: O=c1c2ccc(F)cc2nc(C(F)(F)F)n1-c1nc(-c2ccc(Br)cc2)cs1] | 3-(4-(4- Bromophenyl)thiazol-2- yl)-7-fluoro-2- (trifluoromethyl) quinazolin-4(3H)-one |
| XH188-135p2 | 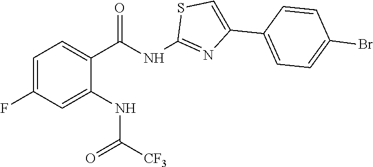 [SMILES: O=C(Nc1nc(-c2ccc(Br)cc2)cs1)c1ccc(F)cc1NC(=O)C(F)(F)F] | N-(4-(4- Bromophenyl)thiazol-2- yl)-4-fluoro-2-(2,2,2- trifluoroacetamido) benzamide |
| XH161-102 (MS102) | 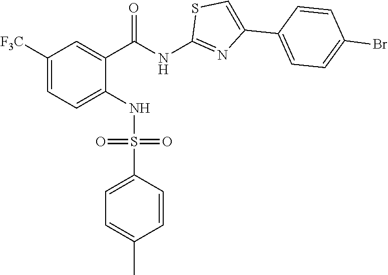 [SMILES: Cc1ccc(S(=O)(=O)Nc2ccc(C(F)(F)F)cc2C(=O)Nc2nc(-c3ccc(Br)cc3)cs2)cc1] | N-(4-(4- bromophenyl)thiazol- 2-yl)-2-((4- methylphenyl) sulfonamido)-5- (trifluoromethyl) benzamide |

and pharmaceutically acceptable salts or tautomers thereof.

25. A pharmaceutical composition, comprising:
a USP2 inhibitor according to claim 1, and a pharmaceutically acceptable carrier.

26. (canceled)

27. A pharmaceutical composition according to claim 25, wherein the composition is formulated for administration by oral, parenteral or transdermal delivery.

28. A method of treating a USP2-mediated disease, comprising administering to a subject in need thereof an USP2 inhibitor according to claim 1.

29. A method according to claim 28, wherein the disease is selected from mesothelioma, hepatocellular cancer, central nervous system neoplasm, lung cancer, bone cancer, pancreatic cancer, skin cancer, head and neck cancer, melanoma, ovarian cancer, colon cancer, rectal cancer, anal cancer, stomach cancer, gastrointestinal cancer, breast cancer), uterine cancer, fallopian tube cancer, endometrial cancer, cervical cancer, vaginal cancer, vulvar cancer, esophageal cancer, gastrointestinal cancer, endocrine cancer, thyroid cancer, parathyroid cancer, adrenal cancer, soft tissue sarcoma, urethral cancer, penile cancer, prostate cancer, testicular cancer, leukemia, lymphoma, bladder cancer, renal cell cancer, brain stem glioma, pituitary cancer, adrenocortical cancer, gallbladder cancer, multiple myeloma, cholangiocarcinoma, fibrosarcoma, neuroblastoma, and retinoblastoma.

30. A method of treating a USP2-mediated virus infection including hCoV-NL63, SARS-CoV-1 and SARS-CoV-2, comprising administering to a subject in need thereof an USP2 inhibitor according to claim 1.

31. A method according to claim 28, wherein the inhibitor is administered orally, parenterally, intradermally, subcutaneously, topically, and/or rectally.

32. A method according to claim 31, further comprising an additional therapeutic regimen selected from of surgery, chemotherapy, radiation therapy, hormone therapy, or immunotherapy.
